SEQUENCE LISTING (325 sequences)

SEQ ID NO: 1 - protein, 102 aa, synthetic construct
  source 1..102: note = KERE sequence no. 1
  REGION 31..35: note = CDR1
  REGION 50..54: note = CDR2
  REGION 87..91: note = CDR3
EVQLVESGGGLVQPGGSLRLSCAASGIPFSXXXXXWFRQAPGKQRDSVAXXXXXRFTISRDNAKNTVYLQMNSLKPEDTAVYRCYFXXXXXWGQGTQVTVSS

SEQ ID NO: 2 - protein, 102 aa, synthetic construct
  source 1..102: note = KERE sequence no. 2
  REGION 31..35: note = CDR1
  REGION 50..54: note = CDR2
  REGION 87..91: note = CDR3
QVKLEESGGGLVQAGGSLRLSCVGSGRTFSXXXXXWFRLAPGKEREFVAXXXXXRFTISRDTASNRGYLHMNNLTPEDTAVYYCAAXXXXXWGQGTQVTVSS

SEQ ID NO: 3 - protein, 102 aa, synthetic construct
  source 1..102: note = KERE sequence no. 3
  REGION 31..35: note = CDR1
  REGION 50..54: note = CDR2
  REGION 87..91: note = CDR3
AVQLVDSGGGLVQAGDSLKLSCALTGGAFTXXXXXWFRQTPGREREFVAXXXXXRFTISRDNAKNMVYLRMNSLIPEDAAVYSCAAXXXXXWGQGTLVTVSS

SEQ ID NO: 4 - protein, 102 aa, synthetic construct
  source 1..102: note = KERE sequence no. 4
  REGION 31..35: note = CDR1
  REGION 50..54: note = CDR2
  REGION 87..91: note = CDR3
QVQLVESGGGLVEAGGSLRLSCTASESPFRXXXXXWFRQTSGQEREFVAXXXXXRFTISRDDAKNTVWLHGSTLKPEDTAVYYCAAXXXXXWGQGTQVTVSS

SEQ ID NO: 5 - protein, 102 aa, synthetic construct
  source 1..102: note = KERE sequence no. 5
  REGION 31..35: note = CDR1
  REGION 50..54: note = CDR2
  REGION 87..91: note = CDR3
AVQLVESGGGLVQGGGSLRLACAASERIFDXXXXXWYRQGPGNERELVAXXXXXRFTISMDYTKQTVYLHMNSLRPEDTGLYYCKIXXXXXWGQGTQVTVSS

SEQ ID NO: 6 - protein, 102 aa, synthetic construct
  source 1..102: note = KERE sequence no. 6
  REGION 31..35: note = CDR1
  REGION 50..54: note = CDR2
  REGION 87..91: note = CDR3
DVKFVESGGGLVQAGGSLRLSCVASGFNFDXXXXXWFRQAPGKEREEVAXXXXXRFTISSEKDKNSVYLQMNSLKPEDTALYICAGXXXXXWGRGTQVTVSS

SEQ ID NO: 7 - protein, 102 aa, synthetic construct
  source 1..102: note = KERE sequence no. 7
  REGION 31..35: note = CDR1
  REGION 50..54: note = CDR2
  REGION 87..91: note = CDR3
QVRLAESGGGLVQSGGSLRLSCVASGSTYTXXXXXWYRQYPGKQRALVAXXXXXRFTIARDSTKDTFCLQMNNLKPEDTAVYYCYAXXXXXWGQGTQVTVSS

SEQ ID NO: 8 - protein, 102 aa, synthetic construct
  source 1..102: note = KERE sequence no. 8
  REGION 31..35: note = CDR1
  REGION 50..54: note = CDR2
  REGION 87..91: note = CDR3
EVQLVESGGGLVQAGGSLRLSCAASGFTSDXXXXXWFRQAPGKPREGVSXXXXXRFTISTDNAKNTVHLLMNRVNAEDTALYYCAVXXXXXWGRGTRVTVSS

SEQ ID NO: 9 - protein, 102 aa, synthetic construct
  source 1..102: note = KERE sequence no. 9
  REGION 31..35: note = CDR1
  REGION 50..54: note = CDR2
  REGION 87..91: note = CDR3
QVQLVESGGGLVQPGGSLRLSCQASGDISTXXXXXWYRQVPGKLREFVAXXXXXRFTISGDNAKRAIYLQMNNLKPDDTAVYYCNRXXXXXWGQGTQVTVSP

SEQ ID NO: 10 - protein, 102 aa, synthetic construct
  source 1..102: note = KERE sequence no. 10
  REGION 31..35: note = CDR1
  REGION 50..54: note = CDR2
  REGION 87..91: note = CDR3
QVPVVESGGGLVQAGDSLRLFCAVPSFTSTXXXXXWFRQAPGKEREFVAXXXXXRFTISRNATKNTLTLRMDSLKPEDTAVYYCAAXXXXXWGQGTQVTVSS

SEQ ID NO: 11 - protein, 102 aa, synthetic construct
  source 1..102: note = KERE sequence no. 11
  REGION 31..35: note = CDR1
  REGION 50..54: note = CDR2
  REGION 87..91: note = CDR3
EVQLVESGGGLVQAGDSLRLFCTVSGGTASXXXXXWFRQAPGEKREFVAXXXXXRFTIARENAGNMVYLQMNNLKPDDTALYTCAAXXXXXWGRGTQVTVSS

SEQ ID NO: 12 - protein, 102 aa, synthetic construct
  source 1..102: note = KERE sequence no. 12
  REGION 31..35: note = CDR1
  REGION 50..54: note = CDR2
  REGION 87..91: note = CDR3
AVQLVESGGDSVQPGDSQTLSCAASGRTNSXXXXXWFRQAPGKERVFLAXXXXXRFTISRDSAKNMMYLQMNNLKPQDTAVYYCAAXXXXXWGQGTQVTVSS

SEQ ID NO: 13 - protein, 102 aa, synthetic construct
  source 1..102: note = KERE sequence no. 13
  REGION 31..35: note = CDR1
  REGION 50..54: note = CDR2
  REGION 87..91: note = CDR3
AVQLVESGGGLVQAGGSLRLSCVVSGLTSSXXXXXWFRQTPWQERDFVAXXXXXRFTISRDNYKDTVLLEMNFLKPEDTAIYYCAAXXXXXWGQGTQVTVSS

SEQ ID NO: 14 - protein, 102 aa, synthetic construct
  source 1..102: note = KERE sequence no. 14
  REGION 31..35: note = CDR1
  REGION 50..54: note = CDR2
  REGION 87..91: note = CDR3
AVQLVESGGGLVQAGASLRLSCATSTRTLDXXXXXWFRQAPGRDREFVAXXXXXRFTVSRDSAENTVALQMNSLKPEDTAVYYCAAXXXXXWGQGTRVTVSS

SEQ ID NO: 15 - protein, 102 aa, synthetic construct
  source 1..102: note = KERE sequence no. 15
  REGION 31..35: note = CDR1
  REGION 50..54: note = CDR2
  REGION 87..91: note = CDR3
QVQLVESGGGLVQPGGSLRLSCTVSRLTAHXXXXXWFRQAPGKEREAVSXXXXXRFTISRDYAGNTAFLQMDSLKPEDTGVYYCATXXXXXWGQGTQVTVSS

SEQ ID NO: 16 - protein, 102 aa, synthetic construct
  source 1..102: note = KERE sequence no. 16
  REGION 31..35: note = CDR1
  REGION 50..54: note = CDR2
  REGION 87..91: note = CDR3
EVQLVESGGELVQAGGSLKLSCTASGRNFVXXXXXWFRRAPGKEREFVAXXXXXRFTVSRDNGKNTAYLRMNSLKPEDTADYYCAVXXXXXLGSGTQVTVSS

SEQ ID NO: 17 - protein, 102 aa, synthetic construct
  source 1..102: note = GLEW sequence no. 1
  REGION 31..35: note = CDR1
  REGION 50..54: note = CDR2
  REGION 87..91: note = CDR3
AVQLVESGGGLVQPGGSLRLSCAASGFTFSXXXXXWVRQAPGKVLEWVSXXXXXRFTISRDNAKNTLYLQMNSLKPEDTAVYYCVKXXXXXGSQGTQVTVSS

SEQ ID NO: 18 - protein, 102 aa, synthetic construct
  source 1..102: note = GLEW sequence no. 2
  REGION 31..35: note = CDR1
  REGION 50..54: note = CDR2
  REGION 87..91: note = CDR3
EVQLVESGGGLVQPGGSLRLSCVCVSSGCTXXXXXWVRQAPGKAEEWVSXXXXXRFKISRDNAKKTLYLQMNSLGPEDTAMYYCQRXXXXXRGQGTQVTVSS

SEQ ID NO: 19 - protein, 102 aa, synthetic construct
  source 1..102: note = GLEW sequence no. 3
  REGION 31..35: note = CDR1
  REGION 50..54: note = CDR2
  REGION 87..91: note = CDR3
EVQLVESGGGLALPGGSLTLSCVFSGSTFSXXXXXWVRHTPGKAEEWVSXXXXXRFTISRDNAKNTLYLEMNSLSPEDTAMYYCGRXXXXXRSKGIQVTVSS

SEQ ID NO: 20 - protein, 102 aa, synthetic construct
  source 1..102: note = P,R,S 103 sequence no. 1
  REGION 31..35: note = CDR1
  REGION 50..54: note = CDR2
  REGION 87..91: note = CDR3
AVQLVESGGGLVQAGGSLRLSCAASGRTFSXXXXXWFRQAPGKEREFVAXXXXXRFTISRDNAKNTVYLQMNSLKPEDTAVYYCAAXXXXXRGQGTQVTVSS

SEQ ID NO: 21 - protein, 102 aa, synthetic construct
  source 1..102: note = P,R,S 103 sequence no. 2
  REGION 31..35: note = CDR1
  REGION 50..54: note = CDR2
  REGION 87..91: note = CDR3
DVQLVESGGDLVQPGGSLRLSCAASGFSFDXXXXXWLRQTPGKGLEWVGXXXXXRFTISRDNAKNMLYLHLNNLKSEDTAVYYCRRXXXXXLGQGTQVTVSS

SEQ ID NO: 22 - protein, 102 aa, synthetic construct; identical to SEQ ID NO: 18
  source 1..102: note = P,R,S 103 sequence no. 3
  REGION 31..35: note = CDR1
  REGION 50..54: note = CDR2
  REGION 87..91: note = CDR3

SEQ ID NO: 23 - protein, 30 aa, synthetic construct
  source 1..30: note = KERE FW1 sequence no. 1
QVQRVESGGGLVQAGGSLRLSCAASGRTSS

SEQ ID NO: 24 - protein, 30 aa, synthetic construct
  source 1..30: note = KERE FW1 sequence no. 2
QVQLVESGGGLVQTGDSLSLSCSASGRTFS

SEQ ID NO: 25 - protein, 30 aa, synthetic construct
  source 1..30: note = KERE FW1 sequence no. 3
QVKLEESGGGLVQAGDSLRLSCAATGRAFG

SEQ ID NO: 26 - protein, 30 aa, synthetic construct
  source 1..30: note = KERE FW1 sequence no. 4
AVQLVESGGGLVQPGESLGLSCVASGRDFV

SEQ ID NO: 27 - protein, 30 aa, synthetic construct
  source 1..30: note = KERE FW1 sequence no. 5
EVQLVESGGGLVQAGGSLRLSCEVLGRTAG

SEQ ID NO: 28 - protein, 30 aa, synthetic construct
  source 1..30: note = KERE FW1 sequence no. 6
QVQLVESGGGWVQPGGSLRLSCAASETILS

SEQ ID NO: 29 - protein, 30 aa, synthetic construct
  source 1..30: note = KERE FW1 sequence no. 7
QVQLVESGGGTVQPGGSLNLSCVASGNTFN

SEQ ID NO: 30 - protein, 30 aa, synthetic construct
  source 1..30: note = KERE FW1 sequence no. 8
EVQLVESGGGLAQPGGSLQLSCSAPGFTLD

SEQ ID NO: 31 - protein, 30 aa, synthetic construct
  source 1..30: note = KERE FW1 sequence no. 9
AQELEESGGGLVQAGGSLRLSCAASGRTFN

SEQ ID NO: 32 - protein, 22 aa, synthetic construct
  source 1..22: note = KERE FW1 sequence no. 10
VESGGGLVQPGGSLRLSCAASG

SEQ ID NO: 33 - protein, 22 aa, synthetic construct
  source 1..22: note = KERE FW1 sequence no. 11
VDSGGGLVQAGDSLKLSCALTG

SEQ ID NO: 34 - protein, 22 aa, synthetic construct
  source 1..22: note = KERE FW1 sequence no. 12
VDSGGGLVQAGDSLRLSCAASG

SEQ ID NO: 35 - protein, 22 aa, synthetic construct
  source 1..22: note = KERE FW1 sequence no. 13
VDSGGGLVEAGGSLRLSCQVSE

SEQ ID NO: 36 - protein, 22 aa, synthetic construct
  source 1..22: note = KERE FW1 sequence no. 14
QDSGGGSVQAGGSLKLSCAASG

SEQ ID NO: 37 - protein, 22 aa, synthetic construct
  source 1..22: note = KERE FW1 sequence no. 15
VQSGGRLVQAGDSLRLSCAASE

SEQ ID NO: 38 - protein, 22 aa, synthetic construct
  source 1..22: note = KERE FW1 sequence no. 16
VESGGTLVQSGDSLKLSCASST

SEQ ID NO: 39 - protein, 22 aa, synthetic construct
  source 1..22: note = KERE FW1 sequence no. 17
MESGGDSVQSGGSLTLSCVASG

SEQ ID NO: 40 - protein, 22 aa, synthetic construct
  source 1..22: note = KERE FW1 sequence no. 18
QASGGGLVQAGGSLRLSCSASV

SEQ ID NO: 41 - protein, 14 aa, synthetic construct
  source 1..14: note = KERE FW2 sequence no. 1
WFRQAPGKEREFVA

SEQ ID NO: 42 - protein, 14 aa, synthetic construct
  source 1..14: note = KERE FW2 sequence no. 2
WFRQTPGREREFVA

SEQ ID NO: 43 - protein, 14 aa, synthetic construct
  source 1..14: note = KERE FW2 sequence no. 3
WYRQAPGKQREMVA

SEQ ID NO: 44 - protein, 14 aa, synthetic construct
  source 1..14: note = KERE FW2 sequence no. 4
WYRQGPGKQRELVA

SEQ ID NO: 45 - protein, 14 aa, synthetic construct
  source 1..14: note = KERE FW2 sequence no. 5
WIRQAPGKEREGVS

SEQ ID NO: 46 - protein, 14 aa, synthetic construct
  source 1..14: note = KERE FW2 sequence no. 6
WFREAPGKEREGIS

SEQ ID NO: 47 - protein, 14 aa, synthetic construct
  source 1..14: note = KERE FW2 sequence no. 7
WYRQAPGKERDLVA

SEQ ID NO: 48 - protein, 14 aa, synthetic construct
  source 1..14: note = KERE FW2 sequence no. 8
WFRQAPGKQREEVS

SEQ ID NO: 49 - protein, 14 aa, synthetic construct
  source 1..14: note = KERE FW2 sequence no. 9
WFRQPPGKVREFVG

SEQ ID NO: 50 - protein, 32 aa, synthetic construct
  source 1..32: note = KERE FW3 sequence no. 1
RFTISRDNAKNTVYLQMNSLKPEDTAVYRCYF

SEQ ID NO: 51 - protein, 32 aa, synthetic construct
  source 1..32: note = KERE FW3 sequence no. 2
RFAISRDNNKNTGYLQMNSLEPEDTAVYYCAA

SEQ ID NO: 52 - protein, 32 aa, synthetic construct
  source 1..32: note = KERE FW3 sequence no. 3
RFTVARNNAKNTVNLEMNSLKPEDTAVYYCAA

SEQ ID NO: 53 - protein, 32 aa, synthetic construct
  source 1..32: note = KERE FW3 sequence no. 4
RFTISRDIAKNTVDLLMNNLEPEDTAVYYCAA

SEQ ID NO: 54 - protein, 32 aa, synthetic construct
  source 1..32: note = KERE FW3 sequence no. 5
RLTISRDNAVDTMYLQMNSLKPEDTAVYYCAA

SEQ ID NO: 55 - protein, 32 aa, synthetic construct
  source 1..32: note = KERE FW3 sequence no. 6
RFTISRDNAKNTVYLQMDNVKPEDTAIYYCAA

SEQ ID NO: 56 - protein, 32 aa, synthetic construct
  source 1..32: note = KERE FW3 sequence no. 7
RFTISKDSGKNTVYLQMTSLKPEDTAVYYCAT

SEQ ID NO: 57 - protein, 32 aa, synthetic construct
  source 1..32: note = KERE FW3 sequence no. 8
RFTISRDSAKNMMYLQMNNLKPQDTAVYYCAA

SEQ ID NO: 58 - protein, 32 aa, synthetic construct
  source 1..32: note = KERE FW3 sequence no. 9
RFTISRENDKSTVYLQLNSLKPEDTAVYYCAA

SEQ ID NO: 59 - protein, 32 aa, synthetic construct
  source 1..32: note = KERE FW3 sequence no. 10
RFTISRDYAGNTAYLQMNSLKPEDTGVYYCAT

SEQ ID NO: 60 - protein, 11 aa, synthetic construct
  source 1..11: note = KERE FW4 sequence no. 1
WGQGTQVTVSS

SEQ ID NO: 61 - protein, 11 aa, synthetic construct
  source 1..11: note = KERE FW4 sequence no. 2
WGKGTLVTVSS

SEQ ID NO: 62 - protein, 11 aa, synthetic construct
  source 1..11: note = KERE FW4 sequence no. 3
RGQGTRVTVSS

SEQ ID NO: 63 - protein, 11 aa, synthetic construct
  source 1..11: note = KERE FW4 sequence no. 4
WGLGTQVTISS

SEQ ID NO: 64 - protein, 30 aa, synthetic construct
  source 1..30: note = GLEW FW1 sequence no. 1
QVQLVESGGGLVQPGGSLRLSCAASGFTFS

SEQ ID NO: 65 - protein, 30 aa, synthetic construct
  source 1..30: note = GLEW FW1 sequence no. 2
EVHLVESGGGLVRPGGSLRLSCAAFGFIFK

SEQ ID NO: 66 - protein, 30 aa, synthetic construct
  source 1..30: note = GLEW FW1 sequence no. 3
QVKLEESGGGLAQPGGSLRLSCVASGFTFS

SEQ ID NO: 67 - protein, 30 aa, synthetic construct
  source 1..30: note = GLEW FW1 sequence no. 4
EVQLVESGGGLVQPGGSLRLSCVCVSSGCT

SEQ ID NO: 68 - protein, 30 aa, synthetic construct
  source 1..30: note = GLEW FW1 sequence no. 5
EVQLVESGGGLALPGGSLTLSCVFSGSTFS

SEQ ID NO: 69 - protein, 22 aa, synthetic construct
  source 1..22: note = GLEW FW1 sequence no. 6
VESGGGLVQPGGSLRLSCAASG

SEQ ID NO: 70 - protein, 22 aa, synthetic construct
  source 1..22: note = GLEW FW1 sequence no. 7
EESGGGLAQPGGSLRLSCVASG

SEQ ID NO: 71 - protein, 22 aa, synthetic construct
  source 1..22: note = GLEW FW1 sequence no. 8
VESGGGLALPGGSLTLSCVFSG

SEQ ID NO: 72 - protein, 14 aa, synthetic construct
  source 1..14: note = GLEW FW2 sequence no. 1
WVRQAPGKVLEWVS

SEQ ID NO: 73 - protein, 14 aa, synthetic construct
  source 1..14: note = GLEW FW2 sequence no. 2
WVRRPPGKGLEWVS

SEQ ID NO: 74 - protein, 14 aa, synthetic construct
  source 1..14: note = GLEW FW2 sequence no. 3
WVRQAPGMGLEWVS

SEQ ID NO: 75 - protein, 14 aa, synthetic construct
  source 1..14: note = GLEW FW2 sequence no. 4
WVRQAPGKEPEWVS

SEQ ID NO: 76 - protein, 14 aa, synthetic construct
  source 1..14: note = GLEW FW2 sequence no. 5
WVRQAPGKDQEWVS

SEQ ID NO: 77 - protein, 14 aa, synthetic construct
  source 1..14: note = GLEW FW2 sequence no. 6
WVRQAPGKAEEWVS

SEQ ID NO: 78 - protein, 14 aa, synthetic construct
  source 1..14: note = GLEW FW2 sequence no. 7
WVRQAPGKGLEWVA

SEQ ID NO: 79 - protein, 14 aa, synthetic construct
  source 1..14: note = GLEW FW2 sequence no. 8
WVRQAPGRATEWVS

SEQ ID NO: 80 - protein, 32 aa, synthetic construct
  source 1..32: note = GLEW FW3 sequence no. 1
RFTISRDNAKNTLYLQMNSLKPEDTAVYYCVK

SEQ ID NO: 81 - protein, 32 aa, synthetic construct
  source 1..32: note = GLEW FW3 sequence no. 2
RFTISRDNARNTLYLQMDSLIPEDTALYYCAR

SEQ ID NO: 82 - protein, 32 aa, synthetic construct
  source 1..32: note = GLEW FW3 sequence no. 3
RFTSSRDNAKSTLYLQMNDLKPEDTALYYCAR

SEQ ID NO: 83 - protein, 32 aa, synthetic construct
  source 1..32: note = GLEW FW3 sequence no. 4
RFIISRDNAKNTLYLQMNSLGPEDTAMYYCQR

SEQ ID NO: 84 - protein, 32 aa, synthetic construct
  source 1..32: note = GLEW FW3 sequence no. 5
RFTASRDNAKNTLYLQMNSLKSEDTARYYCAR

SEQ ID NO: 85 - protein, 32 aa, synthetic construct
  source 1..32: note = GLEW FW3 sequence no. 6
RFTISRDNAKNTLYLQMDDLQSEDTAMYYCGR

SEQ ID NO: 86 - protein, 11 aa, synthetic construct
  source 1..11: note = GLEW FW4 sequence no. 1
GSQGTQVTVSS

SEQ ID NO: 87 - protein, 11 aa, synthetic construct
  source 1..11: note = GLEW FW4 sequence no. 2
LRGGTQVTVSS

SEQ ID NO: 88 - protein, 11 aa, synthetic construct
  source 1..11: note = GLEW FW4 sequence no. 3
RGQGTLVTVSS

SEQ ID NO: 89 - protein, 11 aa, synthetic construct
  source 1..11: note = GLEW FW4 sequence no. 4
RSRGIQVTVSS

SEQ ID NO: 90 - protein, 11 aa, synthetic construct
  source 1..11: note = GLEW FW4 sequence no. 5
WGKGTQVTVSS

SEQ ID NO: 91 - protein, 11 aa, synthetic construct
  source 1..11: note = GLEW FW4 sequence no. 6
WGQGTQVTVSS

SEQ ID NO: 92 - protein, 30 aa, synthetic construct
  source 1..30: note = P,R,S 103 FW1 sequence no. 1
AVQLVESGGGLVQAGGSLRLSCAASGRTFS

SEQ ID NO: 93 - protein, 30 aa, synthetic construct
  source 1..30: note = P,R,S 103 FW1 sequence no. 2
QVQLQESGGGMVQPGGSLRLSCAASGFDFG

SEQ ID NO: 94 - protein, 30 aa, synthetic construct; identical to SEQ ID NO: 65
  source 1..30: note = P,R,S 103 FW1 sequence no. 3

SEQ ID NO: 95 - protein, 30 aa, synthetic construct
  source 1..30: note = P,R,S 103 FW1 sequence no. 4
QVQLAESGGGLVQPGGSLKLSCAASRTIVS

SEQ ID NO: 96 - protein, 30 aa, synthetic construct
  source 1..30: note = P,R,S 103 FW1 sequence no. 5
QEHLVESGGGLVDIGGSLRLSCAASERIFS

SEQ ID NO: 97 - protein, 30 aa, synthetic construct; identical to SEQ ID NO: 66
  source 1..30: note = P,R,S 103 FW1 sequence no. 6

SEQ ID NO: 98 - protein, 30 aa, synthetic construct; identical to SEQ ID NO: 67
  source 1..30: note = P,R,S 103 FW1 sequence no. 7

SEQ ID NO: 99 - protein, 30 aa, synthetic construct; identical to SEQ ID NO: 68
  source 1..30: note = P,R,S 103 FW1 sequence no. 8

SEQ ID NO: 100 - protein, 22 aa, synthetic construct
  source 1..22: note = P,R,S 103 FW1 sequence no. 9
VESGGGLVQAGGSLRLSCAASG

SEQ ID NO: 101 - protein, 22 aa, synthetic construct
  source 1..22: note = P,R,S 103 FW1 sequence no. 10
AESGGGLVQPGGSLKLSCAASR

SEQ ID NO: 102 - protein, 14 aa, synthetic construct
  source 1..14: note = P,R,S 103 FW2 sequence no. 1
WFRQAPGKEREFVA

SEQ ID NO: 103 - protein, 14 aa, synthetic construct
  source 1..14: note = P,R,S 103 FW2 sequence no. 2
WVRQAPGKVLEWVS

SEQ ID NO: 104 - protein, 14 aa, synthetic construct
  source 1..14: note = P,R,S 103 FW2 sequence no. 3
WVRRPPGKGLEWVS

SEQ ID NO: 105 - protein, 14 aa, synthetic construct
  source 1..14: note = P,R,S 103 FW2 sequence no. 4
WIRQAPGKEREGVS

SEQ ID NO: 106 - protein, 14 aa, synthetic construct
  source 1..14: note = P,R,S 103 FW2 sequence no. 5
WVRQYPGKEPEWVS

SEQ ID NO: 107 - protein, 14 aa, synthetic construct
  source 1..14: note = P,R,S 103 FW2 sequence no. 6
WFRQPPGKEHEFVA

SEQ ID NO: 108 - protein, 14 aa, synthetic construct
  source 1..14: note = P,R,S 103 FW2 sequence no. 7
WYRQAPGKRTELVA

SEQ ID NO: 109 - protein, 14 aa, synthetic construct
  source 1..14: note = P,R,S 103 FW2 sequence no. 8
WLRQAPGQGLEWVS

SEQ ID NO: 110 - protein, 14 aa, synthetic construct
  source 1..14: note = P,R,S 103 FW2 sequence no. 9
WLRQTPGKGLEWVG

SEQ ID NO: 111 - protein, 14 aa, synthetic construct
  source 1..14: note = P,R,S 103 FW2 sequence no. 10
WVRQAPGKAEEFVS

SEQ ID NO: 112 - protein, 32 aa, synthetic construct
  source 1..32: note = P,R,S 103 FW3 sequence no. 1
RFTISRDNAKNTVYLQMNSLKPEDTAVYYCAA

SEQ ID NO: 113 - protein, 32 aa, synthetic construct; identical to SEQ ID NO: 81
  source 1..32: note = P,R,S 103 FW3 sequence no. 2

SEQ ID NO: 114 - protein, 32 aa, synthetic construct
  source 1..32: note = P,R,S 103 FW3 sequence no. 3
RFTISRDNAKNEMYLQMNNLKTEDTGVYWCGA

SEQ ID NO: 115 - protein, 32 aa, synthetic construct
  source 1..32: note = P,R,S 103 FW3 sequence no. 4
RFTISSDSNRNMIYLQMNNLKPEDTAVYYCAA

SEQ ID NO: 116 - protein, 32 aa, synthetic construct
  source 1..32: note = P,R,S 103 FW3 sequence no. 5
RFTISRDNAKNMLYLHLNNLKSEDTAVYYCRR

SEQ ID NO: 117 - protein, 32 aa, synthetic construct
  source 1..32: note = P,R,S 103 FW3 sequence no. 6
RFTISRDNAKKTVYLRLNSLNPEDTAVYSCNL

SEQ ID NO: 118 - protein, 32 aa, synthetic construct
  source 1..32: note = P,R,S 103 FW3 sequence no. 7
RFKISRDNAKKTLYLQMNSLGPEDTAMYYCQR

SEQ ID NO: 119 - protein, 32 aa, synthetic construct
  source 1..32: note = P,R,S 103 FW3 sequence no. 8
RFTVSRDNGKNTAYLRMNSLKPEDTADYYCAV

SEQ ID NO: 120 - protein, 11 aa, synthetic construct
  source 1..11: note = P,R,S 103 FW4 sequence no. 1
RGQGTQVTVSS

SEQ ID NO: 121 - protein, 11 aa, synthetic construct
  source 1..11: note = P,R,S 103 FW4 sequence no. 2
LRGGTQVTVSS

SEQ ID NO: 122 - protein, 11 aa, synthetic construct
  source 1..11: note = P,R,S 103 FW4 sequence no. 3
GNKGTLVTVSS

SEQ ID NO: 123 - protein, 11 aa, synthetic construct
  source 1..11: note = P,R,S 103 FW4 sequence no. 4
SSPGTQVTVSS

SEQ ID NO: 124 - protein, 11 aa, synthetic construct
  source 1..11: note = P,R,S 103 FW4 sequence no. 5
SSQGTLVTVSS

SEQ ID NO: 125 - protein, 11 aa, synthetic construct
  source 1..11: note = P,R,S 103 FW4 sequence no. 6
RSRGIQVTVSS

SEQ ID NO: 126 - protein, 30 aa, synthetic construct
  source 1..30: note = pN0150 (FR1)
QVQLVESGGGLVQPGGSLRLSCAASGRFQS

SEQ ID NO: 127 - protein, 30 aa, synthetic construct
  source 1..30: note = pN0152 (FR1)
QVQLVESGGGLVQPGGSLKLSCAASGSISS

SEQ ID NO: 128 - protein, 30 aa, synthetic construct
  source 1..30: note = pN0162 (FR1)
QVQLVESGGGLVQPGGSLRLSCAASGSAFS

SEQ ID NO: 129 - protein, 30 aa, synthetic construct; identical to SEQ ID NO: 128
  source 1..30: note = pN2059 (FR1)

SEQ ID NO: 130 - protein, 30 aa, synthetic construct
  source 1..30: note = pN2060 (FR1)
QVQLVESGGGLVQPGGSLRLSCAASGSAFR

SEQ ID NO: 131 - protein, 30 aa, synthetic construct; identical to SEQ ID NO: 130
  source 1..30: note = pN2061 (FR1)

SEQ ID NO: 132 - protein, 30 aa, synthetic construct; identical to SEQ ID NO: 130
  source 1..30: note = pN2062 (FR1)

SEQ ID NO: 133 - protein, 30 aa, synthetic construct; identical to SEQ ID NO: 128
  source 1..30: note = pN2121 (FR1)

SEQ ID NO: 134 - protein, 5 aa, synthetic construct
  source 1..5: note = pN0150 (CDR1)
IMSMG

SEQ ID NO: 135 - protein, 5 aa, synthetic construct
  source 1..5: note = pN0152 (CDR1)
KSDWG

SEQ ID NO: 136 - protein, 5 aa, synthetic construct
  source 1..5: note = pN0162 (CDR1)
LNVMG

SEQ ID NO: 137 - protein, 5 aa, synthetic construct
  source 1..5: note = pN2059 (CDR1)
LRWMG

SEQ ID NO: 138 - protein, 5 aa, synthetic construct
  source 1..5: note = pN2060 (CDR1)
LRVMG

SEQ ID NO: 139 - protein, 5 aa, synthetic construct
  source 1..5: note = pN2061 (CDR1)
LRVMG

SEQ ID NO: 140 - protein, 5 aa, synthetic construct
  source 1..5: note = pN2062 (CDR1)
LHVMG

SEQ ID NO: 141 - protein, 5 aa, synthetic construct
  source 1..5: note = pN2121 (CDR1)
LHWMG

SEQ ID NO: 142 - protein, 14 aa, synthetic construct
  source 1..14: note = pN0150 (FR2)
WYRQAPGKQRESVA

SEQ ID NO: 143 - protein, 14 aa, synthetic construct
  source 1..14: note = pN0152 (FR2)
WYRQAPGKERELVA

SEQ ID NO: 144 - protein, 14 aa, synthetic construct
  source 1..14: note = pN0162 (FR2)
WYRQTPGNQRALVA

SEQ ID NO: 145 - protein, 14 aa, synthetic construct
  source 1..14: note = pN2059 (FR2)
WYRQTPGKQRELVA

SEQ ID NO: 146 - protein, 14 aa, synthetic construct
  source 1..14: note = pN2060 (FR2)
WYRQTPGKQRELVA

SEQ ID NO: 147 - protein, 14 aa, synthetic construct
  source 1..14: note = pN2061 (FR2)
WYRQTPGKQRELVA

SEQ ID NO: 148 - protein, 14 aa, synthetic construct
  source 1..14: note = pN2062 (FR2)
WYRQTPGKQRELVA

SEQ ID NO: 149 - protein, 14 aa, synthetic construct
  source 1..14: note = pN2121 (FR2)
WYRQTPGKQRELVA

SEQ ID NO: 150 - protein, 16 aa, synthetic construct
  source 1..16: note = pN0150 (CDR2)
SISSGGSTNYADSVKG

SEQ ID NO: 151 - protein, 16 aa, synthetic construct
  source 1..16: note = pN0152 (CDR2)
QITSGGNLKYASAVTG

SEQ ID NO: 152 - protein, 16 aa, synthetic construct
  source 1..16: note = pN0162 (CDR2)
EITKDGSTNYADSAKG

SEQ ID NO: 153 - protein, 16 aa, synthetic construct
  source 1..16: note = pN2059 (CDR2)
EITKSGSTNYADSAKG

SEQ ID NO: 154 - protein, 16 aa, synthetic construct
  source 1..16: note = pN2060 (CDR2)
EITKDGSTNYADSAKG

SEQ ID NO: 155 - protein, 16 aa, synthetic construct
  source 1..16: note = pN2061 (CDR2)
EITKSGSTNYADSAKG

SEQ ID NO: 156 - protein, 16 aa, synthetic construct
  source 1..16: note = pN2062 (CDR2)
EITKSGSTNYADSAKG

SEQ ID NO: 157 - protein, 16 aa, synthetic construct
  source 1..16: note = pN2121 (CDR2)
EITKSGSTNYADSAKG

SEQ ID NO: 158 - protein, 32 aa, synthetic construct
  source 1..32: note = pN0150 (FR3)
RFTISRDNAKNLMYLQMNSLKPEDTAVYYCNA

SEQ ID NO: 159 - protein, 32 aa, synthetic construct
  source 1..32: note = pN0152 (FR3)
RFTISRDNAKNTVNLLMSRLKPEDTAIYFCNA

SEQ ID NO: 160 - protein, 32 aa, synthetic construct
  source 1..32: note = pN0162 (FR3)
RFTISRDNAKNTVHLQMDSLKPEDTAVYYCKA

SEQ ID NO: 161 - protein, 32 aa, synthetic construct
  source 1..32: note = pN2059 (FR3)
RFTISRDNAKNTVYLQMNSLRPEDTAVYYCKA

SEQ ID NO: 162 - protein, 32 aa, synthetic construct; identical to SEQ ID NO: 161
  source 1..32: note = pN2060 (FR3)

SEQ ID NO: 163 - protein, 32 aa, synthetic construct; identical to SEQ ID NO: 161
  source 1..32: note = pN2061 (FR3)

SEQ ID NO: 164 - protein, 32 aa, synthetic construct; identical to SEQ ID NO: 161
  source 1..32: note = pN2062 (FR3)

SEQ ID NO: 165 - protein, 32 aa, synthetic construct; identical to SEQ ID NO: 161
  source 1..32: note = pN2121 (FR3)

SEQ ID NO: 166 - protein, 15 aa, synthetic construct
  source 1..15: note = pN0150 (CDR3)
QVKSTPLQFQSTRNY

SEQ ID NO: 167 - protein, 11 aa, synthetic construct
  source 1..11: note = pN0152 (CDR3)
RTWRLVRYYDY

SEQ ID NO: 168 - protein, 11 aa, synthetic construct
  source 1..11: note = pN0162 (CDR3)
RTGRIVRPLDY

SEQ ID NO: 169 - protein, 11 aa, synthetic construct
  source 1..11: note = pN2059 (CDR3)
RTGRIVRPVDY

SEQ ID NO: 170 - protein, 11 aa, synthetic construct
  source 1..11: note = pN2060 (CDR3)
RTGRIVRPVDY

SEQ ID NO: 171 - protein, 11 aa, synthetic construct
  source 1..11: note = pN2061 (CDR3)
RTGRIVRPLDY

SEQ ID NO: 172 - protein, 11 aa, synthetic construct
  source 1..11: note = pN2062 (CDR3)
RTGRIVRPVDY

SEQ ID NO: 173 - protein, 11 aa, synthetic construct
  source 1..11: note = pN2121 (CDR3)
RTGRIVRPVDY

SEQ ID NO: 174 - protein, 11 aa, synthetic construct
  source 1..11: note = pN0150 (FR4)
WGQGTQVTVSS

SEQ ID NO: 175 - protein, 11 aa, synthetic construct
  source 1..11: note = pN0152 (FR4)
WGQGTQVTVSS

SEQ ID NO: 176 - protein, 11 aa, synthetic construct
  source 1..11: note = pN0162 (FR4)
WGQGTQVTVSS

SEQ ID NO: 177 - protein, 11 aa, synthetic construct
  source 1..11: note = pN2059 (FR4)
WGQGTLVTVSS

SEQ ID NO: 178 - protein, 11 aa, synthetic construct
  source 1..11: note = pN2060 (FR4)
WGQGTLVTVSS

SEQ ID NO: 179 - protein, 11 aa, synthetic construct
  source 1..11: note = pN2061 (FR4)
WGQGTLVTVSS

SEQ ID NO: 180 - protein, 11 aa, synthetic construct
  source 1..11: note = pN2062 (FR4)
WGQGTLVTVSS

SEQ ID NO: 181 - protein, 11 aa, synthetic construct
  source 1..11: note = pN2121 (FR4)
WGQGTLVTVSS

SEQ ID NO: 182 - protein, 123 aa, synthetic construct
  source 1..123: note = pN0150
QVQLVESGGGLVQPGGSLRLSCAASGRFQSIMSMGWYRQAPGKQRESVASISSGGSTNYADSVKGRFTISRDNAKNLMYLQMNSLKPEDTAVYYCNAQVKSTPLQFQSTRNYWGQGTQVTVSS

SEQ ID NO: 183 - protein, 119 aa, synthetic construct
  source 1..119: note = pN0152
QVQLVESGGGLVQPGGSLKLSCAASGSISSKSDWGWYRQAPGKERELVAQITSGGNLKYASAVTGRFTISRDNAKNTVNLLMSRLKPEDTAIYFCNARTWRLVRYYDYWGQGTQVTVSS

SEQ ID NO: 184 - protein, 119 aa, synthetic construct
  source 1..119: note = pN0162
QVQLVESGGGLVQPGGSLRLSCAASGSAFSLNVMGWYRQTPGNQRALVAEITKDGSTNYADSAKGRFTISRDNAKNTVHLQMDSLKPEDTAVYYCKARTGRIVRPLDYWGQGTQVTVSS

SEQ ID NO: 185 - protein, 119 aa, synthetic construct
  source 1..119: note = pN2059
QVQLVESGGGLVQPGGSLRLSCAASGSAFSLRWMGWYRQTPGKQRELVAEITKSGSTNYADSAKGRFTISRDNAKNTVYLQMNSLRPEDTAVYYCKARTGRIVRPVDYWGQGTLVTVSS

SEQ ID NO: 186 - protein, 119 aa, synthetic construct
  source 1..119: note = pN2060
QVQLVESGGGLVQPGGSLRLSCAASGSAFRLRVMGWYRQTPGKQRELVAEITKDGSTNYADSAKGRFTISRDNAKNTVYLQMNSLRPEDTAVYYCKARTGRIVRPVDYWGQGTLVTVSS

SEQ ID NO: 187 - protein, 119 aa, synthetic construct
  source 1..119: note = pN2061
QVQLVESGGGLVQPGGSLRLSCAASGSAFRLRVMGWYRQTPGKQRELVAEITKSGSTNYADSAKGRFTISRDNAKNTVYLQMNSLRPEDTAVYYCKARTGRIVRPLDYWGQGTLVTVSS

SEQ ID NO: 188 - protein, 119 aa, synthetic construct
  source 1..119: note = pN2062
QVQLVESGGGLVQPGGSLRLSCAASGSAFRLHVMGWYRQTPGKQRELVAEITKSGSTNYADSAKGRFTISRDNAKNTVYLQMNSLRPEDTAVYYCKARTGRIVRPVDYWGQGTLVTVSS

SEQ ID NO: 189 - protein, 119 aa, synthetic construct
  source 1..119: note = pN2121
QVQLVESGGGLVQPGGSLRLSCAASGSAFSLHWMGWYRQTPGKQRELVAEITKSGSTNYADSAKGRFTISRDNAKNTVYLQMNSLRPEDTAVYYCKARTGRIVRPVDYWGQGTLVTVSS

SEQ ID NO: 190 - protein, 346 aa, synthetic construct
  source 1..346: note = pN0162-huFcIgG1_N297A
QVQLVESGGGLVQPGGSLRLSCAASGSAFSLNVMGWYRQTPGNQRALVAEITKDGSTNYADSAKGRFTISRDNAKNTVHLQMDSLKPEDTAVYYCKARTGRIVRPLDYWGQGTQVTVSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 346 aa, synthetic construct
  source 1..346: note = pN2037 (huFc IgG1 L234A _L235A)
DVQLVESGGGLVQPGGSLRLSCAASGSAFRLHVMGWYRQTPGKQRELVAEITKSGSTNYADSAKGRFTISRDNAKNTVYLQMNSLRPEDTAVYYCKARTGRIVRPVDYWGQGTLVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 346 aa, synthetic construct
  source 1..346: note = pN2112 (huFc_IgG1 L234A _L235A)
QVQLVESGGGLVQPGGSLRLSCAASGSAFRLHWMGWYRQTPGKQRELVAEITKDGSTNYADSAKGRFTISRDNAKNTVYLQMNSLRPEDTAVYYCKARTGRIVRPVDYWGQGTLVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 346 aa, synthetic construct
  source 1..346: note = pN2113 = pN2121-huFc_IgG1 L234A _L235A
QVQLVESGGGLVQPGGSLRLSCAASGSAFSLHWMGWYRQTPGKQRELVAEITKSGSTNYADSAKGRFTISRDNAKNTVYLQMNSLRPEDTAVYYCKARTGRIVRPVDYWGQGTLVTVSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 30 aa, synthetic construct
  source 1..30: note = PN0148 (FR1)
QVQLVESGGGSVQAGGSLRLSCAASGITFS

SEQ ID NO: 195 - protein, 30 aa, synthetic construct
  source 1..30: note = PN0149 (FR1)
QVQLVESGGGLVQPGGSLRLSCAASGFVFG

SEQ ID NO: 196 - protein, 30 aa, synthetic construct
  source 1..30: note = PN0151 (FR1)
QVQLVESGGGLVQAEGSLSLSCAASGNFFS

SEQ ID NO: 197 - protein, 30 aa, synthetic construct
  source 1..30: note = PN0153 (FR1)
QVQLVESGGGLVQAGGSLRLSCAVSRSIFS

SEQ ID NO: 198 - protein, 30 aa, synthetic construct
  source 1..30: note = PN0160 (FR1)
QVQLVESGGGLVQAGGSLRLSCAASGSIFS

SEQ ID NO: 199 - protein, 30 aa, synthetic construct
  source 1..30: note = PN0155 (FR1)
QVQLVESGGGLVQAGGSLRLSCAVSGNIRT

SEQ ID NO: 200 - protein, 30 aa, synthetic construct; identical to SEQ ID NO: 199
  source 1..30: note = PN0156 (FR1)

SEQ ID NO: 201 - protein, 30 aa, synthetic construct; identical to SEQ ID NO: 198
  source 1..30: note = PN0157 (FR1)

SEQ ID NO: 202 - protein, 31 aa, synthetic construct
  source 1..31: note = PN0161 (FR1)
QVQLVESGGGLVQAGGSLRLSCAASGSISRT

SEQ ID NO: 203 - protein, 30 aa, synthetic construct
  source 1..30: note = PN0163 (FR1)
QVQLVESGGGSVQAGGSLRLSCAASGSIFN

SEQ ID NO: 204 - protein, 30 aa, synthetic construct
  source 1..30: note = PN0164 (FR1)
QVQLVESGGGLVQAGGSLRLSCTASGRIFS

SEQ ID NO: 205 - protein, 30 aa, synthetic construct
  source 1..30: note = PN0159 (FR1)
QVQLVESGGGLVQPGGSLRLSCAASGSISS

SEQ ID NO: 206 - protein, 30 aa, synthetic construct
  source 1..30: note = PN0165 (FR1)
QVQLVESGGGLVQAGGSLRLSCGASGSIFM

SEQ ID NO: 207 - protein, 30 aa, synthetic construct
  source 1..30: note = PN0166 (FR1)
QVQLVESGGGPVLAGGSLRLSCAASGRVLV

SEQ ID NO: 208 - protein, 30 aa, synthetic construct
  source 1..30: note = PN0167 (FR1)
QVQLVESGGGLVQAGGSLRLSCAGSGSIRS

SEQ ID NO: 209 - protein, 30 aa, synthetic construct
  source 1..30: note = PN0145 (FR1)
QVQLVESGGGLVQAGGSLRLSCAVPERMRS

SEQ ID NO: 210 - protein, 5 aa, synthetic construct
  source 1..5: note = PN0148 (CDR1)
SNNLG

SEQ ID NO: 211 - protein, 5 aa, synthetic construct
  source 1..5: note = PN0149 (CDR1)
TSNMA

SEQ ID NO: 212 - protein, 5 aa, synthetic construct
  source 1..5: note = PN0151 (CDR1)
IVDMG

SEQ ID NO: 213 - protein, 5 aa, synthetic construct
  source 1..5: note = PN0153 (CDR1)
INAMG

SEQ ID NO: 214 - protein, 5 aa, synthetic construct
  source 1..5: note = PN0160 (CDR1)
AISMG

SEQ ID NO: 215 - protein, 5 aa, synthetic construct
  source 1..5: note = PN0155 (CDR1)
ISVMG

SEQ ID NO: 216 - protein, 5 aa, synthetic construct
  source 1..5: note = PN0156 (CDR1)
TNVMG

SEQ ID NO: 217 - protein, 5 aa, synthetic construct
  source 1..5: note = PN0157 (CDR1)
INVMG

SEQ ID NO: 218 - protein, 4 aa, synthetic construct
  source 1..4: note = PN0161 (CDR1)
NGMG

SEQ ID NO: 219 - protein, 5 aa, synthetic construct
  source 1..5: note = PN0163 (CDR1)
RAVMG

SEQ ID NO: 220 - protein, 5 aa, synthetic construct
  source 1..5: note = PN0164 (CDR1)
INAMA

SEQ ID NO: 221 - protein, 5 aa, synthetic construct
  source 1..5: note = PN0159 (CDR1)
DYTMG

SEQ ID NO: 222 - protein, 5 aa, synthetic construct
  source 1..5: note = PN0165 (CDR1)
INVMG

SEQ ID NO: 223 - protein, 5 aa, synthetic construct
  source 1..5: note = PN0166 (CDR1)
INTMG

SEQ ID NO: 224 - protein, 5 aa, synthetic construct
  source 1..5: note = PN0167 (CDR1)
FNSMA

SEQ ID NO: 225 - protein, 5 aa, synthetic construct
  source 1..5: note = PN0145 (CDR1)
INVLA

SEQ ID NO: 226 - protein, 14 aa, synthetic construct
  source 1..14: note = PN0148 (FR2)
WYRQAPGKQRELVA

SEQ ID NO: 227 - protein, 14 aa, synthetic construct
  source 1..14: note = PN0149 (FR2)
WYRQAPGKQRELVA

SEQ ID NO: 228 - protein, 14 aa, synthetic construct
  source 1..14: note = PN0151 (FR2)
WYRQAPGKQRELVA

SEQ ID NO: 229 - protein, 14 aa, synthetic construct
  source 1..14: note = PN0153 (FR2)
WYRQAPGKQRELVA

SEQ ID NO: 230 - protein, 14 aa, synthetic construct
  source 1..14: note = PN0160 (FR2)
WYRQAPGKQRELVA

SEQ ID NO: 231 - protein, 14 aa, synthetic construct
  source 1..14: note = PN0155 (FR2)
WYRQAPEKQREWVA

SEQ ID NO: 232 - protein, 14 aa, synthetic construct
  source 1..14: note = PN0156 (FR2)
WHRQAPEKQREWVA

SEQ ID NO: 233 - protein, 14 aa, synthetic construct
  source 1..14: note = PN0157 (FR2)
WYRQVPGKQREFVA

SEQ ID NO: 234 - protein, 14 aa, synthetic construct
  source 1..14: note = PN0161 (FR2)
WYRQAPGKQRELVA

SEQ ID NO: 235 - protein, 14 aa, synthetic construct
  source 1..14: note = PN0163 (FR2)
WYRQAPGKQRESVA

SEQ ID NO: 236 - protein, 14 aa, synthetic construct
  source 1..14: note = PN0164 (FR2)
WYRQAPGKQRELVA

SEQ ID NO: 237 - protein, 14 aa, synthetic construct
  source 1..14: note = PN0159 (FR2)
WYRQAPGKQRELVA

SEQ ID NO: 238 - protein, 14 aa, synthetic construct
  source 1..14: note = PN0165 (FR2)
WYRQAPGKERELVA

SEQ ID NO: 239 - protein, 14 aa, synthetic construct
  source 1..14: note = PN0166 (FR2)
WYRQTPGQQRELVA

SEQ ID NO: 240 - protein, 14 aa, synthetic construct
  source 1..14: note = PN0167 (FR2)
WYRQAPGKQRELVA

SEQ ID NO: 241 - protein, 14 aa, synthetic construct
  source 1..14: note = PN0145 (FR2)
WYRQAPGKQRELVA

SEQ ID NO: 242 - protein, 16 aa, synthetic construct
  source 1..16: note = PN0148 (CDR2)
AITVGGTTTYVDSVKG

SEQ ID NO: 243 - protein, 16 aa, synthetic construct
  source 1..16: note = PN0149 (CDR2)
STTPGGTINSANSVKG

SEQ ID NO: 244 - protein, 16 aa, synthetic construct
  source 1..16: note = PN0151 (CDR2)
TITSGSNTNYADSVKG

SEQ ID NO: 245 - protein, 16 aa, synthetic construct
  source 1..16: note = PN0153 (CDR2)
AITSGGSTDLVDFVKG

SEQ ID NO: 246 - protein, 16 aa, synthetic construct
  source 1..16: note = PN0160 (CDR2)
TITRGDSTNYADSVKG

SEQ ID NO: 247 - protein, 16 aa, synthetic construct
  source 1..16: note = PN0155 (CDR2)
TITTGGNTNYADSVKG

SEQ ID NO: 248 - protein, 16 aa, synthetic construct
  source 1..16: note = PN0156 (CDR2)
TITTGGNTNYVDSVKG

SEQ ID NO: 249 - protein, 16 aa, synthetic construct
  source 1..16: note = PN0157 (CDR2)
DIRRGGVTNYADFAKG

SEQ ID NO: 250 - protein, 16 aa, synthetic construct
  source 1..16: note = PN0161 (CDR2)
AITTGGSTNYANSAKG

SEQ ID NO: 251 - protein, 16 aa, synthetic construct
  source 1..16: note = PN0163 (CDR2)
TITRGGSANYADSVKG

SEQ ID NO: 252 - protein, 16 aa, synthetic construct
  source 1..16: note = PN0164 (CDR2)
TITSGGSTNYADSVKG

SEQ ID NO: 253 - protein, 16 aa, synthetic construct
  source 1..16: note = PN0159 (CDR2)
SITSAGTTNYADSVKG

SEQ ID NO: 254 - protein, 16 aa, synthetic construct
  source 1..16: note = PN0165 (CDR2)
RINSGGTTNYADSAKG

SEQ ID NO: 255 - protein, 16 aa, synthetic construct
  source 1..16: note = PN0166 (CDR2)
TSTNSGKVTYSDSVKG

SEQ ID NO: 256 - protein, 16 aa, synthetic construct
  source 1..16: note = PN0167 (CDR2)
RIRTGGRTEYADSVKG

SEQ ID NO: 257 - protein, 16 aa, synthetic construct
  source 1..16: note = PN0145 (CDR2)
GITSGDSTNYSEAVKG

SEQ ID NO: 258 - protein, 32 aa, synthetic construct
  source 1..32: note = PN0148 (FR3)
RFIISRDNTKSTLFLQMNSLEPEDTAIYYCNA

SEQ ID NO: 259 - protein, 32 aa, synthetic construct
  source 1..32: note = PN0149 (FR3)
RFTISRDNVKNMVSLQMNSLKPEDTAVYYCNA

SEQ ID NO: 260 - protein, 32 aa, synthetic construct
  source 1..32: note = PN0151 (FR3)
RFTISRDNFKNTVYLQMNSLKPEDTAVYYCNA

SEQ ID NO: 261 - protein, 32 aa, synthetic construct
  source 1..32: note = PN0153 (FR3)
RFTISRDDVKNTVDLQMNSLKPEDTAVYYCNA

SEQ ID NO: 262 - protein, 32 aa, synthetic construct
  source 1..32: note = PN0160 (FR3)
RFTISRDSAKNTVYLQMNSLKPEDTAVYYCNA

SEQ ID NO: 263 - protein, 32 aa, synthetic construct
  source 1..32: note = PN0155 (FR3)
RFTASRDASKNMVYLQMNSLKPEDTAVYFCNT

SEQ ID NO: 264 - protein, 32 aa, synthetic construct
  source 1..32: note = PN0156 (FR3)
RFTISRDATKNMVYLQMNSLKPEDTAVYFCNT

SEQ ID NO: 265 - protein, 32 aa, synthetic construct
  source 1..32: note = PN0157 (FR3)
RFTISRDNAKNTVYLQMNSLKPEDTAVYYCNV

SEQ ID NO: 266 - protein, 32 aa, synthetic construct
  source 1..32: note = PN0161 (FR3)
RFTISRDNAKNTVYLQMNSLKPEDTAVYYCNA

SEQ ID NO: 267 - protein, 32 aa, synthetic construct
  source 1..32: note = PN0163 (FR3)
RFTISRVNAKNMVYLQMNSLEPEDTAVYYCNA

SEQ ID NO: 268 - protein, 32 aa, synthetic construct
  source 1..32: note = PN0164 (FR3)
RFAISRDNGKNTVFLQMNSLKPEDTAVYYCNA

SEQ ID NO: 269 - protein, 32 aa, synthetic construct
  source 1..32: note = PN0159 (FR3)
RFTISRDNAKNTVYLIMNSLKPEDTAVYYCNT

SEQ ID NO: 270 - protein, 32 aa, synthetic construct; identical to SEQ ID NO: 265
  source 1..32: note = PN0165 (FR3)

SEQ ID NO: 271 - protein, 32 aa, synthetic construct
  source 1..32: note = PN0166 (FR3)
RFTISRDNSKKTVDLQMNSLKSEDTAVYYCYA

SEQ ID NO: 272 - protein, 32 aa, synthetic construct
  source 1..32: note = PN0167 (FR3)
RFTISRDNAKNTVYLQMNTLRPEDTAVYYCNA

SEQ ID NO: 273 - protein, 32 aa, synthetic construct
  source 1..32: note = PN0145 (FR3)
RFTISRDNAKNTVYLQMNSLKPEDTAVYYCHV

SEQ ID NO: 274 - protein, 11 aa, synthetic construct
  source 1..11: note = PN0148 (CDR3)
KSTRVVRPVMY

SEQ ID NO: 275 - protein, 11 aa, synthetic construct
  source 1..11: note = PN0149 (CDR3)
EVRRGIARVNY

SEQ ID NO: 276 - protein, 13 aa, synthetic construct
  source 1..13: note = PN0151 (CDR3)
GVRTNFISQRRNY

SEQ ID NO: 277 - protein, 12 aa, synthetic construct
  source 1..12: note = PN0153 (CDR3)
RVRDWRFREKDY

SEQ ID NO: 278 - protein, 11 aa, synthetic construct
  source 1..11: note = PN0160 (CDR3)
KIRRGIVPINY

SEQ ID NO: 279 - protein, 14 aa, synthetic construct
  source 1..14: note = PN0155 (CDR3)
KTLQNPITRRTNEY

SEQ ID NO: 280 - protein, 14 aa, synthetic construct
  source 1..14: note = PN0156 (CDR3)
KTLQNPLTRRTNEY

SEQ ID NO: 281 - protein, 14 aa, synthetic construct
  source 1..14: note = PN0157 (CDR3)
QGLFRFPRANVFNK

SEQ ID NO: 282 - protein, 14 aa, synthetic construct
  source 1..14: note = PN0161 (CDR3)
RYGSSSFRWGDFEV

SEQ ID NO: 283 - protein, 10 aa, synthetic construct
  source 1..10: note = PN0163 (CDR3)
RSGLPYPNVY

SEQ ID NO: 284 - protein, 14 aa, synthetic construct
  source 1..14: note = PN0164 (CDR3)
DGVRGSFVSQGRVY

SEQ ID NO: 285 - protein, 11 aa, synthetic construct
  source 1..11: note = PN0159 (CDR3)
KKFRIVGYRDY

SEQ ID NO: 286 - protein, 14 aa, synthetic construct
  source 1..14: note = PN0165 (CDR3)
NARRNFISNRYNDY

SEQ ID NO: 287 - protein, 11 aa, synthetic construct
  source 1..11: note = PN0166 (CDR3)
GVRIGPATNAY

SEQ ID NO: 288 - protein, 10 aa, synthetic construct
  source 1..10: note = PN0167 (CDR3)
DTLFPRPVNY

SEQ ID NO: 289 - protein, 11 aa, synthetic construct
  source 1..11: note = PN0145 (CDR3)
EGRIRLNWGSY

SEQ ID NO: 290 - protein, 11 aa, synthetic construct
  source 1..11: note = PN0148 (FR4)
WGQGTQVTVSS

SEQ ID NO: 291 - protein, 11 aa, synthetic construct
  source 1..11: note = PN0149 (FR4)
WGQGTQVTVSS

SEQ ID NO: 292 - protein, 11 aa, synthetic construct
  source 1..11: note = PN0151 (FR4)
WGQGTQVTVSS

SEQ ID NO: 293 - protein, 11 aa, synthetic construct
  source 1..11: note = PN0153 (FR4)
WGQGTQVTVSS

SEQ ID NO: 294 - protein, 11 aa, synthetic construct
  source 1..11: note = PN0160 (FR4)
WGQGTQVTVSS

SEQ ID NO: 295 - protein, 11 aa, synthetic construct
  source 1..11: note = PN0155 (FR4)
WGQGTQVTVSS

SEQ ID NO: 296 - protein, 11 aa, synthetic construct
  source 1..11: note = PN0156 (FR4)
WGQGTQVTVSS

SEQ ID NO: 297 - protein, 11 aa, synthetic construct
  source 1..11: note = PN0157 (FR4)
WGQGTQVTVSS

SEQ ID NO: 298 - protein, 11 aa, synthetic construct
  source 1..11: note = PN0161 (FR4)
WGQGTQVTVSS

SEQ ID NO: 299 - protein, 11 aa, synthetic construct
  source 1..11: note = PN0163 (FR4)
WGQGTQVTVSS

SEQ ID NO: 300 - protein, 11 aa, synthetic construct
  source 1..11: note = PN0164 (FR4)
WGQGTQVTVSS

SEQ ID NO: 301 - protein, 11 aa, synthetic construct
  source 1..11: note = PN0159 (FR4)
WGQGTQVTVSS

SEQ ID NO: 302 - protein, 11 aa, synthetic construct
  source 1..11: note = PN0165 (FR4)
WGQGTQVTVSS

SEQ ID NO: 303 - protein, 11 aa, synthetic construct
  source 1..11: note = PN0166 (FR4)
WGRGTQVTVSS

SEQ ID NO: 304 - protein, 11 aa, synthetic construct
  source 1..11: note = PN0167 (FR4)
WGQGTQVTVSS

SEQ ID NO: 305 - protein, 11 aa, synthetic construct
  source 1..11: note = PN0145 (FR4)
WGQGTQVTVSS

SEQ ID NO: 306 - protein, 119 aa, synthetic construct
  source 1..119: note = PN0148
QVQLVESGGGSVQAGGSLRLSCAASGITFSSNNLGWYRQAPGKQRELVAAITVGGTTTYVDSVKGRFIISRDNTKSTLFLQMNSLEPEDTAIYYCNAKSTRVVRPVMYWGQGTQVTVSS

SEQ ID NO: 307 - protein, 108 aa, synthetic construct
  source 1..108: note = PN0149
QVQLVESGGGLVQPGGSLRLSCAASGFVFGTSNMAWYRQAPGKQRELVASTTPGGTINSANSVKGRFTISRDNVKNMVSLQMNSLKPEDTAVYYCNAEVRRGIARVNY

SEQ ID NO: 308 - protein, 121 aa, synthetic construct
  source 1..121: note = PN0151
QVQLVESGGGLVQAEGSLSLSCAASGNFFSIVDMGWYRQAPGKQRELVATITSGSNTNYADSVKGRFTISRDNFKNTVYLQMNSLKPEDTAVYYCNAGVRTNFISQRRNYWGQGTQVTVSS

SEQ ID NO: 309 - protein, 120 aa, synthetic construct
  source 1..120: note = PN0153
QVQLVESGGGLVQAGGSLRLSCAVSRSIFSINAMGWYRQAPGKQRELVAAITSGGSTDLVDFVKGRFTISRDDVKNTVDLQMNSLKPEDTAVYYCNARVRDWRFREKDYWGQGTQVTVSS

SEQ ID NO: 310 - protein, 119 aa, synthetic construct
  source 1..119: note = PN0160
QVQLVESGGGLVQAGGSLRLSCAASGSIFSAISMGWYRQAPGKQRELVATITRGDSTNYADSVKGRFTISRDSAKNTVYLQMNSLKPEDTAVYYCNAKIRRGIVPINYWGQGTQVTVSS

SEQ ID NO: 311 - protein, 122 aa, synthetic construct
  source 1..122: note = PN0155
QVQLVESGGGLVQAGGSLRLSCAVSGNIRTISVMGWYRQAPEKQREWVATITTGGNTNYADSVKGRFTASRDASKNMVYLQMNSLKPEDTAVYFCNTKTLQNPITRRTNEYWGQGTQVTVSS

SEQ ID NO: 312 - protein, 122 aa, synthetic construct
  source 1..122: note = PN0156
QVQLVESGGGLVQAGGSLRLSCAVSGNIRTTNVMGWHRQAPEKQREWVATITTGGNTNYVDSVKGRFTISRDATKNMVYLQMNSLKPEDTAVYFCNTKTLQNPLTRRTNEYWGQGTQVTVSS

SEQ ID NO: 313 - protein, 122 aa, synthetic construct
  source 1..122: note = PN0157
QVQLVESGGGLVQAGGSLRLSCAASGSIFSINVMGWYRQVPGKQREFVADIRRGGVTNYADFAKGRFTISRDNAKNTVYLQMNSLKPEDTAVYYCNVQGLFRFPRANVFNKWGQGTQVTVSS

SEQ ID NO: 314 - protein, 122 aa, synthetic construct
  source 1..122: note = PN0161
QVQLVESGGGLVQAGGSLRLSCAASGSISRTNGMGWYRQAPGKQRELVAAITTGGSTNYANSAKGRFTISRDNAKNTVYLQMNSLKPEDTAVYYCNARYGSSSFRWGDFEVWGQGTQVTVSS

SEQ ID NO: 315 - protein, 118 aa, synthetic construct
  source 1..118: note = PN0163
QVQLVESGGGSVQAGGSLRLSCAASGSIFNRAVMGWYRQAPGKQRESVATITRGGSANYADSVKGRFTISRVNAKNMVYLQMNSLEPEDTAVYYCNARSGLPYPNVYWGQGTQVTVSS

SEQ ID NO: 316 - protein, 122 aa, synthetic construct
  source 1..122: note = PN0164
QVQLVESGGGLVQAGGSLRLSCTASGRIFSINAMAWYRQAPGKQRELVATITSGGSTNYADSVKGRFAISRDNGKNTVFLQMNSLKPEDTAVYYCNADGVRGSFVSQGRVYWGQGTQVTVSS

SEQ ID NO: 317 - protein, 119 aa, synthetic construct
  source 1..119: note = PN0159
QVQLVESGGGLVQPGGSLRLSCAASGSISSDYTMGWYRQAPGKQRELVASITSAGTTNYADSVKGRFTISRDNAKNTVYLIMNSLKPEDTAVYYCNTKKFRIVGYRDYWGQGTQVTVSS

SEQ ID NO: 318 - protein, 122 aa, synthetic construct
  source 1..122: note = PN0165
QVQLVESGGGLVQAGGSLRLSCGASGSIFMINVMGWYRQAPGKERELVARINSGGTTNYADSAKGRFTISRDNAKNTVYLQMNSLKPEDTAVYYCNVNARRNFISNRYNDYWGQGTQVTVSS

SEQ ID NO: 319 - protein, 119 aa, synthetic construct
  source 1..119: note = PN0166
QVQLVESGGGPVLAGGSLRLSCAASGRVLVINTMGWYRQTPGQQRELVATSTNSGKVTYSDSVKGRFTISRDNSKKTVDLQMNSLKSEDTAVYYCYAGVRIGPATNAYWGRGTQVTVSS

SEQ ID NO: 320 - protein, 118 aa, synthetic construct
  source 1..118: note = PN0167
QVQLVESGGGLVQAGGSLRLSCAGSGSIRSFNSMAWYRQAPGKQRELVARIRTGGRTEYADSVKGRFTISRDNAKNTVYLQMNTLRPEDTAVYYCNADTLFPRPVNYWGQGTQVTVSS

SEQ ID NO: 321 - protein, 119 aa, synthetic construct
  source 1..119: note = PN0145
QVQLVESGGGLVQAGGSLRLSCAVPERMRSINVLAWYRQAPGKQRELVAGITSGDSTNYSEAVKGRFTISRDNAKNTVYLQMNSLKPEDTAVYYCHVEGRIRLNWGSYWGQGTQVTVSS

SEQ ID NO: 322 - protein, 227 aa, synthetic construct
  source 1..227: note = huFcIgG1
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 227 aa, synthetic construct; identical to SEQ ID NO: 322
  source 1..227: note = huFcIgG1_N297A

SEQ ID NO: 324 - protein, 227 aa, synthetic construct
  source 1..227: note = pN2037 (huFc IgG1 L234A _L235A)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 325 - protein, 18 aa, synthetic construct
  source 1..18: note = hinge sequence
DKTHTCPPCPAPELLGGP

(19) United States
(12) Patent Application Publication
(10) Pub. No.: US 2026/0103529 A1
(43) Pub. Date: Apr. 16, 2026

(54) Amino Acid Sequences Directed Against the Melanocortin 4 Receptor and Polypeptides Comprising the Same for the Treatment of MC4R-Related Diseases and Disorders

(71) Applicant: Confo Therapeutics N.V., Zwijnaarde (BE)

(72) Inventors: Christel Menet, Brussel (BE); Maarten Van Roy, Merelbeke (BE); Toon Laeremans, Dworp (BE); Rosa Barrocco, Brakel (BE); Stephanie Staelens, Wevelgem (BE); Veli-Pekka Jaakola, Zwijnaarde (BE); Kamila Skieterska, Gent (BE); Thomas Fontaine, Saint-Gilles (BE)

(21) Appl. No.: 19/122,123

(22) PCT Filed: Oct. 17, 2023
(86) PCT No.: PCT/EP2023/078855

§ 371 Date: Apr. 17, 2025

Related U.S. Application Data
(60) Provisional application No. 63/417,006, filed on Oct. 18, 2022.

(51) Int. Cl. C07K 16/28; A61K 39/00

(52) U.S. Cl. CPC C07K 16/2869; A61K 2039/505; C07K 2317/24; C07K 2317/52; C07K 2317/565; C07K 2317/569; C07K 2317/75

(57) ABSTRACT

The present invention relates to agonist VHH that are specific for (as defined herein) melanocortin 4 receptor (“MC4R”), as well as to proteins and polypeptides, that comprise or essentially consist of one or more such VHH sequences and medical uses to reduce body weight.

### CROSS REFERENCE TO RELATED APPLICATIONS

This application is a national phase entry under 35 U.S.C. § 371 of International Patent Application PCT/EP2023/078855, filed Oct. 17, 2023, designating the United States of America and published in English as International Patent Publication WO2024/083843 on Apr. 25, 2024, which claims the benefit under Article 8 of the Patent Cooperation Treaty to U.S. Provisional Application Ser. No. 63/417,006, filed Oct. 18, 2022, the entireties of which are hereby incorporated by reference.

### INCORPORATION BY REFERENCE

The ST.26 XML Sequence listing named “ST26SequenceListing”, created on Nov. 28, 2023, and having a size of 413,696 bytes, is hereby incorporated herein by this reference in its entirety.

### FIELD OF THE INVENTION

The present invention relates to amino acid sequences that are directed against (as defined herein) melanocortin 4 receptor (“MC4R”), as well as to compounds or constructs, and in particular proteins and polypeptides, that comprise or essentially consist of one or more such amino acid sequences (also referred to herein as “amino acid sequences of the invention”, “compounds of the invention”, and “polypeptides of the invention” and, in the case of a polypeptide or protein construct “constructs of the invention”, respectively).

The invention also relates to nucleic acids encoding such amino acid sequences and polypeptides (also referred to herein as “nucleic acids of the invention” or “nucleotide sequences of the invention”); to methods for preparing such amino acid sequences and polypeptides; to host cells expressing or capable of expressing such amino acid sequences or polypeptides; to compositions, and in particular to pharmaceutical compositions, that comprise such amino acid sequences, polypeptides, nucleic acids and/or host cells; and to uses of such amino acid sequences or polypeptides, nucleic acids, host cells and/or compositions, in particular for prophylactic, therapeutic or diagnostic purposes, such as the prophylactic, therapeutic or diagnostic purposes mentioned herein.

Other aspects, embodiments, advantages and applications of the invention will become clear from the further description herein.

The melanocortin 4 receptor (“MC4R”) is a Class A GPCR. It is an integral membrane protein with a short N-terminal domain and tiny extracellular loops. MC4R is predominantly expressed in the hypothalamus and is a key component for hunger and satiety signals. In particular, MC4R acts as a key switch in the leptin-melanocortin molecular axis that controls hunger and satiety. Brain-produced hormones such as α-melanocyte-stimulating hormone (agonist) and agouti-related peptide (inverse agonist) regulate the molecular communication of the MC4R axis but are promiscuous for melanocortin receptor subtypes and induce a wide array of biological effects. It has been described that impaired MC4R signalling (e.g. POMC deficiency) causes hyperphagia and dysregulated energy homeostasis which results in early-onset obesity. Prevalence of pathogenic MC4R mutations is estimated between 0.5 and 1% in obese adults (BMI>30). MC4R mutations are the most common form of monogenic obesity and have been implicated in 1% to 6% of early-onset severe obesity.

More in particular, based on pharmacologic and human genetic evidence, the hypothalamic melanocortin-4 receptor (MC4R) appears as a clinically validated key modulator of energy expenditure, satiety and thus body weight. Human subjects carrying loss-of-function MC4R mutations or showing deficiency in proopiomelanocortin (POMC), the prohormone processed into bio-active melanocortin receptor peptide agonists, often show hyperphagia and severe (early-onset) obesity (see for example Clément et al., Nat. Med. 24, 551-555 (2018) and Kühnen et al., N. Engl. J. Med. 375, 240-246 (2016)). Conversely, subjects carrying gain-of-function MC4R mutations show increased satiety and reduced food uptake, resulting in lower body weight (see for example Lotta. et al., Cell 177, 597-607 (2019)). Therapeutically intervening with MC4R agonists similarly increases satiety and reduces food uptake. MC4R is a rhodopsin-like class A peptide G-protein coupled receptor (GPCR) and belongs to the melanocortin receptor family which comprises 5 receptor subtypes (MC1R to MC5R). The main biological effects of melanocortin receptor stimulation are skin melanocyte pigmentation and immune regulation (MC1R), adrenal gland steroidogenesis (MC2R), fat mass regulation, control of growth and puberty (MC3R) and regulation of energy homeostasis and food uptake (MC4R) (see for example Mountjoy, et al. Science. 257, 1248-1251 (1992), Maaser, et al., Ann. N. Y. Acad. Sci. 2006, 1072, 123-134 (2006), Chida et al., Proc. Natl. Acad. Sci. U.S.A. 104, 18205-18210 (2007), Yanik and Durhan, J Clin Res Pediatr Endocrinol. 15, 1-6 (2023), Cone, Nat. Neurosci. 8, 571-578 (2005) and Gautron et al., Cell 161, 133-145 (2015)). The role of MC5R is less elucidated but seems to manage immune reaction and inflammatory response, thermoregulation, and exocrine secretion. With the exception of MC2R which is solely activated by the (endogenous) adrenocorticotropic hormone (ACTH), endogenous signal transduction is induced mainly by melanocyte-stimulating hormone isoforms (α-, β- and γ-MSH). MC1R, MC2R and MC5R can also be stimulated by ACTH. MC3R, MC4R and MC5R signaling is blocked by the naturally occurring agouti-related peptide (AGRP). All peptide agonists to MC1R, MC3R, MC4R and MC5R share the amino acid motif H×RW, which presents key interaction residues required to induce endogenous melanocortin signaling. MC4R primarily couples to the stimulatory G-protein transducer (Gs) to activate adenylyl cyclase (AC), resulting in intracellular cAMP production and downstream protein kinase A (PKA) activation. Besides Gs stimulation, MC4R has been shown to recruit other G-protein dependent cytosolic signal transducers such as Gi and Gq/11. In addition, MC4R also couples to the G-protein independent transducer protein β-arrestin or the ion channel Kir7.1 (see for example Xu et al., CMLS, 77, 3831-3840 (2020) and Kühnen et al., Trends Mol. Med. 25:136-148 (2019)). Setmelanotide (also known as RM-493 or under its brand name Imcivrec®) is a cyclic, eight amino acid peptide MC4R agonist that contains the H×RW motif (HFRW) and also activates MC3R, MC1R13 and, albeit with lower potency, MC5R (see references in14). While setmelanotide can induce Gs signaling, the clinical efficacy of setmelanotide on body weight regulation is hypothesized to be driven by its Gq/11-phospholipase C pathway bias (see for example Kühnen et al., Trends Mol. Med. 25:136-148 (2019) and Liu and Hruby., Journal of cellular and molecular medicine, 26, 4125-4136 (2022)). Setmelanotide was approved in 2020 for rare genetic obesity disorders after positive phase 3 clinical studies reported significant body weight loss and reduced hunger score 16 in leptin receptor (LEPR)- and pro-opiomelanocortin (POMC)-deficient patients. Patients carrying such genetic mutations also show impaired MC4R signaling. While setmelanotide therapy showed a positive clinical outcome, undesired side effects were demonstrated due to a lack of melanocortin receptor specificity. The most commonly reported off-target effect of setmelanotide is skin and hair hyperpigmentation due to the activation of MC1R17. As a consequence of these off-target effects, there is still keen interest in the discovery of MC4R specific agonist ligands as potentially safer anti-obesity therapeutics.

Agonists of MC4R are known in the art, and have been suggested for the treatment of genetic obesity, such as rare diseases caused by a deficiency in MC4R signalling or the MC4R pathway, such as for example Proopiomelanocortin (POMC) deficiency, diseases caused by deficiency in Proprotein Convertase Subtilisin/Kexin Type 1 (PCSK1), Steroid receptor coactivator-1 (SRC1), SH2B adapter protein 1 (SH2B1) or leptin receptor (LEPR), and the Alström, Smith-Magenis and Bardet-Biedl syndromes, where an MC4R agonist can substitute for the missing MSH signal.

For example, one MC4R agonist (setmelanotide, IMCRIVEE) has approved in the United States in 2020 and in Europe in 2021 for the treatment of genetic obesity. Reference is for example made to WO2007008704 and to Chen et al., The Journal of Clinical Endocrinology and Metabolism. 100 (4): 1639-45. doi: 10.1210/jc.2014-4024. PMC 4399297. PMID 25675384; Kievit et al., Diabetes. 62 (2): 490-7. doi: 10.2337/db12-0598. PMC 3554387. PMID 23048186. However, in addition to agonizing MC4R, setmelanotide is known to also agonize other melanocortin receptors (such as MC1R) leading to undesired side effects such as skin hyperpigmentation.

Another melanocortin receptor agonist (bremelanotide, VYLEESI) was approved in 2019 in the United States for treatment of low sexual desire in women. Bremelanotide has also been suggested as a possible treatment for erectile disfunction (King et al., Current Topics in Medicinal Chemistry. 7 (11): 1098-1106. doi: 10.2174/1568026610707011111. PMC 2694735. PMID 17584130). Bremelanotide is considered to be a non-selective agonist acting through multiple melanocortin receptors and primarily through MC3R and MC4R.

Generally, however, MC4R peptide agonists such as setmelanotide lack receptor selectivity and show off-target effects and one aim of the invention is to provide MC4R agonists that are more specific for MC4R and hence can be a more suitable agent for therapeutic intervention via MC4R, such as anti-obesity therapeutic intervention via MC4R.

MC4R agonists have for example also been suggested for the treatment of Prader-Willi syndrome (see for example WO2017/059076).

Other diseases and disorders associated with MC4R will be clear to the skilled person, based on the disclosure herein and the further references cited herein.

The polypeptides and compositions of the present invention can generally be used as agonists of MC4R, of MC4R-mediated signalling, of the biological pathways in which MC4R and/or MC4R-related signalling are involved, and/or more generally to modulate the biological mechanisms, responses and effects associated with such signalling or these pathways.

As such, the polypeptides and compositions of the present invention can be used for the prevention and treatment (as defined herein) of MC4R-related diseases and disorders. Generally, “MC4R-related diseases and disorders” can be defined as diseases and disorders that can be prevented and/or treated, respectively, by suitably administering to a subject in need thereof (i.e. having the disease or disorder or at least one symptom thereof and/or at risk of attracting or developing the disease or disorder) of either a polypeptide or composition of the invention (and in particular, of a pharmaceutically active amount thereof) and/or of a known active principle active against MC4R or a biological pathway or mechanism in which MC4R is involved (and in particular, of a pharmaceutically active amount thereof). Examples of such MC4R-related diseases and disorders will be clear to the skilled person based on the disclosure herein, and for example include the MC4R-related diseases and disorders mentioned in the prior art cited herein relating to MC4R-related diseases.

In particular, “MC4R-related diseases and disorders” as defined herein are diseases and disorders that can be prevented or treated by administering, to a subject in need thereof, of a therapeutically active amount of an MC4R agonist (such as setmelanotide, bremelanotide or another MC4R agonist described for therapeutic purposes) and/or of a polypeptides or composition of the present invention. Thus, it is envisaged that the polypeptides or composition of the present invention could be used for the prevention or treatment of any disease or disorder for which the use of setmelanotide and/or bremelanotide as a treatment has been approved or suggested.

Some specific but non-limiting examples of MC4R-related diseases and disorders that can be prevented or treated with a polypeptides or composition of the present invention include obesity (in particular genetic obesity), feeding disorders, Prader-Willi Syndrome, low sexual desire in women and erectile disorders. Reference is again for example made to the prior art cited herein relating to MC4R-related diseases and disorders.

Thus, without being limited thereto, the amino acid sequences and polypeptides of the invention can for example be used to prevent and/or to treat all diseases and disorders that are currently being prevented or treated with active principles that can modulate MC4R-mediated signalling, such as those mentioned in the prior art cited above. It is also envisaged that the polypeptides of the invention can be used to prevent and/or to treat all diseases and disorders for which treatment with such active principles is currently being developed, has been proposed, or will be proposed or developed in future. In addition, it is envisaged that, because of their favourable properties as further described herein, the polypeptides of the present invention may be used for the prevention and treatment of other diseases and disorders than those for which these known active principles are being used or will be proposed or developed; and/or that the polypeptides of the present invention may provide new methods and regimens for treating the diseases and disorders described herein.

Other applications and uses of the amino acid sequences and polypeptides of the invention will become clear to the skilled person from the further disclosure herein.

Generally, it is an object of the invention to provide pharmacologically active agents, as well as compositions comprising the same, that can be used in the diagnosis, prevention and/or treatment of MC4R-related diseases and disorders and of the further diseases and disorders mentioned herein; and to provide methods for the diagnosis, prevention and/or treatment of such diseases and disorders that involve the administration and/or use of such agents and compositions.

In particular, it is an object of the invention to provide such pharmacologically active agents, compositions and/or methods that have certain advantages compared to the agents, compositions and/or methods that are currently used and/or known in the art. These advantages will become clear from the further description below.

More in particular, it is an object of the invention to provide therapeutic proteins that can be used as pharmacologically active agents, as well as compositions comprising the same, for the diagnosis, prevention and/or treatment of MC4R-related diseases and disorders and of the further diseases and disorders mentioned herein; and to provide methods for the diagnosis, prevention and/or treatment of such diseases and disorders that involve the administration and/or the use of such therapeutic proteins and compositions.

Accordingly, it is a specific object of the present invention to provide amino acid sequences that are directed against (as defined herein) MC4R, in particular against MC4R from a warm-blooded animal, more in particular against MC4R from a mammal, and especially against human MC4R; and to provide proteins and polypeptides comprising or essentially consisting of at least one such amino acid sequence.

In particular, it is a specific object of the present invention to provide such amino acid sequences and such proteins and/or polypeptides that are suitable for prophylactic, therapeutic and/or diagnostic use in a warm-blooded animal, and in particular in a mammal, and more in particular in a human being.

More in particular, it is a specific object of the present invention to provide such amino acid sequences and such proteins and/or polypeptides that can be used for the prevention, treatment, alleviation and/or diagnosis of one or more diseases, disorders or conditions associated with MC4R and/or mediated by MC4R (such as the diseases, disorders and conditions mentioned herein) in a warm-blooded animal, in particular in a mammal, and more in particular in a human being.

It is also a specific object of the invention to provide such amino acid sequences and such proteins and/or polypeptides that can be used in the preparation of pharmaceutical or veterinary compositions for the prevention and/or treatment of one or more diseases, disorders or conditions associated with and/or mediated by MC4R (such as the diseases, disorders and conditions mentioned herein) in a warm-blooded animal, in particular in a mammal, and more in particular in a human being.

In the invention, generally, these objects are achieved by the use of the amino acid sequences, proteins, polypeptides and compositions that are described herein.

In general, the invention provides amino acid sequences that are directed against (as defined herein) and/or can specifically bind (as defined herein) to MC4R; as well as compounds and constructs, and in particular proteins and polypeptides, that comprise at least one such amino acid sequence.

More in particular, the invention provides amino acid sequences that can bind to MC4R with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein; as well as compounds and constructs, and in particular proteins and polypeptides, that comprise at least one such amino acid sequence.

Preferably, a monovalent amino acid sequence of the invention (or a polypeptide that contains only one amino acid sequence of the invention) is preferably such that it will bind to MC4R with an affinity less than 500 nM, preferably less than 200 nM, more preferably less than 10 nM, such as less than 500 pM.

Some preferred IC50 values for binding of the amino acid sequences or polypeptides of the invention to MC4R will become clear from the further description and examples herein.

For binding to MC4R, an amino acid sequence of the invention will usually contain within its amino acid sequence one or more amino acid residues or one or more stretches of amino acid residues (i.e. with each “stretch” comprising two or amino acid residues that are adjacent to each other or in close proximity to each other, i.e. in the primary or tertiary structure of the amino acid sequence) via which the amino acid sequence of the invention can bind to MC4R, which amino acid residues or stretches of amino acid residues thus form the “site” for binding to MC4R (also referred to herein as the “antigen binding site”).

The amino acid sequences provided by the invention are preferably in essentially isolated form (as defined herein), or form part of a protein or polypeptide of the invention (as defined herein), which may comprise or essentially consist of one or more amino acid sequences of the invention and which may optionally further comprise one or more further amino acid sequences (all optionally linked via one or more suitable linkers). For example, and without limitation, the one or more amino acid sequences of the invention may be used as a binding unit in such a protein or polypeptide, which may optionally contain one or more further amino acid sequences that can serve as a binding unit (i.e. against one or more other targets than MC4R), so as to provide a monovalent, multivalent or multispecific polypeptide of the invention, respectively, all as described herein. Such a protein or polypeptide may also be in essentially isolated form (as defined herein).

The amino acid sequences and polypeptides of the invention as such preferably essentially consist of a single amino acid chain that is not linked via disulphide bridges to any other amino acid sequence or chain (but that may or may not contain one or more intramolecular disulphide bridges. For example, it is known that Nanobodies—as described herein—may sometimes contain a disulphide bridge between CDR3 and CDR1 or FR2). However, it should be noted that one or more amino acid sequences of the invention may be linked to each other and/or to other amino acid sequences (e.g. via disulphide bridges) to provide peptide constructs that may also be useful in the invention (for example Fab′ fragments, F(ab′)₂ fragments, ScFv constructs, “diabodies” and other multispecific constructs. Reference is for example made to the review by Holliger and Hudson, Nat Biotechnol. 2005 September; 23 (9): 1126-36).

Generally, when an amino acid sequence of the invention (or a compound, construct or polypeptide comprising the same) is intended for administration to a subject (for example for therapeutic and/or diagnostic purposes as described herein), it is preferably either an amino acid sequence that does not occur naturally in said subject; or, when it does occur naturally in said subject, in essentially isolated form (as defined herein).

It will also be clear to the skilled person that for pharmaceutical use, the amino acid sequences of the invention (as well as compounds, constructs and polypeptides comprising the same) are preferably directed against human MC4R; whereas for veterinary purposes, the amino acid sequences and polypeptides of the invention are preferably directed against MC4R from the species to be treated, or at least cross-reactive with MC4R from the species to be treated.

Furthermore, an amino acid sequence of the invention may optionally, and in addition to the at least one binding site for binding against MC4R, contain one or more further binding sites for binding against other antigens, proteins or targets.

The efficacy of the amino acid sequences and polypeptides of the invention, and of compositions comprising the same, can be tested using any suitable in vitro assay, cell-based assay, in vivo assay and/or animal model known per se, or any combination thereof, depending on the specific disease or disorder involved. Suitable assays and animal models will be clear to the skilled person, and for example include the diet-induced obese (DIO) mouse models used in the Experimental Part below (reference is for example made to Kumar et al., Peptides. 2009 October; 30 (10): 1892-1900; Clemmensen et al, EMBO Mol Med (2015) 7:288-298; and Strader et al, The Journal of Pharmacology and Experimental Therapeutics, 2007, Vol. 322, No. 3, 1153-1161) as well as the assays and animal models used in the experimental part below and in the prior art cited herein.

Also, according to the invention, amino acid sequences and polypeptides that are directed against MC4R from a first species of warm-blooded animal may or may not show cross-reactivity with MC4R from one or more other species of warm-blooded animal. For example, amino acid sequences and polypeptides directed against human MC4R may or may not show cross reactivity with MC4R from one or more other species of primates (such as, without limitation, monkeys from the genus Macaca (such as, and in particular, cynomolgous monkeys (Macaca fascicularis) and/or rhesus monkeys (Macaca mulatta)) and baboon (Papio ursinus)) and/or with MC4R from one or more species of animals that are often used in animal models for diseases (for example mouse, rat, rabbit, pig or dog), and in particular in animal models for diseases and disorders associated with MC4R (such as the species and animal models mentioned herein). In this respect, it will be clear to the skilled person that such cross-reactivity, when present, may have advantages from a drug development point of view, since it allows the amino acid sequences and polypeptides against human MC4R to be tested in such disease models.

More generally, amino acid sequences and polypeptides of the invention that are cross-reactive with MC4R from multiple species of mammal will usually be advantageous for use in veterinary applications, since it will allow the same amino acid sequence or polypeptide to be used across multiple species. Thus, it is also encompassed within the scope of the invention that amino acid sequences and polypeptides directed against MC4R from one species of animal (such as amino acid sequences and polypeptides against human MC4R) can be used in the treatment of another species of animal, as long as the use of the amino acid sequences and/or polypeptides provide the desired effects in the species to be treated.

The present invention is in its broadest sense also not particularly limited to or defined by a specific antigenic determinant, epitope, part, domain, subunit or confirmation (where applicable) of MC4R against which the amino acid sequences and polypeptides of the invention are directed. For example, the amino acid sequences and polypeptides may or may not be directed against an “interaction site” (as defined herein). In one specific, but non-limiting aspect, the amino acid sequences and polypeptides of the invention are preferably (at least partly (meaning with at least one CDR, such as with CDR3) directed against an interaction site (as defined herein), and in particular against the binding site of a natural ligand, and more in particular against the binding site of alpha-MSH (e.g. such that at least one CDR, such as CDR3, binds to and/or overlaps with the binding site of a natural ligand, and in particular with the binding site of alpha-MSH).

As further described herein, a polypeptide of the invention may contain two or more amino acid sequences of the invention that are directed against MC4R. Generally, such polypeptides will bind to MC4R with increased avidity compared to a single amino acid sequence of the invention. Such a polypeptide may for example comprise two amino acid sequences of the invention that are directed against the same antigenic determinant, epitope, part, domain, subunit or confirmation (where applicable) of MC4R (which may or may not be an interaction site); or comprise at least one “first” amino acid sequence of the invention that is directed against a first same antigenic determinant, epitope, part, domain, subunit or confirmation (where applicable) of MC4R (which may or may not be an interaction site); and at least one “second” amino acid sequence of the invention that is directed against a second antigenic determinant, epitope, part, domain, subunit or confirmation (where applicable) different from the first (and which again may or may not be an interaction site). Preferably, in such “biparatopic” polypeptides of the invention, at least one amino acid sequence of the invention is directed against an interaction site (as defined herein), although the invention in its broadest sense is not limited thereto.

Also, when the target is part of a binding pair (for example, a receptor-ligand binding pair), the amino acid sequences and polypeptides may be such that they compete with the cognate binding partner (e.g. the ligand, receptor or other binding partner, as applicable) for binding to the target, and/or such that they (fully or partially) neutralize binding of the binding partner to the target.

It is also within the scope of the invention that, where applicable, an amino acid sequence of the invention can bind to two or more antigenic determinants, epitopes, parts, domains, subunits or confirmations of MC4R. In such a case, the antigenic determinants, epitopes, parts, domains or subunits of MC4R to which the amino acid sequences and/or polypeptides of the invention bind may be essentially the same (for example, if MC4R contains repeated structural motifs or occurs in a multimeric form) or may be different (and in the latter case, the amino acid sequences and polypeptides of the invention may bind to such different antigenic determinants, epitopes, parts, domains, subunits of MC4R with an affinity and/or specificity which may be the same or different). Also, for example, when MC4R exists in an activated conformation and in an inactive conformation, the amino acid sequences and polypeptides of the invention may bind to either one of these confirmation, or may bind to both these confirmations (i.e. with an affinity and/or specificity which may be the same or different). Also, for example, the amino acid sequences and polypeptides of the invention may bind to a conformation of MC4R in which it is bound to a pertinent ligand, may bind to a conformation of MC4R in which it not bound to a pertinent ligand, or may bind to both such conformations (again with an affinity and/or specificity which may be the same or different).

It is also expected that the amino acid sequences and polypeptides of the invention will generally bind to all naturally occurring or synthetic analogs, variants, mutants, alleles, parts and fragments of MC4R; or at least to those analogs, variants, mutants, alleles, parts and fragments of MC4R that contain one or more antigenic determinants or epitopes that are essentially the same as the antigenic determinant(s) or epitope(s) to which the amino acid sequences and polypeptides of the invention bind in MC4R (e.g. in wild-type MC4R). Again, in such a case, the amino acid sequences and polypeptides of the invention may bind to such analogs, variants, mutants, alleles, parts and fragments with an affinity and/or specificity that are the same as, or that are different from (i.e. higher than or lower than), the affinity and specificity with which the amino acid sequences of the invention bind to (wild-type) MC4R. It is also included within the scope of the invention that the amino acid sequences and polypeptides of the invention bind to some analogs, variants, mutants, alleles, parts and fragments of MC4R, but not to others.

When MC4R exists in a monomeric form and in one or more multimeric forms, it is within the scope of the invention that the amino acid sequences and polypeptides of the invention only bind to MC4R in monomeric form, only bind to MC4R in multimeric form, or bind to both the monomeric and the multimeric form. Again, in such a case, the amino acid sequences and polypeptides of the invention may bind to the monomeric form with an affinity and/or specificity that are the same as, or that are different from (i.e. higher than or lower than), the affinity and specificity with which the amino acid sequences of the invention bind to the multimeric form.

Also, when MC4R can associate with other proteins or polypeptides to form protein complexes (e.g. with multiple subunits), it is within the scope of the invention that the amino acid sequences and polypeptides of the invention bind to MC4R in its non-associated state, bind to MC4R in its associated state, or bind to both. In all these cases, the amino acid sequences and polypeptides of the invention may bind to such multimers or associated protein complexes with an affinity and/or specificity that may be the same as or different from (i.e. higher than or lower than) the affinity and/or specificity with which the amino acid sequences and polypeptides of the invention bind to MC4R in its monomeric and non-associated state.

Also, as will be clear to the skilled person, proteins or polypeptides that contain two or more amino acid sequences directed against MC4R may bind with higher avidity to MC4R than the corresponding monomeric amino acid sequence(s). For example, and without limitation, proteins or polypeptides that contain two or more amino acid sequences directed against different epitopes of MC4R may (and usually will) bind with higher avidity than each of the different monomers, and proteins or polypeptides that contain two or more amino acid sequences directed against MC4R may (and usually will) bind also with higher avidity to a multimer of MC4R.

Generally, amino acid sequences and polypeptides of the invention will at least bind to those forms of MC4R (including monomeric, multimeric and associated forms) that are the most relevant from a biological and/or therapeutic point of view, as will be clear to the skilled person.

It is also within the scope of the invention to use parts, fragments, analogs, mutants, variants, alleles and/or derivatives of the amino acid sequences and polypeptides of the invention, and/or to use proteins or polypeptides comprising or essentially consisting of one or more of such parts, fragments, analogs, mutants, variants, alleles and/or derivatives, as long as these are suitable for the uses envisaged herein. Such parts, fragments, analogs, mutants, variants, alleles and/or derivatives will usually contain (at least part of) a functional antigen-binding site for binding against MC4R; and more preferably will be capable of specific binding to MC4R, and even more preferably capable of binding to MC4R with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein. Some non-limiting examples of such parts, fragments, analogs, mutants, variants, alleles, derivatives, proteins and/or polypeptides will become clear from the further description herein. Additional fragments or polypeptides of the invention may also be provided by suitably combining (i.e. by linking or genetic fusion) one or more (smaller) parts or fragments as described herein.

In one specific, but non-limiting aspect of the invention, which will be further described herein, such analogs, mutants, variants, alleles, derivatives have an increased half-life in serum (as further described herein) compared to the amino acid sequence from which they have been derived. For example, an amino acid sequence of the invention may be linked (chemically or otherwise) to one or more groups or moieties that extend the half-life (such as PEG), so as to provide a derivative of an amino acid sequence of the invention with increased half-life.

In one specific, but non-limiting aspect, the amino acid sequence of the invention may be an amino acid sequence that comprises an immunoglobulin fold or may be an amino acid sequence that, under suitable conditions (such as physiological conditions) is capable of forming an immunoglobulin fold (i.e. by folding). Reference is inter alia made to the review by Halaby et al., J. (1999) Protein Eng. 12, 563-71. Preferably, when properly folded so as to form an immunoglobulin fold, such an amino acid sequence is capable of specific binding (as defined herein) to MC4R; and more preferably capable of binding to MC4R with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein. Also, parts, fragments, analogs, mutants, variants, alleles and/or derivatives of such amino acid sequences are preferably such that they comprise an immunoglobulin fold or are capable for forming, under suitable conditions, an immunoglobulin fold.

In particular, but without limitation, the amino acid sequences of the invention may be amino acid sequences that essentially consist of 4 framework regions (FR1 to FR4 respectively) and 3 complementarity determining regions (CDR1 to CDR3 respectively); or any suitable fragment of such an amino acid sequence (which will then usually contain at least some of the amino acid residues that form at least one of the CDR's, as further described herein).

The amino acid sequences of the invention may in particular be an immunoglobulin sequence or a suitable fragment thereof, and more in particular be an immunoglobulin variable domain sequence or a suitable fragment thereof, such as light chain variable domain sequence (e.g. a V_{L}-sequence) or a suitable fragment thereof; or a heavy chain variable domain sequence (e.g. a V_{H}-sequence) or a suitable fragment thereof. When the amino acid sequence of the invention is a heavy chain variable domain sequence, it may be a heavy chain variable domain sequence that is derived from a conventional four-chain antibody (such as, without limitation, a V_{H} sequence that is derived from a human antibody) or be a so-called V_{HH}-Sequence (as defined herein) that is derived from a so-called “heavy chain antibody” (as defined herein).

However, it should be noted that the invention is not limited as to the origin of the amino acid sequence of the invention (or of the nucleotide sequence of the invention used to express it), nor as to the way that the amino acid sequence or nucleotide sequence of the invention is (or has been) generated or obtained. Thus, the amino acid sequences of the invention may be naturally occurring amino acid sequences (from any suitable species) or synthetic or semi-synthetic amino acid sequences. In a specific but non-limiting aspect of the invention, the amino acid sequence is a naturally occurring immunoglobulin sequence (from any suitable species) or a synthetic or semi-synthetic immunoglobulin sequence, including but not limited to “humanized” (as defined herein) immunoglobulin sequences (such as partially or fully humanized mouse or rabbit immunoglobulin sequences, and in particular partially or fully humanized V_{HH} sequences or Nanobodies), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing. Reference is for example made to the standard handbooks, as well as to the further description and prior art mentioned herein.

The term “potency maturation” is used herein to generally refer to methods and techniques for identifying and/or generating variants of a (parental) sequence by making one or more variants (and usually a collection or library of variants) in which each variant has one or more amino acid changes in one or more of the CDRs compared to the starting sequence and testing the variant for potency (i.e. using a suitable potency assay) in order to identify variants with improved potency compared to the parental/starting sequence or one or more other (related) sequence. As will be clear to the skilled person, such potency maturation can generally be performed in a manner that is essentially the same as or analogous to affinity maturation, but by measuring and comparing potency of the resulting variants (i.e. using a suitable potency assay) instead of measuring and comparing affinity.

Similarly, the nucleotide sequences of the invention may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

The amino acid sequence of the invention may in particular be a domain antibody (or an amino acid sequence that is suitable for use as a domain antibody), a single domain antibody (or an amino acid sequence that is suitable for use as a single domain antibody), a “dAb” (or an amino acid sequence that is suitable for use as a dAb) or a Nanobody™ (as defined herein, and including but not limited to a V_{HH} sequence); other single variable domains, or any suitable fragment of any one thereof. For a general description of (single) domain antibodies, reference is also made to the prior art cited above, as well as to EP 0 368 684. For the term “dAb's”, reference is for example made to Ward et al. (Nature 1989 Oct. 12; 341 (6242): 544-6), to Holt et al., Trends Biotechnol., 2003, 21 (11): 484-490; as well as to for example WO 06/030220, WO 06/003388 and other published patent applications of Domantis Ltd. It should also be noted that, although less preferred in the context of the present invention because they are not of mammalian origin, single domain antibodies or single variable domains can be derived from certain species of shark (for example, the so-called “IgNAR domains”, see for example WO 05/18629).

In particular, the amino acid sequence of the invention may be a Nanobody® (as defined herein) or a suitable fragment thereof. [Note: Nanobody®, Nanobodies® and Nanoclone® are registered trademarks of Ablynx N.V./Such Nanobodies directed against MC4R will also be referred to herein as “Nanobodies of the invention”.

For a general description of Nanobodies, reference is made to the further description below, as well as to the prior art cited herein. In this respect, it should however be noted that this description and the prior art mainly described Nanobodies of the so-called “V_{H}3 class” (i.e. Nanobodies with a high degree of sequence homology to human germline sequences of the V_{H}3 class such as DP-47, DP-51 or DP-29), which Nanobodies form a preferred aspect of this invention. It should however be noted that the invention in its broadest sense generally covers any type of Nanobody directed against MC4R, and for example also covers the Nanobodies belonging to the so-called “V_{H}4 class” (i.e. Nanobodies with a high degree of sequence homology to human germline sequences of the V_{H}4 class such as DP-78), as for example described in WO 07/118670.

Generally, Nanobodies (in particular V_{HH} sequences and partially humanized Nanobodies) can in particular be characterized by the presence of one or more “Hallmark residues” (as described herein) in one or more of the framework sequences (again as further described herein).

Thus, generally, a Nanobody can be defined as an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody can be an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody can be an amino acid sequence with the (general) structure
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and in which:
i) preferably one or more of the amino acid residues at positions 11, 37, 44, 45, 47, 83, 84, 103, 104 and 108 according to the Kabat numbering are chosen from the Hallmark residues mentioned in Table A-3 below;
   and in which:
ii) said amino acid sequence has at least 80% amino acid identity with at least one of the amino acid sequences of SEQ ID NO's: 1 to 22, in which for the purposes of determining the degree of amino acid identity, the amino acid residues that form the CDR sequences (indicated with X in the sequences of SEQ ID NO's: 1 to 22) are disregarded.

In these Nanobodies, the CDR sequences are generally as further defined herein.

Thus, the invention also relates to such Nanobodies that can bind to (as defined herein) and/or are directed against MC4R, to suitable fragments thereof, as well as to polypeptides that comprise or essentially consist of one or more of such Nanobodies and/or suitable fragments.

SEQ ID NO's: 182 to 189 and 306 to 321 give the amino acid sequences of a number of V_{HH} sequences that have been raised against MC4R. Of these, SEQ ID NO's: 185 to 189 are preferred but non-limiting examples of VHH sequences that have been obtained through potency maturation of the corresponding parental VHH (i.e. pN0162; SEQ ID NO:128). Thus, generally, in the invention, the CDR sequences that are present in the VHH sequences of SEQ ID NO's: 185 to 189 will generally be preferred over the CDR sequences that are present in the VHH sequences of SEQ ID NO's: 182 to 184. In particular, in the invention, the respective combinations of CDR1, CDR2 and CDR3 that are present in each of the VHH sequences of SEQ ID NO's: 185 to 189, respectively, will be preferred over the combinations of CDR1, CDR2 and CDR3 that are present in the VHH sequences of SEQ ID NO's: 182 to 184, respectively. For these CDR sequences and combinations of CDR sequences, reference is also made to Table A-2 below.

Also, the VHHs of SEQ ID NO's: 185 to 189 have been humanized compared to the parental V_{HH} (i.e. pN0162; SEQ ID NO:128) and the humanizing substitutions that are present in the VHHs of SEQ ID NO's: 185 to 189 are some non-limiting examples of humanizing substitutions that are present in the VHH sequences of the invention.

Also, in the invention, the VHH sequence of SEQ ID NO: 189 (pN2121) is particularly preferred, as are each of the CDR1, CDR2 and CDR3 sequences present in the preferred VHH sequence of SEQ ID NO: 189 (pN2121) as well as the combination of CDR1, CDR2 and CDR3 sequences that is present in the preferred VHH sequence of SEQ ID NO: 189 (pN2121). Reference is again made to Table A-2 below.

In particular, the invention in some specific aspects provides:
amino acid sequences that are directed against (as defined herein) MC4R and that have at least 80%, preferably at least 85%, such as 90% or 95% or more sequence identity with at least one of the amino acid sequences of SEQ ID NO's: 182 to 189 and 306 to 321. These amino acid sequences may further be such that they neutralize binding of the cognate ligand to MC4R; and/or compete with the cognate ligand for binding to MC4R; and/or are directed against an interaction site (as defined herein) on MC4R (such as the ligand binding site);
amino acid sequences that cross-block (as defined herein) the binding of at least one of the amino acid sequences of SEQ ID NO's: 182 to 189 and 306 to 321 to MC4R and/or that compete with at least one of the amino acid sequences of SEQ ID NO's: 182 to 189 and 306 to 321 for binding to MC4R. Again, these amino acid sequences may further be such that they neutralize binding of the cognate ligand to MC4R; and/or compete with the cognate ligand for binding to MC4R; and/or are directed against an interaction site (as defined herein) on MC4R (such as the ligand binding site);
   which amino acid sequences may be as further described herein (and may for example be Nanobodies); as well as polypeptides of the invention that comprise one or more of such amino acid sequences (which may be as further described herein, and may for example be bispecific and/or biparatopic polypeptides as described herein), and nucleic acid sequences that encode such amino acid sequences and polypeptides. Such amino acid sequences and polypeptides do not include any naturally occurring ligands.

In some other specific aspects, the invention provides amino acid sequences of the invention that are specific for (as defined herein) MC4R compared to MC1R, MC2R and MC3R, for example as determined using the methodology described in Example 4 below; which amino acid sequences of the invention may be as further described herein (and may for example be Nanobodies); as well as polypeptides of the invention that comprise one or more of such amino acid sequences (which may be as further described herein, and may for example be bispecific and/or biparatopic polypeptides as described herein or the Fc based constructs which are preferred according to one specific aspect of the invention), and nucleic acid sequences that encode such amino acid sequences and polypeptides. Such amino acid sequences and polypeptides do not include any naturally occurring ligands.

Accordingly, some particularly preferred Nanobodies of the invention are Nanobodies which can bind (as further defined herein) to and/or are directed against to MC4R and which:
i) have at least 80% amino acid identity with at least one of the amino acid sequences of SEQ ID NO's: 182 to 189 and 306 to 321, in which for the purposes of determining the degree of amino acid identity, the amino acid residues that form the CDR sequences are disregarded. In this respect, reference is also made to Table A-1, which lists the framework 1 sequences (SEQ ID NO's: 126 to 133 and 194 to 207), framework 2 sequences (SEQ ID NO's: 142 to 149 and 226 to 241), framework 3 sequences (SEQ ID NO's: 158 to 165 and 258 to 273) and framework 4 sequences (SEQ ID NO's: 174 to 181 and 290 to 305) of the Nanobodies of SEQ ID NO's: 182 to 189 and 306 to 321 (with respect to the amino acid residues at positions 1 to 4 and 27 to 30 of the framework 1 sequences, reference is also made to the comments made below. Thus, for determining the degree of amino acid identity, these residues are preferably disregarded);
   and in which:
ii) preferably one or more of the amino acid residues at positions 11, 37, 44, 45, 47, 83, 84, 103, 104 and 108 according to the Kabat numbering are chosen from the Hallmark residues mentioned in Table A-3 below.

In these Nanobodies, the CDR sequences are generally as further defined herein.

Also, as mentioned, the Nanobodies described herein and compounds, constructs and polypeptides comprising the same (also as further described herein) are agonists of MC4R, as further described herein (for example, as determined using a cAMP assay).

Again, such Nanobodies may be derived in any suitable manner and from any suitable source, and may for example be naturally occurring V_{HH} sequences (i.e. from a suitable species of Camelid) or synthetic or semi-synthetic amino acid sequences, including but not limited to “humanized” (as defined herein) Nanobodies, “camelized” (as defined herein) immunoglobulin sequences (and in particular camelized heavy chain variable domain sequences), as well as Nanobodies that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein. Also, when a Nanobody comprises a V_{HH} sequence, said Nanobody may be suitably humanized, as further described herein, so as to provide one or more further (partially or fully) humanized Nanobodies of the invention. Similarly, when a Nanobody comprises a synthetic or semi-synthetic sequence (such as a partially humanized sequence), said Nanobody may optionally be further suitably humanized, again as described herein, again so as to provide one or more further (partially or fully) humanized Nanobodies of the invention.

In particular, humanized Nanobodies may be amino acid sequences that are as generally defined for Nanobodies in the previous paragraphs, but in which at least one amino acid residue is present (and in particular, in at least one of the framework residues) that is and/or that corresponds to a humanizing substitution (as defined herein). Some preferred, but non-limiting humanizing substitutions (and suitable combinations thereof) will become clear to the skilled person based on the disclosure herein. In addition, or alternatively, other potentially useful humanizing substitutions can be ascertained by comparing the sequence of the framework regions of a naturally occurring V_{HH} sequence with the corresponding framework sequence of one or more closely related human V_{H} sequences, after which one or more of the potentially useful humanizing substitutions (or combinations thereof) thus determined can be introduced into said V_{HH} sequence (in any manner known per se, as further described herein) and the resulting humanized V_{HH} sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person based on the disclosure herein. Also, based on the foregoing, (the framework regions of) a Nanobody may be partially humanized or fully humanized.

Some particularly preferred humanized Nanobodies of the invention are humanized variants of the Nanobodies of SEQ ID NO's: 182 to 189 and 306 to 321, of which the amino acid sequences of SEQ ID NO's: 185 to 189 are some especially preferred examples.

Thus, some other preferred Nanobodies of the invention are Nanobodies which can bind (as further defined herein) to MC4R and which:
i) are a humanized variant of one of the amino acid sequences of SEQ ID NO's: 182 to 189 and 306 to 321; and/or
ii) have at least 80% amino acid identity with at least one of the amino acid sequences of SEQ ID NO's: 182 to 189 and 306 to 321 and/or at least one of the amino acid sequences of SEQ ID NO's: 185 to 189, in which for the purposes of determining the degree of amino acid identity, the amino acid residues that form the CDR sequences are disregarded;
   and in which:
i) preferably one or more of the amino acid residues at positions 11, 37, 44, 45, 47, 83, 84, 103, 104 and 108 according to the Kabat numbering are chosen from the Hallmark residues mentioned in Table A-3 below.

According to another specific aspect of the invention, the invention provides a number of stretches of amino acid residues (i.e. small peptides) that are particularly suited for binding to MC4R. These stretches of amino acid residues may be present in, and/or may be incorporated into, an amino acid sequence of the invention, in particular in such a way that they form (part of) the antigen binding site of an amino acid sequence of the invention. As these stretches of amino acid residues were first generated as CDR sequences of heavy chain antibodies or V_{HH} sequences that were raised against MC4R (or may be based on and/or derived from such CDR sequences, as further described herein), they will also generally be referred to herein as “CDR sequences” (i.e. as CDR1 sequences, CDR2 sequences and CDR3 sequences, respectively). It should however be noted that the invention in its broadest sense is not limited to a specific structural role or function that these stretches of amino acid residues may have in an amino acid sequence of the invention, as long as these stretches of amino acid residues allow the amino acid sequence of the invention to bind to MC4R. Thus, generally, the invention in its broadest sense comprises any amino acid sequence that is capable of binding to MC4R and that comprises one or more CDR sequences as described herein, and in particular a suitable combination of two or more such CDR sequences, that are suitably linked to each other via one or more further amino acid sequences, such that the entire amino acid sequence forms a binding domain and/or binding unit that is capable of binding to MC4R. It should however also be noted that the presence of only one such CDR sequence in an amino acid sequence of the invention may by itself already be sufficient to provide an amino acid sequence of the invention that is capable of binding to MC4R; reference is for example again made to the so-called “Expedite fragments” described in WO 03/050531.

Thus, in another specific, but non-limiting aspect, the amino acid sequence of the invention may be an amino acid sequence that comprises at least one amino acid sequence that is chosen from the group consisting of the CDR1 sequences, CDR2 sequences and CDR3 sequences that are described herein (or any suitable combination thereof). In particular, an amino acid sequence of the invention may be an amino acid sequence that comprises at least one antigen binding site, wherein said antigen binding site comprises at least one amino acid sequence that is chosen from the group consisting of the CDR1 sequences, CDR2 sequences and CDR3 sequences that are described herein (or any suitable combination thereof).

Generally, in this aspect of the invention, the amino acid sequence of the invention may be any amino acid sequence that comprises at least one stretch of amino acid residues, in which said stretch of amino acid residues has an amino acid sequence that corresponds to the sequence of at least one of the CDR sequences described herein. Such an amino acid sequence may or may not comprise an immunoglobulin fold. For example, and without limitation, such an amino acid sequence may be a suitable fragment of an immunoglobulin sequence that comprises at least one such CDR sequence, but that is not large enough to form a (complete) immunoglobulin fold (reference is for example again made to the “Expedite fragments” described in WO 03/050531). Alternatively, such an amino acid sequence may be a suitable “protein scaffold” that comprises least one stretch of amino acid residues that corresponds to such a CDR sequence (i.e. as part of its antigen binding site). Suitable scaffolds for presenting amino acid sequences will be clear to the skilled person, and for example comprise, without limitation, to binding scaffolds based on or derived from immunoglobulins (i.e. other than the immunoglobulin sequences already described herein), protein scaffolds derived from protein A domains (such as Affibodies™), tendamistat, fibronectin, lipocalin, CTLA-4, T-cell receptors, designed ankyrin repeats, avimers and PDZ domains (Binz et al., Nat. Biotech 2005, Vol 23:1257), and binding moieties based on DNA or RNA including but not limited to DNA or RNA aptamers (Ulrich et al., Comb Chem High Throughput Screen 2006 9 (8): 619-32).

Again, any amino acid sequence of the invention that comprises one or more of these CDR sequences is preferably such that it can specifically bind (as defined herein) to MC4R, and more in particular such that it can bind to MC4R with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein), that is as defined herein.

More in particular, the amino acid sequences according to this aspect of the invention may be any amino acid sequence that comprises at least one antigen binding site, wherein said antigen binding site comprises at least two amino acid sequences that are chosen from the group consisting of the CDR1 sequences described herein, the CDR2 sequences described herein and the CDR3 sequences described herein, such that (i) when the first amino acid sequence is chosen from the CDR1 sequences described herein, the second amino acid sequence is chosen from the CDR2 sequences described herein or the CDR3 sequences described herein; (ii) when the first amino acid sequence is chosen from the CDR2 sequences described herein, the second amino acid sequence is chosen from the CDR1 sequences described herein or the CDR3 sequences described herein; or (iii) when the first amino acid sequence is chosen from the CDR3 sequences described herein, the second amino acid sequence is chosen from the CDR1 sequences described herein or the CDR3 sequences described herein.

Even more in particular, the amino acid sequences of the invention may be amino acid sequences that comprise at least one antigen binding site, wherein said antigen binding site comprises at least three amino acid sequences that are chosen from the group consisting of the CDR1 sequences described herein, the CDR2 sequences described herein and the CDR3 sequences described herein, such that the first amino acid sequence is chosen from the CDR1 sequences described herein, the second amino acid sequence is chosen from the CDR2 sequences described herein, and the third amino acid sequence is chosen from the CDR3 sequences described herein. Preferred combinations of CDR1, CDR2 and CDR3 sequences will become clear from the further description herein. As will be clear to the skilled person, such an amino acid sequence is preferably an immunoglobulin sequence (as further described herein), but it may for example also be any other amino acid sequence that comprises a suitable scaffold for presenting said CDR sequences.

Thus, in one specific, but non-limiting aspect, the invention relates to an amino acid sequence directed against MC4R, that comprises one or more stretches of amino acid residues chosen from the group consisting of:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;
d) the amino acid sequences of SEQ ID NO's: 150 to 157 and 242 to 257;
e) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
g) the amino acid sequences of SEQ ID NO's: 166 to 173 and 274 to 289;
h) amino acid sequences that have at least 80% amino acid identity with at least one of the amino acid sequences of SEQ ID NO's: 166 to 173 and 274 to 289;
i) amino acid sequences that have 3, 2, or 1 amino acid difference with at least one of the amino acid sequences of SEQ ID NO's: 166 to 173 and 274 to 289;
or any suitable combination thereof.

When an amino acid sequence of the invention contains one or more amino acid sequences according to b) and/or c):
i) any amino acid substitution in such an amino acid sequence according to b) and/or c) is preferably, and compared to the corresponding amino acid sequence according to a), a conservative amino acid substitution, (as defined herein);
and/or
ii) the amino acid sequence according to b) and/or c) preferably only contains amino acid substitutions, and no amino acid deletions or insertions, compared to the corresponding amino acid sequence according to a);
and/or
iii) the amino acid sequence according to b) and/or c) may be an amino acid sequence that is derived from an amino acid sequence according to a) by means of affinity maturation using one or more techniques of affinity maturation known per se.

Similarly, when an amino acid sequence of the invention contains one or more amino acid sequences according to e) and/or f):
i) any amino acid substitution in such an amino acid sequence according to e) and/or f) is preferably, and compared to the corresponding amino acid sequence according to d), a conservative amino acid substitution, (as defined herein);
and/or
ii) the amino acid sequence according to e) and/or f) preferably only contains amino acid substitutions, and no amino acid deletions or insertions, compared to the corresponding amino acid sequence according to d);
and/or
iii) the amino acid sequence according to e) and/or f) may be an amino acid sequence that is derived from an amino acid sequence according to d) by means of affinity maturation using one or more techniques of affinity maturation known per se.

Also, similarly, when an amino acid sequence of the invention contains one or more amino acid sequences according to h) and/or i):
i) any amino acid substitution in such an amino acid sequence according to h) and/or i) is preferably, and compared to the corresponding amino acid sequence according to g), a conservative amino acid substitution, (as defined herein);
and/or
ii) the amino acid sequence according to h) and/or i) preferably only contains amino acid substitutions, and no amino acid deletions or insertions, compared to the corresponding amino acid sequence according to g);
and/or
iii) the amino acid sequence according to h) and/or i) may be an amino acid sequence that is derived from an amino acid sequence according to g) by means of affinity maturation using one or more techniques of affinity maturation known per se.

It should be understood that the last preceding paragraphs also generally apply to any amino acid sequences of the invention that comprise one or more amino acid sequences according to b), c), e), f), h) or i), respectively.

In this specific aspect, the amino acid sequence preferably comprises one or more stretches of amino acid residues chosen from the group consisting of:
i) the amino acid sequences of SEQ ID NO's: 134 to 141 and 210 to 225;
ii) the amino acid sequences of SEQ ID NO's: 150 to 157 and 242 to 257; and
iii) the amino acid sequences of SEQ ID NO's: 166 to 173 and 274 to 289;
or any suitable combination thereof.

Also, preferably, in such an amino acid sequence, at least one of said stretches of amino acid residues forms part of the antigen binding site for binding against MC4R.

In a more specific, but again non-limiting aspect, the invention relates to an amino acid sequence directed against MC4R, that comprises two or more stretches of amino acid residues chosen from the group consisting of:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;
d) the amino acid sequences of SEQ ID NO's: 150 to 157 and 242 to 257;
e) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
g) the amino acid sequences of SEQ ID NO's: 166 to 173 and 274 to 289;
h) amino acid sequences that have at least 80% amino acid identity with at least one of the amino acid sequences of SEQ ID NO's: 166 to 173 and 274 to 289;
i) amino acid sequences that have 3, 2, or 1 amino acid difference with at least one of the amino acid sequences of SEQ ID NO's: 166 to 173 and 274 to 289;
such that (i) when the first stretch of amino acid residues corresponds to one of the amino acid sequences according to a), b) or c), the second stretch of amino acid residues corresponds to one of the amino acid sequences according to d), e), f), g), h) or i); (ii) when the first stretch of amino acid residues corresponds to one of the amino acid sequences according to d), e) or f), the second stretch of amino acid residues corresponds to one of the amino acid sequences according to a), b), c), g), h) or i); or (iii) when the first stretch of amino acid residues corresponds to one of the amino acid sequences according to g), h) or i), the second stretch of amino acid residues corresponds to one of the amino acid sequences according to a), b), c), d), c) or f).

In this specific aspect, the amino acid sequence preferably comprises two or more stretches of amino acid residues chosen from the group consisting of:
i) the amino acid sequences of SEQ ID NO's: 134 to 141 and 210 to 225;
ii) the amino acid sequences of SEQ ID NO's: 150 to 157 and 242 to 257; and
iii) the amino acid sequences of SEQ ID NO's: 166 to 173 and 274 to 289;
such that, (i) when the first stretch of amino acid residues corresponds to one of the amino acid sequences of SEQ ID NO's: 134 to 141 and 210 to 225, the second stretch of amino acid residues corresponds to one of the amino acid sequences of SEQ ID NO's: 150 to 157 and 242 to 257 or of SEQ ID NO's: 166 to 173 and 274 to 289; (ii) when the first stretch of amino acid residues corresponds to one of the amino acid sequences of SEQ ID NO's: 150 to 157 and 242 to 257, the second stretch of amino acid residues corresponds to one of the amino acid sequences of SEQ ID NO's: 134 to 141 and 210 to 225 or of SEQ ID NO's: 166 to 173 and 274 to 289; or (iii) when the first stretch of amino acid residues corresponds to one of the amino acid sequences of SEQ ID NO's: 166 to 173 and 274 to 289, the second stretch of amino acid residues corresponds to one of the amino acid sequences of SEQ ID NO's: 134 to 141 and 210 to 225 or of SEQ ID NO's: 150 to 157 and 242 to 257.

Also, in such an amino acid sequence, the at least two stretches of amino acid residues again preferably form part of the antigen binding site for binding against MC4R.

In an even more specific, but non-limiting aspect, the invention relates to an amino acid sequence directed against MC4R, that comprises three or more stretches of amino acid residues, in which the first stretch of amino acid residues is chosen from the group consisting of:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;
the second stretch of amino acid residues is chosen from the group consisting of:
d) the amino acid sequences of SEQ ID NO's: 150 to 157 and 242 to 257;
e) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
and the third stretch of amino acid residues is chosen from the group consisting of:
g) the amino acid sequences of SEQ ID NO's: 166 to 173 and 274 to 289;
h) amino acid sequences that have at least 80% amino acid identity with at least one of the amino acid sequences of SEQ ID NO's: 166 to 173 and 274 to 289;
i) amino acid sequences that have 3, 2, or 1 amino acid difference with at least one of the amino acid sequences of SEQ ID NO's: 166 to 173 and 274 to 289.

Preferably, in this specific aspect, the first stretch of amino acid residues is chosen from the group consisting of the amino acid sequences of SEQ ID NO's: 134 to 141 and 210 to 225; the second stretch of amino acid residues is chosen from the group consisting of the amino acid sequences of SEQ ID NO's: 150 to 157 and 242 to 257; and the third stretch of amino acid residues is chosen from the group consisting of the amino acid sequences of SEQ ID NO's: 166 to 173 and 274 to 289.

Even more preferably, in the Nanobodies of the invention, CDR1 is chosen from the amino acid sequences of SEQ ID NO's: 136 to 141 (and may in particular be the amino acid sequence of SEQ ID NO: 141); CDR2 is chosen from the amino acid sequences of SEQ ID NO's: 152 to 157 (and may in particular be the amino acid sequence of SEQ ID NO:157); and CDR3 is chosen from the amino acid sequences of SEQ ID NO's: 168 to 173 (and may in particular be the amino acid sequence of SEQ ID NO: 173).

Again, preferably, in such an amino acid sequence, the at least three stretches of amino acid residues forms part of the antigen binding site for binding against MC4R.

Preferred combinations of such stretches of amino acid sequences will become clear from the further disclosure herein.

Preferably, in such amino acid sequences the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 182 to 189 and 306 to 321. This degree of amino acid identity can for example be determined by determining the degree of amino acid identity (in a manner described herein) between said amino acid sequence and one or more of the sequences of SEQ ID NO's: 182 to 189 and 306 to 321, in which the amino acid residues that form the framework regions are disregarded. Also, such amino acid sequences of the invention can be as further described herein.

Also, such amino acid sequences are preferably such that they can specifically bind (as defined herein) to MC4R; and more in particular bind to MC4R with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

When the amino acid sequence of the invention essentially consists of 4 framework regions (FR1 to FR4, respectively) and 3 complementarity determining regions (CDR1 to CDR3, respectively), the amino acid sequence of the invention is preferably such that:
CDR1 is chosen from the group consisting of:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;
   and/or
CDR2 is chosen from the group consisting of:
d) the amino acid sequences of SEQ ID NO's: 150 to 157 and 242 to 257;
e) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
and/or
CDR3 is chosen from the group consisting of:
g) the amino acid sequences of SEQ ID NO's: 166 to 173 and 274 to 289;
h) amino acid sequences that have at least 80% amino acid identity with at least one of the amino acid sequences of SEQ ID NO's: 166 to 173 and 274 to 289;
i) amino acid sequences that have 3, 2, or 1 amino acid difference with at least one of the amino acid sequences of SEQ ID NO's: 166 to 173 and 274 to 289.

In particular, such an amino acid sequence of the invention may be such that CDR1 is chosen from the amino acid sequences of SEQ ID NO's: 136 to 141 (and may in particular be the amino acid sequence of SEQ ID NO:141); CDR2 is chosen from the amino acid sequences of SEQ ID NO's: 152 to 157 (and may in particular be the amino acid sequence of SEQ ID NO:157); and CDR3 is chosen from the amino acid sequences of SEQ ID NO's: 168 to 173 (and may in particular be the amino acid sequence of SEQ ID NO: 173).

In particular, when the amino acid sequence of the invention essentially consists of 4 framework regions (FR1 to FR4, respectively) and 3 complementarity determining regions (CDR1 to CDR3, respectively), the amino acid sequence of the invention is preferably such that:
CDR 1 is chosen from the group consisting of:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;

and
CDR2 is chosen from the group consisting of:
d) the amino acid sequences of SEQ ID NO's: 150 to 157 and 242 to 257;
e) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
and
CDR3 is chosen from the group consisting of:
g) the amino acid sequences of SEQ ID NO's: 166 to 173 and 274 to 289;
h) amino acid sequences that have at least 80% amino acid identity with at least one of the amino acid sequences of SEQ ID NO's: 166 to 173 and 274 to 289;
i) amino acid sequences that have 3, 2, or 1 amino acid difference with at least one of the amino acid sequences of SEQ ID NO's: 166 to 173 and 274 to 289; or any suitable fragment of such an amino acid sequence

In particular, such an amino acid sequence of the invention may be such that CDR1 is chosen from the amino acid sequences of SEQ ID NO's: 136 to 141 (and may in particular be the amino acid sequence of SEQ ID NO:141); CDR2 is chosen from the amino acid sequences of SEQ ID NO's: 152 to 157 (and may in particular be the amino acid sequence of SEQ ID NO:157); and CDR3 is chosen from the amino acid sequences of SEQ ID NO's: 168 to 173 (and may in particular be the amino acid sequence of SEQ ID NO: 173).

Again, preferred combinations of CDR sequences will become clear from the further description herein.

According to a particularly preferred aspect, in the Nanobodies of the invention, CDR 1 is chosen from the amino acid sequences of SEQ ID NO's: 136 to 141 (and may in particular be the amino acid sequence of SEQ ID NO:141); CDR2 is chosen from the amino acid sequences of SEQ ID NO's: 152 to 157 (and may in particular be the amino acid sequence of SEQ ID NO:157); and CDR3 is chosen from the amino acid sequences of SEQ ID NO's: 168 to 173 (and may in particular be the amino acid sequence of SEQ ID NO: 173).

Also, such amino acid sequences are preferably such that they can specifically bind (as defined herein) to MC4R; and more in particular bind to MC4R with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In one preferred, but non-limiting aspect, the invention relates to an amino acid sequence that essentially consists of 4 framework regions (FR1 to FR4, respectively) and 3 complementarity determining regions (CDR1 to CDR3, respectively), in which the CDR sequences of said amino acid sequence have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 182 to 189 and 306 to 321. This degree of amino acid identity can for example be determined by determining the degree of amino acid identity (in a manner described herein) between said amino acid sequence and one or more of the sequences of SEQ ID NO's: 182 to 189 and 306 to 321, in which the amino acid residues that form the framework regions are disregarded. Such amino acid sequences of the invention can be as further described herein.

In such an amino acid sequence of the invention, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

The framework sequences are preferably such that the amino acid sequence of the invention is a domain antibody (or an amino acid sequence that is suitable for use as a domain antibody); is a single domain antibody (or an amino acid sequence that is suitable for use as a single domain antibody); is a “dAb” (or an amino acid sequence that is suitable for use as a dAb); or is a Nanobody™ (including but not limited to V_{HH} sequence). Again, suitable framework sequences will be clear to the skilled person, for example on the basis the standard handbooks and the further disclosure and prior art mentioned herein.

In particular, the framework sequences present in the amino acid sequences of the invention may contain one or more of Hallmark residues (as defined herein), such that the amino acid sequence of the invention is a Nanobody™. Some preferred, but non-limiting examples of (suitable combinations of) such framework sequences will become clear from the further disclosure herein.

Again, as generally described herein for the amino acid sequences of the invention, it is also possible to use suitable fragments (or combinations of fragments) of any of the foregoing, such as fragments that contain one or more CDR sequences, suitably flanked by and/or linked via one or more framework sequences (for example, in the same order as these CDR's and framework sequences may occur in the full-sized immunoglobulin sequence from which the fragment has been derived). Such fragments may also again be such that they comprise or can form an immunoglobulin fold, or alternatively be such that they do not comprise or cannot form an immunoglobulin fold.

In one specific aspect, such a fragment comprises a single CDR sequence as described herein (and in particular a CDR3 sequence), that is flanked on each side by (part of) a framework sequence (and in particular, part of the framework sequence(s) that, in the immunoglobulin sequence from which the fragment is derived, are adjacent to said CDR sequence. For example, a CDR3 sequence may be preceded by (part of) a FR3 sequence and followed by (part of) a FR4 sequence). Such a fragment may also contain a disulphide bridge, and in particular a disulphide bridge that links the two framework regions that precede and follow the CDR sequence, respectively (for the purpose of forming such a disulphide bridge, cysteine residues that naturally occur in said framework regions may be used, or alternatively cysteine residues may be synthetically added to or introduced into said framework regions). For a further description of these “Expedite fragments”, reference is again made to WO 03/050531, as well as to the US provisional application of Ablynx N.V. entitled “Peptides capable of binding to serum proteins” of Ablynx N.V. (inventors: Revets, Hilde Adi Pierrette; Kolkman, Joost Alexander; and Hoogenboom, Hendricus Renerus Jacobus Mattheus) filed on Dec. 5, 2006 (see also PCT/EP2007/063348).

In another aspect, the invention relates to a compound or construct, and in particular a protein or polypeptide (also referred to herein as a “compound of the invention” or “polypeptide of the invention”, respectively) that comprises or essentially consists of one or more amino acid sequences of the invention (or suitable fragments thereof), and optionally further comprises one or more other groups, residues, moieties or binding units. As will become clear to the skilled person from the further disclosure herein, such further groups, residues, moieties, binding units or amino acid sequences may or may not provide further functionality to the amino acid sequence of the invention (and/or to the compound or construct in which it is present) and may or may not modify the properties of the amino acid sequence of the invention.

For example, such further groups, residues, moieties or binding units may be one or more additional amino acid sequences, such that the compound or construct is a (fusion) protein or (fusion) polypeptide. In a preferred but non-limiting aspect, said one or more other groups, residues, moieties or binding units are immunoglobulin sequences. Even more preferably, said one or more other groups, residues, moieties or binding units are chosen from the group consisting of domain antibodies, amino acid sequences that are suitable for use as a domain antibody, single domain antibodies, amino acid sequences that are suitable for use as a single domain antibody, “dAb”'s, amino acid sequences that are suitable for use as a dAb, or Nanobodies.

Alternatively, such groups, residues, moieties or binding units may for example be chemical groups, residues, moieties, which may or may not by themselves be biologically and/or pharmacologically active. For example, and without limitation, such groups may be linked to the one or more amino acid sequences of the invention so as to provide a “derivative” of an amino acid sequence or polypeptide of the invention, as further described herein.

Also within the scope of the present invention are compounds or constructs, that comprises or essentially consists of one or more derivatives as described herein, and optionally further comprises one or more other groups, residues, moieties or binding units, optionally linked via one or more linkers. Preferably, said one or more other groups, residues, moieties or binding units are amino acid sequences.

In the compounds or constructs described above, the one or more amino acid sequences of the invention and the one or more groups, residues, moieties or binding units may be linked directly to each other and/or via one or more suitable linkers or spacers. For example, when the one or more groups, residues, moieties or binding units are amino acid sequences, the linkers may also be amino acid sequences, so that the resulting compound or construct is a fusion (protein) or fusion (polypeptide).

As will be clear from the further description above and herein, this means that the amino acid sequences of the invention can be used as “building blocks” to form polypeptides of the invention, i.e. by suitably combining them with other groups, residues, moieties or binding units, in order to form compounds or constructs as described herein (such as, without limitations, the biparatopic. bi/multivalent and bi/multispecific polypeptides of the invention described herein) which combine within one molecule one or more desired properties or biological functions.

The compounds or polypeptides of the invention can generally be prepared by a method which comprises at least one step of suitably linking the one or more amino acid sequences of the invention to the one or more further groups, residues, moieties or binding units, optionally via the one or more suitable linkers, so as to provide the compound or polypeptide of the invention. Polypeptides of the invention can also be prepared by a method which generally comprises at least the steps of providing a nucleic acid that encodes a polypeptide of the invention, expressing said nucleic acid in a suitable manner, and recovering the expressed polypeptide of the invention. Such methods can be performed in a manner known per se, which will be clear to the skilled person, for example on the basis of the methods and techniques further described herein.

The process of designing/selecting and/or preparing a compound or polypeptide of the invention, starting from an amino acid sequence of the invention, is also referred to herein as “formatting” said amino acid sequence of the invention; and an amino acid of the invention that is made part of a compound or polypeptide of the invention is said to be “formatted” or to be “in the format of” said compound or polypeptide of the invention. Examples of ways in which an amino acid sequence of the invention can be formatted and examples of such formats will be clear to the skilled person based on the disclosure herein; and such formatted amino acid sequences form a further aspect of the invention.

In one specific aspect of the invention, a compound of the invention or a polypeptide of the invention may have an increased half-life, compared to the corresponding amino acid sequence of the invention. Some preferred, but non-limiting examples of such compounds and polypeptides will become clear to the skilled person based on the further disclosure herein, and for example comprise amino acid sequences or polypeptides of the invention that have been chemically modified to increase the half-life thereof (for example, by means of pegylation); amino acid sequences of the invention that comprise at least one additional binding site for binding to a serum protein (such as serum albumin); or polypeptides of the invention that comprise at least one amino acid sequence of the invention that is linked to at least one moiety (and in particular at least one amino acid sequence) that increases the half-life of the amino acid sequence of the invention. Examples of polypeptides of the invention that comprise such half-life extending moieties or amino acid sequences will become clear to the skilled person based on the further disclosure herein; and for example include, without limitation, polypeptides in which the one or more amino acid sequences of the invention are suitable linked to one or more serum proteins or fragments thereof (such as (human) serum albumin or suitable fragments thereof) or to one or more binding units that can bind to scrum proteins (such as, for example, domain antibodies, amino acid sequences that are suitable for us as a domain antibody, single domain antibodies, amino acid sequences that are suitable for use as a single domain antibody, “dAb”'s, amino acid sequences that are suitable for use as a dAb, or Nanobodies that can bind to serum proteins such as serum albumin (such as human serum albumin), serum immunoglobulins such as IgG, or transferrin; reference is made to the further description and references mentioned herein); polypeptides in which an amino acid sequence of the invention is linked to an Fc portion (such as a human Fc) or a suitable part or fragment thereof; or polypeptides in which the one or more amino acid sequences of the invention are suitable linked to one or more small proteins or peptides that can bind to serum proteins (such as, without limitation, the proteins and peptides described in WO 91/01743, WO 01/45746, WO 02/076489 and to the US provisional application of Ablynx N.V. entitled “Peptides capable of binding to serum proteins” of Ablynx N.V. filed on Dec. 5, 2006 (see also PCT/EP2007/063348).

Generally, the compounds or polypeptides of the invention with increased half-life preferably have a half-life that is at least 1.5 times, preferably at least 2 times, such as at least 5 times, for example at least 10 times or more than 20 times, greater than the half-life of the corresponding amino acid sequence of the invention per se. For example, the compounds or polypeptides of the invention with increased half-life may have a half-life that is increased with more than 1 hours, preferably more than 2 hours, more preferably more than 6 hours, such as more than 12 hours, or even more than 24, 48 or 72 hours, compared to the corresponding amino acid sequence of the invention per se.

In a preferred, but non-limiting aspect of the invention, such compounds or polypeptides of the invention have a serum half-life that is increased with more than 1 hours, preferably more than 2 hours, more preferably more than 6 hours, such as more than 12 hours, or even more than 24, 48 or 72 hours, compared to the corresponding amino acid sequence of the invention per se.

In another preferred, but non-limiting aspect of the invention, such compounds or polypeptides of the invention exhibit a serum half-life in human of at least about 12 hours, preferably at least 24 hours, more preferably at least 48 hours, even more preferably at least 72 hours or more. For example, compounds or polypeptides of the invention may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

In another aspect, the invention relates to a nucleic acid that encodes an amino acid sequence of the invention or a polypeptide of the invention (or a suitable fragment thereof). Such a nucleic acid will also be referred to herein as a “nucleic acid of the invention” and may for example be in the form of a genetic construct, as further described herein.

In another aspect, the invention relates to a host or host cell that expresses (or that under suitable circumstances is capable of expressing) an amino acid sequence of the invention and/or a polypeptide of the invention; and/or that contains a nucleic acid of the invention. Some preferred but non-limiting examples of such hosts or host cells will become clear from the further description herein.

The invention further relates to a product or composition containing or comprising at least one amino acid sequence of the invention, at least one polypeptide of the invention (or a suitable fragment thereof) and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will become clear from the further description herein.

The invention also relates to the use of an amino acid sequence, Nanobody or polypeptide of the invention, or of a composition comprising the same, in (methods or compositions for) modulating MC4R, either in vitro (e.g. in an in vitro or cellular assay) or in vivo (e.g. in an a single cell or in a multicellular organism, and in particular in a mammal, and more in particular in a human being, such as in a human being that is at risk of or suffers from MC4R-related diseases and disorders (as described herein). In particular, as mentioned herein, the amino acid sequences, compounds and polypeptides of the invention are and/or can be used as agonists for MC4R and/or MC4R-mediated signalling and/or the pathways and/or biological processes in which MC4R and/or MC4R-mediated signalling is involved.

The invention also relates to methods for modulating MC4R, either in vitro (e.g. in an in vitro or cellular assay) or in vivo (e.g. in an a single cell or multicellular organism, and in particular in a mammal, and more in particular in a human being, such as in a human being that is at risk of or suffers from a MC4R-related diseases and disorders (as described herein), which method comprises at least the step of contacting MC4R with at least one amino acid sequence, Nanobody or polypeptide of the invention, or with a composition comprising the same, in a manner and in an amount suitable to modulate MC4R, with at least one amino acid sequence, Nanobody or polypeptide of the invention.

The invention also relates to the use of an one amino acid sequence, Nanobody or polypeptide of the invention in the preparation of a composition (such as, without limitation, a pharmaceutical composition or preparation as further described herein) for modulating MC4R, either in vitro (e.g. in an in vitro or cellular assay) or in vivo (e.g. in an a single cell or multicellular organism, and in particular in a mammal, and more in particular in a human being, such as in a human being that is at risk of or suffers from a MC4R-related diseases and disorders).

In the context of the present invention, “modulating” or “to modulate” generally means either reducing or inhibiting the activity of, or alternatively increasing the activity of, MC4R, as measured using a suitable in vitro, cellular or in vivo assay (such as those mentioned herein). In particular, “modulating” or “to modulate” may mean either reducing or inhibiting the activity of, or alternatively increasing the activity of MC4R, as measured using a suitable in vitro, cellular or in vivo assay (such as those mentioned herein), by at least 1%, preferably at least 5%, such as at least 10% or at least 25%, for example by at least 50%, at least 60%, at least 70%, at least 80%, or 90% or more, compared to activity of MC4R in the same assay under the same conditions but without the presence of the amino acid sequence, Nanobody or polypeptide of the invention.

As will be clear to the skilled person, “modulating” may also involve effecting a change (which may either be an increase or a decrease) in affinity, avidity, specificity and/or selectivity of MC4R for one or more of its targets, ligands or substrates; and/or effecting a change (which may either be an increase or a decrease) in the sensitivity of MC4R for one or more conditions in the medium or surroundings in which MC4R is present (such as pH, ion strength, the presence of co-factors, etc.), compared to the same conditions but without the presence of the amino acid sequence, Nanobody or polypeptide of the invention. As will be clear to the skilled person, this may again be determined in any suitable manner and/or using any suitable assay known per se, such as the assays described herein or in the prior art cited herein.

“Modulating” may also mean effecting a change (i.e. an activity as an agonist or as an antagonist, respectively) with respect to one or more biological or physiological mechanisms, effects, responses, functions, pathways or activities in which MC4R (or in which its substrate(s), ligand(s) or pathway(s) are involved, such as its signalling pathway or metabolic pathway and their associated biological or physiological effects) is involved. Again, as will be clear to the skilled person, such an action as an agonist or an antagonist may be determined in any suitable manner and/or using any suitable (in vitro and usually cellular or in assay) assay known per se, such as the assays described herein or in the prior art cited herein. In particular, an action as an agonist or antagonist may be such that an intended biological or physiological activity is increased or decreased, respectively, by at least 1%, preferably at least 5%, such as at least 10% or at least 25%, for example by at least 50%, at least 60%, at least 70%, at least 80%, or 90% or more, compared to the biological or physiological activity in the same assay under the same conditions but without the presence of the amino acid sequence, Nanobody or polypeptide of the invention.

Modulating may for example involve reducing or inhibiting the binding of MC4R to one of its substrates or ligands and/or competing with a natural ligand, substrate for binding to MC4R. Modulating may also involve activating MC4R or the mechanism or pathway in which it is involved. Modulating may be reversible or irreversible, but for pharmaceutical and pharmacological purposes will usually be in a reversible manner.

In particular, as mentioned herein, the amino acid sequences, compounds, polypeptides and compositions of the present invention are and can be used as agonists of MC4R, of MC4R-mediated signalling, of the biological pathways in which MC4R and/or MC4R-related signalling are involved, and/or more generally to modulate the biological mechanisms, responses and effects associated with such signalling or these pathways. Thus, according to a preferred aspect of the invention, “modulating” consists of such agonist action (i.e. generally of the kind known for MC4R agonists described in the art such as setmelanotide).

The invention further relates to methods for preparing or generating the amino acid sequences, polypeptides, nucleic acids, host cells, products and compositions described herein. Some preferred but non-limiting examples of such methods will become clear from the further description herein.

Generally, these methods may comprise the steps of:
a) providing a set, collection or library of amino acid sequences; and
b) screening said set, collection or library of amino acid sequences for amino acid sequences that can bind to and/or have affinity for MC4R;
and
c) isolating the amino acid sequence(s) that can bind to and/or have affinity for MC4R.

In such a method, the set, collection or library of amino acid sequences may be any suitable set, collection or library of amino acid sequences. For example, the set, collection or library of amino acid sequences may be a set, collection or library of immunoglobulin sequences (as described herein), such as a naïve set, collection or library of immunoglobulin sequences; a synthetic or semi-synthetic set, collection or library of immunoglobulin sequences; and/or a set, collection or library of immunoglobulin sequences that have been subjected to affinity maturation.

Also, in such a method, the set, collection or library of amino acid sequences may be a set, collection or library of heavy chain variable domains (such as V_{H} domains or V_{HH} domains) or of light chain variable domains. For example, the set, collection or library of amino acid sequences may be a set, collection or library of domain antibodies or single domain antibodies, or may be a set, collection or library of amino acid sequences that are capable of functioning as a domain antibody or single domain antibody.

In a preferred aspect of this method, the set, collection or library of amino acid sequences may be an immune set, collection or library of immunoglobulin sequences, for example derived from a mammal that has been suitably immunized with MC4R or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art, for example on the basis of the further disclosure herein. Reference is also made to the review by Hoogenboom in Nature Biotechnology, 23, 9, 1105-1116 (2005).

In another aspect, the method for generating amino acid sequences comprises at least the steps of:
a) providing a collection or sample of cells expressing amino acid sequences;
b) screening said collection or sample of cells for cells that express an amino acid sequence that can bind to and/or have affinity for MC4R;
and
c) either (i) isolating said amino acid sequence; or (ii) isolating from said cell a nucleic acid sequence that encodes said amino acid sequence, followed by expressing said amino acid sequence.

For example, when the desired amino acid sequence is an immunoglobulin sequence, the collection or sample of cells may for example be a collection or sample of B-cells. Also, in this method, the sample of cells may be derived from a mammal that has been suitably immunized with MC4R or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The above method may be performed in any suitable manner, as will be clear to the skilled person. Reference is for example made to EP 0 542 810, WO 05/19824, WO 04/051268 and WO 04/106377. The screening of step b) is preferably performed using a flow cytometry technique such as FACS. For this, reference is for example made to Lieby et al., Blood, Vol. 97, No. 12, 3820 (2001).

In another aspect, the method for generating an amino acid sequence directed against MC4R may comprise at least the steps of:
a) providing a set, collection or library of nucleic acid sequences encoding amino acid sequences;
b) screening said set, collection or library of nucleic acid sequences for nucleic acid sequences that encode an amino acid sequence that can bind to and/or has affinity for MC4R;
and
c) isolating said nucleic acid sequence, followed by expressing said amino acid sequence.

In such a method, the set, collection or library of nucleic acid sequences encoding amino acid sequences may for example be a set, collection or library of nucleic acid sequences encoding a naïve set, collection or library of immunoglobulin sequences; a set, collection or library of nucleic acid sequences encoding a synthetic or semi-synthetic set, collection or library of immunoglobulin sequences; and/or a set, collection or library of nucleic acid sequences encoding a set, collection or library of immunoglobulin sequences that have been subjected to affinity maturation.

Also, in such a method, the set, collection or library of nucleic acid sequences may encode a set, collection or library of heavy chain variable domains (such as V_{H} domains or V_{HH} domains) or of light chain variable domains. For example, the set, collection or library of nucleic acid sequences may encode a set, collection or library of domain antibodies or single domain antibodies, or a set, collection or library of amino acid sequences that are capable of functioning as a domain antibody or single domain antibody.

In a preferred aspect of this method, the set, collection or library of amino acid sequences may be an immune set, collection or library of nucleic acid sequences, for example derived from a mammal that has been suitably immunized with MC4R or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The set, collection or library of nucleic acid sequences may for example encode an immune set, collection or library of heavy chain variable domains or of light chain variable domains. In one specific aspect, the set, collection or library of nucleotide sequences may encode a set, collection or library of V_{HH} sequences.

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to the review by Hoogenboom in Nature Biotechnology, 23, 9, 1105-1116 (2005).

The invention also relates to amino acid sequences that are obtained by the above methods, or alternatively by a method that comprises the one of the above methods and in addition at least the steps of determining the nucleotide sequence or amino acid sequence of said immunoglobulin sequence; and of expressing or synthesizing said amino acid sequence in a manner known per se, such as by expression in a suitable host cell or host organism or by chemical synthesis.

Also, following the steps above, one or more amino acid sequences of the invention may be suitably humanized (or alternatively camelized); and/or the amino acid sequence(s) thus obtained may be linked to each other or to one or more other suitable amino acid sequences (optionally via one or more suitable linkers) so as to provide a polypeptide of the invention. Also, a nucleic acid sequence encoding an amino acid sequence of the invention may be suitably humanized (or alternatively camelized) and suitably expressed; and/or one or more nucleic acid sequences encoding an amino acid sequence of the invention may be linked to each other or to one or more nucleic acid sequences that encode other suitable amino acid sequences (optionally via nucleotide sequences that encode one or more suitable linkers), after which the nucleotide sequence thus obtained may be suitably expressed so as to provide a polypeptide of the invention.

The invention further relates to applications and uses of the amino acid sequences, compounds, constructs, polypeptides, nucleic acids, host cells, products and compositions described herein, as well as to methods for the prevention and/or treatment for diseases and disorders associated with MC4R. Some preferred but non-limiting applications and uses will become clear from the further description herein.

The invention also relates to the amino acid sequences, compounds, constructs, polypeptides, nucleic acids, host cells, products and compositions described herein for use in therapy.

In particular, the invention also relates to the amino acid sequences, compounds, constructs, polypeptides, nucleic acids, host cells, products and compositions described herein for use in therapy of a disease or disorder that can be prevented or treated by administering, to a subject in need thereof, of (a pharmaceutically effective amount of) an amino acid sequence, compound, construct or polypeptide as described herein.

More in particular, the invention relates to the amino acid sequences, compounds, constructs, polypeptides, nucleic acids, host cells, products and compositions described herein for use in therapy of MC4R-related diseases and disorders.

Other aspects, embodiments, advantages and applications of the invention will also become clear from the further description herein, in which the invention will be described and discussed in more detail with reference to the Nanobodies of the invention and polypeptides of the invention comprising the same, which form some of the preferred aspects of the invention.

As will become clear from the further description herein, Nanobodies generally offer certain advantages (outlined herein) compared to “dAb's” or similar (single) domain antibodies or immunoglobulin sequences, which advantages are also provided by the Nanobodies of the invention. However, it will be clear to the skilled person that the more general aspects of the teaching below can also be applied (either directly or analogously) to other amino acid sequences of the invention.

### DETAILED DESCRIPTION OF THE INVENTION

In the present description, examples and claims:
Unless indicated or defined otherwise, all terms used have their usual meaning in the art, which will be clear to the skilled person. Reference is for example made to the standard handbooks mentioned in paragraph a) on page 46 of WO 08/020079
Unless indicated otherwise, the terms “immunoglobulin sequence”, “sequence”, “nucleotide sequence” and “nucleic acid” are as described in paragraph b) on page 46 of WO 08/020079
c) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta, Adv. Drug Deliv. Rev. 2006, 58 (5-6): 640-56; Levin and Weiss, Mol. Biosyst. 2006, 2 (1): 49-57; Irving et al., J. Immunol. Methods, 2001, 248 (1-2), 31-45; Schmitz et al., Placenta, 2000, 21 Suppl. A, S106-12, Gonzales et al., Tumour Biol., 2005, 26 (1), 31-43, which describe techniques for protein engineering, such as affinity maturation and other techniques for improving the specificity and other desired properties of proteins such as immunoglobulins.
d) Amino acid residues will be indicated according to the standard three-letter or one-letter amino acid code. Reference is made to Table A-2 on page 48 of the International application WO 08/020079 of Ablynx N.V. entitled “Amino acid sequences directed against IL-6R and polypeptides comprising the same for the treatment of diseases and disorders associated with Il-6 mediated signalling”.
e) For the purposes of comparing two or more nucleotide sequences, the percentage of “sequence identity” between a first nucleotide sequence and a second nucleotide sequence may be calculated or determined as described in paragraph c) on page 49 of WO 08/020079 (incorporated herein by reference), such as by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position); or using a suitable computer algorithm or technique, again as described in paragraph c) on pages 49 of WO 08/020079 (incorporated herein by reference).
f) For the purposes of comparing two or more amino acid sequences, the percentage of “sequence identity” between a first amino acid sequence and a second amino acid sequence (also referred to herein as “amino acid identity”) may be calculated or determined as described in paragraph f) on pages 49 and 50 of WO 08/020079 (incorporated herein by reference), such as by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein; or using a suitable computer algorithm or technique, again as described in paragraph f) on pages 49 and 50 of WO 08/020079 (incorporated herein by reference).
   Also, in determining the degree of sequence identity between two amino acid sequences, the skilled person may take into account so-called “conservative” amino acid substitutions, as described on page 50 of WO 08/020079.
   Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13:211, 1974 and Adv. Enzymol., 47:45-149, 1978, and on the analysis of hydrophobicity patterns in proteins developed by Eisenberg et al., Proc. Nad. Acad Sci. USA 81:140-144, 1984; Kyte & Doolittle; J Molec. Biol. 157:105-132, 198 1, and Goldman et al., Ann. Rev. Biophys. Chem. 15:321-353, 1986, all incorporated herein in their entirety by reference. Information on the primary, secondary and tertiary structure of Nanobodies is given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional V_{H} domains form the V_{H}/V_{L} interface and potential camelizing substitutions on these positions can be found in the prior art cited above.
g) Amino acid sequences and nucleic acid sequences are said to be “exactly the same” if they have 100% sequence identity (as defined herein) over their entire length;
h) When comparing two amino acid sequences, the term “amino acid difference” refers to an insertion, deletion or substitution of a single amino acid residue on a position of the first sequence, compared to the second sequence; it being understood that two amino acid sequences can contain one, two or more such amino acid differences;
i) When a nucleotide sequence or amino acid sequence is said to “comprise” another nucleotide sequence or amino acid sequence, respectively, or to “essentially consist of” another nucleotide sequence or amino acid sequence, this has the meaning given in paragraph i) on pages 51-52 of WO 08/020079.

j) The term “in essentially isolated form” has the meaning given to it in paragraph j) on pages 52 and 53 of WO 08/020079.
The terms “domain” and “binding domain” have the meanings given to it in paragraph k) on page 53 of WO 08/020079.
The terms “antigenic determinant” and “epitope”, which may also be used interchangeably herein, have the meanings given to it in paragraph 1) on page 53 of WO 08/020079.
m) As further described in paragraph m) on page 53 of WO 08/020079, an amino acid sequence (such as a Nanobody, an antibody, a polypeptide of the invention, or generally an antigen binding protein or polypeptide or a fragment thereof) that can (specifically) bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
n) The term “specificity” has the meaning given to it in paragraph n) on pages 53-56 of WO 08/020079; and as mentioned therein refers to the number of different types of antigens or antigenic determinants to which a particular antigen-binding molecule or antigen-binding protein (such as a Nanobody or a polypeptide of the invention) molecule can bind. The specificity of an antigen-binding protein can be determined based on affinity and/or avidity, as described on pages 53-56 of WO 08/020079 (incorporated herein by reference), which also describes some preferred techniques for measuring binding between an antigen-binding molecule (such as a Nanobody or polypeptide of the invention) and the pertinent antigen. Typically, antigen-binding proteins (particularly those that are used as antagonists) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 104 mol/liter (or any K_{A} value lower than 10⁴ M⁻¹) liters/mol is generally considered to indicate non-specific binding. Preferably, a monovalent immunoglobulin sequence of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.
   As will be clear to the skilled person, and as described on pages 53-56 of WO 08/020079, the dissociation constant may be the actual or apparent dissociation constant: Methods for determining the dissociation constant will be clear to the skilled person, and for example include the techniques mentioned on pages 53-56 of WO 08/020079
   The half-life of an amino acid sequence, compound or polypeptide of the invention can generally be defined as described in paragraph o) on page 57 of WO 08/020079 and as mentioned therein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art, and may for example generally be as described in paragraph o) on page 57 of WO 08/020079. As also mentioned in paragraph o) on page 57 of WO 08/020079, the half-life can be expressed using parameters such as the t1/2-alpha, t1/2-beta and the area under the curve (AUC). Reference is for example made to the Experimental Part below, as well as to the standard handbooks, such as Kenneth, A et al: Chemical Stability of Pharmaceuticals: A Handbook for Pharmacists and Peters et al, Pharmacokinete analysis: A Practical Approach (1996). Reference is also made to “Pharmacokinetics”, M Gibaldi & D Perron, published by Marcel Dekker, 2nd Rev. edition (1982). The terms “increase in half-life” or “increased half-life” as also as defined in paragraph o) on page 57 of WO 08/020079 and in particular refer to an increase in the t1/2-beta, either with or without an increase in the t1/2-alpha and/or the AUC or both.
o) In the context of the present invention, “modulating” or “to modulate” generally means either reducing or inhibiting the activity of, or alternatively increasing the activity of, a target or antigen, as measured using a suitable in vitro, cellular or in vivo assay. In particular, “modulating” or “to modulate” may mean either reducing or inhibiting the activity of, or alternatively increasing a (relevant or intended) biological activity of, a target or antigen, as measured using a suitable in vitro, cellular or in vivo assay (which will usually depend on the target or antigen involved), by at least 1%, preferably at least 5%, such as at least 10% or at least 25%, for example by at least 50%, at least 60%, at least 70%, at least 80%, or 90% or more, compared to activity of the target or antigen in the same assay under the same conditions but without the presence of the construct of the invention.
   As will be clear to the skilled person, “modulating” may also involve effecting a change (which may either be an increase or a decrease) in affinity, avidity, specificity and/or selectivity of a target or antigen for one or more of its ligands, binding partners, partners for association into a homomultimeric or heteromultimeric form, or substrates; and/or effecting a change (which may either be an increase or a decrease) in the sensitivity of the target or antigen for one or more conditions in the medium or surroundings in which the target or antigen is present (such as pH, ion strength, the presence of co-factors, etc.), compared to the same conditions but without the presence of the construct of the invention. As will be clear to the skilled person, this may again be determined in any suitable manner and/or using any suitable assay known per se, depending on the target or antigen involved.
   “Modulating” may also mean effecting a change (i.e. an activity as an agonist, as an antagonist or as a reverse agonist, respectively, depending on the target or antigen and the desired biological or physiological effect) with respect to one or more biological or physiological mechanisms, effects, responses, functions, pathways or activities in which the target or antigen (or in which its substrate(s), ligand(s) or pathway(s) are involved, such as its signalling pathway or metabolic pathway and their associated biological or physiological effects) is involved. Again, as will be clear to the skilled person, such an action as an agonist or an antagonist may be determined in any suitable manner and/or using any suitable (in vitro and usually cellular or in assay) assay known per se, depending on the target or antigen involved. In particular, an action as an agonist or antagonist may be such that an intended biological or physiological activity is increased or decreased, respectively, by at least 1%, preferably at least 5%, such as at least 10% or at least 25%, for example by at least 50%, at least 60%, at least 70%, at least 80%, or 90% or more, compared to the biological or physiological activity in the same assay under the same conditions but without the presence of the construct of the invention.
   Modulating may for example also involve allosteric modulation of the target or antigen; and/or reducing or inhibiting the binding of the target or antigen to one of its substrates or ligands and/or competing with a natural ligand, substrate for binding to the target or antigen. Modulating may also involve activating the target or antigen or the mechanism or pathway in which it is involved. Modulating may for example also involve effecting a change in respect of the folding or confirmation of the target or antigen, or in respect of the ability of the target or antigen to fold, to change its confirmation (for example, upon binding of a ligand), to associate with other (sub) units, or to disassociate. Modulating may for example also involve effecting a change in the ability of the target or antigen to transport other compounds or to serve as a channel for other compounds (such as ions).
   Modulating may be reversible or irreversible, but for pharmaceutical and pharmacological purposes will usually be in a reversible manner.
p) In respect of a target or antigen, the term “interaction site” on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multimerisation (such as homomerization or heterodimerization) of the target or antigen; or any other site, epitope, antigenic determinant, part, domain or stretch of amino acid residues on the target or antigen that is involved in a biological action or mechanism of the target or antigen. More generally, an “interaction site” can be any site, epitope, antigenic determinant, part, domain or stretch of amino acid residues on the target or antigen to which an amino acid sequence or polypeptide of the invention can bind such that the target or antigen (and/or any pathway, interaction, signalling, biological mechanism or biological effect in which the target or antigen is involved) is modulated (as defined herein).
q) An amino acid sequence or polypeptide is said to be “specific for” a first target or antigen compared to a second target or antigen when is binds to the first antigen with an affinity (as described above, and suitably expressed as a K_{D} value, K_{A} value, K_{off} rate and/or Kₒₙ rate) that is at least 10 times, such as at least 100 times, and preferably at least 1000 times, and up to 10.000 times or more better than the affinity with which said amino acid sequence or polypeptide binds to the second target or polypeptide. For example, the first antigen may bind to the target or antigen with a K_{D} value that is at least 10 times less, such as at least 100 times less, and preferably at least 1000 times less, such as 10.000 times less or even less than that, than the K_{D} with which said amino acid sequence or polypeptide binds to the second target or polypeptide. Preferably, when an amino acid sequence or polypeptide is “specific for” a first target or antigen compared to a second target or antigen, it is directed against (as defined herein) said first target or antigen, but not directed against said second target or antigen.
r) The terms “cross-block”, “cross-blocked” and “cross-blocking” are used interchangeably herein to mean the ability of an amino acid sequence or other binding agents (such as a polypeptide of the invention) to interfere with the binding of other amino acid sequences or binding agents of the invention to a given target. The extend to which an amino acid sequence or other binding agents of the invention is able to interfere with the binding of another to [target], and therefore whether it can be said to cross-block according to the invention, can be determined using competition binding assays. One particularly suitable quantitative assay uses a Biacore machine which can measure the extent of interactions using surface plasmon resonance technology. Another suitable quantitative cross-blocking assay uses an ELISA-based approach to measure competition between amino acid sequence or another binding agents in terms of their binding to the target.
   The following generally describes a suitable Biacore assay for determining whether an amino acid sequence or other binding agent cross-blocks or is capable of cross-blocking according to the invention. It will be appreciated that the assay can be used with any of the amino acid sequence or other binding agents described herein. The Biacore machine (for example the Biacore 3000) is operated in line with the manufacturer's recommendations. Thus in one cross-blocking assay, the target protein is coupled to a CM5 Biacore chip using standard amine coupling chemistry to generate a surface that is coated with the target. Typically 200-800 resonance units of the target would be coupled to the chip (an amount that gives easily measurable levels of binding but that is readily saturable by the concentrations of test reagent being used). Two test amino acid sequences (termed A* and B*) to be assessed for their ability to cross-block each other are mixed at a one to one molar ratio of binding sites in a suitable buffer to create the test mixture. When calculating the concentrations on a binding site basis the molecular weight of an amino acid sequence is assumed to be the total molecular weight of the amino acid sequence divided by the number of target binding sites on that amino acid sequence. The concentration of each amino acid sequence in the test mix should be high enough to readily saturate the binding sites for that amino acid sequence on the target molecules captured on the Biacore chip. The amino acid sequences in the mixture are at the same molar concentration (on a binding basis) and that concentration would typically be between 1.00 and 1.5 micromolar (on a binding site basis). Separate solutions containing A* alone and B* alone are also prepared. A* and B* in these solutions should be in the same buffer and at the same concentration as in the test mix. The test mixture is passed over the target-coated Biacore chip and the total amount of binding recorded. The chip is then treated in such a way as to remove the bound amino acid sequences without damaging the chip-bound target. Typically this is done by treating the chip with 30 mM HCl for 60 seconds. The solution of A* alone is then passed over the target-coated surface and the amount of binding recorded. The chip is again treated to remove all of the bound amino acid sequences without damaging the chip-bound target. The solution of B* alone is then passed over the target-coated surface and the amount of binding recorded. The maximum theoretical binding of the mixture of A* and B* is next calculated, and is the sum of the binding of each amino acid sequence when passed over the target surface alone. If the actual recorded binding of the mixture is less than this theoretical maximum then the two amino acid sequences are cross-blocking each other. Thus, in general, a cross-blocking amino acid sequence or other binding agent according to the invention is one which will bind to the target in the above Biacore cross-blocking assay such that during the assay and in the presence of a second amino acid sequence or other binding agent of the invention the recorded binding is between 80% and 0.1% (e.g. 80% to 4%) of the maximum theoretical binding, specifically between 75% and 0.1% (e.g. 75% to 4%) of the maximum theoretical binding, and more specifically between 70% and 0.1% (e.g. 70% to 4%) of maximum theoretical binding (as just defined above) of the two amino acid sequences or binding agents in combination. The Biacore assay described above is a primary assay used to determine if amino acid sequences or other binding agents cross-block each other according to the invention. On rare occasions particular amino acid sequences or other binding agents may not bind to target coupled via amine chemistry to a CM5 Biacore chip (this usually occurs when the relevant binding site on target is masked or destroyed by the coupling to the chip). In such cases cross-blocking can be determined using a tagged version of the target, for example a N-terminal His-tagged version (R & D Systems, Minneapolis, MN, USA; 2005 cat #1406-ST-025). In this particular format, an anti-His amino acid sequence would be coupled to the Biacore chip and then the His-tagged target would be passed over the surface of the chip and captured by the anti-His amino acid sequence. The cross blocking analysis would be carried out essentially as described above, except that after each chip regeneration cycle, new His-tagged target would be loaded back onto the anti-His amino acid sequence coated surface. In addition to the example given using N-terminal His-tagged [target], C-terminal His-tagged target could alternatively be used. Furthermore, various other tags and tag binding protein combinations that are known in the art could be used for such a cross-blocking analysis (e.g. HA tag with anti-HA antibodies; FLAG tag with anti-FLAG antibodies; biotin tag with streptavidin).
   The following generally describes an ELISA assay for determining whether an amino acid sequence or other binding agent directed against a target cross-blocks or is capable of cross-blocking as defined herein. It will be appreciated that the assay can be used with any of the amino acid sequences (or other binding agents such as polypeptides of the invention) described herein. The general principal of the assay is to have an amino acid sequence or binding agent that is directed against the target coated onto the wells of an ELISA plate. An excess amount of a second, potentially cross-blocking, anti-target amino acid sequence is added in solution (i.e. not bound to the ELISA plate). A limited amount of the target is then added to the wells. The coated amino acid sequence and the amino acid sequence in solution compete for binding of the limited number of target molecules. The plate is washed to remove excess target that has not been bound by the coated amino acid sequence and to also remove the second, solution phase amino acid sequence as well as any complexes formed between the second, solution phase amino acid sequence and target. The amount of bound target is then measured using a reagent that is appropriate to detect the target. An amino acid sequence in solution that is able to cross-block the coated amino acid sequence will be able to cause a decrease in the number of target molecules that the coated amino acid sequence can bind relative to the number of target molecules that the coated amino acid sequence can bind in the absence of the second, solution phase, amino acid sequence. In the instance where the first amino acid sequence, e.g. an Ab-X, is chosen to be the immobilized amino acid sequence, it is coated onto the wells of the ELISA plate, after which the plates are blocked with a suitable blocking solution to minimize non-specific binding of reagents that are subsequently added. An excess amount of the second amino acid sequence, i.e. Ab-Y, is then added to the ELISA plate such that the moles of Ab-Y [target] binding sites per well are at least 10 fold higher than the moles of Ab-X [target] binding sites that were used, per well, during the coating of the ELISA plate. [target] is then added such that the moles of [target] added per well are at least 25-fold lower than the moles of Ab-X [target] binding sites that were used for coating each well. Following a suitable incubation period the ELISA plate is washed and a reagent for detecting the target is added to measure the amount of target specifically bound by the coated anti-[target] amino acid sequence (in this case Ab-X). The background signal for the assay is defined as the signal obtained in wells with the coated amino acid sequence (in this case Ab-X), second solution phase amino acid sequence (in this case Ab-Y), [target] buffer only (i.e. no target) and target detection reagents. The positive control signal for the assay is defined as the signal obtained in wells with the coated amino acid sequence (in this case Ab-X), second solution phase amino acid sequence buffer only (i.e. no second solution phase amino acid sequence), target and target detection reagents. The ELISA assay may be run in such a manner so as to have the positive control signal be at least 6 times the background signal. To avoid any artefacts (e.g. significantly different affinities between Ab-X and Ab-Y for [target]) resulting from the choice of which amino acid sequence to use as the coating amino acid sequence and which to use as the second (competitor) amino acid sequence, the cross-blocking assay may to be run in two formats: 1) format 1 is where Ab-X is the amino acid sequence that is coated onto the ELISA plate and Ab-Y is the competitor amino acid sequence that is in solution and 2) format 2 is where Ab-Y is the amino acid sequence that is coated onto the ELISA plate and Ab-X is the competitor amino acid sequence that is in solution. Ab-X and Ab-Y are defined as cross-blocking if, either in format 1 or in format 2, the solution phase anti-target amino acid sequence is able to cause a reduction of between 60% and 100%, specifically between 70% and 100%, and more specifically between 80% and 100%, of the target detection signal {i.e. the amount of target bound by the coated amino acid sequence) as compared to the target detection signal obtained in the absence of the solution phase anti-target amino acid sequence (i.e. the positive control wells).
s) As further described herein, the total number of amino acid residues in a Nanobody can be in the region of 110-120, is preferably 112-115, and is most preferably 113. It should however be noted that parts, fragments, analogs or derivatives (as further described herein) of a Nanobody are not particularly limited as to their length and/or size, as long as such parts, fragments, analogs or derivatives meet the further requirements outlined herein and are also preferably suitable for the purposes described herein;
   As further described in paragraph q) on pages 58 and 59 of WO 08/020079 (incorporated herein by reference), the amino acid residues of a Nanobody are numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, J. Immunol. Methods 2000 Jun. 23; 240 (1-2): 185-195 (see for example FIG. 2 of this publication), and accordingly FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113.
t) The Figures, Sequence Listing and the Experimental Part/Examples are only given to further illustrate the invention and should not be interpreted or construed as limiting the scope of the invention and/or of the appended claims in any way, unless explicitly indicated otherwise herein.

For a general description of heavy chain antibodies and the variable domains thereof, reference is inter alia made to the prior art cited herein, as well as to the prior art mentioned on page 59 of WO 08/020079 and to the list of references mentioned on pages 41-43 of the International application WO 06/040153, which prior art and references are incorporated herein by reference.

In accordance with the terminology used in the art (see the above references), the variable domains present in naturally occurring heavy chain antibodies will also be referred to as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”).

As mentioned in the prior art referred to above, V_{HH} domains have a number of unique structural characteristics and functional properties which make isolated V_{HH} domains (as well as Nanobodies based thereon, which share these structural characteristics and functional properties with the naturally occurring V_{HH} domains) and proteins containing the same highly advantageous for use as functional antigen-binding domains or proteins. In particular, and without being limited thereto, V_{HH} domains (which have been “designed” by nature to functionally bind to an antigen without the presence of, and without any interaction with, a light chain variable domain) and Nanobodies can function as a single, relatively small, functional antigen-binding structural unit, domain or protein. This distinguishes the V_{HH} domains from the V_{H} and V_{L} domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv's fragments, which consist of a V_{H} domain covalently linked to a V_{L} domain).

Because of these unique properties, the use of V_{HH} domains and Nanobodies as single antigen-binding proteins or as antigen-binding domains (i.e. as part of a larger protein or polypeptide) offers a number of significant advantages over the use of conventional V_{H} and V_{L} domains, scFv's or conventional antibody fragments (such as Fab- or F(ab′)₂-fragments), including the advantages that are listed on pages 60 and 61 of WO 08/020079.

In a specific and preferred aspect, the invention provides Nanobodies against MC4R, and in particular Nanobodies against MC4R from a warm-blooded animal, and more in particular Nanobodies against MC4R from a mammal, and especially Nanobodies against human MC4R; as well as proteins and/or polypeptides comprising at least one such Nanobody.

In particular, the invention provides Nanobodies against MC4R, and proteins and/or polypeptides comprising the same, that have improved therapeutic and/or pharmacological properties and/or other advantageous properties (such as, for example, improved ease of preparation and/or reduced costs of goods), compared to conventional antibodies against MC4R or fragments thereof, compared to constructs that could be based on such conventional antibodies or antibody fragments (such as Fab′ fragments, F(ab′)₂ fragments, ScFv constructs, “diabodies” and other multispecific constructs (see for example the review by Holliger and Hudson, Nat Biotechnol. 2005 September; 23 (9): 1126-36)), and also compared to the so-called “dAb's” or similar (single) domain antibodies that may be derived from variable domains of conventional antibodies. These improved and advantageous properties will become clear from the further description herein, and for example include, without limitation, one or more of:
increased affinity and/or avidity for MC4R, either in a monovalent format, in an Fc format (as further described herein), in a multivalent format (for example in a bivalent format) and/or in a multispecific format (for example one of the multispecific formats described hereinbelow);
better suitability for formatting in an Fc format (as further described herein) or in a multivalent format (for example in a bivalent format);
better suitability for formatting in a multispecific format (for example one of the multispecific formats described hereinbelow);
improved suitability or susceptibility for “humanizing” substitutions (as defined herein);
less immunogenicity, either in a monovalent format, in an Fc format (as further described herein), in a multivalent format (for example in a bivalent format) and/or in a multispecific format (for example one of the multispecific formats described hereinbelow);
increased stability, either in a monovalent format, in an Fc format (as further described herein), in a multivalent format (for example in a bivalent format) and/or in a multispecific format (for example one of the multispecific formats described hereinbelow);
increased specificity towards MC4R, either in a monovalent format, in an Fc format (as further described herein), in a multivalent format (for example in a bivalent format) and/or in a multispecific format (for example one of the multispecific formats described hereinbelow);
decreased or where desired increased cross-reactivity with MC4R from different species;
   and/or
one or more other improved properties desirable for pharmaceutical use (including prophylactic use and/or therapeutic use) and/or for diagnostic use (including but not limited to use for imaging purposes), either in a monovalent format, in a multivalent format (for example in a bivalent format) and/or in a multispecific format (for example one of the multispecific formats described hereinbelow).

As generally described herein for the amino acid sequences of the invention, the Nanobodies of the invention are preferably in essentially isolated form (as defined herein), or form part of a protein or polypeptide of the invention (as defined herein), which may comprise or essentially consist of one or more Nanobodies of the invention and which may optionally further comprise one or more further amino acid sequences (all optionally linked via one or more suitable linkers). For example, and without limitation, the one or more amino acid sequences of the invention may be used as a binding unit in such a protein or polypeptide, which may optionally contain one or more further amino acid sequences that can serve as a binding unit (i.e. against one or more other targets than MC4R), so as to provide a monovalent, multivalent or multispecific polypeptide of the invention, respectively, all as described herein. In particular, such a protein or polypeptide may comprise or essentially consist of one or more Nanobodies of the invention and optionally one or more (other) Nanobodies (i.e. directed against other targets than MC4R), all optionally linked via one or more suitable linkers, so as to provide a monovalent, multivalent or multispecific Nanobody construct, respectively, as further described herein. Such proteins or polypeptides may also be in essentially isolated form (as defined herein).

In a Nanobody of the invention, the binding site for binding against MC4R is preferably formed by the CDR sequences. Optionally, a Nanobody of the invention may also, and in addition to the at least one binding site for binding against MC4R, contain one or more further binding sites for binding against other antigens, proteins or targets. For methods and positions for introducing such second binding sites, reference is for example made to Keck and Huston, Biophysical Journal, 71, October 1996, 2002-2011, EP 0 640 130; and WO 06/07260.

As generally described herein for the amino acid sequences of the invention, when a Nanobody of the invention (or a polypeptide of the invention comprising the same) is intended for administration to a subject (for example for therapeutic and/or diagnostic purposes as described herein), it is preferably directed against human MC4R; whereas for veterinary purposes, it is preferably directed against MC4R from the species to be treated. Also, as with the amino acid sequences of the invention, a Nanobody of the invention may or may not be cross-reactive (i.e. directed against MC4R from two or more species of mammal, such as against human MC4R and MC4R from at least one of the species of mammal mentioned herein).

Also, again as generally described herein for the amino acid sequences of the invention, the Nanobodies of the invention may generally be directed against any antigenic determinant, epitope, part, domain, subunit or confirmation (where applicable) of MC4R. However, it is generally assumed and preferred that the Nanobodies of the invention (and polypeptides comprising the same) are directed against the binding site of a natural ligand, and more in particular against the binding site of alpha-MSH (e.g. such that at least one CDR, such as CDR3, binds to and/or overlaps with the binding site of a natural ligand, and in particular with the binding site of alpha-MSH).

As already described herein, the amino acid sequence and structure of a Nanobody can be considered—without however being limited thereto—to be comprised of four framework regions or “FR's” (or sometimes also referred to as “FW's”), which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively. Some preferred framework sequences and CDR's (and combinations thereof) that are present in the Nanobodies of the invention are as described herein. Other suitable CDR sequences can be obtained by the methods described herein.

Preferably, (the CDR sequences present in) the Nanobodies of the invention are such that: a monovalent Nanobody of the invention (or a polypeptide that contains only one Nanobody of the invention) is preferably such that it will bind to MC4R with an affinity less than 500 nM, preferably less than 200 nM, more preferably less than 10 nM, such as less than 500 pM.

The affinity of the Nanobody of the invention against MC4R can be determined in a manner known per se, for example using the general techniques for measuring K_{D}. K_{A}, K_{off} or kₒₙ mentioned herein, as well as some of the specific assays described herein.

Some preferred IC50 values for binding of the Nanobodies of the invention (and of polypeptides comprising the same) to MC4R will become clear from the further description and examples herein.

In a preferred but non-limiting aspect, the invention relates to a Nanobody (as defined herein) against MC4R, which consists of 4 framework regions (FR1 to FR4 respectively) and 3 complementarity determining regions (CDR1 to CDR3 respectively), in which:
CDR1 is chosen from the group consisting of:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;
   and/or
CDR2 is chosen from the group consisting of:
d) the amino acid sequences of SEQ ID NO's: 150 to 157 and 242 to 257;
e) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
   and/or
CDR3 is chosen from the group consisting of:
g) the amino acid sequences of SEQ ID NO's: 166 to 173 and 274 to 289;
h) amino acid sequences that have at least 80% amino acid identity with at least one of the amino acid sequences of SEQ ID NO's: 166 to 173 and 274 to 289;
i) amino acid sequences that have 3, 2, or 1 amino acid difference with at least one of the amino acid sequences of SEQ ID NO's: 166 to 173 and 274 to 289;
   or any suitable fragment of such an amino acid sequence.

In particular, according to this preferred but non-limiting aspect, the invention relates to a Nanobody (as defined herein) against MC4R, which consists of 4 framework regions (FR1 to FR4 respectively) and 3 complementarity determining regions (CDR1 to CDR3 respectively), in which:
CDR1 is chosen from the group consisting of:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;
   and
CDR2 is chosen from the group consisting of:
d) the amino acid sequences of SEQ ID NO's: 150 to 157 and 242 to 257;
e) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
   and
CDR3 is chosen from the group consisting of:
g) the amino acid sequences of SEQ ID NO's: 166 to 173 and 274 to 289;
h) amino acid sequences that have at least 80% amino acid identity with at least one of the amino acid sequences of SEQ ID NO's: 166 to 173 and 274 to 289;
i) amino acid sequences that have 3, 2, or 1 amino acid difference with at least one of the amino acid sequences of SEQ ID NO's: 166 to 173 and 274 to 289;
   or any suitable fragment of such an amino acid sequences.

As generally mentioned herein for the amino acid sequences of the invention, when a Nanobody of the invention contains one or more CDR1 sequences according to b) and/or c):
i) any amino acid substitution in such a CDR according to b) and/or c) is preferably, and compared to the corresponding CDR according to a), a conservative amino acid substitution (as defined herein);
and/or
ii) the CDR according to b) and/or c) preferably only contains amino acid substitutions, and no amino acid deletions or insertions, compared to the corresponding CDR according to a);
and/or
iii) the CDR according to b) and/or c) may be a CDR that is derived from a CDR according to a) by means of affinity maturation using one or more techniques of affinity maturation known per se.

Similarly, when a Nanobody of the invention contains one or more CDR2 sequences according to e) and/or f):
i) any amino acid substitution in such a CDR according to e) and/or f) is preferably, and compared to the corresponding CDR according to d), a conservative amino acid substitution (as defined herein);
and/or
ii) the CDR according to e) and/or f) preferably only contains amino acid substitutions, and no amino acid deletions or insertions, compared to the corresponding CDR according to d);
and/or
iii) the CDR according to e) and/or f) may be a CDR that is derived from a CDR according to d) by means of affinity maturation using one or more techniques of affinity maturation known per se.

Also, similarly, when a Nanobody of the invention contains one or more CDR3 sequences according to h) and/or i):
i) any amino acid substitution in such a CDR according to h) and/or i) is preferably, and compared to the corresponding CDR according to g), a conservative amino acid substitution (as defined herein);
and/or
ii) the CDR according to h) and/or i) preferably only contains amino acid substitutions, and no amino acid deletions or insertions, compared to the corresponding CDR according to g);
and/or
iii) the CDR according to h) and/or i) may be a CDR that is derived from a CDR according to g) by means of affinity maturation using one or more techniques of affinity maturation known per se.

It should be understood that the last three paragraphs generally apply to any Nanobody of the invention that comprises one or more CDR1 sequences, CDR2 sequences and/or CDR3 sequences according to b), c), e), f), h) or i), respectively.

Of the Nanobodies of the invention, Nanobodies comprising one or more of the CDR's explicitly listed above are particularly preferred; Nanobodies comprising two or more of the CDR's explicitly listed above are more particularly preferred; and Nanobodies comprising three of the CDR's explicitly listed above are most particularly preferred.

Some particularly preferred, but non-limiting combinations of CDR sequences, as well as preferred combinations of CDR sequences and framework sequences, are mentioned in Table A-1 below, which lists the CDR sequences and framework sequences that are present in a number of preferred (but non-limiting) Nanobodies of the invention. As will be clear to the skilled person, a combination of CDR1, CDR2 and CDR3 sequences that occur in the same clone (i.e. CDR1, CDR2 and CDR3 sequences that are mentioned on the same line in Table A-1) will usually be preferred (although the invention in its broadest sense is not limited thereto, and also comprises other suitable combinations of the CDR sequences mentioned in Table A-1). Also, a combination of CDR sequences and framework sequences that occur in the same clone (i.e. CDR sequences and framework sequences that are mentioned on the same line in Table A-1) will usually be preferred (although the invention in its broadest sense is not limited thereto, and also comprises other suitable combinations of the CDR sequences and framework sequences mentioned in Table A-1, as well as combinations of such CDR sequences and other suitable framework sequences, e.g. as further described herein).

Also, in the Nanobodies of the invention that comprise the combinations of CDR's mentioned in Table A-1, each CDR can be replaced by a CDR chosen from the group consisting of amino acid sequences that have at least 80%, preferably at least 90%, more preferably at least 95%, even more preferably at least 99% sequence identity (as defined herein) with the mentioned CDR's; in which:
i) any amino acid substitution in such a CDR is preferably, and compared to the corresponding CDR sequence mentioned in Table A-1, a conservative amino acid substitution (as defined herein);
and/or
ii) any such CDR sequence preferably only contains amino acid substitutions, and no amino acid deletions or insertions, compared to the corresponding CDR sequence mentioned in Table A-1;
and/or
iii) any such CDR sequence is a CDR that is derived by means of a technique for affinity maturation known per se, and in particular starting from the corresponding CDR sequence mentioned in Table A-1.

However, as will be clear to the skilled person, the (combinations of) CDR sequences, as well as (the combinations of) CDR sequences and framework sequences mentioned in Table A-1 will generally be preferred.

According to a particularly preferred aspect, in the Nanobodies of the invention, CDR 1 is chosen from the amino acid sequences of SEQ ID NO's: 136 to 141 (and may in particular be the amino acid sequence of SEQ ID NO: 141); CDR2 is chosen from the amino acid sequences of SEQ ID NO's: 152 to 157 (and may in particular be the amino acid sequence of SEQ ID NO: 157); and CDR3 is chosen from the amino acid sequences of SEQ ID NO's: 168 to 173 (and may in particular be the amino acid sequence of SEQ ID NO: 173).

Table A-2 gives the sequences of some preferred but non-limiting amino acid sequences and polypeptides of the invention. SEQ ID NO's: 182 to 189 and 306 to 321 are preferred but non-limiting examples of agonistic VHHs of the invention (of which SEQ ID NO's: 185 to 189 are more preferred and SEQ ID NO: 189 is particularly preferred) and SEQ ID NO's: 190 to 193 are preferred but non-limiting examples of polypeptides containing at least one such agonistic VVH sequence (with SEQ ID NO: 193 being particularly preferred).

More generally, as described herein, one preferred but not limiting aspect of the invention relates to amino acids sequences, compounds or polypeptides of the invention that comprise at least one agonistic VHH chosen from SEQ ID NO's: 182 to 189 and 306 to 321 (and in particular SEQ ID NO's: 185 to 189), with amino acids sequences, compounds or polypeptides of the invention comprising at least one VHH that is SEQ ID NO: 189 being particularly preferred.

As also mentioned herein, according to one preferred but non-limiting aspect, the VHH sequences and Nanobodies described may be formatted as an Fc construct in which VHH sequence/Nanobody is linked, directly or via a suitable linker (such as a hinge sequence) to an Fc portion (which, as further described herein, may be an Fc portion with or without C_{H}1 domain). As also further described herein, said Fc portion may be a naturally occurring Fc portion (such as a human Fc portion, again with or without C_{H}1 domain, with a specific example being the sequence of SEQ ID NO:322) or may be a synthetic or semi-synthetic Fc portion (which is preferably derived from a human Fc portion) such as an Fc portion with reduced effector function (with the Fc portions of SEQ ID NOs: 323 and 324-which comprise the hinge sequence of SEQ ID NO:325) being some particularly preferred but non-limiting examples).

It will also be clear to the skilled person that such Fc constructs will usually comprise two chains (each comprising a VHH and suitable Fc portion, optionally linked via a suitable linker or hinge), but that it may also be possible to use a suitable “monomeric” Fc portion (as further mentioned herein).

Also, as will be clear to the skilled person, the polypeptides of SEQ ID NO's: 190 to 193 are some specific (and specifically preferred) examples of heavy-chain only antibody constructs comprising two agonistic VHH of the invention linked to an Fc tail (as further described herein, i.e. comprising C_{H}2 and C_{H}3 domains with the VHH being directly linked to the C_{H}2 domain without a C_{H}1 domain). As mentioned herein, such Fc-based constructs form a particularly preferred aspect of the invention.

TABLE A-1
Preferred combinations of CDR sequences, preferred combinations of
framework sequences, and preferred combinations of framework and CDR sequences.
(“ID” refers to the SEQ ID NO in the attached sequence listing)

| Clone | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|
| pN0150 | 126 | QVQLV | 134 | IMSMG | 142 | WYRQ | 150 | SISSG | 158 | RFTIS | 166 | QVKST | 174 | WGQGTQ |
| | | ESGG | | | | APGK | | GSTNY | | RDNAK | | PLQFQ | | VTVSS |
| | | GLVQP | | | | QRESV | | ADSVK | | NLMYL | | STRNY | | |
| | | GGSLR | | | | A | | G | | QMNS | | | | |
| | | LSCAA | | | | | | | | LKPED | | | | |
| | | SGRF | | | | | | | | TAVYY | | | | |
| | | QS | | | | | | | | CNA | | | | |
| pN0152 | 127 | QVQLV | 135 | KSDW | 143 | WYRQ | 151 | QITSG | 159 | RFTIS | 167 | RTWR | 175 | WGQGTQ |
| | | ESGG | | G | | APGKE | | GNLKY | | RDNAK | | LVRYY | | VTVSS |
| | | GLVQP | | | | RELVA | | ASAVT | | NTVNL | | DY | | |
| | | GGSLK | | | | | | G | | LMSRL | | | | |
| | | LSCAA | | | | | | | | KPEDT | | | | |
| | | SGSIS | | | | | | | | AIYFC | | | | |
| | | S | | | | | | | | NA | | | | |
| pN0162 | 128 | QVQLV | 136 | LNVM | 144 | WYRQ | 152 | EITKD | 160 | RFTIS | 168 | RTGRI | 176 | WGQGTQ |
| | | ESGG | | G | | TPGN | | GSTNY | | RDNAK | | VRPLD | | VTVSS |
| | | GLVQP | | | | QRALV | | ADSAK | | NTVHL | | Y | | |
| | | GGSLR | | | | A | | G | | QMDS | | | | |
| | | LSCAA | | | | | | | | LKPED | | | | |
| | | SGSAF | | | | | | | | TAVYY | | | | |
| | | S | | | | | | | | CKA | | | | |
| pN2059 | 129 | QVQLV | 137 | LRWM | 145 | WYRQ | 153 | EITKS | 161 | RFTIS | 169 | RTGRI | 177 | WGQGTL |
| | | ESGG | | G | | TPGKQ | | GSTNY | | RDNAK | | VRPVD | | VTVSS |
| | | GLVQP | | | | RELVA | | ADSAK | | NTVYL | | Y | | |
| | | GGSLR | | | | | | G | | QMNS | | | | |
| | | LSCAA | | | | | | | | LRPED | | | | |
| | | SGSAF | | | | | | | | TAVYY | | | | |
| | | S | | | | | | | | CKA | | | | |
| pN2060 | 130 | QVQLV | 138 | LRVM | 146 | WYRQ | 154 | EITKD | 162 | RFTIS | 170 | RTGRI | 178 | WGQGTL |
| | | ESGG | | G | | TPGKQ | | GSTNY | | RDNAK | | VRPVD | | VTVSS |
| | | GLVQP | | | | RELVA | | ADSAK | | NTVYL | | Y | | |
| | | GGSLR | | | | | | G | | QMNS | | | | |
| | | LSCAA | | | | | | | | LRPED | | | | |
| | | SGSAF | | | | | | | | TAVYY | | | | |
| | | R | | | | | | | | CKA | | | | |
| pN2061 | 131 | QVQL | 139 | LRVM | 147 | WYRQ | 155 | EITKS | 163 | RFTIS | 171 | RTGRI | 179 | WGQ |
| | | VESG | | G | | TPGK | | GSTN | | RDNA | | VRPL | | GTLV |
| | | GGLV | | | | QREL | | YADS | | KNTV | | DY | | TVSS |
| | | QPGG | | | | VA | | AKG | | YLQM | | | | |
| | | SLRLS | | | | | | | | NSLR | | | | |
| | | CAAS | | | | | | | | PEDT | | | | |
| | | GSAF | | | | | | | | AVYY | | | | |
| | | R | | | | | | | | CKA | | | | |
| pN2062 | 132 | QVQL | 140 | LHVM | 148 | WYRQ | 156 | EITKS | 164 | RFTIS | 172 | RTGRI | 180 | WGQ |
| | | VESG | | G | | TPGK | | GSTN | | RDNA | | VRPV | | GTLV |
| | | GGLV | | | | QREL | | YADS | | KNTV | | DY | | TVSS |
| | | QPGG | | | | VA | | AKG | | YLQM | | | | |
| | | SLRLS | | | | | | | | NSLR | | | | |
| | | CAAS | | | | | | | | PEDT | | | | |
| | | GSAF | | | | | | | | AVYY | | | | |
| | | R | | | | | | | | CKA | | | | |
| pN2121 | 133 | QVQL | 141 | LHWM | 149 | WYRQ | 157 | EITKS | 165 | RFTIS | 173 | RTGRI | 181 | WGQ |
| | | VESG | | G | | TPGK | | GSTN | | RDNA | | VRPV | | GTLV |
| | | GGLV | | | | QREL | | YADS | | KNTV | | DY | | TVSS |
| | | QPGG | | | | VA | | AKG | | YLQM | | | | |
| | | SLRLS | | | | | | | | NSLR | | | | |
| | | CAAS | | | | | | | | PEDT | | | | |
| | | GSAF | | | | | | | | AVYY | | | | |
| | | S | | | | | | | | CKA | | | | |
| PN0148 | 194 | QVQL | 210 | SNNL | 226 | WYRQ | 242 | AITVG | 258 | RFIIS | 274 | KSTR | 290 | WGQ |
| | | VESG | | G | | APGK | | GTTT | | RDNT | | VVRP | | GTQV |
| | | GGSV | | | | QREL | | YVDS | | KSTLF | | VMY | | TVSS |
| | | QAGG | | | | VA | | VKG | | LQMN | | | | |
| | | SLRLS | | | | | | | | SLEP | | | | |
| | | CAAS | | | | | | | | EDTAI | | | | |
| | | GITFS | | | | | | | | YYCN | | | | |
| | | | | | | | | | | A | | | | |
| PN0149 | 195 | QVQL | 211 | TSNM | 227 | WYRQ | 243 | STTP | 259 | RFTIS | 275 | EVRR | 291 | WGQ |
| | | VESG | | A | | APGK | | GGTIN | | RDNV | | GIARV | | GTQV |
| | | GGLV | | | | QREL | | SANS | | KNMV | | NY | | TVSS |
| | | QPGG | | | | VA | | VKG | | SLQM | | | | |
| | | SLRLS | | | | | | | | NSLK | | | | |
| | | CAAS | | | | | | | | PEDT | | | | |
| | | GFVF | | | | | | | | AVYY | | | | |
| | | G | | | | | | | | CNA | | | | |
| PN0151 | 196 | QVQL | 212 | IVDM | 228 | WYRQ | 244 | TITSG | 260 | RFTIS | 276 | GVRT | 292 | WGQ |
| | | VESG | | G | | APGK | | SNTN | | RDNF | | NFISQ | | GTQV |
| | | GGLV | | | | QREL | | YADS | | KNTV | | RRNY | | TVSS |
| | | QAEG | | | | VA | | VKG | | YLQM | | | | |
| | | SLSLS | | | | | | | | NSLK | | | | |
| | | CAAS | | | | | | | | PEDT | | | | |
| | | GNFF | | | | | | | | AVYY | | | | |
| | | S | | | | | | | | CNA | | | | |
| PN0153 | 197 | QVQL | 213 | INAM | 229 | WYRQ | 245 | AITSG | 261 | RFTIS | 277 | RVRD | 293 | WGQ |
| | | VESG | | G | | APGK | | GSTD | | RDDV | | WRFR | | GTQV |
| | | GGLV | | | | QREL | | LVDF | | KNTV | | EKDY | | TVSS |
| | | QAGG | | | | VA | | VKG | | DLQM | | | | |
| | | SLRLS | | | | | | | | NSLK | | | | |
| | | CAVS | | | | | | | | PEDT | | | | |
| | | RSIFS | | | | | | | | AVYY | | | | |
| | | | | | | | | | | CNA | | | | |
| PN0160 | 198 | QVQL | 214 | AISM | 230 | WYRQ | 246 | TITRG | 262 | RFTIS | 278 | KIRRG | 294 | WGQ |
| | | VESG | | G | | APGK | | DSTN | | RDSA | | IVPIN | | GTQV |
| | | GGLV | | | | QREL | | YADS | | KNTV | | Y | | TVSS |
| | | QAGG | | | | VA | | VKG | | YLQM | | | | |
| | | SLRLS | | | | | | | | NSLK | | | | |
| | | CAAS | | | | | | | | PEDT | | | | |
| | | GSIFS | | | | | | | | AVYY | | | | |
| | | | | | | | | | | CNA | | | | |
| PN0155 | 199 | QVQL | 215 | ISVM | 231 | WYRQ | 247 | TITTG | 263 | RFTA | 279 | KTLQ | 295 | WGQ |
| | | VESG | | G | | APEK | | GNTN | | SRDA | | NPITR | | GTQV |
| | | GGLV | | | | QREW | | YADS | | SKNM | | RTNE | | TVSS |
| | | QAGG | | | | VA | | VKG | | VYLQ | | Y | | |
| | | SLRLS | | | | | | | | MNSL | | | | |
| | | CAVS | | | | | | | | KPED | | | | |
| | | GNIRT | | | | | | | | TAVY | | | | |
| | | | | | | | | | | FCNT | | | | |
| PN0156 | 200 | QVQL | 216 | TNVM | 232 | WHRQ | 248 | TITTG | 264 | RFTIS | 280 | KTLQ | 296 | WGQ |
| | | VESG | | G | | APEK | | GNTN | | RDAT | | NPLT | | GTQV |
| | | GGLV | | | | QREW | | YVDS | | KNMV | | RRTN | | TVSS |
| | | QAGG | | | | VA | | VKG | | YLQM | | EY | | |
| | | SLRLS | | | | | | | | NSLK | | | | |
| | | CAVS | | | | | | | | PEDT | | | | |
| | | GNIRT | | | | | | | | AVYF | | | | |
| | | | | | | | | | | CNT | | | | |
| PN0157 | 201 | QVQL | 217 | INVM | 233 | WYRQ | 249 | DIRR | 265 | RFTIS | 281 | QGLF | 297 | WGQ |
| | | VESG | | G | | VPGK | | GGVT | | RDNA | | RFPR | | GTQV |
| | | GGLV | | | | QREF | | NYAD | | KNTV | | ANVF | | TVSS |
| | | QAGG | | | | VA | | FAKG | | YLQM | | NK | | |
| | | SLRLS | | | | | | | | NSLK | | | | |
| | | CAAS | | | | | | | | PEDT | | | | |
| | | GSIFS | | | | | | | | AVYY | | | | |
| | | | | | | | | | | CNV | | | | |
| PN0161 | 202 | QVQL | 218 | NGMG | 234 | WYRQ | 250 | AITTG | 266 | RFTIS | 282 | RYGS | 298 | WGQ |
| | | VESG | | | | APGK | | GSTN | | RDNA | | SSFR | | GTQV |
| | | GGLV | | | | QREL | | YANS | | KNTV | | WGDF | | TVSS |
| | | QAGG | | | | VA | | AKG | | YLQM | | EV | | |
| | | SLRLS | | | | | | | | NSLK | | | | |
| | | CAAS | | | | | | | | PEDT | | | | |
| | | GSISR | | | | | | | | AVYY | | | | |
| | | T | | | | | | | | CNA | | | | |
| PN0163 | 203 | QVQL | 219 | RAVM | 235 | WYRQ | 251 | TITRG | 267 | RFTIS | 283 | RSGL | 299 | WGQ |
| | | VESG | | G | | APGK | | GSAN | | RVNA | | PYPN | | GTQV |
| | | GGSV | | | | QRES | | YADS | | KNMV | | VY | | TVSS |
| | | QAGG | | | | VA | | VKG | | YLQM | | | | |
| | | SLRLS | | | | | | | | NSLE | | | | |
| | | CAAS | | | | | | | | PEDT | | | | |
| | | GSIFN | | | | | | | | AVYY | | | | |
| | | | | | | | | | | CNA | | | | |
| PN0164 | 204 | QVQL | 220 | INAMA | 236 | WYRQ | 252 | TITSG | 268 | RFAIS | 284 | DGVR | 300 | WGQ |
| | | VESG | | | | APGK | | GSTN | | RDNG | | GSFV | | GTQV |
| | | GGLV | | | | QREL | | YADS | | KNTV | | SQGR | | TVSS |
| | | QAGG | | | | VA | | VKG | | FLQM | | VY | | |
| | | SLRLS | | | | | | | | NSLK | | | | |
| | | CTAS | | | | | | | | PEDT | | | | |
| | | GRIFS | | | | | | | | AVYY | | | | |
| | | | | | | | | | | CNA | | | | |
| PN0159 | 205 | QVQL | 221 | DYTM | 267 | WYRQ | 253 | SITSA | 269 | RFTIS | 285 | KKFRI | 301 | WGQ |
| | | VESG | | G | | APGK | | GTTN | | RDNA | | VGYR | | GTQV |
| | | GGLV | | | | QREL | | YADS | | KNTV | | DY | | TVSS |
| | | QPGG | | | | VA | | VKG | | YLIMN | | | | |
| | | SLRLS | | | | | | | | SLKP | | | | |
| | | CAAS | | | | | | | | EDTA | | | | |
| | | GSISS | | | | | | | | VYYC | | | | |
| | | | | | | | | | | NT | | | | |
| PN0165 | 206 | QVQL | 222 | INVM | 238 | WYRQ | 254 | RINSG | 270 | RFTIS | 286 | NARR | 302 | WGQ |
| | | VESG | | G | | APGK | | GTTN | | RDNA | | NFISN | | GTQV |
| | | GGLV | | | | EREL | | YADS | | KNTV | | RYND | | TVSS |
| | | QAGG | | | | VA | | AKG | | YLQM | | Y | | |
| | | SLRLS | | | | | | | | NSLK | | | | |
| | | CGAS | | | | | | | | PEDT | | | | |
| | | GSIFM | | | | | | | | AVYY | | | | |
| | | | | | | | | | | CNV | | | | |
| PN0166 | 207 | QVQL | 223 | INTM | 239 | WYRQ | 255 | TSTN | 271 | RFTIS | 287 | GVRI | 303 | WGR |
| | | VESG | | G | | TPGQ | | SGKV | | RDNS | | GPAT | | GTQV |
| | | GGPV | | | | QREL | | TYSD | | KKTV | | NAY | | TVSS |
| | | LAGG | | | | VA | | SVKG | | DLQM | | | | |
| | | SLRLS | | | | | | | | NSLK | | | | |
| | | CAAS | | | | | | | | SEDT | | | | |
| | | GRVL | | | | | | | | AVYY | | | | |
| | | V | | | | | | | | CYA | | | | |
| PN0167 | 208 | QVQL | 224 | FNSM | 240 | WYRQ | 256 | RIRTG | 272 | RFTIS | 288 | DTLFP | 304 | WGQ |
| | | VESG | | A | | APGK | | GRTE | | RDNA | | RPVN | | GTQV |
| | | GGLV | | | | QREL | | YADS | | KNTV | | Y | | TVSS |
| | | QAGG | | | | VA | | VKG | | YLQM | | | | |
| | | SLRLS | | | | | | | | NTLR | | | | |
| | | CAGS | | | | | | | | PEDT | | | | |
| | | GSIRS | | | | | | | | AVYY | | | | |
| | | | | | | | | | | CNA | | | | |
| PN0145 | 209 | QVQL | 225 | INVLA | 241 | WYRQ | 257 | GITSG | 273 | RFTIS | 289 | EGRIR | 305 | WGQ |
| | | VESG | | | | APGK | | DSTN | | RDNA | | LNWG | | GTQV |
| | | GGLV | | | | QREL | | YSEA | | KNTV | | SY | | TVSS |
| | | QAGG | | | | VA | | VKG | | YLQM | | | | |
| | | SLRLS | | | | | | | | NSLK | | | | |
| | | CAVP | | | | | | | | PEDT | | | | |
| | | ERMR | | | | | | | | AVYY | | | | |
| | | S | | | | | | | | CHV | | | | |

TABLE A-2
Examples of preferred but non-limiting amino acid sequences and
polypeptides of the invention

| | SEQ | |
|---|---|---|
| Name | ID | SEQUENCE |
| pN0150 | 182 | QVQLVESGGGLVQPGGSLRLSCAASGRFQSIMSMGWYRQAPG |
| | | KQRESVASISSGGSTNYADSVKGRFTISRDNAKNLMYLQMNSLK |
| | | PEDTAVYYCNAQVKSTPLQFQSTRNYWGQGTQVTVSS |
| pN0152 | 183 | QVQLVESGGGLVQPGGSLKLSCAASGSISSKSDWGWYRQAPGK |
| | | ERELVAQITSGGNLKYASAVTGRFTISRDNAKNTVNLLMSRLKPE |
| | | DTAIYFCNARTWRLVRYYDYWGQGTQVTVSS |
| pN0162 | 184 | QVQLVESGGGLVQPGGSLRLSCAASGSAFSLNVMGWYRQTPG |
| | | NQRALVAEITKDGSTNYADSAKGRFTISRDNAKNTVHLQMDSLKP |
| | | EDTAVYYCKARTGRIVRPLDYWGQGTQVTVSS |
| pN2059 | 185 | QVQLVESGGGLVQPGGSLRLSCAASGSAFSLRWMGWYRQTPG |
| | | KQRELVAEITKSGSTNYADSAKGRFTISRDNAKNTVYLQMNSLRP |
| | | EDTAVYYCKARTGRIVRPVDYWGQGTLVTVSS |
| pN2060 | 186 | QVQLVESGGGLVQPGGSLRLSCAASGSAFRLRVMGWYRQTPG |
| | | KQRELVAEITKDGSTNYADSAKGRFTISRDNAKNTVYLQMNSLRP |
| | | EDTAVYYCKARTGRIVRPVDYWGQGTLVTVSS |
| pN2061 | 187 | QVQLVESGGGLVQPGGSLRLSCAASGSAFRLRVMGWYRQTPG |
| | | KQRELVAEITKSGSTNYADSAKGRFTISRDNAKNTVYLQMNSLRP |
| | | EDTAVYYCKARTGRIVRPLDYWGQGTLVTVSS |
| pN2062 | 188 | QVQLVESGGGLVQPGGSLRLSCAASGSAFRLHVMGWYRQTPG |
| | | KQRELVAEITKSGSTNYADSAKGRFTISRDNAKNTVYLQMNSLRP |
| | | EDTAVYYCKARTGRIVRPVDYWGQGTLVTVSS |
| pN2121 | 189 | QVQLVESGGGLVQPGGSLRLSCAASGSAFSLHWMGWYRQTPG |
| | | KQRELVAEITKSGSTNYADSAKGRFTISRDNAKNTVYLQMNSLRP |
| | | EDTAVYYCKARTGRIVRPVDYWGQGTLVTVSS |
| pN0162- | 190 | QVQLVESGGGLVQPGGSLRLSCAASGSAFSLNVMGWYRQTPG |
| huFcIgG1_ | | NQRALVAEITKDGSTNYADSAKGRFTISRDNAKNTVHLQMDSLKP |
| N297A | | EDTAVYYCKARTGRIVRPLDYWGQGTQVTVSSDKTHTCPPCPAP |
| | | ELLGGPSVFLFPPKPKDTLMISRTPEVTCVVVDVSHEDPEVKFNW |
| | | YVDGVEVHNAKTKPREEQYASTYRVVSVLTVLHQDWLNGKEYK |
| | | CKVSNKALPAPIEKTISKAKGQPREPQVYTLPPSRDELTKNQVSL |
| | | TCLVKGFYPSDIAVEWESNGQPENNYKTTPPVLDSDGSFFLYSKL |
| | | TVDKSRWQQGNVFSCSVMHEALHNHYTQKSLSLSPGK |
| pN2037 | 191 | DVQLVESGGGLVQPGGSLRLSCAASGSAFRLHVMGWYRQTPG |
| (huFc | | KQRELVAEITKSGSTNYADSAKGRFTISRDNAKNTVYLQMNSLRP |
| IgG1 | | EDTAVYYCKARTGRIVRPVDYWGQGTLVTVSSDKTHTCPPCPAP |
| L234A_ | | EAAGGPSVFLFPPKPKDTLMISRTPEVTCVVVDVSHEDPEVKFN |
| L235A) | | WYVDGVEVHNAKTKPREEQYNSTYRVVSVLTVLHQDWLNGKEY |
| | | KCKVSNKALPAPIEKTISKAKGQPREPQVYTLPPSRDELTKNQVS |
| | | LTCLVKGFYPSDIAVEWESNGQPENNYKTTPPVLDSDGSFFLYSK |
| | | LTVDKSRWQQGNVFSCSVMHEALHNHYTQKSLSLSPGK |
| pN2112 | 192 | QVQLVESGGGLVQPGGSLRLSCAASGSAFRLHWMGWYRQTPG |
| (huFc_ | | KQRELVAEITKDGSTNYADSAKGRFTISRDNAKNTVYLQMNSLRP |
| IgG1 | | EDTAVYYCKARTGRIVRPVDYWGQGTLVTVSSDKTHTCPPCPAP |
| L234A_ | | EAAGGPSVFLFPPKPKDTLMISRTPEVTCVVVDVSHEDPEVKFN |
| L235A) | | WYVDGVEVHNAKTKPREEQYNSTYRVVSVLTVLHQDWLNGKEY |
| | | KCKVSNKALPAPIEKTISKAKGQPREPQVYTLPPSRDELTKNQVS |
| | | LTCLVKGFYPSDIAVEWESNGQPENNYKTTPPVLDSDGSFFLYSK |
| | | LTVDKSRWQQGNVFSCSVMHEALHNHYTQKSLSLSPGK |
| pN2113 = | 193 | QVQLVESGGGLVQPGGSLRLSCAASGSAFSLHWMGWYRQTPG |
| pN2121- | | KQRELVAEITKSGSTNYADSAKGRFTISRDNAKNTVYLQMNSLRP |
| huFc_ | | EDTAVYYCKARTGRIVRPVDYWGQGTLVTVSSDKTHTCPPCPAP |
| IgG1 | | EAAGGPSVFLFPPKPKDTLMISRTPEVTCVVVDVSHEDPEVKFN |
| L234A_ | | WYVDGVEVHNAKTKPREEQYNSTYRVVSVLTVLHQDWLNGKEY |
| L235A | | KCKVSNKALPAPIEKTISKAKGQPREPQVYTLPPSRDELTKNQVS |
| | | LTCLVKGFYPSDIAVEWESNGQPENNYKTTPPVLDSDGSFFLYSK |
| | | LTVDKSRWQQGNVFSCSVMHEALHNHYTQKSLSLSPGK |
| PN0148 | 306 | QVQLVESGGGSVQAGGSLRLSCAASGITFSSNNLGWYRQAPGK |
| | | QRELVAAITVGGTTTYVDSVKGRFIISRDNTKSTLFLQMNSLEPED |
| | | TAIYYCNAKSTRVVRPVMYWGQGTQVTVSS |
| PN0149 | 307 | QVQLVESGGGLVQPGGSLRLSCAASGFVFGTSNMAWYRQAPG |
| | | KQRELVASTTPGGTINSANSVKGRFTISRDNVKNMVSLQMNSLK |
| | | PEDTAVYYCNAEVRRGIARVNY |
| PN0151 | 308 | QVQLVESGGGLVQAEGSLSLSCAASGNFFSIVDMGWYRQAPGK |
| | | QRELVATITSGSNTNYADSVKGRFTISRDNFKNTVYLQMNSLKPE |
| | | DTAVYYCNAGVRTNFISQRRNYWGQGTQVTVSS |
| PN0153 | 309 | QVQLVESGGGLVQAGGSLRLSCAVSRSIFSINAMGWYRQAPGKQRELV |
| | | AAITSGGSTDLVDFVKGRFTISRDDVKNTVDLQMNSLKPEDTAVYYCNA |
| | | RVRDWRFREKDYWGQGTQVTVSS |
| PN0160 | 310 | QVQLVESGGGLVQAGGSLRLSCAASGSIFSAISMGWYRQAPGKQRELV |
| | | ATITRGDSTNYADSVKGRFTISRDSAKNTVYLQMNSLKPEDTAVYYCNA |
| | | KIRRGIVPINYWGQGTQVTVSS |
| PN0155 | 311 | QVQLVESGGGLVQAGGSLRLSCAVSGNIRTISVMGWYRQAPEKQREW |
| | | VATITTGGNTNYADSVKGRFTASRDASKNMVYLQMNSLKPEDTAVYFC |
| | | NTKTLQNPITRRTNEYWGQGTQVTVSS |
| PN0156 | 312 | QVQLVESGGGLVQAGGSLRLSCAVSGNIRTTNVMGWHRQAPEKQREW |
| | | VATITTGGNTNYVDSVKGRFTISRDATKNMVYLQMNSLKPEDTAVYFCN |
| | | TKTLQNPLTRRTNEYWGQGTQVTVSS |
| PN0157 | 313 | QVQLVESGGGLVQAGGSLRLSCAASGSIFSINVMGWYRQVPGKQREFV |
| | | ADIRRGGVTNYADFAKGRFTISRDNAKNTVYLQMNSLKPEDTAVYYCNV |
| | | QGLFRFPRANVFNKWGQGTQVTVSS |
| PN0161 | 314 | QVQLVESGGGLVQAGGSLRLSCAASGSISRTNGMGWYRQAPGKQREL |
| | | VAAITTGGSTNYANSAKGRFTISRDNAKNTVYLQMNSLKPEDTAVYYCN |
| | | ARYGSSSFRWGDFEVWGQGTQVTVSS |
| PN0163 | 315 | QVQLVESGGGSVQAGGSLRLSCAASGSIFNRAVMGWYRQAPGKQRES |
| | | VATITRGGSANYADSVKGRFTISRVNAKNMVYLQMNSLEPEDTAVYYCN |
| | | ARSGLPYPNVYWGQGTQVTVSS |
| PN0164 | 316 | QVQLVESGGGLVQAGGSLRLSCTASGRIFSINAMAWYRQAPGKQRELV |
| | | ATITSGGSTNYADSVKGRFAISRDNGKNTVFLQMNSLKPEDTAVYYCNA |
| | | DGVRGSFVSQGRVYWGQGTQVTVSS |
| PN0159 | 317 | QVQLVESGGGLVQPGGSLRLSCAASGSISSDYTMGWYRQAPGKQREL |
| | | VASITSAGTTNYADSVKGRFTISRDNAKNTVYLIMNSLKPEDTAVYYCNT |
| | | KKFRIVGYRDYWGQGTQVTVSS |
| PN0165 | 318 | QVQLVESGGGLVQAGGSLRLSCGASGSIFMINVMGWYRQAPGKERELV |
| | | ARINSGGTTNYADSAKGRFTISRDNAKNTVYLQMNSLKPEDTAVYYCNV |
| | | NARRNFISNRYNDYWGQGTQVTVSS |
| PN0166 | 319 | QVQLVESGGGPVLAGGSLRLSCAASGRVLVINTMGWYRQTPGQQREL |
| | | VATSTNSGKVTYSDSVKGRFTISRDNSKKTVDLQMNSLKSEDTAVYYCY |
| | | AGVRIGPATNAYWGRGTQVTVSS |
| PN0167 | 320 | QVQLVESGGGLVQAGGSLRLSCAGSGSIRSFNSMAWYRQAPGKQREL |
| | | VARIRTGGRTEYADSVKGRFTISRDNAKNTVYLQMNTLRPEDTAVYYCN |
| | | ADTLFPRPVNYWGQGTQVTVSS |
| PN0145 | 32 | QVQLVESGGGLVQAGGSLRLSCAVPERMRSINVLAWYRQAPGKQREL |
| | | VAGITSGDSTNYSEAVKGRFTISRDNAKNTVYLQMNSLKPEDTAVYYCH |
| | | VEGRIRLNWGSYWGQGTQVTVSS |

Thus, in the Nanobodies of the invention, at least one of the CDR1, CDR2 and CDR3 sequences present is suitably chosen from the group consisting of the CDR1, CDR2 and CDR3 sequences, respectively, listed in Table A-1; or from the group of CDR1, CDR2 and CDR3 sequences, respectively, that have at least 80%, preferably at least 90%, more preferably at least 95%, even more preferably at least 99% “sequence identity” (as defined herein) with at least one of the CDR1, CDR2 and CDR3 sequences, respectively, listed in Table A-1; and/or from the group consisting of the CDR1, CDR2 and CDR3 sequences, respectively, that have 3, 2 or only 1 “amino acid difference(s)” (as defined herein) with at least one of the CDR1, CDR2 and CDR3 sequences, respectively, listed in Table A-1.

In this context, by “suitably chosen” is meant that, as applicable, a CDR1 sequence is chosen from suitable CDR1 sequences (i.e. as defined herein), a CDR2 sequence is chosen from suitable CDR2 sequences (i.e. as defined herein), and a CDR3 sequence is chosen from suitable CDR3 sequence (i.e. as defined herein), respectively. More in particular, the CDR sequences are preferably chosen such that the Nanobodies of the invention bind to MC4R with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein.

In particular, in the Nanobodies of the invention, at least the CDR3 sequence present is suitably chosen from the group consisting of the CDR3 sequences listed in Table A-1 or from the group of CDR3 sequences that have at least 80%, preferably at least 90%, more preferably at least 95%, even more preferably at least 99% sequence identity with at least one of the CDR3 sequences listed in Table A-1; and/or from the group consisting of the CDR3 sequences that have 3, 2 or only 1 amino acid difference(s) with at least one of the CDR3 sequences listed in Table A-1.

Preferably, in the Nanobodies of the invention, at least two of the CDR1, CDR2 and CDR3 sequences present are suitably chosen from the group consisting of the CDR1, CDR2 and CDR3 sequences, respectively, listed in Table A-1 or from the group consisting of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1; and/or from the group consisting of the CDR1, CDR2 and CDR3 sequences, respectively, that have 3, 2 or only 1 “amino acid difference(s)” with at least one of the CDR1, CDR2 and CDR3 sequences, respectively, listed in Table A-1.

In particular, in the Nanobodies of the invention, at least the CDR3 sequence present is suitably chosen from the group consisting of the CDR3 sequences listed in Table A-1 or from the group of CDR3 sequences that have at least 80%, preferably at least 90%, more preferably at least 95%, even more preferably at least 99% sequence identity with at least one of the CDR3 sequences listed in Table A-1, respectively; and at least one of the CDR1 and CDR2 sequences present is suitably chosen from the group consisting of the CDR1 and CDR2 sequences, respectively, listed in Table A-1 or from the group of CDR1 and CDR2 sequences, respectively, that have at least 80%, preferably at least 90%, more preferably at least 95%, even more preferably at least 99% sequence identity with at least one of the CDR1 and CDR2 sequences, respectively, listed in Table A-1; and/or from the group consisting of the CDR1 and CDR2 sequences, respectively, that have 3, 2 or only 1 amino acid difference(s) with at least one of the CDR1 and CDR2 sequences, respectively, listed in Table A-1.

Most preferably, in the Nanobodies of the invention, all three CDR1, CDR2 and CDR3 sequences present are suitably chosen from the group consisting of the CDR1, CDR2 and CDR3 sequences, respectively, listed in Table A-1 or from the group of CDR1, CDR2 and CDR3 sequences, respectively, that have at least 80%, preferably at least 90%, more preferably at least 95%, even more preferably at least 99% sequence identity with at least one of the CDR1, CDR2 and CDR3 sequences, respectively, listed in Table A-1; and/or from the group consisting of the CDR1, CDR2 and CDR3 sequences, respectively, that have 3, 2 or only 1 amino acid difference(s) with at least one of the CDR1, CDR2 and CDR3 sequences, respectively, listed in Table A-1.

Even more preferably, in the Nanobodies of the invention, at least one of the CDR1, CDR2 and CDR3 sequences present is suitably chosen from the group consisting of the CDR1, CDR2 and CDR3 sequences, respectively, listed in Table A-1. Preferably, in this aspect, at least one or preferably both of the other two CDR sequences present are suitably chosen from CDR sequences that have at least 80%, preferably at least 90%, more preferably at least 95%, even more preferably at least 99% sequence identity with at least one of the corresponding CDR sequences, respectively, listed in Table A-1; and/or from the group consisting of the CDR sequences that have 3, 2 or only 1 amino acid difference(s) with at least one of the corresponding sequences, respectively, listed in Table A-1.

In particular, in the Nanobodies of the invention, at least the CDR3 sequence present is suitably chosen from the group consisting of the CDR3 listed in Table A-1. Preferably, in this aspect, at least one and preferably both of the CDR1 and CDR2 sequences present are suitably chosen from the groups of CDR1 and CDR2 sequences, respectively, that have at least 80%, preferably at least 90%, more preferably at least 95%, even more preferably at least 99% sequence identity with the CDR1 and CDR2 sequences, respectively, listed in Table A-1; and/or from the group consisting of the CDR1 and CDR2 sequences, respectively, that have 3, 2 or only 1 amino acid difference(s) with at least one of the CDR1 and CDR2 sequences, respectively, listed in Table A-1.

Even more preferably, in the Nanobodies of the invention, at least two of the CDR1, CDR2 and CDR3 sequences present are suitably chosen from the group consisting of the CDR1, CDR2 and CDR3 sequences, respectively, listed in Table A-1. Preferably, in this aspect, the remaining CDR sequence present is suitably chosen from the group of CDR sequences that have at least 80%, preferably at least 90%, more preferably at least 95%, even more preferably at least 99% sequence identity with at least one of the corresponding CDR sequences listed in Table A-1; and/or from the group consisting of CDR sequences that have 3, 2 or only 1 amino acid difference(s) with at least one of the corresponding sequences listed in Table A-1.

In particular, in the Nanobodies of the invention, at least the CDR3 sequence is suitably chosen from the group consisting of the CDR3 sequences listed in Table A-1, and either the CDR1 sequence or the CDR2 sequence is suitably chosen from the group consisting of the CDR1 and CDR2 sequences, respectively, listed in Table A-1. Preferably, in this aspect, the remaining CDR sequence present is suitably chosen from the group of CDR sequences that have at least 80%, preferably at least 90%, more preferably at least 95%, even more preferably at least 99% sequence identity with at least one of the corresponding CDR sequences listed in Table A-1; and/or from the group consisting of CDR sequences that have 3, 2 or only 1 amino acid difference(s) with the corresponding CDR sequences listed in Table A-1.

Even more preferably, in the Nanobodies of the invention, all three CDR1, CDR2 and CDR3 sequences present are suitably chosen from the group consisting of the CDR1, CDR2 and CDR3 sequences, respectively, listed in Table A-1.

Also, generally, the combinations of CDR's listed in Table A-1 (i.e. those mentioned on the same line in Table A-1) are preferred. Thus, it is generally preferred that, when a CDR in a Nanobody of the invention is a CDR sequence mentioned in Table A-1 or is suitably chosen from the group of CDR sequences that have at least 80%, preferably at least 90%, more preferably at least 95%, even more preferably at least 99% sequence identity with a CDR sequence listed in Table A-1; and/or from the group consisting of CDR sequences that have 3, 2 or only 1 amino acid difference(s) with a CDR sequence listed in Table A-1, that at least one and preferably both of the other CDR's are suitably chosen from the CDR sequences that belong to the same combination in Table A-1 (i.e. mentioned on the same line in Table A-1) or are suitably chosen from the group of CDR sequences that have at least 80%, preferably at least 90%, more preferably at least 95%, even more preferably at least 99% sequence identity with the CDR sequence(s) belonging to the same combination and/or from the group consisting of CDR sequences that have 3, 2 or only 1 amino acid difference(s) with the CDR sequence(s) belonging to the same combination. The other preferences indicated in the above paragraphs also apply to the combinations of CDR's mentioned in Table A-1.

Thus, by means of non-limiting examples, a Nanobody of the invention can for example comprise a CDR1 sequence that has more than 80% sequence identity with one of the CDR1 sequences mentioned in Table A-1, a CDR2 sequence that has 3, 2 or 1 amino acid difference with one of the CDR2 sequences mentioned in Table A-1 (but belonging to a different combination), and a CDR3 sequence.

Some preferred Nanobodies of the invention may for example comprise: (1) a CDR1 sequence that has more than 80% sequence identity with one of the CDR1 sequences mentioned in Table A-1; a CDR2 sequence that has 3, 2 or 1 amino acid difference with one of the CDR2 sequences mentioned in Table A-1 (but belonging to a different combination); and a CDR3 sequence that has more than 80% sequence identity with one of the CDR3 sequences mentioned in Table A-1 (but belonging to a different combination); or (2) a CDR1 sequence that has more than 80% sequence identity with one of the CDR1 sequences mentioned in Table A-1; a CDR2 sequence, and one of the CDR3 sequences listed in Table A-1; or (3) a CDR1 sequence; a CDR2 sequence that has more than 80% sequence identity with one of the CDR2 sequence listed in Table A-1; and a CDR3 sequence that has 3, 2 or 1 amino acid differences with the CDR3 sequence mentioned in Table A-1 that belongs to the same combination as the CDR2 sequence.

Some particularly preferred Nanobodies of the invention may for example comprise: (1) a CDR1 sequence that has more than 80% sequence identity with one of the CDR1 sequences mentioned in Table A-1; a CDR2 sequence that has 3, 2 or 1 amino acid difference with the CDR2 sequence mentioned in Table A-1 that belongs to the same combination; and a CDR3 sequence that has more than 80% sequence identity with the CDR3 sequence mentioned in Table A-1 that belongs to the same combination; (2) a CDR1 sequence; a CDR 2 listed in Table A-1 and a CDR3 sequence listed in Table A-1 (in which the CDR2 sequence and CDR3 sequence may belong to different combinations).

Some even more preferred Nanobodies of the invention may for example comprise: (1) a CDR1 sequence that has more than 80% sequence identity with one of the CDR1 sequences mentioned in Table A-1; the CDR2 sequence listed in Table A-1 that belongs to the same combination; and a CDR3 sequence mentioned in Table A-1 that belongs to a different combination; or (2) a CDR1 sequence mentioned in Table A-1; a CDR2 sequence that has 3, 2 or 1 amino acid differences with the CDR2 sequence mentioned in Table A-1 that belongs to the same combination; and a CDR3 sequence that has more than 80% sequence identity with the CDR3 sequence listed in Table A-1 that belongs to the same or a different combination.

Particularly preferred Nanobodies of the invention may for example comprise a CDR1 sequence mentioned in Table A-1, a CDR2 sequence that has more than 80% sequence identity with the CDR2 sequence mentioned in Table A-1 that belongs to the same combination; and the CDR3 sequence mentioned in Table A-1 that belongs to the same combination.

In the most preferred Nanobodies of the invention, the CDR1, CDR2 and CDR3 sequences present are suitably chosen from one of the combinations of CDR1, CDR2 and CDR3 sequences, respectively, listed in Table A-1.

According to another preferred, but non-limiting aspect of the invention (a) CDR1 has a length of between 1 and 12 amino acid residues, and usually between 2 and 9 amino acid residues, such as 5, 6 or 7 amino acid residues; and/or (b) CDR2 has a length of between 13 and 24 amino acid residues, and usually between 15 and 21 amino acid residues, such as 16 and 17 amino acid residues; and/or (c) CDR3 has a length of between 2 and 35 amino acid residues, and usually between 3 and 30 amino acid residues, such as between 6 and 23 amino acid residues.

In another preferred, but non-limiting aspect, the invention relates to a Nanobody in which the CDR sequences (as defined herein) have more than 80%, preferably more than 90%, more preferably more than 95%, such as 99% or more sequence identity (as defined herein) with the CDR sequences of at least one of the amino acid sequences of SEQ ID NO's: 182 to 189 and 306 to 321.

Generally, Nanobodies with the above CDR sequences may be as further described herein, and preferably have framework sequences that are also as further described herein. Thus, for example and as mentioned herein, such Nanobodies may be naturally occurring Nanobodies (from any suitable species), naturally occurring V_{HH} sequences (i.e. from a suitable species of Camelid) or synthetic or semi-synthetic amino acid sequences or Nanobodies, including but not limited to partially humanized Nanobodies or V_{HH} sequences, fully humanized Nanobodies or V_{HH} sequences, camelized heavy chain variable domain sequences, as well as Nanobodies that have been obtained by the techniques mentioned herein.

Thus, in one specific, but non-limiting aspect, the invention relates to a humanized Nanobody, which consists of 4 framework regions (FR1 to FR4 respectively) and 3 complementarity determining regions (CDR1 to CDR3 respectively), in which CDR1 to CDR3 are as defined herein and in which said humanized Nanobody comprises at least one humanizing substitution (as defined herein), and in particular at least one humanizing substitution in at least one of its framework sequences (as defined herein).

In another preferred, but non-limiting aspect, the invention relates to a Nanobody in which the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 182 to 189 and 306 to 321. This degree of amino acid identity can for example be determined by determining the degree of amino acid identity (in a manner described herein) between said Nanobody and one or more of the sequences of SEQ ID NO's: 182 to 189 and 306 to 321, in which the amino acid residues that form the framework regions are disregarded. Such Nanobodies can be as further described herein.

In another preferred, but non-limiting aspect, the invention relates to a Nanobody with an amino acid sequence that is chosen from the group consisting of SEQ ID NO's: 182 to 189 and 306 to 321 or from the group consisting of from amino acid sequences that have more than 80%, preferably more than 90%, more preferably more than 95%, such as 99% or more sequence identity (as defined herein) with at least one of the amino acid sequences of SEQ ID NO's: 182 to 189 and 306 to 321.

Another preferred, but non-limiting aspect of the invention relates to humanized variants of the Nanobodies of SEQ ID NO's: 182 to 189 and 306 to 321, that comprise, compared to the corresponding native V_{HH} sequence, at least one humanizing substitution (as defined herein), and in particular at least one humanizing substitution in at least one of its framework sequences (as defined herein). Some preferred, but non-limiting examples of such humanized variants are the humanized Nanobodies of SEQ ID NO's: 185 to 189. Thus, the invention also relates to a humanized Nanobody with an amino acid sequence that is chosen from the group consisting of SEQ ID NO's: 185 to 189 or from the group consisting of from amino acid sequences that have more than 80%, preferably more than 90%, more preferably more than 95%, such as 99% or more sequence identity (as defined herein) with at least one of the amino acid sequences of SEQ ID NO's: 185 to 189 (in which amino acid sequences that are chosen from the latter group of amino acid sequences may contain a greater number or a smaller number of humanizing substitutions compared to the corresponding sequence of SEQ ID NO's: 185 to 189, as long as they retain at least one of the humanizing substitutions present in the corresponding sequence of SEQ ID NO's: 185 to 189).

The polypeptides of the invention comprise or essentially consist of at least one Nanobody of the invention. Some preferred, but non-limiting examples of polypeptides of the invention are given in SEQ ID NO's: 190 to 193.

It will be clear to the skilled person that the Nanobodies that are mentioned herein as “preferred” (or “more preferred”, “even more preferred”, etc.) are also preferred (or more preferred, or even more preferred, etc.) for use in the polypeptides described herein. Thus, polypeptides that comprise or essentially consist of one or more “preferred” Nanobodies of the invention will generally be preferred, and polypeptides that comprise or essentially consist of one or more “more preferred” Nanobodies of the invention will generally be more preferred, etc.

Generally, proteins or polypeptides that comprise or essentially consist of a single Nanobody (such as a single Nanobody of the invention) will be referred to herein as “monovalent” proteins or polypeptides or as “monovalent constructs”. Proteins and polypeptides that comprise or essentially consist of two or more Nanobodies (such as at least two Nanobodies of the invention or at least one Nanobody of the invention and at least one other Nanobody) will be referred to herein as “multivalent” proteins or polypeptides or as “multivalent constructs”, and these may provide certain advantages compared to the corresponding monovalent Nanobodies of the invention. Some non-limiting examples of such multivalent constructs will become clear from the further description herein.

According to one specific, but non-limiting aspect, a polypeptide of the invention comprises or essentially consists of at least two Nanobodies of the invention, such as two or three Nanobodies of the invention. As further described herein, such multivalent constructs can provide certain advantages compared to a protein or polypeptide comprising or essentially consisting of a single Nanobody of the invention, such as a much improved avidity for MC4R. Such multivalent constructs will be clear to the skilled person based on the disclosure herein.

According to another specific, but non-limiting aspect, a polypeptide of the invention comprises or essentially consists of at least one Nanobody of the invention and at least one other binding unit (i.e. directed against another epitope, antigen, target, protein or polypeptide), which is preferably also a Nanobody. Such proteins or polypeptides are also referred to herein as “multispecific” proteins or polypeptides or as ‘multispecific constructs”, and these may provide certain advantages compared to the corresponding monovalent Nanobodies of the invention (as will become clear from the further discussion herein of some preferred, but-nonlimiting multispecific constructs). Such multispecific constructs will be clear to the skilled person based on the disclosure herein.

According to yet another specific, but non-limiting aspect, a polypeptide of the invention comprises or essentially consists of at least one Nanobody of the invention, optionally one or more further Nanobodies, and at least one other amino acid sequence (such as a protein or polypeptide) that confers at least one desired property to the Nanobody of the invention and/or to the resulting fusion protein. Again, such fusion proteins may provide certain advantages compared to the corresponding monovalent Nanobodies of the invention. Some non-limiting examples of such amino acid sequences and of such fusion constructs will become clear from the further description herein.

It is also possible to combine two or more of the above aspects, for example to provide a trivalent bispecific construct comprising two Nanobodies of the invention and one other Nanobody, and optionally one or more other amino acid sequences. Further non-limiting examples of such constructs, as well as some constructs that are particularly preferred within the context of the present invention, will become clear from the further description herein.

In the above constructs, the one or more Nanobodies and/or other amino acid sequences may be directly linked to each other and/or suitably linked to each other via one or more linker sequences. Some suitable but non-limiting examples of such linkers will become clear from the further description herein.

In one specific aspect of the invention, a Nanobody of the invention or a compound, construct or polypeptide of the invention comprising at least one Nanobody of the invention may have an increased half-life, compared to the corresponding amino acid sequence of the invention. Some preferred, but non-limiting examples of such Nanobodies, compounds and polypeptides will become clear to the skilled person based on the further disclosure herein, and for example comprise Nanobodies sequences or polypeptides of the invention that have been chemically modified to increase the half-life thereof (for example, by means of pegylation); amino acid sequences of the invention that comprise at least one additional binding site for binding to a serum protein (such as serum albumin, see for example EP 0 368 684 B1, page 4); or polypeptides of the invention that comprise at least one Nanobody of the invention that is linked to at least one moiety (and in particular at least one amino acid sequence) that increases the half-life of the Nanobody of the invention. Examples of polypeptides of the invention that comprise such half-life extending moieties or amino acid sequences will become clear to the skilled person based on the further disclosure herein; and for example include, without limitation, polypeptides in which the one or more Nanobodies of the invention are suitable linked to one or more serum proteins or fragments thereof (such as serum albumin or suitable fragments thereof) or to one or more binding units that can bind to serum proteins (such as, for example, Nanobodies or (single) domain antibodies that can bind to serum proteins such as serum albumin, serum immunoglobulins such as IgG, or transferrine); polypeptides in which a Nanobody of the invention is linked to an Fc portion (such as a human Fc) or a suitable part or fragment thereof; or polypeptides in which the one or more Nanobodies of the invention are suitable linked to one or more small proteins or peptides that can bind to serum proteins (such as, without limitation, the proteins and peptides described in WO 91/01743, WO 01/45746, WO 02/076489 and to the US provisional application of Ablynx N.V. entitled “Peptides capable of binding to serum proteins” of Ablynx N.V. filed on Dec. 5, 2006 (see also PCT/EP/2007/063348).

Again, as will be clear to the skilled person, such Nanobodies, compounds, constructs or polypeptides may contain one or more additional groups, residues, moieties or binding units, such as one or more further amino acid sequences and in particular one or more additional Nanobodies (i.e. not directed against MC4R), so as to provide a tri- of multispecific Nanobody construct.

Generally, the Nanobodies of the invention (or compounds, constructs or polypeptides comprising the same) with increased half-life preferably have a half-life that is at least 1.5 times, preferably at least 2 times, such as at least 5 times, for example at least 10 times or more than 20 times, greater than the half-life of the corresponding amino acid sequence of the invention per se. For example, the Nanobodies, compounds, constructs or polypeptides of the invention with increased half-life may have a half-life that is increased with more than 1 hours, preferably more than 2 hours, more preferably more than 6 hours, such as more than 12 hours, or even more than 24, 48 or 72 hours, compared to the corresponding amino acid sequence of the invention per se.

In a preferred, but non-limiting aspect of the invention, such Nanobodies, compound, constructs or polypeptides of the invention exhibit a serum half-life in human of at least about 12 hours, preferably at least 24 hours, more preferably at least 48 hours, even more preferably at least 72 hours or more. For example, compounds or polypeptides of the invention may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

In another one aspect of the invention, a polypeptide of the invention comprises one or more (such as two or preferably one) Nanobodies of the invention linked (optionally via one or more suitable linker sequences) to one or more (such as two and preferably one) amino acid sequences that allow the resulting polypeptide of the invention to cross the blood brain barrier. In particular, said one or more amino acid sequences that allow the resulting polypeptides of the invention to cross the blood brain barrier may be one or more (such as two and preferably one) Nanobodies, such as the Nanobodies described in WO 02/057445, of which FC44 (SEQ ID NO: 189 of WO 06/040153) and FC5 (SEQ ID NO: 190 of WO 06/040154) are preferred examples.

Preferably, a polypeptide that contains only one amino acid sequence of the invention is preferably such that it will bind to MC4R with an affinity less than 500 nM, preferably less than 200 nM, more preferably less than 10 nM, such as less than 500 pM. In this respect, it will be clear to the skilled person that a polypeptide that contains two or more Nanobodies of the invention may bind to MC4R with an increased avidity, compared to a polypeptide that contains only one amino acid sequence of the invention.

Some preferred IC₅₀ values for binding of the amino acid sequences or polypeptides of the invention to MC4R will become clear from the further description and examples herein.

Other polypeptides according to this preferred aspect of the invention may for example be chosen from the group consisting of amino acid sequences that have more than 80%, preferably more than 90%, more preferably more than 95%, such as 99% or more “sequence identity” (as defined herein) with one or more of the amino acid sequences of SEQ ID NO's: 190 to 193, in which the Nanobodies comprised within said amino acid sequences are preferably as further defined herein.

Another aspect of this invention relates to a nucleic acid that encodes an amino acid sequence of the invention (such as a Nanobody of the invention) or a polypeptide of the invention comprising the same. Again, as generally described herein for the nucleic acids of the invention, such a nucleic acid may be in the form of a genetic construct, as defined herein.

In another aspect, the invention relates to host or host cell that expresses or that is capable of expressing an amino acid sequence (such as a Nanobody) of the invention and/or a polypeptide of the invention comprising the same; and/or that contains a nucleic acid of the invention. Some preferred but non-limiting examples of such hosts or host cells will become clear from the further description herein.

Another aspect of the invention relates to a product or composition containing or comprising at least one amino acid sequence of the invention, at least one polypeptide of the invention and/or at least one nucleic acid of the invention, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will become clear from the further description herein.

The invention further relates to methods for preparing or generating the amino acid sequences, compounds, constructs, polypeptides, nucleic acids, host cells, products and compositions described herein. Some preferred but non-limiting examples of such methods will become clear from the further description herein.

The invention further relates to applications and uses of the amino acid sequences, compounds, constructs, polypeptides, nucleic acids, host cells, products and compositions described herein, as well as to methods for the prevention and/or treatment for diseases and disorders associated with MC4R. Some preferred but non-limiting applications and uses will become clear from the further description herein.

Other aspects, embodiments, advantages and applications of the invention will also become clear from the further description hereinbelow.

Generally, it should be noted that the term Nanobody as used herein in its broadest sense is not limited to a specific biological source or to a specific method of preparation. For example, as will be discussed in more detail below, the Nanobodies of the invention can generally be obtained by any of the techniques (1) to (8) mentioned on pages 61 and 62 of WO 08/020079, or any other suitable technique known per se. One preferred class of Nanobodies corresponds to the V_{HH} domains of naturally occurring heavy chain antibodies directed against MC4R. As further described herein, such V_{HH} sequences can generally be generated or obtained by suitably immunizing a species of Camelid with MC4R (i.e. so as to raise an immune response and/or heavy chain antibodies directed against MC4R), by obtaining a suitable biological sample from said Camelid (such as a blood sample, serum sample or sample of B-cells), and by generating V_{HH} sequences directed against MC4R, starting from said sample, using any suitable technique known per se. Such techniques will be clear to the skilled person and/or are further described herein.

Alternatively, such naturally occurring V_{HH} domains against MC4R, can be obtained from naïve libraries of Camelid V_{HH} sequences, for example by screening such a library using MC4R, or at least one part, fragment, antigenic determinant or epitope thereof using one or more screening techniques known per se. Such libraries and techniques are for example described in WO 99/37681, WO 01/90190, WO 03/025020 and WO 03/035694. Alternatively, improved synthetic or semi-synthetic libraries derived from naïve V_{HH} libraries may be used, such as V_{HH} libraries obtained from naïve V_{HH} libraries by techniques such as random mutagenesis and/or CDR shuffling, as for example described in WO 00/43507.

Thus, in another aspect, the invention relates to a method for generating Nanobodies, that are directed against MC4R. In one aspect, said method at least comprises the steps of:
a) providing a set, collection or library of Nanobody sequences; and
b) screening said set, collection or library of Nanobody sequences for Nanobody sequences that can bind to and/or have affinity for MC4R;
and
c) isolating the amino acid sequence(s) that can bind to and/or have affinity for MC4R.

In such a method, the set, collection or library of Nanobody sequences may be a naïve set, collection or library of Nanobody sequences; a synthetic or semi-synthetic set, collection or library of Nanobody sequences; and/or a set, collection or library of Nanobody sequences that have been subjected to affinity maturation.

In a preferred aspect of this method, the set, collection or library of Nanobody sequences may be an immune set, collection or library of Nanobody sequences, and in particular an immune set, collection or library of V_{HH} sequences, that have been derived from a species of Camelid that has been suitably immunized with MC4R or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of Nanobody or V_{HH} sequences may be displayed on a phage, phagemid, ribosome or suitable micro-organism (such as yeast), such as to facilitate screening. Suitable methods, techniques and host organisms for displaying and screening (a set, collection or library of) Nanobody sequences will be clear to the person skilled in the art, for example on the basis of the further disclosure herein. Reference is also made to WO 03/054016 and to the review by Hoogenboom in Nature Biotechnology, 23, 9, 1105-1116 (2005).

In another aspect, the method for generating Nanobody sequences comprises at least the steps of:
a) providing a collection or sample of cells derived from a species of Camelid that express immunoglobulin sequences;
b) screening said collection or sample of cells for (i) cells that express an immunoglobulin sequence that can bind to and/or have affinity for MC4R; and (ii) cells that express heavy chain antibodies, in which substeps (i) and (ii) can be performed essentially as a single screening step or in any suitable order as two separate screening steps, so as to provide at least one cell that expresses a heavy chain antibody that can bind to and/or has affinity for MC4R;
and
c) either (i) isolating from said cell the V_{HH} sequence present in said heavy chain antibody; or (ii) isolating from said cell a nucleic acid sequence that encodes the V_{HH} sequence present in said heavy chain antibody, followed by expressing said V_{HH} domain.

In the method according to this aspect, the collection or sample of cells may for example be a collection or sample of B-cells. Also, in this method, the sample of cells may be derived from a Camelid that has been suitably immunized with MC4R or a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

The above method may be performed in any suitable manner, as will be clear to the skilled person. Reference is for example made to EP 0 542 810, WO 05/19824, WO 04/051268 and WO 04/106377. The screening of step b) is preferably performed using a flow cytometry technique such as FACS. For this, reference is for example made to Lieby et al., Blood, Vol. 97, No. 12, 3820. Particular reference is made to the so-called “Nanoclone™” technique described in International application WO 06/079372 by Ablynx N.V.

In another aspect, the method for generating an amino acid sequence directed against MC4R may comprise at least the steps of:
a) providing a set, collection or library of nucleic acid sequences encoding heavy chain antibodies or Nanobody sequences;
b) screening said set, collection or library of nucleic acid sequences for nucleic acid sequences that encode a heavy chain antibody or a Nanobody sequence that can bind to and/or has affinity for MC4R;
and
c) isolating said nucleic acid sequence, followed by expressing the V_{HH} sequence present in said heavy chain antibody or by expressing said Nanobody sequence, respectively.

In such a method, the set, collection or library of nucleic acid sequences encoding heavy chain antibodies or Nanobody sequences may for example be a set, collection or library of nucleic acid sequences encoding a naïve set, collection or library of heavy chain antibodies or V_{HH} sequences; a set, collection or library of nucleic acid sequences encoding a synthetic or semi-synthetic set, collection or library of Nanobody sequences; and/or a set, collection or library of nucleic acid sequences encoding a set, collection or library of Nanobody sequences that have been subjected to affinity maturation.

In a preferred aspect of this method, the set, collection or library of amino acid sequences may be an immune set, collection or library of nucleic acid sequences encoding heavy chain antibodies or V_{HH} sequences derived from a Camelid that has been suitably immunized with MC4R or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to WO 03/054016 and to the review by Hoogenboom in Nature Biotechnology, 23, 9, 1105-1116 (2005).

As will be clear to the skilled person, the screening step of the methods described herein can also be performed as a selection step. Accordingly the term “screening” as used in the present description can comprise selection, screening or any suitable combination of selection and/or screening techniques. Also, when a set, collection or library of sequences is used, it may contain any suitable number of sequences, such as 1, 2, 3 or about 5, 10, 50, 100, 500, 1000, 5000, 10⁴, 10⁵, 10⁶, 10⁷, 10⁸ or more sequences.

Also, one or more or all of the sequences in the above set, collection or library of amino acid sequences may be obtained or defined by rational, or semi-empirical approaches such as computer modelling techniques or biostatics or datamining techniques.

Furthermore, such a set, collection or library can comprise one, two or more sequences that are variants from one another (e.g. with designed point mutations or with randomized positions), compromise multiple sequences derived from a diverse set of naturally diversified sequences (e.g. an immune library)), or any other source of diverse sequences (as described for example in Hoogenboom et al, Nat Biotechnol 23:1105, 2005 and Binz et al, Nat Biotechnol 2005, 23:1247). Such set, collection or library of sequences can be displayed on the surface of a phage particle, a ribosome, a bacterium, a yeast cell, a mammalian cell, and linked to the nucleotide sequence encoding the amino acid sequence within these carriers. This makes such set, collection or library amenable to selection procedures to isolate the desired amino acid sequences of the invention. More generally, when a sequence is displayed on a suitable host or host cell, it is also possible (and customary) to first isolate from said host or host cell a nucleotide sequence that encodes the desired sequence, and then to obtain the desired sequence by suitably expressing said nucleotide sequence in a suitable host organism. Again, this can be performed in any suitable manner known per se, as will be clear to the skilled person.

Yet another technique for obtaining V_{HH} sequences or Nanobody sequences directed against MC4R involves suitably immunizing a transgenic mammal that is capable of expressing heavy chain antibodies (i.e. so as to raise an immune response and/or heavy chain antibodies directed against MC4R), obtaining a suitable biological sample from said transgenic mammal that contains (nucleic acid sequences encoding) said V_{HH} sequences or Nanobody sequences (such as a blood sample, serum sample or sample of B-cells), and then generating V_{HH} sequences directed against MC4R, starting from said sample, using any suitable technique known per se (such as any of the methods described herein or a hybridoma technique). For example, for this purpose, the heavy chain antibody-expressing mice and the further methods and techniques described in WO 02/085945, WO 04/049794 and WO 06/008548 and Janssens et al., Proc. Natl. Acad. Sci. USA. 2006 Oct. 10; 103 (41): 15130-5 can be used. For example, such heavy chain antibody expressing mice can express heavy chain antibodies with any suitable (single) variable domain, such as (single) variable domains from natural sources (e.g. human (single) variable domains, Camelid (single) variable domains or shark (single) variable domains), as well as for example synthetic or semi-synthetic (single) variable domains.

The invention also relates to the V_{HH} sequences or Nanobody sequences that are obtained by the above methods, or alternatively by a method that comprises the one of the above methods and in addition at least the steps of determining the nucleotide sequence or amino acid sequence of said V_{HH} sequence or Nanobody sequence; and of expressing or synthesizing said V_{HH} sequence or Nanobody sequence in a manner known per se, such as by expression in a suitable host cell or host organism or by chemical synthesis.

As mentioned herein, a particularly preferred class of Nanobodies of the invention comprises Nanobodies with an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a Vu domain from a conventional 4-chain antibody from a human being (e.g. indicated above), as further described on, and using the techniques mentioned on, page 63 of WO 08/020079. Another particularly preferred class of Nanobodies of the invention comprises Nanobodies with an amino acid sequence that corresponds to the amino acid sequence of a naturally occurring V_{H} domain, but that has been “camelized”, i.e. by replacing one or more amino acid residues in the amino acid sequence of a naturally occurring Vu domain from a conventional 4-chain antibody by one or more of the amino acid residues that occur at the corresponding position(s) in a V_{HH} domain of a heavy chain antibody, as further described on, and using the techniques mentioned on, page 63 of WO 08/020079.

Other suitable methods and techniques for obtaining the Nanobodies of the invention and/or nucleic acids encoding the same, starting from naturally occurring V_{H} sequences or preferably V_{HH} sequences, will be clear from the skilled person, and may for example include the techniques that are mentioned on page 64 of WO 08/00279. As mentioned herein, Nanobodies may in particular be characterized by the presence of one or more “Hallmark residues” (as described herein) in one or more of the framework sequences.

Thus, according to one preferred, but non-limiting aspect of the invention, a Nanobody in its broadest sense can be generally defined as a polypeptide comprising:
a) an amino acid sequence that is comprised of four framework regions/sequences interrupted by three complementarity determining regions/sequences, in which the amino acid residue at position 108 according to the Kabat numbering is Q;
and/or:
b) an amino acid sequence that is comprised of four framework regions/sequences interrupted by three complementarity determining regions/sequences, in which the amino acid residue at position 45 according to the Kabat numbering is a charged amino acid (as defined herein) or a cysteine residue, and position 44 is preferably an E; and/or:
c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, in a first preferred, but non-limiting aspect, a Nanobody of the invention may have the structure

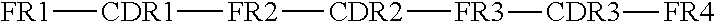

in which FR1 to FR4 refer to framework regions 1 to 4, respectively, and in which CDR1 to CDR3 refer to the complementarity determining regions 1 to 3, respectively, and in which
a) the amino acid residue at position 108 according to the Kabat numbering is Q;
and/or in which:
b) the amino acid residue at position 45 according to the Kabat numbering is a charged amino acid or a cysteine and the amino acid residue at position 44 according to the Kabat numbering is preferably E;
and/or in which:
c) the amino acid residue at position 103 according to the Kabat numbering is chosen from the group consisting of P, R and S, and is in particular chosen from the group consisting of R and S;
and in which:
d) CDR1, CDR2 and CDR3 are as defined herein, and are preferably as defined according to one of the preferred aspects herein, and are more preferably as defined according to one of the more preferred aspects herein.

In particular, a Nanobody in its broadest sense can be generally defined as a polypeptide comprising:
a) an amino acid sequence that is comprised of four framework regions/sequences interrupted by three complementarity determining regions/sequences, in which the amino acid residue at position 108 according to the Kabat numbering is Q;
and/or:
b) an amino acid sequence that is comprised of four framework regions/sequences interrupted by three complementarity determining regions/sequences, in which the amino acid residue at position 44 according to the Kabat numbering is E and in which the amino acid residue at position 45 according to the Kabat numbering is an R;
and/or:
c) an amino acid sequence that is comprised of four framework regions/sequences interrupted by three complementarity determining regions/sequences, in which the amino acid residue at position 103 according to the Kabat numbering is chosen from the group consisting of P, R and S, and is in particular chosen from the group consisting of R and S.

Thus, according to a preferred, but non-limiting aspect, a Nanobody of the invention may have the structure

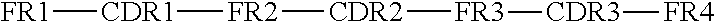

in which FR1 to FR4 refer to framework regions 1 to 4, respectively, and in which CDR1 to CDR3 refer to the complementarity determining regions 1 to 3, respectively, and in which
a) the amino acid residue at position 108 according to the Kabat numbering is Q; and/or in which:
b) the amino acid residue at position 44 according to the Kabat numbering is E and in
   which the amino acid residue at position 45 according to the Kabat numbering is an R;
and/or in which:
c) the amino acid residue at position 103 according to the Kabat numbering is chosen from the group consisting of P, R and S, and is in particular chosen from the group consisting of R and S;
and in which:
d) CDR1, CDR2 and CDR3 are as defined herein, and are preferably as defined according to one of the preferred aspects herein, and are more preferably as defined according to one of the more preferred aspects herein.

In particular, a Nanobody against MC4R according to the invention may have the structure:

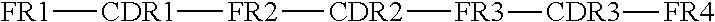

in which FR1 to FR4 refer to framework regions 1 to 4, respectively, and in which CDR1 to CDR3 refer to the complementarity determining regions 1 to 3, respectively, and in which
a) the amino acid residue at position 108 according to the Kabat numbering is Q; and/or in which:
b) the amino acid residue at position 44 according to the Kabat numbering is E and in which the amino acid residue at position 45 according to the Kabat numbering is an R;
and/or in which:
c) the amino acid residue at position 103 according to the Kabat numbering is chosen from the group consisting of P, R and S, and is in particular chosen from the group consisting of R and S;
and in which:
d) CDR1, CDR2 and CDR3 are as defined herein, and are preferably as defined according to one of the preferred aspects herein, and are more preferably as defined according to one of the more preferred aspects herein.

In particular, according to one preferred, but non-limiting aspect of the invention, a Nanobody can generally be defined as a polypeptide comprising an amino acid sequence that is comprised of four framework regions/sequences interrupted by three complementarity determining regions/sequences, in which;
a-1) the amino acid residue at position 44 according to the Kabat numbering is chosen from the group consisting of A, G, E, D, G, Q, R, S, L; and is preferably chosen from the group consisting of G, E or Q; and
a-2) the amino acid residue at position 45 according to the Kabat numbering is chosen from the group consisting of L, R or C; and is preferably chosen from the group consisting of L or R; and
a-3) the amino acid residue at position 103 according to the Kabat numbering is chosen from the group consisting of W, R or S; and is preferably W or R, and is most preferably W;
a-4) the amino acid residue at position 108 according to the Kabat numbering is Q;
or in which:
b-1) the amino acid residue at position 44 according to the Kabat numbering is chosen from the group consisting of E and Q; and
b-2) the amino acid residue at position 45 according to the Kabat numbering is R; and
b-3) the amino acid residue at position 103 according to the Kabat numbering is chosen from the group consisting of W, R and S; and is preferably W;
b-4) the amino acid residue at position 108 according to the Kabat numbering is chosen from the group consisting of Q and L; and is preferably Q;
or in which:
c-1) the amino acid residue at position 44 according to the Kabat numbering is chosen from the group consisting of A, G, E, D, Q, R, S and L; and is preferably chosen from the group consisting of G, E and Q; and
c-2) the amino acid residue at position 45 according to the Kabat numbering is chosen from the group consisting of L, R and C; and is preferably chosen from the group consisting of L and R; and
c-3) the amino acid residue at position 103 according to the Kabat numbering is chosen from the group consisting of P, R and S; and is in particular chosen from the group consisting of R and S; and
c-4) the amino acid residue at position 108 according to the Kabat numbering is chosen from the group consisting of Q and L; is preferably Q;
and in which
d) CDR1, CDR2 and CDR3 are as defined herein, and are preferably as defined according to one of the preferred aspects herein, and are more preferably as defined according to one of the more preferred aspects herein.

Thus, in another preferred, but non-limiting aspect, a Nanobody of the invention may have the structure

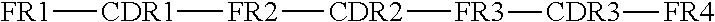

in which FR1 to FR4 refer to framework regions 1 to 4, respectively, and in which CDR1 to CDR3 refer to the complementarity determining regions 1 to 3, respectively, and in which:
a-1) the amino acid residue at position 44 according to the Kabat numbering is chosen from the group consisting of A, G, E, D, G, Q, R, S, L; and is preferably chosen from the group consisting of G, E or Q;
and in which:
a-2) the amino acid residue at position 45 according to the Kabat numbering is chosen from the group consisting of L, R or C; and is preferably chosen from the group consisting of L or R;
and in which:
a-3) the amino acid residue at position 103 according to the Kabat numbering is chosen from the group consisting of W, R or S; and is preferably W or R, and is most preferably W;
and in which
a-4) the amino acid residue at position 108 according to the Kabat numbering is Q;
and in which:
d) CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may have the structure

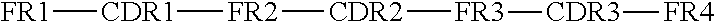

in which FR1 to FR4 refer to framework regions 1 to 4, respectively, and in which CDR1 to CDR3 refer to the complementarity determining regions 1 to 3, respectively, and in which:
b-1) the amino acid residue at position 44 according to the Kabat numbering is chosen from the group consisting of E and Q;
and in which:
b-2) the amino acid residue at position 45 according to the Kabat numbering is R;
and in which:
b-3) the amino acid residue at position 103 according to the Kabat numbering is chosen from the group consisting of W, R and S; and is preferably W;
and in which:
b-4) the amino acid residue at position 108 according to the Kabat numbering is chosen from the group consisting of Q and L; and is preferably Q;
and in which:
d) CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may have the structure

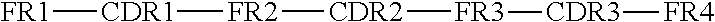

in which FR1 to FR4 refer to framework regions 1 to 4, respectively, and in which CDR1 to CDR3 refer to the complementarity determining regions 1 to 3, respectively, and in which:
c-1) the amino acid residue at position 44 according to the Kabat numbering is chosen from the group consisting of A, G, E, D, Q, R, S and L; and is preferably chosen from the group consisting of G, E and Q;
and in which:
c-2) the amino acid residue at position 45 according to the Kabat numbering is chosen from the group consisting of L, R and C; and is preferably chosen from the group consisting of L and R;
and in which:
c-3) the amino acid residue at position 103 according to the Kabat numbering is chosen from the group consisting of P, R and S; and is in particular chosen from the group consisting of R and S;
and in which:
c-4) the amino acid residue at position 108 according to the Kabat numbering is chosen from the group consisting of Q and L; is preferably Q;
and in which:
d) CDR1, CDR2 and CDR3 are as defined herein, and are preferably as defined according to one of the preferred aspects herein, and are more preferably as defined according to one of the more preferred aspects herein.

Two particularly preferred, but non-limiting groups of the Nanobodies of the invention are those according to a) above; according to (a-1) to (a-4) above; according to b) above; according to (b-1) to (b-4) above; according to (c) above; and/or according to (c-1) to (c-4) above, in which either:
i) the amino acid residues at positions 44-47 according to the Kabat numbering form the sequence GLEW (or a GLEW-like sequence as described herein) and the amino acid residue at position 108 is Q;
or in which:
ii) the amino acid residues at positions 43-46 according to the Kabat numbering form the sequence KERE or KQRE (or a KERE-like sequence as described) and the amino acid residue at position 108 is Q or L, and is preferably Q.

Thus, in another preferred, but non-limiting aspect, a Nanobody of the invention may have the structure

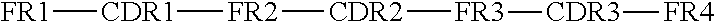

in which FR1 to FR4 refer to framework regions 1 to 4, respectively, and in which CDR1 to CDR3 refer to the complementarity determining regions 1 to 3, respectively, and in which:
i) the amino acid residues at positions 44-47 according to the Kabat numbering form the sequence GLEW (or a GLEW-like sequence as defined herein) and the amino acid residue at position 108 is Q;
and in which:
ii) CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may have the structure

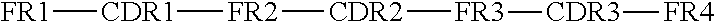

in which FR1 to FR4 refer to framework regions 1 to 4, respectively, and in which CDR1 to CDR3 refer to the complementarity determining regions 1 to 3, respectively, and in which:
i) the amino acid residues at positions 43-46 according to the Kabat numbering form the sequence KERE or KQRE (or a KERE-like sequence) and the amino acid residue at position 108 is Q or L, and is preferably Q;
and in which:
ii) CDR1, CDR2 and CDR3 are as defined herein, and are preferably as defined according to one of the preferred aspects herein, and are more preferably as defined according to one of the more preferred aspects herein.

In the Nanobodies of the invention in which the amino acid residues at positions 43-46 according to the Kabat numbering form the sequence KERE or KQRE, the amino acid residue at position 37 is most preferably F. In the Nanobodies of the invention in which the amino acid residues at positions 44-47 according to the Kabat numbering form the sequence GLEW, the amino acid residue at position 37 is chosen from the group consisting of Y, H, I, L, V or F, and is most preferably V.

Thus, without being limited hereto in any way, on the basis of the amino acid residues present on the positions mentioned above, the Nanobodies of the invention can generally be classified on the basis of the following three groups:
i) The “GLEW-group”: Nanobodies with the amino acid sequence GLEW at positions 44-47 according to the Kabat numbering and Q at position 108 according to the Kabat numbering. As further described herein, Nanobodies within this group usually have a V at position 37, and can have a W, P, R or S at position 103, and preferably have a W at position 103. The GLEW group also comprises some GLEW-like sequences such as those mentioned in Table A-3 below. More generally, and without limitation, Nanobodies belonging to the GLEW-group can be defined as Nanobodies with a G at position 44 and/or with a W at position 47, in which position 46 is usually E and in which preferably position 45 is not a charged amino acid residue and not cysteine;
ii) The “KERE-group”: Nanobodies with the amino acid sequence KERE or KQRE (or another KERE-like sequence) at positions 43-46 according to the Kabat numbering and Q or L at position 108 according to the Kabat numbering. As further described herein, Nanobodies within this group usually have a F at position 37, an L or F at position 47; and can have a W, P, R or S at position 103, and preferably have a W at position 103. More generally, and without limitation, Nanobodies belonging to the KERE-group can be defined as Nanobodies with a K, Q or R at position 44 (usually K) in which position 45 is a charged amino acid residue or cysteine, and position 47 is as further defined herein;
iii) The “103 P, R, S-group”: Nanobodies with a P, R or S at position 103. These Nanobodies can have either the amino acid sequence GLEW at positions 44-47 according to the Kabat numbering or the amino acid sequence KERE or KQRE at positions 43-46 according to the Kabat numbering, the latter most preferably in combination with an F at position 37 and an L or an F at position 47 (as defined for the KERE-group); and can have Q or L at position 108 according to the Kabat numbering, and preferably have Q.

Also, where appropriate, Nanobodies may belong to (i.e. have characteristics of) two or more of these classes. For example, one specifically preferred group of Nanobodies has GLEW or a GLEW-like sequence at positions 44-47; P, R or S (and in particular R) at position 103; and Q at position 108 (which may be humanized to L).

More generally, it should be noted that the definitions referred to above describe and apply to Nanobodies in the form of a native (i.e. non-humanized) V_{HH} sequence, and that humanized variants of these Nanobodies may contain other amino acid residues than those indicated above (i.e. one or more humanizing substitutions as defined herein). For example, and without limitation, in some humanized Nanobodies of the GLEW-group or the 103 P, R, S-group, Q at position 108 may be humanized to 108L. As already mentioned herein, other humanizing substitutions (and suitable combinations thereof) will become clear to the skilled person based on the disclosure herein. In addition, or alternatively, other potentially useful humanizing substitutions can be ascertained by comparing the sequence of the framework regions of a naturally occurring V_{HH} sequence with the corresponding framework sequence of one or more closely related human V_{H} sequences, after which one or more of the potentially useful humanizing substitutions (or combinations thereof) thus determined can be introduced into said V_{HH} sequence (in any manner known per se, as further described herein) and the resulting humanized V_{HH} sequences can be tested for affinity for the target, for stability, for case and level of expression, and/or for other desired properties. In this way, by means of a limited degree of trial and error, other suitable humanizing substitutions (or suitable combinations thereof) can be determined by the skilled person based on the disclosure herein. Also, based on the foregoing, (the framework regions of) a Nanobody may be partially humanized or fully humanized.

Thus, in another preferred, but non-limiting aspect, a Nanobody of the invention may be a Nanobody belonging to the GLEW-group (as defined herein), and in which CDR1, CDR2 and CDR3 are as defined herein, and are preferably as defined according to one of the preferred aspects herein, and are more preferably as defined according to one of the more preferred aspects herein.

In another preferred, but non-limiting aspect, a Nanobody of the invention may be a Nanobody belonging to the KERE-group (as defined herein), and CDR1, CDR2 and CDR3 are as defined herein, and are preferably as defined according to one of the preferred aspects herein, and are more preferably as defined according to one of the more preferred aspects herein.

Thus, in another preferred, but non-limiting aspect, a Nanobody of the invention may be a Nanobody belonging to the 103 P, R, S-group (as defined herein), and in which CDR1, CDR2 and CDR3 are as defined herein, and are preferably as defined according to one of the preferred aspects herein, and are more preferably as defined according to one of the more preferred aspects herein.

Also, more generally and in addition to the 108Q, 43E/44R and 103 P,R,S residues mentioned above, the Nanobodies of the invention can contain, at one or more positions that in a conventional V_{H} domain would form (part of) the V_{H}/V_{L} interface, one or more amino acid residues that are more highly charged than the amino acid residues that naturally occur at the same position(s) in the corresponding naturally occurring V_{H} sequence, and in particular one or more charged amino acid residues (as mentioned in Table A-2 of WO 08/020079). Such substitutions include, but are not limited to, the GLEW-like sequences mentioned in Table A-3 below; as well as the substitutions that are described in the International Application WO 00/29004 for so-called “microbodies”, e.g. so as to obtain a Nanobody with Q at position 108 in combination with KLEW at positions 44-47. Other possible substitutions at these positions will be clear to the skilled person based upon the disclosure herein.

In one aspect of the Nanobodies of the invention, the amino acid residue at position 83 is chosen from the group consisting of L, M, S, V and W; and is preferably L.

Also, in one aspect of the Nanobodies of the invention, the amino acid residue at position 83 is chosen from the group consisting of R, K, N, E, G, I, T and Q; and is most preferably either K or E (for Nanobodies corresponding to naturally occurring V_{HH} domains) or R (for “humanized” Nanobodies, as described herein). The amino acid residue at position 84 is chosen from the group consisting of P, A, R, S, D T, and V in one aspect, and is most preferably P (for Nanobodies corresponding to naturally occurring V_{HH} domains) or R (for “humanized” Nanobodies, as described herein).

Furthermore, in one aspect of the Nanobodies of the invention, the amino acid residue at position 104 is chosen from the group consisting of G and D; and is most preferably G.

Collectively, the amino acid residues at positions 11, 37, 44, 45, 47, 83, 84, 103, 104 and 108, which in the Nanobodies are as mentioned above, will also be referred to herein as the “Hallmark Residues”. The Hallmark Residues and the amino acid residues at the corresponding positions of the most closely related human V_{H} domain, V_{H}3, are summarized in Table A-3.

Some especially preferred but non-limiting combinations of these Hallmark Residues as occur in naturally occurring V_{HH} domains are mentioned in Table A-4. For comparison, the corresponding amino acid residues of the human V_{H}3 called DP-47 have been indicated in italics.

TABLE A-3
Hallmark Residues in Nanobodies

| Position | Human V_{H}3 | Hallmark Residues |
|---|---|---|
| 11 | L, V; predominantly L | L, M, S, V, W; preferably L |
| 37 | V, I, F; usually V | F⁽¹⁾, Y, H, I, L or V, preferably F⁽¹⁾ or Y |
| 44⁽⁸⁾ | G | G⁽²⁾, E⁽³⁾, A, D, Q, R, S, L; |
| | | preferably G⁽²⁾, E⁽³⁾ or Q; |
| | | most preferably G⁽²⁾ or E⁽³⁾. |
| 45⁽⁸⁾ | L | L⁽²⁾, R⁽³⁾, C, I, L, P, Q, V; preferably L⁽²⁾ |
| | | or R⁽³⁾ |
| 47⁽⁸⁾ | W, Y | W⁽²⁾, L⁽¹⁾ or F⁽¹⁾, A, G, I, M, R, S, V or |
| | | Y; preferably W⁽²⁾ , L⁽¹⁾, F⁽¹⁾ or R |
| 83 | R or K; usually R | R, K⁽⁵⁾, N, E⁽⁵⁾, G, I, M, Q or T; |
| | | preferably K or R; most preferably K |
| 84 | A, T, D; predominantly A | P⁽⁵⁾, A, L, R, S, T, D, V; preferably P |
| 103 | W | W⁽⁴⁾, P⁽⁶⁾ , R⁽⁶⁾, S; preferably W |
| 104 | G | G or D; preferably G |
| 108 | L, M or T; predominantly L | Q, L⁽⁷⁾ or R; preferably Q or L⁽⁷⁾ |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF or KEREG at positions 43-47. Alternatively, also sequences such as TERE (for example TEREL), KECE (for example KECEL or KECER), RERE (for example REREG), QERE (for example QEREG), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q in (non-humanized) V_{HH} sequences that also contain a W at 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

TABLE A-4
Some preferred but non-limiting combinations of
Hallmark Residues in naturally occurring Nanobodies.
For humanization of these combinations,
reference is made to the specification.

| | 11 | 37 | 44 | 45 | 47 | 83 | 84 | 103 | 104 | 108 |
|---|---|---|---|---|---|---|---|---|---|---|
| DP-47 (human) | M | V | G | L | W | R | A | W | G | L |
| “KERE” group | L | F | E | R | L | K | P | W | G | Q |
| | L | F | E | R | F | E | P | W | G | Q |
| | L | F | E | R | F | K | P | W | G | Q |
| | L | Y | Q | R | L | K | P | W | G | Q |
| | L | F | L | R | V | K | P | Q | G | Q |
| | L | F | Q | R | L | K | P | W | G | Q |
| | L | F | E | R | F | K | P | W | G | Q |
| “GLEW” group | L | V | G | L | W | K | S | W | G | Q |
| | M | V | G | L | W | K | P | R | G | Q |

In the Nanobodies, each amino acid residue at any other position than the Hallmark Residues can be any amino acid residue that naturally occurs at the corresponding position (according to the Kabat numbering) of a naturally occurring V_{HH} domain.

Such amino acid residues will be clear to the skilled person. Tables A-5 to A-8 mention some non-limiting residues that can be present at each position (according to the Kabat numbering) of the FR1, FR2, FR3 and FR4 of naturally occurring V_{HH} domains. For each position, the amino acid residue that most frequently occurs at each position of a naturally occurring V_{HH} domain (and which is the most preferred amino acid residue for said position in a Nanobody) is indicated in bold; and other preferred amino acid residues for each position have been underlined (note: the number of amino acid residues that are found at positions 26-30 of naturally occurring V_{HH} domains supports the hypothesis underlying the numbering by Chothia (supra) that the residues at these positions already form part of CDR1.)

In Tables A-5-A-8, some of the non-limiting residues that can be present at each position of a human V_{H}3 domain have also been mentioned. Again, for each position, the amino acid residue that most frequently occurs at each position of a naturally occurring human V_{H}3 domain is indicated in bold; and other preferred amino acid residues have been underlined.

For reference only, Tables A-5-A-8 also contain data on the V_{HH} entropy (“V_{HH} Ent.”) and V_{HH} variability (“V_{HH} Var.”) at each amino acid position for a representative sample of 1118 V_{HH} sequences (data kindly provided by David Lutje Hulsing and Prof. Theo Verrips of Utrecht University). The values for the V_{HH} entropy and the V_{HH} variability provide a measure for the variability and degree of conservation of amino acid residues between the 1118 V_{HH} sequences analyzed: low values (i.e. <1, such as <0.5) indicate that an amino acid residue is highly conserved between the V_{HH} sequences (i.e. little variability). For example, the G at position 8 and the G at position 9 have values for the V_{HH} entropy of 0.1 and 0 respectively, indicating that these residues are highly conserved and have little variability (and in case of position 9 is G in all 1118 sequences analysed), whereas for residues that form part of the CDR's generally values of 1.5 or more are found (data not shown). Note that (1) the amino acid residues listed in the second column of Tables A-5-A-8 are based on a bigger sample than the 1118 V_{HH} sequences that were analysed for determining the V_{HH} entropy and V_{HH} variability referred to in the last two columns; and (2) the data represented below support the hypothesis that the amino acid residues at positions 27-30 and maybe even also at positions 93 and 94 already form part of the CDR's (although the invention is not limited to any specific hypothesis or explanation, and as mentioned above, herein the numbering according to Kabat is used). For a general explanation of sequence entropy, sequence variability and the methodology for determining the same, see Oliveira et al., PROTEINS: Structure. Function and Genetics, 52:544-552 (2003).

TABLE A-5
Non-limiting examples of amino acid residues in FR1
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 1 | E, Q | Q, A, E | — | — |
| 2 | V | V | 0.2 | 1 |
| 3 | Q | Q, K | 0.3 | 2 |
| 4 | L | L | 0.1 | 1 |
| 5 | V, L | Q, E, L, V | 0.8 | 3 |
| 6 | E | E, D, Q, A | 0.8 | 4 |
| 7 | S, T | S, F | 0.3 | 2 |
| 8 | G, R | G | 0.1 | 1 |
| 9 | G | G | 0 | 1 |
| 10 | G, V | G, D, R | 0.3 | 2 |
| 11 | Hallmark residue: L, M, S, V, W; preferably L | | 0.8 | 2 |
| 12 | V, I | V, A | 0.2 | 2 |
| 13 | Q, K, R | Q, E, K, P, R | 0.4 | 4 |
| 14 | P | A, Q, A, G, P, S, T, V | 1 | 5 |
| 15 | G | G | 0 | 1 |
| 16 | G, R | G, A, E, D | 0.4 | 3 |
| 17 | S | S, F | 0.5 | 2 |
| 18 | L | L, V | 0.1 | 1 |
| 19 | R, K | R, K, L, N, S, T | 0.6 | 4 |
| 20 | L | L, F, I, V | 0.5 | 4 |
| 21 | S | S, A, F, T | 0.2 | 3 |
| 22 | C | C | 0 | 1 |
| 23 | A, T | A, D, E, P, S, T, V | 1.3 | 5 |
| 24 | A | A, I, L, S, T, V | 1 | 6 |
| 25 | S | S, A, F, P, T | 0.5 | 5 |
| 26 | G | G, A, D, E, R, S, T, V | 0.7 | 7 |
| 27 | F | S, F, R, L, P, G, N, | 2.3 | 13 |
| 28 | T | N, T, E, D, S, I, R, A, G, R, F, | 1.7 | 11 |
| | | Y | | |
| 29 | F, V | F, L, D, S, I, G, V, A | 1.9 | 11 |
| 30 | S, D, G | N, S, E, G, A, D, M, T | 1.8 | 11 |

TABLE A-6
Non-limiting examples of amino acid residues in FR2
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 36 | W | W | 0.1 | 1 |
| 37 | Hallmark residue: F⁽¹⁾, H, I, L, Y or V, preferably F⁽¹⁾ or Y | | 1.1 | 6 |
| 38 | R | R | 0.2 | 1 |
| 39 | Q | Q, H, P, R | 0.3 | 2 |
| 40 | A | A, F, G, L, P, T, V | 0.9 | 7 |
| 41 | P, S, T | P, A, L, S | 0.4 | 3 |
| 42 | G | G, E | 0.2 | 2 |
| 43 | K | K, D, E, N, Q, R, T, V | 0.7 | 6 |
| 44 | Hallmark residue: G⁽²⁾, E⁽³⁾, A, D, Q, R, S, L; preferably G⁽²⁾, E⁽³⁾ | | 1.3 | 5 |
| | or Q; most preferably G⁽²⁾ or E⁽³⁾. | | | |
| 45 | Hallmark residue: L⁽²⁾, R⁽³⁾, C, I, L, P, Q, V; preferably L⁽²⁾ or | | 0.6 | 4 |
| | R⁽³⁾ | | | |
| 46 | E, V | E, D, K, Q, V | 0.4 | 2 |
| 47 | Hallmark residue: W⁽²⁾, L⁽¹⁾ or F⁽¹⁾, A, G, I, M, R, S, V or Y; | | 1.9 | 9 |
| | preferably W⁽²⁾, L⁽¹⁾, F⁽¹⁾ or R | | | |
| 48 | V | V, I, L | 0.4 | 3 |
| 49 | S, A, G | A, S, G, T, V | 0.8 | 3 |

TABLE A-7
Non-limiting examples of amino acid residues in FR3
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 66 | R | R | 0.1 | 1 |
| 67 | F | F, L, V | 0.1 | 1 |
| 68 | T | T, A, N, S | 0.5 | 4 |
| 69 | I | I, L, M, V | 0.4 | 4 |
| 70 | S | S, A, F, T | 0.3 | 4 |
| 71 | R | R, G, H, I, L, K, Q, S, T, W | 1.2 | 8 |
| 72 | D, E | D, E, G, N, V | 0.5 | 4 |
| 73 | N, D, G | N, A, D, F, I, K, L, R, S, T, V, Y | 1.2 | 9 |
| 74 | A, S | A, D, G, N, P, S, T, V | 1 | 7 |
| 75 | K | K, A, E, K, L, N, Q, R | 0.9 | 6 |
| 76 | N, S | N, D, K, R, S, T, Y | 0.9 | 6 |
| 77 | S, T, I | T, A, E, I, M, P, S | 0.8 | 5 |
| 78 | L, A | V, L, A, F, G, I, M | 1.2 | 5 |
| 79 | Y, H | Y, A, D, F, H, N, S, T | 1 | 7 |
| 80 | L | L, F, V | 0.1 | 1 |
| 81 | Q | Q, E, I, L, R, T | 0.6 | 5 |
| 82 | M | M, I, L, V | 0.2 | 2 |
| 82a | N, G | N, D, G, H, S, T | 0.8 | 4 |
| 82b | S | S, N, D, G, R, T | 1 | 6 |
| 82c | L | L, P, V | 0.1 | 2 |
| 83 | Hallmark residue: R, K⁽⁵⁾, N, E⁽⁵⁾, G, I, M, Q or T; | | 0.9 | 7 |
| | preferably K or R; most preferably K | | | |
| 84 | Hallmark residue: P⁽⁵⁾, A, D, L, R, S, T, V; preferably P | | 0.7 | 6 |
| 85 | E, G | E, D, G, Q | 0.5 | 3 |
| 86 | D | D | 0 | 1 |
| 87 | T, M | T, A, S | 0.2 | 3 |
| 88 | A | A, G, S | 0.3 | 2 |
| 89 | V, L | V, A, D, I, L, M, N, R, T | 1.4 | 6 |
| 90 | Y | Y, F | 0 | 1 |
| 91 | Y, H | Y, D, F, H, L, S, T, V | 0.6 | 4 |
| 92 | C | C | 0 | 1 |
| 93 | A, K, T | A, N, G, H, K, N, R, S, T, V, Y | 1.4 | 10 |
| 94 | K, R, T | A, V, C, F, G, I, K, L, R, S or T | 1.6 | 9 |

TABLE A-8
Non-limiting examples of amino acid residues in FR4
(for the footnotes, see the footnotes to Table A-3)

| | Amino acid residue(s): | | V_{HH} | V_{HH} |
|---|---|---|---|---|
| Pos. | Human V_{H}3 | Camelid V_{HH}'s | Ent. | Var. |
| 103 | Hallmark residue: W⁽⁴⁾, P⁽⁶⁾, R⁽⁶⁾, S; preferably W | | 0.4 | 2 |
| 104 | Hallmark residue: G or D; preferably G | | 0.1 | 1 |
| 105 | Q, R | Q, E, K, P, R | 0.6 | 4 |
| 106 | G | G | 0.1 | 1 |
| 107 | T | T, A, I | 0.3 | 2 |
| 108 | Hallmark residue: Q, L⁽⁷⁾ or R; preferably Q or L⁽⁷⁾ | | 0.4 | 3 |
| 109 | V | V | 0.1 | 1 |
| 110 | T | T, I, A | 0.2 | 1 |
| 111 | V | V, A, I | 0.3 | 2 |
| 112 | S | S, F | 0.3 | 1 |
| 113 | S | S, A, L, P, T | 0.4 | 3 |

Thus, in another preferred, but not limiting aspect, a Nanobody of the invention can be defined as an amino acid sequence with the (general) structure

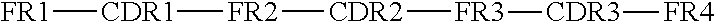

in which FR1 to FR4 refer to framework regions 1 to 4, respectively, and in which CDR1 to CDR3 refer to the complementarity determining regions 1 to 3, respectively, and in which:
i) one or more of the amino acid residues at positions 11, 37, 44, 45, 47, 83, 84, 103, 104 and 108 according to the Kabat numbering are chosen from the Hallmark residues mentioned in Table A-3;
and in which:
ii) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

In particular, a Nanobody of the invention can be an amino acid sequence with the (general) structure

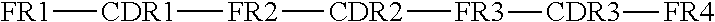

in which FR1 to FR4 refer to framework regions 1 to 4, respectively, and in which CDR1 to CDR3 refer to the complementarity determining regions 1 to 3, respectively, and in which:
i) (preferably) one or more of the amino acid residues at positions 11, 37, 44, 45, 47, 83, 84, 103, 104 and 108 according to the Kabat numbering are chosen from the Hallmark residues mentioned in Table A-3 (it being understood that V_{HH} sequences will contain one or more Hallmark residues; and that partially humanized Nanobodies will usually, and preferably, [still] contain one or more Hallmark residues [although it is also within the scope of the invention to provide—where suitable in accordance with the invention—partially humanized Nanobodies in which all Hallmark residues, but not one or more of the other amino acid residues, have been humanized]; and that in fully humanized Nanobodies, where suitable in accordance with the invention, all amino acid residues at the positions of the Hallmark residues will be amino acid residues that occur in a human V_{H}3 sequence. As will be clear to the skilled person based on the disclosure herein that such V_{HH} Sequences, such partially humanized Nanobodies with at least one Hallmark residue, such partially humanized Nanobodies without Hallmark residues and such fully humanized Nanobodies all form aspects of this invention);
and in which:
ii) said amino acid sequence has at least 80% amino acid identity with at least one of the amino acid sequences of SEQ ID NO's: 1 to 22, in which for the purposes of determining the degree of amino acid identity, the amino acid residues that form the CDR sequences (indicated with X in the sequences of SEQ ID NO's: 1 to 22) are disregarded;
and in which:
iii) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

TABLE A-9
Representative amino acid sequences for Nanobodies of the KERE, GLEW
and P, R, S 103 group.
The CDR's are indicated with XXXX

| | | |
|---|---|---|
| KERE sequence no. 1 | SEQ ID NO: 1 | EVQLVESGGGLVQPGGSLRLSCAASGIPFSXXXXXWFRQAPGKQR |
| | | DSVAXXXXXRFTISRDNAKNTVYLQMNSLKPEDTAVYRCYFXXXXX |
| | | WGQGTQVTVSS |
| KERE sequence no. 2 | SEQ ID NO: 2 | QVKLEESGGGLVQAGGSLRLSCVGSGRTFSXXXXXWFRLAPGKER |
| | | EFVAXXXXXRFTISRDTASNRGYLHMNNLTPEDTAVYYCAAXXXXX |
| | | WGQGTQVTVSS |
| KERE sequence no. 3 | SEQ ID NO: 3 | AVQLVDSGGGLVQAGDSLKLSCALTGGAFTXXXXXWFRQTPGRER |
| | | EFVAXXXXXRFTISRDNAKNMVYLRMNSLIPEDAAVYSCAAXXXXX |
| | | WGQGTLVTVSS |
| KERE sequence no. 4 | SEQ ID NO: 4 | QVQLVESGGGLVEAGGSLRLSCTASESPFRXXXXXWFRQTSGQER |
| | | EFVAXXXXXRFTISRDDAKNTVWLHGSTLKPEDTAVYYCAAXXXXX |
| | | WGQGTQVTVSS |
| KERE sequence no. 5 | SEQ ID NO: 5 | AVQLVESGGGLVQGGGSLRLACAASERIFDXXXXXWYRQGPGNER |
| | | ELVAXXXXXRFTISMDYTKQTVYLHMNSLRPEDTGLYYCKIXXXXXW |
| | | GQGTQVTVSS |
| KERE sequence no. 6 | SEQ ID NO: 6 | DVKFVESGGGLVQAGGSLRLSCVASGFNFDXXXXXWFRQAPGKER |
| | | EEVAXXXXXRFTISSEKDKNSVYLQMNSLKPEDTALYICAGXXXXXW |
| | | GRGTQVTVSS |
| KERE sequence no. 7 | SEQ ID NO: 7 | QVRLAESGGGLVQSGGSLRLSCVASGSTYTXXXXXWYRQYPGKQ |
| | | RALVAXXXXXRFTIARDSTKDTFCLQMNNLKPEDTAVYYCYAXXXXX |
| | | WGQGTQVTVSS |
| KERE sequence no. 8 | SEQ ID NO: 8 | EVQLVESGGGLVQAGGSLRLSCAASGFTSDXXXXXWFRQAPGKPR |
| | | EGVSXXXXXRFTISTDNAKNTVHLLMNRVNAEDTALYYCAVXXXXX |
| | | WGRGTRVTVSS |
| KERE sequence no. 9 | SEQ ID NO: 9 | QVQLVESGGGLVQPGGSLRLSCQASGDISTXXXXXWYRQVPGKLR |
| | | EFVAXXXXXRFTISGDNAKRAIYLQMNNLKPDDTAVYYCNRXXXXX |
| | | WGQGTQVTVSP |
| KERE sequence no. 10 | SEQ ID NO: 10 | QVPVVESGGGLVQAGDSLRLFCAVPSFTSTXXXXXWFRQAPGKER |
| | | EFVAXXXXXRFTISRNATKNTLTLRMDSLKPEDTAVYYCAAXXXXXW |
| | | GQGTQVTVSS |
| KERE sequence no. 11 | SEQ ID NO: 11 | EVQLVESGGGLVQAGDSLRLFCTVSGGTASXXXXXWFRQAPGEKR |
| | | EFVAXXXXXRFTIARENAGNMVYLQMNNLKPDDTALYTCAAXXXXX |
| | | WGRGTQVTVSS |
| KERE sequence no. 12 | SEQ ID NO: 12 | AVQLVESGGDSVQPGDSQTLSCAASGRTNSXXXXXWFRQAPGKE |
| | | RVFLAXXXXXRFTISRDSAKNMMYLQMNNLKPQDTAVYYCAAXXXX |
| | | XWGQGTQVTVSS |
| KERE sequence no. 13 | SEQ ID NO: 13 | AVQLVESGGGLVQAGGSLRLSCVVSGLTSSXXXXXWFRQTPWQE |
| | | RDFVAXXXXXRFTISRDNYKDTVLLEMNFLKPEDTAIYYCAAXXXXX |
| | | WGQGTQVTVSS |
| KERE sequence no. 14 | SEQ ID NO: 14 | AVQLVESGGGLVQAGASLRLSCATSTRTLDXXXXXWFRQAPGRDR |
| | | EFVAXXXXXRFTVSRDSAENTVALQMNSLKPEDTAVYYCAAXXXXX |
| | | WGQGTRVTVSS |
| KERE sequence no. 15 | SEQ ID NO: 15 | QVQLVESGGGLVQPGGSLRLSCTVSRLTAHXXXXXWFRQAPGKER |
| | | EAVSXXXXXRFTISRDYAGNTAFLQMDSLKPEDTGVYYCATXXXXX |
| | | WGQGTQVTVSS |
| KERE sequence no. 16 | SEQ ID NO: 16 | EVQLVESGGELVQAGGSLKLSCTASGRNFVXXXXXWFRRAPGKER |
| | | EFVAXXXXXRFTVSRDNGKNTAYLRMNSLKPEDTADYYCAVXXXXX |
| | | LGSGTQVTVSS |
| GLEW sequence no. 1 | SEQ ID NO: 17 | AVQLVESGGGLVQPGGSLRLSCAASGFTFSXXXXXWVRQAPGKVL |
| | | EWVSXXXXXRFTISRDNAKNTLYLQMNSLKPEDTAVYYCVKXXXXX |
| | | GSQGTQVTVSS |
| GLEW sequence no. 2 | SEQ ID NO: 18 | EVQLVESGGGLVQPGGSLRLSCVCVSSGCTXXXXXWVRQAPGKA |
| | | EEWVSXXXXXRFKISRDNAKKTLYLQMNSLGPEDTAMYYCQRXXX |
| | | XXRGQGTQVTVSS |
| GLEW sequence no. 3 | SEQ ID NO: 19 | EVQLVESGGGLALPGGSLTLSCVFSGSTFSXXXXXWVRHTPGKAE |
| | | EWVSXXXXXRFTISRDNAKNTLYLEMNSLSPEDTAMYYCGRXXXXX |
| | | RSKGIQVTVSS |
| P, R, S 103 sequence | SEQ ID NO: 20 | AVQLVESGGGLVQAGGSLRLSCAASGRTFSXXXXXWFRQAPGKER |
| no. 1 | | EFVAXXXXXRFTISRDNAKNTVYLQMNSLKPEDTAVYYCAAXXXXX |
| | | RGQGTQVTVSS |
| P, R, S 103 sequence | SEQ ID NO: 21 | DVQLVESGGDLVQPGGSLRLSCAASGFSFDXXXXXWLRQTPGKGL |
| no. 2 | | EWVGXXXXXRFTISRDNAKNMLYLHLNNLKSEDTAVYYCRRXXXXX |
| | | LGQGTQVTVSS |
| P, R, S 103 sequence | SEQ ID NO: 22 | EVQLVESGGGLVQPGGSLRLSCVCVSSGCTXXXXXWVRQAPGKA |
| no. 3 | | EEWVSXXXXXRFKISRDNAKKTLYLQMNSLGPEDTAMYYCQRXXX |
| | | XXRGQGTQVTVSS |

In particular, a Nanobody of the invention of the KERE group can be an amino acid sequence with the (general) structure

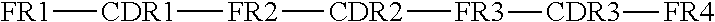

in which:
i) the amino acid residue at position 45 according to the Kabat numbering is a charged amino acid (as defined herein) or a cysteine residue, and position 44 is preferably an E;
and in which:
ii) FR1 is an amino acid sequence that has at least 80% amino acid identity with at least one of the following amino acid sequences:

TABLE A-10
Representative FW1 sequences for Nanobodies of the KERE-group.

| | | |
|---|---|---|
| KERE FW1 sequence no. 1 | SEQ ID NO: 23 | QVQRVESGGGLVQAGGSLRLSCAASGRTSS |
| KERE FW1 sequence no. 2 | SEQ ID NO: 24 | QVQLVESGGGLVQTGDSLSLSCSASGRTFS |
| KERE FW1 sequence no. 3 | SEQ ID NO: 25 | QVKLEESGGGLVQAGDSLRLSCAATGRAFG |
| KERE FW1 sequence no. 4 | SEQ ID NO: 26 | AVQLVESGGGLVQPGESLGLSCVASGRDFV |
| KERE FW1 sequence no. 5 | SEQ ID NO: 27 | EVQLVESGGGLVQAGGSLRLSCEVLGRTAG |
| KERE FW1 sequence no. 6 | SEQ ID NO: 28 | QVQLVESGGGWVQPGGSLRLSCAASETILS |
| KERE FW1 sequence no. 7 | SEQ ID NO: 29 | QVQLVESGGGTVQPGGSLNLSCVASGNTFN |
| KERE FW1 sequence no. 8 | SEQ ID NO: 30 | EVQLVESGGGLAQPGGSLQLSCSAPGFTLD |
| KERE FW1 sequence no. 9 | SEQ ID NO: 31 | AQELEESGGGLVQAGGSLRLSCAASGRTFN |

and in which:
iii) FR2 is an amino acid sequence that has at least 80% amino acid identity with at least one of the following amino acid sequences:

TABLE A-11
Representative FW2 sequences for Nanobodies of the KERE-group.

| | | |
|---|---|---|
| KERE FW2 sequence no. 1 | SEQ ID NO: 41 | WFRQAPGKEREFVA |
| KERE FW2 sequence no. 2 | SEQ ID NO: 42 | WFRQTPGREREFVA |
| KERE FW2 sequence no. 3 | SEQ ID NO: 43 | WYRQAPGKQREMVA |
| KERE FW2 sequence no. 4 | SEQ ID NO: 44 | WYRQGPGKQRELVA |
| KERE FW2 sequence no. 5 | SEQ ID NO: 45 | WIRQAPGKEREGVS |
| KERE FW2 sequence no. 6 | SEQ ID NO: 46 | WFREAPGKEREGIS |
| KERE FW2 sequence no. 7 | SEQ ID NO: 47 | WYRQAPGKERDLVA |
| KERE FW2 sequence no. 8 | SEQ ID NO: 48 | WFRQAPGKQREEVS |
| KERE FW2 sequence no. 9 | SEQ ID NO: 49 | WFRQPPGKVREFVG |

and in which:
iv) PR3 is an amino acid sequence that has at least 80% amino acid identity with at least one of the following amino acid sequences:

TABLE A-12
Representative FW3 sequences for Nanobodies of the KERE-group.

| | | |
|---|---|---|
| KERE FW3 sequence no. 1 | SEQ ID NO: 50 | RFTISRDNAKNTVYLQMNSLKPEDTAVYRCYF |
| KERE FW3 sequence no. 2 | SEQ ID NO: 51 | RFAISRDNNKNTGYLQMNSLEPEDTAVYYCAA |
| KERE FW3 sequence no. 3 | SEQ ID NO: 52 | RFTVARNNAKNTVNLEMNSLKPEDTAVYYCAA |
| KERE FW3 sequence no. 4 | SEQ ID NO: 53 | RFTISRDIAKNTVDLLMNNLEPEDTAVYYCAA |
| KERE FW3 sequence no. 5 | SEQ ID NO: 54 | RLTISRDNAVDTMYLQMNSLKPEDTAVYYCAA |
| KERE FW3 sequence no. 6 | SEQ ID NO: 55 | RFTISRDNAKNTVYLQMDNVKPEDTAIYYCAA |
| KERE FW3 sequence no. 7 | SEQ ID NO: 56 | RFTISKDSGKNTVYLQMTSLKPEDTAVYYCAT |
| KERE FW3 sequence no. 8 | SEQ ID NO: 57 | RFTISRDSAKNMMYLQMNNLKPQDTAVYYCAA |
| KERE FW3 sequence no. 9 | SEQ ID NO: 58 | RFTISRENDKSTVYLQLNSLKPEDTAVYYCAA |
| KERE FW3 sequence no. 10 | SEQ ID NO: 59 | RFTISRDYAGNTAYLQMNSLKPEDTGVYYCAT |

and in which:
v) FR4 is an amino acid sequence that has at least 80% amino acid identity with at least one of the following amino acid sequences:

TABLE A-13
Representative FW4 sequences for Nanobodies of the KERE-group.

| | | |
|---|---|---|
| KERE FW4 sequence no. 1 | SEQ ID NO: 60 | WGQGTQVTVSS |
| KERE FW4 sequence no. 2 | SEQ ID NO: 61 | WGKGTLVTVSS |
| KERE FW4 sequence no. 3 | SEQ ID NO: 62 | RGQGTRVTVSS |
| KERE FW4 sequence no. 4 | SEQ ID NO: 63 | WGLGTQVTISS |

and in which:
vi) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are V_{HH} sequences or partially humanized Nanobodies).

Also, the above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

With regard to framework 1, it will be clear to the skilled person that, when an amino acid sequence as outlined above is generated by expression of a nucleotide sequence, the first four amino acid sequences (i.e. amino acid residues 1˜4 according to the Kabat numbering) may often be determined by the primer(s) that have been used to generate said nucleic acid. Thus, for determining the degree of amino acid identity, the first four amino acid residues are preferably disregarded.

Also, with regard to framework 1, and although amino acid positions 27 to 30 are according to the Kabat numbering considered to be part of the framework regions (and not the CDR's), it has been found by analysis of a database of more than 1000 V_{HH} sequences that the positions 27 to 30 have a variability (expressed in terms of V_{HH} entropy and V_{HH} variability-see Tables A-5 to A-8) that is much greater than the variability on positions 1 to 26. Because of this, for determining the degree of amino acid identity, the amino acid residues at positions 27 to 30 are preferably also disregarded.

In view of this, a Nanobody of the KERE class may be an amino acid sequence that is comprised of four framework regions/sequences interrupted by three complementarity determining regions/sequences, in which:
i) the amino acid residue at position 45 according to the Kabat numbering is a charged amino acid (as defined herein) or a cysteine residue, and position 44 is preferably an E;
and in which:
ii) FR1 is an amino acid sequence that, on positions 5 to 26 of the Kabat numbering, has at least 80% amino acid identity with at least one of the following amino acid sequences:

TABLE A-14
Representative FW1 sequences (amino acid residues 5 to 26) for Nanobodies
of the KERE-group.

| | | |
|---|---|---|
| KERE FW1 sequence no. 10 | SEQ ID NO: 32 | VESGGGLVQPGGSLRLSCAASG |
| KERE FW1 sequence no. 11 | SEQ ID NO: 33 | VDSGGGLVQAGDSLKLSCALTG |
| KERE FW1 sequence no. 12 | SEQ ID NO: 34 | VDSGGGLVQAGDSLRLSCAASG |
| KERE FW1 sequence no. 13 | SEQ ID NO: 35 | VDSGGGLVEAGGSLRLSCQVSE |
| KERE FW1 sequence no. 14 | SEQ ID NO: 36 | QDSGGGSVQAGGSLKLSCAASG |
| KERE FW1 sequence no. 15 | SEQ ID NO: 37 | VQSGGRLVQAGDSLRLSCAASE |
| KERE FW1 sequence no. 16 | SEQ ID NO: 38 | VESGGTLVQSGDSLKLSCASST |
| KERE FW1 sequence no. 17 | SEQ ID NO: 39 | MESGGDSVQSGGSLTLSCVASG |
| KERE FW1 sequence no. 18 | SEQ ID NO: 40 | QASGGGLVQAGGSLRLSCSASV |

and in which:
iii) FR2, FR3 and FR4 are as mentioned herein for FR2, FR3 and FR4 of Nanobodies of the KERE-class;
and in which:
iv) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

A Nanobody of the GLEW class may be an amino acid sequence that is comprised of four framework regions/sequences interrupted by three complementarity determining regions/sequences, in which
i) preferably, when the Nanobody of the GLEW-class is a non-humanized Nanobody, the amino acid residue in position 108 is Q;
ii) FR1 is an amino acid sequence that has at least 80% amino acid identity with at least one of the following amino acid sequences:

TABLE A-15
Representative FW1 sequences for Nanobodies of the GLEW-group.

| | | |
|---|---|---|
| GLEW FW1 sequence no. 1 | SEQ ID NO: 64 | QVQLVESGGGLVQPGGSLRLSCAASGFTFS |
| GLEW FW1 sequence no. 2 | SEQ ID NO: 65 | EVHLVESGGGLVRPGGSLRLSCAAFGFIFK |
| GLEW FW1 sequence no. 3 | SEQ ID NO: 66 | QVKLEESGGGLAQPGGSLRLSCVASGFTFS |
| GLEW FW1 sequence no. 4 | SEQ ID NO: 67 | EVQLVESGGGLVQPGGSLRLSCVCVSSGCT |
| GLEW FW1 sequence no. 5 | SEQ ID NO: 68 | EVQLVESGGGLALPGGSLTLSCVFSGSTFS |

and in which:
iii) FR2 is an amino acid sequence that has at least 80% amino acid identity with at least one of the following amino acid sequences:

TABLE A-16
Representative FW2 sequences for Nanobodies of the GLEW-group.

| | | |
|---|---|---|
| GLEW FW2 sequence no. 1 | SEQ ID NO: 72 | WVRQAPGKVLEWVS |
| GLEW FW2 sequence no. 2 | SEQ ID NO: 73 | WVRRPPGKGLEWVS |
| GLEW FW2 sequence no. 3 | SEQ ID NO: 74 | WVRQAPGMGLEWVS |
| GLEW FW2 sequence no. 4 | SEQ ID NO: 75 | WVRQAPGKEPEWVS |
| GLEW FW2 sequence no. 5 | SEQ ID NO: 76 | WVRQAPGKDQEWVS |
| GLEW FW2 sequence no. 6 | SEQ ID NO: 77 | WVRQAPGKAEEWVS |
| GLEW FW2 sequence no. 7 | SEQ ID NO: 78 | WVRQAPGKGLEWVA |
| GLEW FW2 sequence no. 8 | SEQ ID NO: 79 | WVRQAPGRATEWVS |

and in which:
iv) FR3 is an amino acid sequence that has at least 80% amino acid identity with at least one of the following amino acid sequences:

TABLE A-17
Representative FW3 sequences for Nanobodies of the GLEW-group.

| | | |
|---|---|---|
| GLEW FW3 sequence no. 1 | SEQ ID NO: 80 | RFTISRDNAKNTLYLQMNSLKPEDTAVYYCVK |
| GLEW FW3 sequence no. 2 | SEQ ID NO: 81 | RFTISRDNARNTLYLQMDSLIPEDTALYYCAR |
| GLEW FW3 sequence no. 3 | SEQ ID NO: 82 | RFTSSRDNAKSTLYLQMNDLKPEDTALYYCAR |
| GLEW FW3 sequence no. 4 | SEQ ID NO: 83 | RFIISRDNAKNTLYLQMNSLGPEDTAMYYCQR |
| GLEW FW3 sequence no. 5 | SEQ ID NO: 84 | RFTASRDNAKNTLYLQMNSLKSEDTARYYCAR |
| GLEW FW3 sequence no. 6 | SEQ ID NO: 85 | RFTISRDNAKNTLYLQMDDLQSEDTAMYYCGR |

and in which:
v) FR4 is an amino acid sequence that has at least 80% amino acid identity with at least one of the following amino acid sequences:

TABLE A-18
Representative FW4 sequences for Nanobodies of the GLEW-group.

| | | |
|---|---|---|
| GLEW FW4 sequence no. 1 | SEQ ID NO: 86 | GSQGTQVTVSS |
| GLEW FW4 sequence no. 2 | SEQ ID NO: 87 | LRGGTQVTVSS |
| GLEW FW4 sequence no. 3 | SEQ ID NO: 88 | RGQGTLVTVSS |
| GLEW FW4 sequence no. 4 | SEQ ID NO: 89 | RSRGIQVTVSS |
| GLEW FW4 sequence no. 5 | SEQ ID NO: 90 | WGKGTQVTVSS |
| GLEW FW4 sequence no. 6 | SEQ ID NO: 91 | WGQGTQVTVSS |

and in which:
vi) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are V_{HH} sequences or partially humanized Nanobodies).

With regard to framework 1, it will again be clear to the skilled person that, for determining the degree of amino acid identity, the amino acid residues on positions 1 to 4 and 27 to 30 are preferably disregarded.

In view of this, a Nanobody of the GLEW class may be an amino acid sequence that is comprised of four framework regions/sequences interrupted by three complementarity determining regions/sequences, in which:
i) preferably, when the Nanobody of the GLEW-class is a non-humanized Nanobody, the amino acid residue in position 108 is Q;
and in which:
ii) FR1 is an amino acid sequence that, on positions 5 to 26 of the Kabat numbering, has at least 80% amino acid identity with at least one of the following amino acid sequences:

TABLE A-19
Representative FW1 sequences (amino acid residues 5 to 26) for Nanobodies
of the KERE-group.

| | | |
|---|---|---|
| GLEW FW1 sequence no. 6 | SEQ ID NO: 69 | VESGGGLVQPGGSLRLSCAASG |
| GLEW FW1 sequence no. 7 | SEQ ID NO: 70 | EESGGGLAQPGGSLRLSCVASG |
| GLEW FW1 sequence no. 8 | SEQ ID NO: 71 | VESGGGLALPGGSLTLSCVFSG |

and in which:
iii) FR2, FR3 and FR4 are as mentioned herein for FR2, FR3 and FR4 of Nanobodies of the GLEW-class;
and in which:
iv) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein. In the above Nanobodies, one or more of the further Hallmark residues are preferably as described herein (for example, when they are V_{HH} sequences or partially humanized Nanobodies).

A Nanobody of the P, R, S 103 class may be an amino acid sequence that is comprised of four framework regions/sequences interrupted by three complementarity determining regions/sequences, in which
i) the amino acid residue at position 103 according to the Kabat numbering is different from W;
and in which:
ii) preferably the amino acid residue at position 103 according to the Kabat numbering is P, R or S, and more preferably R;
and in which:
iii) FR1 is an amino acid sequence that has at least 80% amino acid identity with at least one of the following amino acid sequences:

TABLE A-20
Representative FW1 sequences for Nanobodies of the P, R, S 103-group.

| | | |
|---|---|---|
| P, R, S 103 FW1 sequence no. 1 | SEQ ID NO: 92 | AVQLVESGGGLVQAGGSLRLSCAASGRTFS |
| P, R, S 103 FW1 sequence no. 2 | SEQ ID NO: 93 | QVQLQESGGGMVQPGGSLRLSCAASGFDFG |
| P, R, S 103 FW1 sequence no. 3 | SEQ ID NO: 94 | EVHLVESGGGLVRPGGSLRLSCAAFGFIFK |
| P, R, S 103 FW1 sequence no. 4 | SEQ ID NO: 95 | QVQLAESGGGLVQPGGSLKLSCAASRTIVS |
| P, R, S 103 FW1 sequence no. 5 | SEQ ID NO: 96 | QEHLVESGGGLVDIGGSLRLSCAASERIFS |
| P, R, S 103 FW1 sequence no. 6 | SEQ ID NO: 97 | QVKLEESGGGLAQPGGSLRLSCVASGFTFS |
| P, R, S 103 FW1 sequence no. 7 | SEQ ID NO: 98 | EVQLVESGGGLVQPGGSLRLSCVCVSSGCT |
| P, R, S 103 FW1 sequence no. 8 | SEQ ID NO: 99 | EVQLVESGGGLALPGGSLTLSCVFSGSTFS |

and in which
iv) FR2 is an amino acid sequence that has at least 80% amino acid identity with at least one of the following amino acid sequences:

| | | |
|---|---|---|
| P, R, S 103 FW2 sequence no. 1 | SEQ ID NO: 102 | WFRQAPGKEREFVA |
| P, R, S 103 FW2 sequence no. 2 | SEQ ID NO: 103 | WVRQAPGKVLEWVS |
| P, R, S 103 FW2 sequence no. 3 | SEQ ID NO: 104 | WVRRPPGKGLEWVS |
| P, R, S 103 FW2 sequence no. 4 | SEQ ID NO: 105 | WIRQAPGKEREGVS |
| P, R, S 103 FW2 sequence no. 5 | SEQ ID NO: 106 | WVRQYPGKEPEWVS |
| P, R, S 103 FW2 sequence no. 6 | SEQ ID NO: 107 | WFRQPPGKEHEFVA |
| P, R, S 103 FW2 sequence no. 7 | SEQ ID NO: 108 | WYRQAPGKRTELVA |
| P, R, S 103 FW2 sequence no. 8 | SEQ ID NO: 109 | WLRQAPGQGLEWVS |
| P, R, S 103 FW2 sequence no. 9 | SEQ ID NO: 110 | WLRQTPGKGLEWVG |
| P, R, S 103 FW2 sequence no. 10 | SEQ ID NO: 111 | WVRQAPGKAEEFVS |

and in which:
10 v) FR3 is an amino acid sequence that has at least 80% amino acid identity with at least one of the following amino acid sequences:

TABLE A-21
Representative FW3 sequences for Nanobodies of the P, R, S 103-group.

| | | |
|---|---|---|
| P, R, S 103 FW3 sequence no. 1 | SEQ ID NO: 112 | RFTISRDNAKNTVYLQMNSLKPEDTAVYYCAA |
| P, R, S 103 FW3 sequence no. 2 | SEQ ID NO: 113 | RFTISRDNARNTLYLQMDSLIPEDTALYYCAR |
| P, R, S 103 FW3 sequence no. 3 | SEQ ID NO: 114 | RFTISRDNAKNEMYLQMNNLKTEDTGVYWCGA |
| P, R, S 103 FW3 sequence no. 4 | SEQ ID NO: 115 | RFTISSDSNRNMIYLQMNNLKPEDTAVYYCAA |
| P, R, S 103 FW3 sequence no. 5 | SEQ ID NO: 116 | RFTISRDNAKNMLYLHLNNLKSEDTAVYYCRR |
| P, R, S 103 FW3 sequence no. 6 | SEQ ID NO: 117 | RFTISRDNAKKTVYLRLNSLNPEDTAVYSCNL |
| P, R, S 103 FW3 sequence no. 7 | SEQ ID NO: 118 | RFKISRDNAKKTLYLQMNSLGPEDTAMYYCQR |
| P, R, S 103 FW3 sequence no. 8 | SEQ ID NO: 119 | RFTVSRDNGKNTAYLRMNSLKPEDTADYYCAV |

and in which:
vi) FR4 is an amino acid sequence that has at least 80% amino acid identity with at least one of the following amino acid sequences:

| | | |
|---|---|---|
| P, R, S 103 FW4 sequence no. 1 | SEQ ID NO: 120 | RGQGTQVTVSS |
| P, R, S 103 FW4 sequence no. 2 | SEQ ID NO: 121 | LRGGTQVTVSS |
| P, R, S 103 FW4 sequence no. 3 | SEQ ID NO: 122 | GNKGTLVTVSS |
| P, R, S 103 FW4 sequence no. 4 | SEQ ID NO: 123 | SSPGTQVTVSS |
| P, R, S 103 FW4 sequence no. 5 | SEQ ID NO: 124 | SSQGTLVTVSS |
| P, R, S 103 FW4 sequence no. 6 | SEQ ID NO: 125 | RSRGIQVTVSS |

and in which:
vii) CDR1, CDR2 and CDR3 are as defined herein, and are preferably as defined according to one of the preferred aspects herein, and are more preferably as defined according to one of the more preferred aspects herein.

In the above Nanobodies, one or more of the further Hallmark residues are preferably as described herein (for example, when they are V_{HH} sequences or partially humanized Nanobodies).

With regard to framework 1, it will again be clear to the skilled person that, for determining the degree of amino acid identity, the amino acid residues on positions 1 to 4 and 27 to 30 are preferably disregarded.

In view of this, a Nanobody of the P,R,S 103 class may be an amino acid sequence that is comprised of four framework regions/sequences interrupted by three complementarity determining regions/sequences, in which:
i) the amino acid residue at position 103 according to the Kabat numbering is different from W;
and in which:
ii) preferably the amino acid residue at position 103 according to the Kabat numbering is P, R or S, and more preferably R;
and in which:
iii) FR1 is an amino acid sequence that, on positions 5 to 26 of the Kabat numbering, has at least 80% amino acid identity with at least one of the following amino acid sequences:

TABLE A-22
Representative FW1 sequences (amino acid residues 5 to 26) for Nanobodies
of the P, R, S 103-group.

| | | |
|---|---|---|
| P, R, S 103 FW1 sequence no. 9 | SEQ ID NO: 100 | VESGGGLVQAGGSLRLSCAASG |
| P, R, S 103 FW1 sequence no. 10 | SEQ ID NO: 101 | AESGGGLVQPGGSLKLSCAASR |

and in which:
iv) FR2, FR3 and FR4 are as mentioned herein for FR2, FR3 and FR4 of Nanobodies of the P,R,S 103 class;
and in which:
v) CDR1, CDR2 and CDR3 are as defined herein, and are preferably as defined according to one of the preferred aspects herein, and are more preferably as defined according to one of the more preferred aspects herein.

The above Nanobodies may for example be V_{HH} sequences or may be humanized Nanobodies. When the above Nanobody sequences are V_{HH} sequences, they may be suitably humanized, as further described herein. When the Nanobodies are partially humanized Nanobodies, they may optionally be further suitably humanized, again as described herein.

In the above Nanobodies, one or more of the further Hallmark residues are preferably as described herein (for example, when they are V_{HH} sequences or partially humanized Nanobodies).

In another preferred, but non-limiting aspect, the invention relates to a Nanobody as described above, in which the CDR sequences have at least 70% amino acid identity, preferably at least 80% amino acid identity, more preferably at least 90% amino acid identity, such as 95% amino acid identity or more or even essentially 100% amino acid identity with the CDR sequences of at least one of the amino acid sequences of SEQ ID NO's: 182 to 189 and 306 to 321. This degree of amino acid identity can for example be determined by determining the degree of amino acid identity (in a manner described herein) between said Nanobody and one or more of the sequences of SEQ ID NO's: 182 to 189 and 306 to 321, in which the amino acid residues that form the framework regions are disregarded. Such Nanobodies can be as further described herein.

As already mentioned herein, another preferred but non-limiting aspect of the invention relates to a Nanobody with an amino acid sequence that is chosen from the group consisting of SEQ ID NO's: 182 to 189 and 306 to 321 or from the group consisting of from amino acid sequences that have more than 80%, preferably more than 90%, more preferably more than 95%, such as 99% or more sequence identity (as defined herein) with at least one of the amino acid sequences of SEQ ID NO's: 182 to 189 and 306 to 321.

Also, in the above Nanobodies:
i) any amino acid substitution (when it is not a humanizing substitution as defined herein) is preferably, and compared to the corresponding amino acid sequence of SEQ ID NO's: 182 to 189 and 306 to 321, a conservative amino acid substitution, (as defined herein);
and/or:
ii) its amino acid sequence preferably contains either only amino acid substitutions, or otherwise preferably no more than 5, preferably no more than 3, and more preferably only 1 or 2 amino acid deletions or insertions, compared to the corresponding amino acid sequence of SEQ ID NO's: 182 to 189 and 306 to 321;
and/or
iii) the CDR's may be CDR's that are derived by means of affinity maturation, for example starting from the CDR's of to the corresponding amino acid sequence of SEQ ID NO's: 182 to 189 and 306 to 321.

Preferably, CDR sequences and FR sequences present in the Nanobodies of the invention are such that the Nanobodies of the invention will bind to MC4R with an affinity less than 500 nM, preferably less than 200 nM, more preferably less than 10 nM, such as less than 500 pM.

According to one non-limiting aspect of the invention, a Nanobody may be as defined herein, but with the proviso that it has at least “one amino acid difference” (as defined herein) in at least one of the framework regions compared to the corresponding framework region of a naturally occurring human V_{H} domain, and in particular compared to the corresponding framework region of DP-47. More specifically, according to one non-limiting aspect of the invention, a Nanobody may be as defined herein, but with the proviso that it has at least “one amino acid difference” (as defined herein) at least one of the Hallmark residues (including those at positions 108, 103 and/or 45) compared to the corresponding framework region of a naturally occurring human V_{H} domain, and in particular compared to the corresponding framework region of DP-47. Usually, a Nanobody will have at least one such amino acid difference with a naturally occurring V_{H} domain in at least one of FR2 and/or FR4, and in particular at least one of the Hallmark residues in FR2 and/or FR4 (again, including those at positions 108, 103 and/or 45).

Also, a humanized Nanobody of the invention may be as defined herein, but with the proviso that it has at least “one amino acid difference” (as defined herein) in at least one of the framework regions compared to the corresponding framework region of a naturally occurring V_{HH} domain. More specifically, according to one non-limiting aspect of the invention, a humanized Nanobody may be as defined herein, but with the proviso that it has at least “one amino acid difference” (as defined herein) at least one of the Hallmark residues (including those at positions 108, 103 and/or 45) compared to the corresponding framework region of a naturally occurring V_{HH} domain. Usually, a humanized Nanobody will have at least one such amino acid difference with a naturally occurring V_{HH} domain in at least one of FR2 and/or FR4, and in particular at least one of the Hallmark residues in FR2 and/or FR4 (again, including those at positions 108, 103 and/or 45).

As will be clear from the disclosure herein, it is also within the scope of the invention to use natural or synthetic analogs, mutants, variants, alleles, homologs and orthologs (herein collectively referred to as “analogs”) of the Nanobodies of the invention as defined herein, and in particular analogs of the Nanobodies of SEQ ID NO's 182 to 189 and 306 to 321. Thus, according to one aspect of the invention, the term “Nanobody of the invention” in its broadest sense also covers such analogs.

Generally, in such analogs, one or more amino acid residues may have been replaced, deleted and/or added, compared to the Nanobodies of the invention as defined herein. Such substitutions, insertions or deletions may be made in one or more of the framework regions and/or in one or more of the CDR's. When such substitutions, insertions or deletions are made in one or more of the framework regions, they may be made at one or more of the Hallmark residues and/or at one or more of the other positions in the framework residues, although substitutions, insertions or deletions at the Hallmark residues are generally less preferred (unless these are suitable humanizing substitutions as described herein).

By means of non-limiting examples, a substitution may for example be a conservative substitution (as described herein) and/or an amino acid residue may be replaced by another amino acid residue that naturally occurs at the same position in another V_{HH} domain (see Tables A-5 to A-8 for some non-limiting examples of such substitutions), although the invention is generally not limited thereto. Thus, any one or more substitutions, deletions or insertions, or any combination thereof, that either improve the properties of the Nanobody of the invention or that at least do not detract too much from the desired properties or from the balance or combination of desired properties of the Nanobody of the invention (i.e. to the extent that the Nanobody is no longer suited for its intended use) are included within the scope of the invention. A skilled person will generally be able to determine and select suitable substitutions, deletions or insertions, or suitable combinations of thereof, based on the disclosure herein and optionally after a limited degree of routine experimentation, which may for example involve introducing a limited number of possible substitutions and determining their influence on the properties of the Nanobodies thus obtained.

For example, and depending on the host organism used to express the Nanobody or polypeptide of the invention, such deletions and/or substitutions may be designed in such a way that one or more sites for post-translational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation (again as described herein).

Also, or in addition, one or more substitution may be introduced that reduce the binding by so-called “pre-existing anti-drug antibodies”, in particular to the C-terminal end of a Nanobody (for example, when a Nanobody has an exposed C-terminal end in the construct in which the Nanobody has been included). Reference is for example made to WO2012/175741 (in the name of Ablynx NV) and/or those described in WO2015/173325 (also in the name of Ablynx NV), which describe such pre-existing anti-drug antibodies and mutations or combinations of mutations that can be included in the sequence of a Nanobody to reduce binding by such pre-existing anti-drug antibodies. As also described in WO2012/175741 and WO2015/173325, when a Nanobody forms the C-terminal end of a protein, polypeptide or construct in which such a Nanobody is present, such mutations or combination of mutations may also be suitably combined with a C-terminal extension of the C-terminal Nanobody, which may for example be a C-terminal alanine residue that is present at or on the C-terminal end of the Nanobody, protein, polypeptide or construct. Again, such mutations that are intended to reduce the binding of pre-existing anti-drug antibodies may be suitably combined with other mutations as described herein. Also, WO2015/173342 by Ablynx describes methods that can be used to test the ability of such mutations(s) or combination of mutations to reduce binding by pre-existing anti-drug antibodies.

As can be seen from the data on the V_{HH} entropy and V_{HH} variability given in Tables A-5 to A-8 above, some amino acid residues in the framework regions are more conserved than others. Generally, although the invention in its broadest sense is not limited thereto, any substitutions, deletions or insertions are preferably made at positions that are less conserved. Also, generally, amino acid substitutions are preferred over amino acid deletions or insertions.

The analogs are preferably such that they can bind to MC4R with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the Nanobodies of the invention.

The analogs are preferably also such that they retain the favourable properties the Nanobodies, as described herein.

Also, according to one preferred aspect, the analogs have a degree of sequence identity of at least 70%, preferably at least 80%, more preferably at least 90%, such as at least 95% or 99% or more; and/or preferably have at most 20, preferably at most 10, even more preferably at most 5, such as 4, 3, 2 or only 1 amino acid difference (as defined herein), with one of the Nanobodies of SEQ ID Nos: 182 to 189 and 306 to 321.

Also, the framework sequences and CDR's of the analogs are preferably such that they are in accordance with the preferred aspects defined herein. More generally, as described herein, the analogs will have (a) a Q at position 108; and/or (b) a charged amino acid or a cysteine residue at position 45 and preferably an E at position 44, and more preferably E at position 44 and R at position 45; and/or (c) P, R or S at position 103.

One preferred class of analogs of the Nanobodies of the invention comprise Nanobodies that have been humanized (i.e. compared to the sequence of a naturally occurring Nanobody of the invention). As mentioned in the background art cited herein, such humanization generally involves replacing one or more amino acid residues in the sequence of a naturally occurring V_{HH} with the amino acid residues that occur at the same position in a human V_{H} domain, such as a human V_{H}3 domain. Examples of possible humanizing substitutions or combinations of humanizing substitutions will be clear to the skilled person, for example from the Tables herein, from the possible humanizing substitutions mentioned in the background art cited herein, and/or from a comparison between the sequence of a Nanobody and the sequence of a naturally occurring human V_{H} domain.

The humanizing substitutions should be chosen such that the resulting humanized Nanobodies still retain the favourable properties of Nanobodies as defined herein, and more preferably such that they are as described for analogs in the preceding paragraphs. A skilled person will generally be able to determine and select suitable humanizing substitutions or suitable combinations of humanizing substitutions, based on the disclosure herein and optionally after a limited degree of routine experimentation, which may for example involve introducing a limited number of possible humanizing substitutions and determining their influence on the properties of the Nanobodies thus obtained.

Generally, as a result of humanization, the Nanobodies of the invention may become more “human-like”, while still retaining the favorable properties of the Nanobodies of the invention as described herein. As a result, such humanized Nanobodies may have several advantages, such as a reduced immunogenicity, compared to the corresponding naturally occurring V_{HH} domains. Again, based on the disclosure herein and optionally after a limited degree of routine experimentation, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring V_{HH} domains on the other hand.

The Nanobodies of the invention may be suitably humanized at any framework residue(s), such as at one or more Hallmark residues (as defined herein) or at one or more other framework residues (i.e. non-Hallmark residues) or any suitable combination thereof. One preferred humanizing substitution for Nanobodies of the “P,R,S-103 group” or the “KERE group” is Q108 into L108. Nanobodies of the “GLEW class” may also be humanized by a Q108 into L108 substitution, provided at least one of the other Hallmark residues contains a camelid (camelizing) substitution (as defined herein). For example, as mentioned above, one particularly preferred class of humanized Nanobodies has GLEW or a GLEW-like sequence at positions 44-47; P, R or S (and in particular R) at position 103, and an L at position 108.

The humanized and other analogs, and nucleic acid sequences encoding the same, can be provided in any manner known per se, for example using one or more of the techniques mentioned on pages 103 and 104 of WO 08/020079.

As mentioned there, it will be also be clear to the skilled person that the Nanobodies of the invention (including their analogs) can be designed and/or prepared starting from human V_{H} sequences (i.e. amino acid sequences or the corresponding nucleotide sequences), such as for example from human Vu3 sequences such as DP-47, DP-51 or DP-29, i.e. by introducing one or more camelizing substitutions (i.e. changing one or more amino acid residues in the amino acid sequence of said human Vu domain into the amino acid residues that occur at the corresponding position in a Vu domain), so as to provide the sequence of a Nanobody of the invention and/or so as to confer the favourable properties of a Nanobody to the sequence thus obtained. Again, this can generally be performed using the various methods and techniques referred to in the previous paragraph, using an amino acid sequence and/or nucleotide sequence for a human Vu domain as a starting point.

Some preferred, but non-limiting camelizing substitutions can be derived from Tables A-5-A-8. It will also be clear that camelizing substitutions at one or more of the Hallmark residues will generally have a greater influence on the desired properties than substitutions at one or more of the other amino acid positions, although both and any suitable combination thereof are included within the scope of the invention. For example, it is possible to introduce one or more camelizing substitutions that already confer at least some the desired properties, and then to introduce further camelizing substitutions that either further improve said properties and/or confer additional favourable properties. Again, the skilled person will generally be able to determine and select suitable camelizing substitutions or suitable combinations of camelizing substitutions, based on the disclosure herein and optionally after a limited degree of routine experimentation, which may for example involve introducing a limited number of possible camelizing substitutions and determining whether the favourable properties of Nanobodies are obtained or improved (i.e. compared to the original Vu domain). Generally, however, such camelizing substitutions are preferably such that the resulting an amino acid sequence at least contains (a) a Q at position 108; and/or (b) a charged amino acid or a cysteine residue at position 45 and preferably also an E at position 44, and more preferably E at position 44 and R at position 45; and/or (c) P, R or S at position 103; and optionally one or more further camelizing substitutions. More preferably, the camelizing substitutions are such that they result in a Nanobody of the invention and/or in an analog thereof (as defined herein), such as in a humanized analog and/or preferably in an analog that is as defined in the preceding paragraphs.

As will also be clear from the disclosure herein, it is also within the scope of the invention to use parts or fragments, or combinations of two or more parts or fragments, of the Nanobodies of the invention as defined herein, and in particular parts or fragments of the Nanobodies of SEQ ID NO's: 182 to 189 and 306 to 321. Thus, according to one aspect of the invention, the term “Nanobody of the invention” in its broadest sense also covers such parts or fragments.

Generally, such parts or fragments of the Nanobodies of the invention (including analogs thereof) have amino acid sequences in which, compared to the amino acid sequence of the corresponding full length Nanobody of the invention (or analog thereof), one or more of the amino acid residues at the N-terminal end, one or more amino acid residues at the C-terminal end, one or more contiguous internal amino acid residues, or any combination thereof, have been deleted and/or removed.

The parts or fragments are preferably such that they can bind to MC4R with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the Nanobodies of the invention.

Any part or fragment is preferably such that it comprises at least 10 contiguous amino acid residues, preferably at least 20 contiguous amino acid residues, more preferably at least 30 contiguous amino acid residues, such as at least 40 contiguous amino acid residues, of the amino acid sequence of the corresponding full length Nanobody of the invention.

Also, any part or fragment is such preferably that it comprises at least one of CDR1, CDR2 and/or CDR3 or at least part thereof (and in particular at least CDR3 or at least part thereof). More preferably, any part or fragment is such that it comprises at least one of the CDR's (and preferably at least CDR3 or part thereof) and at least one other CDR (i.e. CDR1 or CDR2) or at least part thereof, preferably connected by suitable framework sequence(s) or at least part thereof. More preferably, any part or fragment is such that it comprises at least one of the CDR's (and preferably at least CDR3 or part thereof) and at least part of the two remaining CDR's, again preferably connected by suitable framework sequence(s) or at least part thereof.

According to another particularly preferred, but non-limiting aspect, such a part or fragment comprises at least CDR3, such as FR3, CDR3 and FR4 of the corresponding full length Nanobody of the invention, i.e. as for example described in the International application WO 03/050531 (Lasters et al.).

As already mentioned above, it is also possible to combine two or more of such parts or fragments (i.e. from the same or different Nanobodies of the invention), i.e. to provide an analog (as defined herein) and/or to provide further parts or fragments (as defined herein) of a Nanobody of the invention. It is for example also possible to combine one or more parts or fragments of a Nanobody of the invention with one or more parts or fragments of a human V_{H} domain.

According to one preferred aspect, the parts or fragments have a degree of sequence identity of at least 50%, preferably at least 60%, more preferably at least 70%, even more preferably at least 80%, such as at least 90%, 95% or 99% or more with one of the Nanobodies of SEQ ID NO's: 182 to 189 and 306 to 321.

The parts and fragments, and nucleic acid sequences encoding the same, can be provided and optionally combined in any manner known per se. For example, such parts or fragments can be obtained by inserting a stop codon in a nucleic acid that encodes a full-sized Nanobody of the invention, and then expressing the nucleic acid thus obtained in a manner known per se (e.g. as described herein). Alternatively, nucleic acids encoding such parts or fragments can be obtained by suitably restricting a nucleic acid that encodes a full-sized Nanobody of the invention or by synthesizing such a nucleic acid in a manner known per se. Parts or fragments may also be provided using techniques for peptide synthesis known per se.

The invention in its broadest sense also comprises derivatives of the Nanobodies of the invention. Such derivatives can generally be obtained by modification, and in particular by chemical and/or biological (e.g enzymatical) modification, of the Nanobodies of the invention and/or of one or more of the amino acid residues that form the Nanobodies of the invention.

Examples of such modifications, as well as examples of amino acid residues within the Nanobody sequence that can be modified in such a manner (i.e. either on the protein backbone but preferably on a side chain), methods and techniques that can be used to introduce such modifications and the potential uses and advantages of such modifications will be clear to the skilled person.

For example, such a modification may involve the introduction (e.g. by covalent linking or in an other suitable manner) of one or more functional groups, residues or moieties into or onto the Nanobody of the invention, and in particular of one or more functional groups, residues or moieties that confer one or more desired properties or functionalities to the Nanobody of the invention. Example of such functional groups will be clear to the skilled person.

For example, such modification may comprise the introduction (e.g. by covalent binding or in any other suitable manner) of one or more functional groups that increase the half-life, the solubility and/or the absorption of the Nanobody of the invention, that reduce the immunogenicity and/or the toxicity of the Nanobody of the invention, that eliminate or attenuate any undesirable side effects of the Nanobody of the invention, and/or that confer other advantageous properties to and/or reduce the undesired properties of the Nanobodies and/or polypeptides of the invention; or any combination of two or more of the foregoing. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including ScFv's and single domain antibodies), for which reference is for example made to Remington's Pharmaceutical Sciences, 16th ed., Mack Publishing Co., Easton, PA (1980). Such functional groups may for example be linked directly (for example covalently) to a Nanobody of the invention, or optionally via a suitable linker or spacer, as will again be clear to the skilled person.

One of the most widely used techniques for increasing the half-life and/or reducing the immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of pegylation can be used, such as the pegylation used in the art for antibodies and antibody fragments (including but not limited to (single) domain antibodies and ScFv's); reference is made to for example Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04/060965. Various reagents for pegylation of proteins are also commercially available, for example from Nektar Therapeutics, USA.

Preferably, site-directed pegylation is used, in particular via a cysteine-residue (see for example Yang et al., Protein Engineering, 16, 10, 761-770 (2003). For example, for this purpose, PEG may be attached to a cysteine residue that naturally occurs in a Nanobody of the invention, a Nanobody of the invention may be modified so as to suitably introduce one or more cysteine residues for attachment of PEG, or an amino acid sequence comprising one or more cysteine residues for attachment of PEG may be fused to the N- and/or C-terminus of a Nanobody of the invention, all using techniques of protein engineering known per se to the skilled person.

Preferably, for the Nanobodies and proteins of the invention, a PEG is used with a molecular weight of more than 5000, such as more than 10,000 and less than 200,000, such as less than 100,000; for example in the range of 20,000-80,000.

Another, usually less preferred modification comprises N-linked or O-linked glycosylation, usually as part of co-translational and/or post-translational modification, depending on the host cell used for expressing the Nanobody or polypeptide of the invention.

Yet another modification may comprise the introduction of one or more detectable labels or other signal-generating groups or moieties, depending on the intended use of the labelled Nanobody. Suitable labels and techniques for attaching, using and detecting them will be clear to the skilled person, and for example include, but are not limited to, the fluorescent labels, phosphorescent labels, chemiluminescent labels, bioluminescent labels, radio-isotopes, metals, metal chelates, metallic cations, chromophores and enzymes, such as those mentioned on page 109 of WO 08/020079. Other suitable labels will be clear to the skilled person, and for example include moieties that can be detected using NMR or ESR spectroscopy.

Such labelled Nanobodies and polypeptides of the invention may for example be used for in vitro, in vivo or in situ assays (including immunoassays known per se such as ELISA, RIA, EIA and other “sandwich assays”, etc.) as well as in vivo diagnostic and imaging purposes, depending on the choice of the specific label.

As will be clear to the skilled person, another modification may involve the introduction of a chelating group, for example to chelate one of the metals or metallic cations referred to above. Suitable chelating groups for example include, without limitation, diethyl-enetriaminepentaacetic acid (DTPA) or ethylenediaminetetraacetic acid (EDTA).

Yet another modification may comprise the introduction of a functional group that is one part of a specific binding pair, such as the biotin-(strept) avidin binding pair. Such a functional group may be used to link the Nanobody of the invention to another protein, polypeptide or chemical compound that is bound to the other half of the binding pair, i.e. through formation of the binding pair. For example, a Nanobody of the invention may be conjugated to biotin, and linked to another protein, polypeptide, compound or carrier conjugated to avidin or streptavidin. For example, such a conjugated Nanobody may be used as a reporter, for example in a diagnostic system where a detectable signal-producing agent is conjugated to avidin or streptavidin. Such binding pairs may for example also be used to bind the Nanobody of the invention to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the Nanobody of the invention.

For some applications, in particular for those applications in which it is intended to kill a cell that expresses the target against which the Nanobodies of the invention are directed (e.g. in the treatment of cancer), or to reduce or slow the growth and/or proliferation such a cell, the Nanobodies of the invention may also be linked to a toxin or to a toxic residue or moiety. Examples of toxic moieties, compounds or residues which can be linked to a Nanobody of the invention to provide—for example—a cytotoxic compound will be clear to the skilled person and can for example be found in the prior art cited above and/or in the further description herein. One example is the so-called ADEPT™ technology described in WO 03/055527.

Other potential chemical and enzymatical modifications will be clear to the skilled person. Such modifications may also be introduced for research purposes (e.g. to study function-activity relationships). Reference is for example made to Lundblad and Bradshaw, Biotechnol. Appl. Biochem., 26, 143-151 (1997).

Preferably, the derivatives are such that they bind to MC4R with an affinity (suitably measured and/or expressed as a K_{D}-value (actual or apparent), a K_{A}-value (actual or apparent), a kₒₙ-rate and/or a k_{off}-rate, or alternatively as an IC₅₀ value, as further described herein) that is as defined herein for the Nanobodies of the invention.

As mentioned above, the invention also relates to proteins or polypeptides that essentially consist of or comprise at least one Nanobody of the invention. By “essentially consist of” is meant that the amino acid sequence of the polypeptide of the invention either is exactly the same as the amino acid sequence of a Nanobody of the invention or corresponds to the amino acid sequence of a Nanobody of the invention which has a limited number of amino acid residues, such as 1-20 amino acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxy terminal end of the amino acid sequence of the Nanobody.

Said amino acid residues may or may not change, alter or otherwise influence the (biological) properties of the Nanobody and may or may not add further functionality to the Nanobody. For example, such amino acid residues:
can comprise an N-terminal Met residue, for example as result of expression in a heterologous host cell or host organism.
may form a signal sequence or leader sequence that directs secretion of the Nanobody from a host cell upon synthesis. Suitable secretory leader peptides will be clear to the skilled person, and may be as further described herein. Usually, such a leader sequence will be linked to the N-terminus of the Nanobody, although the invention in its broadest sense is not limited thereto;
may form a sequence or signal that allows the Nanobody to be directed towards and/or to penetrate or enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such amino acid sequences will be clear to the skilled person and include those mentioned in paragraph c) on page 112 of WO 08/020079
may form a “tag”, for example an amino acid sequence or residue that allows or facilitates the purification of the Nanobody, for example using affinity techniques directed against said sequence or residue. Thereafter, said sequence or residue may be removed (e.g. by chemical or enzymatical cleavage) to provide the Nanobody sequence (for this purpose, the tag may optionally be linked to the Nanobody sequence via a cleavable linker sequence or contain a cleavable motif). Some preferred, but non-limiting examples of such residues are multiple histidine residues, glutatione residues and a myc-tag (see for example SEQ ID NO:31 of WO 06/12282).
may be one or more amino acid residues that have been functionalized and/or that can serve as a site for attachment of functional groups. Suitable amino acid residues and functional groups will be clear to the skilled person and include, but are not limited to, the amino acid residues and functional groups mentioned herein for the derivatives of the Nanobodies of the invention;
may form a C-terminal extension that reduces binding by pre-existing anti-drug antibodies, as described in WO2012/175741 (in the name of Ablynx NV) and/or those described in WO2015/173325 (also in the name of Ablynx NV), in particular when the Nanobody forms the C-terminal end of the protein, polypeptide or construct in which it is present. As also mentioned herein and in WO2012/175741 and WO2015/173325, such a C-terminal extension may be suitable combined with one or more further mutations that reduce the binding of such pre-existing anti-drug antibodies. WO2015/173342 by Ablynx describes methods that can be used to test the ability of such a C-terminal extension, optionally in combination with one or more mutations(s) as described in WO2012/175741 and/or WO2015/173325 to reduce binding by pre-existing anti-drug antibodies.

According to another aspect, a polypeptide of the invention comprises a Nanobody of the invention, which is fused at its amino terminal end, at its carboxy terminal end, or both at its amino terminal end and at its carboxy terminal end to at least one further amino acid sequence, i.e. so as to provide a fusion protein comprising said Nanobody of the invention and the one or more further amino acid sequences. Such a fusion will also be referred to herein as a “Nanobody fusion”.

The one or more further amino acid sequence may be any suitable and/or desired amino acid sequences. The further amino acid sequences may or may not change, alter or otherwise influence the (biological) properties of the Nanobody, and may or may not add further functionality to the Nanobody or the polypeptide of the invention. Preferably, the further amino acid sequence is such that it confers one or more desired properties or functionalities to the Nanobody or the polypeptide of the invention.

For example, the further amino acid sequence may also provide a second binding site, which binding site may be directed against any desired protein, polypeptide, antigen, antigenic determinant or epitope (including but not limited to the same protein, polypeptide, antigen, antigenic determinant or epitope against which the Nanobody of the invention is directed, or a different protein, polypeptide, antigen, antigenic determinant or epitope).

Example of such amino acid sequences will be clear to the skilled person, and may generally comprise all amino acid sequences that are used in peptide fusions based on conventional antibodies and fragments thereof (including but not limited to ScFv's and single domain antibodies). Reference is for example made to the review by Holliger and Hudson, Nature Biotechnology, 23, 9, 1126-1136 (2005).

For example, such an amino acid sequence may be an amino acid sequence that increases the half-life, the solubility, or the absorption, reduces the immunogenicity or the toxicity, eliminates or attenuates undesirable side effects, and/or confers other advantageous properties to and/or reduces the undesired properties of the polypeptides of the invention, compared to the Nanobody of the invention per se. Some non-limiting examples of such amino acid sequences are serum proteins, such as human serum albumin (see for example WO 00/27435) or haptenic molecules (for example haptens that are recognized by circulating antibodies, see for example WO 98/22141).

In particular, it has been described in the art that linking fragments of immunoglobulins (such as V_{H} domains) to serum albumin or to fragments thereof can be used to increase the half-life. Reference is for made to WO 00/27435 and WO 01/077137). According to the invention, the Nanobody of the invention is preferably either directly linked to serum albumin (or to a suitable fragment thereof) or via a suitable linker, and in particular via a suitable peptide linked so that the polypeptide of the invention can be expressed as a genetic fusion (protein). According to one specific aspect, the Nanobody of the invention may be linked to a fragment of serum albumin that at least comprises the domain III of serum albumin or part thereof. Reference is for example made to WO 07/112940 of Ablynx N.V

Alternatively, the further amino acid sequence may provide a second binding site or binding unit that is directed against a serum protein (such as, for example, human serum albumin or another serum protein such as IgG), so as to provide increased half-life in serum. Such amino acid sequences for example include the Nanobodies described below, as well as the small peptides and binding proteins described in WO 91/01743, WO 01/45746 and WO 02/076489 and the dAb's described in WO 03/002609 and WO 04/003019. Reference is also made to Harmsen et al., Vaccine, 23 (41); 4926-42, 2005, as well as to EP 0 368 684, as well as to the following the U.S. provisional applications 60/843,349 (see also PCT/EP2007/059475), 60/850,774 (see also PCT/EP2007/060849), 60/850,775 (see also PCT/EP2007/060850) by Ablynx N.V. mentioned herein and US provisional application of Ablynx N.V. entitled “Peptides capable of binding to serum proteins” filed on Dec. 5, 2006 ((see also PCT/EP2007/063348).

Such amino acid sequences may in particular be directed against serum albumin (and more in particular human serum albumin) and/or against IgG (and more in particular human IgG). For example, such amino acid sequences may be amino acid sequences that are directed against (human) serum albumin and amino acid sequences that can bind to amino acid residues on (human) serum albumin that are not involved in binding of serum albumin to FcRn (see for example WO 06/0122787) and/or amino acid sequences that are capable of binding to amino acid residues on serum albumin that do not form part of domain III of serum albumin (see again for example WO 06/0122787); amino acid sequences that have or can provide an increased half-life (see for example WO 08/028977 by Ablynx N.V.); amino acid sequences against human serum albumin that are cross-reactive with serum albumin from at least one species of mammal, and in particular with at least one species of primate (such as, without limitation, monkeys from the genus Macaca (such as, and in particular, cynomologus monkeys (Macaca fascicularis) and/or rhesus monkeys (Macaca mulatta)) and baboon (Papio ursinus), reference is again made to the U.S. provisional application 60/843,349 and PCT/EP2007/059475); amino acid sequences that can bind to serum albumin in a pH independent manner (see for example the U.S. provisional application 60/850,774 by Ablynx N.V. entitled “Amino acid sequences that bind to serum proteins in a manner that is essentially independent of the pH, compounds comprising the same, and uses thereof”, filed on Oct. 11, 2006; see also and PCT/EP2007/059475) and/or amino acid sequences that are conditional binders (see for example the U.S. provisional application 60/850,775 by Ablynx N.V. entitled “Amino acid sequences that bind to a desired molecule in a conditional manner”, filed on Oct. 11, 2006; see also PCT/EP2007/060850).

According to another aspect, the one or more further amino acid sequences may comprise one or more parts, fragments or domains of conventional 4-chain antibodies (and in particular human antibodies) and/or of heavy chain antibodies. For example, although usually less preferred, a Nanobody of the invention may be linked to a conventional (preferably human) V_{H} or V_{L} domain or to a natural or synthetic analog of a Vu or V_{L} domain, again optionally via a linker sequence (including but not limited to other (single) domain antibodies, such as the dAb's described by Ward et al.).

The at least one Nanobody may also be linked to one or more (preferably human) C_{H}1, C_{H}2 and/or C_{H}3 domains, optionally via a linker sequence. For instance, a Nanobody linked to a suitable C_{H}1 domain could for example be used—together with suitable light chains—to generate antibody fragments/structures analogous to conventional Fab fragments or F(ab′)₂ fragments, but in which one or (in case of an F(ab′)₂ fragment) one or both of the conventional V_{H} domains have been replaced by a Nanobody of the invention. Also, two Nanobodies could be linked to a C_{H}3 domain (optionally via a linker) to provide a construct with increased half-life in vivo.

According to one specific aspect of a polypeptide of the invention, one or more Nanobodies of the invention may be linked (optionally via a suitable linker or hinge region) to one or more constant domains (for example, 2 or 3 constant domains that can be used as part of/to form an Fc portion), to an Fc portion and/or to one or more antibody parts, fragments or domains that confer one or more effector functions to the polypeptide of the invention and/or may confer the ability to bind to one or more Fc receptors. For example, for this purpose, and without being limited thereto, the one or more further amino acid sequences may comprise one or more C_{H}2 and/or C_{H}3 domains of an antibody, such as from a heavy chain antibody (as described herein) and more preferably from a conventional human 4-chain antibody; and/or may form (part of) and Fc region, for example from IgG (e.g. from IgG1, IgG2, IgG3 or IgG4), from IgE or from another human Ig such as IgA, IgD or IgM. For example, WO 94/04678 describes heavy chain antibodies comprising a Camelid V_{HH} domain or a humanized derivative thereof (i.e. a Nanobody), in which the Camelidac C_{H}2 and/or C_{H}3 domain have been replaced by human C_{H}2 and C_{H}3 domains, so as to provide an immunoglobulin that consists of 2 heavy chains each comprising a Nanobody and human C_{H}2 and C_{H}3 domains (but no C_{H}1 domain), which immunoglobulin has the effector function provided by the C_{H}2 and C_{H}3 domains and which immunoglobulin can function without the presence of any light chains. Other amino acid sequences that can be suitably linked to the Nanobodies of the invention so as to provide an effector function will be clear to the skilled person, and may be chosen on the basis of the desired effector function(s). Reference is for example made to WO 04/058820, WO 99/42077, WO 02/056910 and WO 05/017148, as well as the review by Holliger and Hudson, supra; and to the non-prepublished US provisional application by Ablynx N.V. entitled “Constructs comprising single variable domains and an Fc portion derived from IgE” which has a filing date of Dec. 4, 2007. Coupling of a Nanobody of the invention to an Fc portion may also lead to an increased half-life, compared to the corresponding Nanobody of the invention. For some applications, the use of an Fc portion and/or of constant domains (i.e. C_{H}2 and/or C_{H}3 domains) that confer increased half-life without any biologically significant effector function may also be suitable or even preferred. Other suitable constructs comprising one or more Nanobodies and one or more constant domains with increased half-life in vivo will be clear to the skilled person, and may for example comprise two Nanobodies linked to a C_{H}3 domain, optionally via a linker sequence.

Such heavy chain-only Fc-based constructs form one particularly preferred aspect of the invention, with the sequence of some preferred but-non-limiting examples thereof being given in Table A-2 as SEQ ID NO's: 190 to 193.

The Fc portions used in the compounds/constructs of the invention (i.e. with or without the C_{H}1 domain) and any C_{H} domains present therein may be naturally occurring Fc portions/domains (and in particular human Fc portions/domains) or may be non-naturally occurring (i.e. synthetic or semi-synthetic) Fc domains, for example Fc domains that contain one or more suitable mutations that confer one or more properties to the Fc portion and/or to the construct(s) of the invention comprising the same (e.g. depending on the desired properties of the resulting construct of the invention and the intended use of the constructs). Such mutations and their associated properties (such as, without limitation, modulated effector functions or altered half-life) will be clear to the skilled person. Reference is for example made to Jacobsen et al., J. Biol Chem, vol. 292, no. 5, pp. 1865-1875 Feb. 3, 2017, to the review by Wang et al., Protein Cell 2018, 9 (1): 63-73 and the further references cited therein.

For example and without limitations, for some applications of the constructs of the invention, it may be desirable that the Fc portion (which again may be an Fc portion with or without C_{H}1 domain) shows reduced effector function compared to the corresponding naturally occurring Fc portion, and the review by Wang et al. (supra) in Table 1 lists some particularly suitable mutations that may be introduced into a human Fc portion to achieve this, such as, without limitation, N297A or N297Q or N297G or the combination L234A/L235A (also known as “LALA”).

Table A-23 below list some examples of preferred but non-limiting human Fc portions for use in the constructs/compounds of the invention. As will be clear to the skilled person, these Fc portions comprise C_{H}2 and C_{H}3 domains preceded by a suitable hinge sequence (DKTHTCPPCPAPELLGGP, SEQ ID NO:325)

TABLE A-23
Preferred but non-limiting human Fc portions for use in the compounds of
the invention.

| | | |
|---|---|---|
| huFcIgG1 | SEQ ID | DKTHTCPPCPAPELLGGPSVFLFPPKPKDTLMISRTPEVTCVVVDVSHEDP |
| | NO: 322 | EVKFNWYVDGVEVHNAKTKPREEQYASTYRVVSVLTVLHQDWLNGKEYK |
| | | CKVSNKALPAPIEKTISKAKGQPREPQVYTLPPSRDELTKNQVSLTCLVKG |
| | | FYPSDIAVEWESNGQPENNYKTTPPVLDSDGSFFLYSKLTVDKSRWQQG |
| | | NVFSCSVMHEALHNHYTQKSLSLSPGK |
| huFcIgG1_ | SEQ ID | DKTHTCPPCPAPELLGGPSVFLFPPKPKDTLMISRTPEVTCVVVDVSHEDP |
| N297A | NO: 323 | EVKFNWYVDGVEVHNAKTKPREEQYASTYRVVSVLTVLHQDWLNGKEYK |
| | | CKVSNKALPAPIEKTISKAKGQPREPQVYTLPPSRDELTKNQVSLTCLVKG |
| | | FYPSDIAVEWESNGQPENNYKTTPPVLDSDGSFFLYSKLTVDKSRWQQG |
| | | NVFSCSVMHEALHNHYTQKSLSLSPGK |
| pN2037 | SEQ ID | DKTHTCPPCPAPEAAGGPSVFLFPPKPKDTLMISRTPEVTCVVVDVSHED |
| (huFc IgG1 | NO: 324 | PEVKFNWYVDGVEVHNAKTKPREEQYNSTYRVVSVLTVLHQDWLNGKEY |
| L234A_ | | KCKVSNKALPAPIEKTISKAKGQPREPQVYTLPPSRDELTKNQVSLTCLVK |
| L235A) | | GFYPSDIAVEWESNGQPENNYKTTPPVLDSDGSFFLYSKLTVDKSRWQQ |
| | | GNVFSCSVMHEALHNHYTQKSLSLSPGK |

Generally, any fusion protein or derivatives with increased half-life will preferably have a molecular weight of more than 50 kD, the cut-off value for renal absorption.

In another one specific, but non-limiting, aspect, in order to form a polypeptide of the invention, one or more amino acid sequences of the invention may be linked (optionally via a suitable linker or hinge region) to naturally occurring, synthetic or semisynthetic constant domains (or analogs, variants, mutants, parts or fragments thereof) that have a reduced (or essentially no) tendency to self-associate into dimers (i.e. compared to constant domains that naturally occur in conventional 4-chain antibodies). Such monomeric (i.e. not self-associating) Fc chain variants, or fragments thereof, will be clear to the skilled person. For example, Helm et al., J Biol Chem 1996 271 7494, describe monomeric Fcε chain variants that can be used in the polypeptide chains of the invention.

Also, such monomeric Fc chain variants are preferably such that they are still capable of binding to the complement or the relevant Fc receptor(s) (depending on the Fc portion from which they are derived), and/or such that they still have some or all of the effector functions of the Fc portion from which they are derived (or at a reduced level still suitable for the intended use). Alternatively, in such a polypeptide chain of the invention, the monomeric Fc chain may be used to confer increased half-life upon the polypeptide chain, in which case the monomeric Fc chain may also have no or essentially no effector functions.

Generally, any construct, compound, fusion protein, or derivative as described herein with increased half-life will preferably have a molecular weight of more than 50 kD, the cut-off value for renal absorption.

Bivalent/multivalent, bispecific/multispecific or biparatopic/multiparatopic polypeptides of the invention may also be linked to Fc portions, in order to provide polypeptide constructs of the type that is described in the non-prepublished US provisional application U.S. 61/005,331 entitled “immunoglobulin constructs” filed on Dec. 4, 2007.

The further amino acid sequences may also form a signal sequence or leader sequence that directs secretion of the Nanobody or the polypeptide of the invention from a host cell upon synthesis (for example to provide a pre-, pro- or prepro-form of the polypeptide of the invention, depending on the host cell used to express the polypeptide of the invention).

The further amino acid sequence may also form a sequence or signal that allows the Nanobody or polypeptide of the invention to be directed towards and/or to penetrate or enter into specific organs, tissues, cells, or parts or compartments of cells, and/or that allows the Nanobody or polypeptide of the invention to penetrate or cross a biological barrier such as a cell membrane, a cell layer such as a layer of epithelial cells, a tumor including solid tumors, or the blood-brain-barrier. Suitable examples of such amino acid sequences will be clear to the skilled person, and for example include, but are not limited to, those mentioned on page 118 of WO 08/020079. For some applications, in particular for those applications in which it is intended to kill a cell that expresses the target against which the Nanobodies of the invention are directed (e.g. in the treatment of cancer), or to reduce or slow the growth and/or proliferation of such a cell, the Nanobodies of the invention may also be linked to a (cyto)toxic protein or polypeptide. Examples of such toxic proteins and polypeptides which can be linked to a Nanobody of the invention to provide—for example—a cytotoxic polypeptide of the invention will be clear to the skilled person and can for example be found in the prior art cited above and/or in the further description herein. One example is the so-called ADEPT™ technology described in WO 03/055527.

According to one preferred, but non-limiting aspect, said one or more further amino acid sequences comprise at least one further Nanobody, so as to provide a polypeptide of the invention that comprises at least two, such as three, four, five or more Nanobodies, in which said Nanobodies may optionally be linked via one or more linker sequences (as defined herein). As described on pages 119 and 120 of WO 08/020079, polypeptides of the invention that comprise two or more Nanobodies, of which at least one is a Nanobody of the invention, will also be referred to herein as “multivalent” polypeptides of the invention, and the Nanobodies present in such polypeptides will also be referred to herein as being in a “multivalent format”. For example, “bivalent” and “trivalent” polypeptides of the invention may be as further described on pages 119 and 120 of WO 08/020079.

Polypeptides of the invention that contain at least two Nanobodies, in which at least one Nanobody is directed against a first antigen (i.e. against MC4R,) and at least one Nanobody is directed against a second antigen (i.e. different from MC4R,), will also be referred to as “multispecific” polypeptides of the invention, and the Nanobodies present in such polypeptides will also be referred to herein as being in a “multispecific format”. Thus, for example, a “bispecific” polypeptide of the invention is a polypeptide that comprises at least one Nanobody directed against a first antigen (i.e. MC4R,) and at least one further Nanobody directed against a second antigen (i.e. different from MC4R,), whereas a “trispecific” polypeptide of the invention is a polypeptide that comprises at least one Nanobody directed against a first antigen (i.e. MC4R,), at least one further Nanobody directed against a second antigen (i.e. different from MC4R,) and at least one further Nanobody directed against a third antigen (i.e. different from both MC4R, and the second antigen); etc.

Accordingly, in its simplest form, a bispecific polypeptide of the invention is a bivalent polypeptide of the invention (as defined herein), comprising a first Nanobody directed against MC4R, and a second Nanobody directed against a second antigen, in which said first and second Nanobody may optionally be linked via a linker sequence (as defined herein); whereas a trispecific polypeptide of the invention in its simplest form is a trivalent polypeptide of the invention (as defined herein), comprising a first Nanobody directed against MC4R, a second Nanobody directed against a second antigen and a third Nanobody directed against a third antigen, in which said first, second and third Nanobody may optionally be linked via one or more, and in particular one and more, in particular two, linker sequences.

However, as will be clear from the description hereinabove, the invention is not limited thereto, in the sense that a multispecific polypeptide of the invention may comprise at least one Nanobody against MC4R, and any number of Nanobodies directed against one or more antigens different from MC4R.

Furthermore, although it is encompassed within the scope of the invention that the specific order or arrangement of the various Nanobodies in the polypeptides of the invention may have some influence on the properties of the final polypeptide of the invention (including but not limited to the affinity, specificity or avidity for MC4R, or against the one or more other antigens), said order or arrangement is usually not critical and may be suitably chosen by the skilled person, optionally after some limited routine experiments based on the disclosure herein. Thus, when reference is made to a specific multivalent or multispecific polypeptide of the invention, it should be noted that this encompasses any order or arrangements of the relevant Nanobodies, unless explicitly indicated otherwise.

Finally, it is also within the scope of the invention that the polypeptides of the invention contain two or more Nanobodies and one or more further amino acid sequences (as mentioned herein).

For multivalent and multispecific polypeptides containing one or more V_{HH} domains and their preparation, reference is also made to Conrath et al., J. Biol. Chem., Vol. 276, 10. 7346-7350, 2001; Muyldermans, Reviews in Molecular Biotechnology 74 (2001), 277-302; as well as to for example WO 96/34103 and WO 99/23221. Some other examples of some specific multispecific and/or multivalent polypeptide of the invention can be found in the applications by Ablynx N.V. referred to herein.

One preferred, but non-limiting example of a multispecific polypeptide of the invention comprises at least one Nanobody of the invention and at least one Nanobody that provides for an increased half-life. Such Nanobodies may for example be Nanobodies that are directed against a serum protein, and in particular a human serum protein, such as human serum albumin, thyroxine-binding protein, (human) transferrin, fibrinogen, an immunoglobulin such as IgG, IgE or IgM, or against one of the serum proteins listed in WO 04/003019. Of these, Nanobodies that can bind to serum albumin (and in particular human serum albumin) or to IgG (and in particular human IgG, see for example Nanobody VH-1 described in the review by Muyldermans, supra) are particularly preferred (although for example, for experiments in mice or primates, Nanobodies against or cross-reactive with mouse serum albumin (MSA) or serum albumin from said primate, respectively, can be used. However, for pharmaceutical use, Nanobodies against human serum albumin or human IgG will usually be preferred). Nanobodies that provide for increased half-life and that can be used in the polypeptides of the invention include the Nanobodies directed against serum albumin that are described in WO 04/041865, in WO 06/122787, in WO/2017/080850, in WO/2017/085172 and in the further patent applications by Ablynx N.V., such as those mentioned above.

For example, the some preferred Nanobodies that provide for increased half-life for use in the present invention include Nanobodies that can bind to amino acid residues on (human) serum albumin that are not involved in binding of serum albumin to FcRn (see for example WO 06/0122787); Nanobodies that are capable of binding to amino acid residues on serum albumin that do not form part of domain III of serum albumin (see for example WO 06/0122787); Nanobodies that have or can provide an increased half-life (see for example the U.S. provisional application 60/843,349 by Ablynx N.V mentioned herein; see also PCT/EP2007/059475); Nanobodies against human serum albumin that are cross-reactive with serum albumin from at least one species of mammal, and in particular with at least one species of primate (such as, without limitation, monkeys from the genus Macaca (such as, and in particular, cynomologus monkeys (Macaca fascicularis) and/or rhesus monkeys (Macaca mulatta)) and baboon (Papio ursinus)) (see for example the U.S. provisional application 60/843,349 by Ablynx N.V; see also PCT/EP2007/059475)); Nanobodies that can bind to serum albumin in a pH independent manner (see for example the U.S. provisional application 60/850,774 by Ablynx N.V. mentioned herein) and/or Nanobodies that are conditional binders (see for example the U.S. provisional application 60/850,775 by Ablynx N.V.; see also PCT/EP2007/060850).

Some particularly preferred Nanobodies that provide for increased half-life and that can be used in the polypeptides of the invention include the Nanobodies ALB-1 to ALB-10 disclosed in WO 06/122787 (see Tables II and III) of which ALB-8 (SEQ ID NO: 62 in WO 06/122787) is particularly preferred.

According to a specific, but non-limiting aspect of the invention, the polypeptides of the invention contain, besides the one or more Nanobodies of the invention, at least one Nanobody against human serum albumin.

Generally, any polypeptides of the invention with increased half-life that contain one or more Nanobodies of the invention, and any derivatives of Nanobodies of the invention or of such polypeptides that have an increased half-life, preferably have a half-life that is at least 1.5 times, preferably at least 2 times, such as at least 5 times, for example at least 10 times or more than 20 times, greater than the half-life of the corresponding Nanobody of the invention per se. For example, such a derivative or polypeptides with increased half-life may have a half-life that is increased with more than 1 hours, preferably more than 2 hours, more preferably more than 6 hours, such as more than 12 hours, or even more than 24, 48 or 72 hours, compared to the corresponding Nanobody of the invention per se.

In a preferred, but non-limiting aspect of the invention, such derivatives or polypeptides may exhibit a serum half-life in human of at least about 12 hours, preferably at least 24 hours, more preferably at least 48 hours, even more preferably at least 72 hours or more. For example, such derivatives or polypeptides may have a half-life of at least 5 days (such as about 5 to 10 days), preferably at least 9 days (such as about 9 to 14 days), more preferably at least about 10 days (such as about 10 to 15 days), or at least about 11 days (such as about 11 to 16 days), more preferably at least about 12 days (such as about 12 to 18 days or more), or more than 14 days (such as about 14 to 19 days).

According to one aspect of the invention the polypeptides are capable of binding to one or more molecules which can increase the half-life of the polypeptide in vivo.

The polypeptides of the invention are stabilised in vivo and their half-life increased by binding to molecules which resist degradation and/or clearance or sequestration. Typically, such molecules are naturally occurring proteins which themselves have a long half-life in vivo.

Another preferred, but non-limiting example of a multispecific polypeptide of the invention comprises at least one Nanobody of the invention and at least one Nanobody that directs the polypeptide of the invention towards, and/or that allows the polypeptide of the invention to penetrate or to enter into specific organs, tissues, cells, or parts or compartments of cells, and/or that allows the Nanobody to penetrate or cross a biological barrier such as a cell membrane, a cell layer such as a layer of epithelial cells, a tumor including solid tumors, or the blood-brain-barrier. Examples of such Nanobodies include Nanobodies that are directed towards specific cell-surface proteins, markers or epitopes of the desired organ, tissue or cell (for example cell-surface markers associated with tumor cells), and the single-domain brain targeting antibody fragments described in WO 02/057445 and WO 06/040153, of which FC44 (SEQ ID NO: 189 of WO 06/040153) and FC5 (SEQ ID NO: 190 of WO 06/040154) are preferred examples.

In the polypeptides of the invention, the one or more Nanobodies and the one or more polypeptides may be directly linked to each other (as for example described in WO 99/23221) and/or may be linked to each other via one or more suitable spacers or linkers, or any combination thereof.

Suitable spacers or linkers for use in multivalent and multispecific polypeptides will be clear to the skilled person, and may generally be any linker or spacer used in the art to link amino acid sequences. Preferably, said linker or spacer is suitable for use in constructing proteins or polypeptides that are intended for pharmaceutical use.

Some particularly preferred spacers include the spacers and linkers that are used in the art to link antibody fragments or antibody domains. These include the linkers mentioned in the general background art cited above, as well as for example linkers that are used in the art to construct diabodies or ScFv fragments (in this respect, however, its should be noted that, whereas in diabodies and in ScFv fragments, the linker sequence used should have a length, a degree of flexibility and other properties that allow the pertinent V_{H} and V_{L} domains to come together to form the complete antigen-binding site, there is no particular limitation on the length or the flexibility of the linker used in the polypeptide of the invention, since each Nanobody by itself forms a complete antigen-binding site).

For example, a linker may be a suitable amino acid sequence, and in particular amino acid sequences of between 1 and 50, preferably between 1 and 30, such as between 1 and 10 amino acid residues. Some preferred examples of such amino acid sequences include gly-ser linkers, for example of the type (glyₓser_{y})_{z}, such as (for example (gly₄ser)₃ or (gly₃ser₂)₃, as described in WO 99/42077 and the GS30, GS15, GS9 and GS7 linkers described in the applications by Ablynx mentioned herein (see for example WO 06/040153 and WO 06/122825), as well as hinge-like regions, such as the hinge regions of naturally occurring heavy chain antibodies or similar sequences (such as described in WO 94/04678).

Some other particularly preferred linkers are poly-alanine (such as AAA), as well as the linkers GS30 (SEQ ID NO: 85 in WO 06/122825) and GS9 (SEQ ID NO: 84 in WO 06/122825).

Other suitable linkers generally comprise organic compounds or polymers, in particular those suitable for use in proteins for pharmaceutical use. For instance, poly(ethyleneglycol) moieties have been used to link antibody domains, see for example WO 04/081026.

It is encompassed within the scope of the invention that the length, the degree of flexibility and/or other properties of the linker(s) used (although not critical, as it usually is for linkers used in ScFv fragments) may have some influence on the properties of the final polypeptide of the invention, including but not limited to the affinity, specificity or avidity for MC4R, or for one or more of the other antigens. Based on the disclosure herein, the skilled person will be able to determine the optimal linker(s) for use in a specific polypeptide of the invention, optionally after some limited routine experiments.

For example, in multivalent polypeptides of the invention that comprise Nanobodies directed against a multimeric antigen (such as a multimeric receptor or other protein), the length and flexibility of the linker are preferably such that it allows each Nanobody of the invention present in the polypeptide to bind to the antigenic determinant on each of the subunits of the multimer. Similarly, in a multispecific polypeptide of the invention that comprises Nanobodies directed against two or more different antigenic determinants on the same antigen (for example against different epitopes of an antigen and/or against different subunits of a multimeric receptor, channel or protein), the length and flexibility of the linker are preferably such that it allows each Nanobody to bind to its intended antigenic determinant. Again, based on the disclosure herein, the skilled person will be able to determine the optimal linker(s) for use in a specific polypeptide of the invention, optionally after some limited routine experiments.

It is also within the scope of the invention that the linker(s) used confer one or more other favourable properties or functionality to the polypeptides of the invention, and/or provide one or more sites for the formation of derivatives and/or for the attachment of functional groups (e.g. as described herein for the derivatives of the Nanobodies of the invention). For example, linkers containing one or more charged amino acid residues (see Table A-2 above) can provide improved hydrophilic properties, whereas linkers that form or contain small epitopes or tags can be used for the purposes of detection, identification and/or purification. Again, based on the disclosure herein, the skilled person will be able to determine the optimal linkers for use in a specific polypeptide of the invention, optionally after some limited routine experiments.

Finally, when two or more linkers are used in the polypeptides of the invention, these linkers may be the same or different. Again, based on the disclosure herein, the skilled person will be able to determine the optimal linkers for use in a specific polypeptide of the invention, optionally after some limited routine experiments.

Usually, for easy of expression and production, a polypeptide of the invention will be a linear polypeptide. However, the invention in its broadest sense is not limited thereto. For example, when a polypeptide of the invention comprises three of more Nanobodies, it is possible to link them by use of a linker with three or more “arms”, which each “arm” being linked to a Nanobody, so as to provide a “star-shaped” construct. It is also possible, although usually less preferred, to use circular constructs.

The invention also comprises derivatives of the polypeptides of the invention, which may be essentially analogous to the derivatives of the Nanobodies of the invention, i.e. as described herein.

The invention also comprises proteins or polypeptides that “essentially consist” of a polypeptide of the invention (in which the wording “essentially consist of” has essentially the same meaning as indicated hereinabove).

According to one aspect of the invention, the polypeptide of the invention is in essentially isolated from, as defined herein.

The amino acid sequences, Nanobodies, polypeptides and nucleic acids of the invention can be prepared in a manner known per se, as will be clear to the skilled person from the further description herein. For example, the Nanobodies and polypeptides of the invention can be prepared in any manner known per se for the preparation of antibodies and in particular for the preparation of antibody fragments (including but not limited to (single) domain antibodies and ScFv fragments). Some preferred, but non-limiting methods for preparing the amino acid sequences, Nanobodies, polypeptides and nucleic acids include the methods and techniques described herein.

As will be clear to the skilled person, one particularly useful method for preparing an amino acid sequence, Nanobody and/or a polypeptide of the invention generally comprises the steps of:
i) the expression, in a suitable host cell or host organism (also referred to herein as a “host of the invention”) or in another suitable expression system of a nucleic acid that encodes said amino acid sequence, Nanobody or polypeptide of the invention (also referred to herein as a “nucleic acid of the invention”), optionally followed by:
ii) isolating and/or purifying the amino acid sequence, Nanobody or polypeptide of the invention thus obtained.

In particular, such a method may comprise the steps of:
i) cultivating and/or maintaining a host of the invention under conditions that are such that said host of the invention expresses and/or produces at least one amino acid sequence, Nanobody and/or polypeptide of the invention; optionally followed by:
ii) isolating and/or purifying the amino acid sequence, Nanobody or polypeptide of the invention thus obtained.

A nucleic acid of the invention can be in the form of single or double stranded DNA or RNA, and is preferably in the form of double stranded DNA. For example, the nucleotide sequences of the invention may be genomic DNA, cDNA or synthetic DNA (such as DNA with a codon usage that has been specifically adapted for expression in the intended host cell or host organism).

According to one aspect of the invention, the nucleic acid of the invention is in essentially isolated from, as defined herein.

The nucleic acid of the invention may also be in the form of, be present in and/or be part of a vector, such as for example a plasmid, cosmid or YAC, which again may be in essentially isolated form.

The nucleic acids of the invention can be prepared or obtained in a manner known per se, based on the information on the amino acid sequences for the polypeptides of the invention given herein, and/or can be isolated from a suitable natural source. To provide analogs, nucleotide sequences encoding naturally occurring V_{HH} domains can for example be subjected to site-directed mutagenesis, so at to provide a nucleic acid of the invention encoding said analog. Also, as will be clear to the skilled person, to prepare a nucleic acid of the invention, also several nucleotide sequences, such as at least one nucleotide sequence encoding a Nanobody and for example nucleic acids encoding one or more linkers can be linked together in a suitable manner.

Techniques for generating the nucleic acids of the invention will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers, using for example a sequence of a naturally occurring form of MC4R as a template. These and other techniques will be clear to the skilled person, and reference is again made to the standard handbooks, such as Sambrook et al. and Ausubel et al., mentioned above, as well as the Examples below.

The nucleic acid of the invention may also be in the form of, be present in and/or be part of a genetic construct, as will be clear to the person skilled in the art and as described on pages 131-134 of WO 08/020079 (incorporated herein by reference). Such genetic constructs generally comprise at least one nucleic acid of the invention that is optionally linked to one or more elements of genetic constructs known per se, such as for example one or more suitable regulatory elements (such as a suitable promoter(s), enhancer(s), terminator(s), etc.) and the further elements of genetic constructs referred to herein. Such genetic constructs comprising at least one nucleic acid of the invention will also be referred to herein as “genetic constructs of the invention”.

The genetic constructs of the invention may be DNA or RNA, and are preferably double-stranded DNA. The genetic constructs of the invention may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system).

In a preferred but non-limiting aspect, a genetic construct of the invention comprises
at least one nucleic acid of the invention; operably connected to
one or more regulatory elements, such as a promoter and optionally a suitable terminator;
   and optionally also
one or more further elements of genetic constructs known per se;
in which the terms “operably connected” and “operably linked” have the meaning given on pages 131-134 of WO 08/020079; and in which the “regulatory elements”, “promoter”, “terminator” and “further elements” are as described on pages 131-134 of WO 08/020079; and in which the genetic constructs may further be as described on pages 131-134 of WO 08/020079.

The nucleic acids of the invention and/or the genetic constructs of the invention may be used to transform a host cell or host organism, i.e. for expression and/or production of the amino acid sequence, Nanobody or polypeptide of the invention. Suitable hosts or host cells will be clear to the skilled person, and may for example be any suitable fungal, prokaryotic or eukaryotic cell or cell line or any suitable fungal, prokaryotic or eukaryotic organism, for example those described on pages 134 and 135 of WO 08/020079; as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al., (1998), supra; Riechmann and Muyldermans, (1999), supra; van der Linden, (2000), supra; Thomassen et al., (2002), supra; Joosten et al., (2003), supra; Joosten et al., (2005), supra; and the further references cited herein.

The amino acid sequences, Nanobodies and polypeptides of the invention can also be introduced and expressed in one or more cells, tissues or organs of a multicellular organism, for example for prophylactic and/or therapeutic purposes (e.g. as a gene therapy), as further described on pages 135 and 136 of in WO 08/020079 and in the further references cited in WO 08/020079.

For expression of the Nanobodies in a cell, they may also be expressed as so-called “intrabodies”, as for example described in WO 94/02610, WO 95/22618 and U.S. Pat. No. 7,004,940; WO 03/014960; in Cattaneo, A. & Biocca, S. (1997) Intracellular Antibodies: Development and Applications. Landes and Springer-Verlag; and in Kontermann, Methods 34, (2004), 163-170.

The amino acid sequences, Nanobodies and polypeptides of the invention can for example also be produced in the milk of transgenic mammals, for example in the milk of rabbits, cows, goats or sheep (see for example U.S. Pat. Nos. 6,741,957, 6,304,489 and 6,849,992 for general techniques for introducing transgenes into mammals), in plants or parts of plants including but not limited to their leaves, flowers, fruits, seed, roots or turbers (for example in tobacco, maize, soybean or alfalfa) or in for example pupae of the silkworm Bombix mori.

Furthermore, the amino acid sequences, Nanobodies and polypeptides of the invention can also be expressed and/or produced in cell-free expression systems, and suitable examples of such systems will be clear to the skilled person. Some preferred, but non-limiting examples include expression in the wheat germ system; in rabbit reticulocyte lysates; or in the E. coli Zubay system.

As mentioned above, one of the advantages of the use of Nanobodies is that the polypeptides based thereon can be prepared through expression in a suitable bacterial system, and suitable bacterial expression systems, vectors, host cells, regulatory elements, etc., will be clear to the skilled person, for example from the references cited above. It should however be noted that the invention in its broadest sense is not limited to expression in bacterial systems.

Preferably, in the invention, an (in vivo or in vitro) expression system, such as a bacterial expression system, is used that provides the polypeptides of the invention in a form that is suitable for pharmaceutical use, and such expression systems will again be clear to the skilled person. As also will be clear to the skilled person, polypeptides of the invention suitable for pharmaceutical use can be prepared using techniques for peptide synthesis.

For production on industrial scale, preferred heterologous hosts for the (industrial) production of Nanobodies or Nanobody-containing protein therapeutics include strains of E. coli, Pichia pastoris, S. cerevisiae that are suitable for large scale expression/production/fermentation, and in particular for large scale pharmaceutical (i.e. GMP grade) expression/production/fermentation. Suitable examples of such strains will be clear to the skilled person. Such strains and production/expression systems are also made available by companies such as Biovitrum (Uppsala, Sweden).

Alternatively, mammalian cell lines, in particular Chinese hamster ovary (CHO) cells, can be used for large scale expression/production/fermentation, and in particular for large scale pharmaceutical expression/production/fermentation. Again, such expression/production systems are also made available by some of the companies mentioned above.

The choice of the specific expression system would depend in part on the requirement for certain post-translational modifications, more specifically glycosylation. The production of a Nanobody-containing recombinant protein for which glycosylation is desired or required would necessitate the use of mammalian expression hosts that have the ability to glycosylate the expressed protein. In this respect, it will be clear to the skilled person that the glycosylation pattern obtained (i.e. the kind, number and position of residues attached) will depend on the cell or cell line that is used for the expression. Preferably, either a human cell or cell line is used (i.e. leading to a protein that essentially has a human glycosylation pattern) or another mammalian cell line is used that can provide a glycosylation pattern that is essentially and/or functionally the same as human glycosylation or at least mimics human glycosylation. Generally, prokaryotic hosts such as E. coli do not have the ability to glycosylate proteins, and the use of lower eukaryotes such as yeast usually leads to a glycosylation pattern that differs from human glycosylation. Nevertheless, it should be understood that all the foregoing host cells and expression systems can be used in the invention, depending on the desired amino acid sequence, Nanobody or polypeptide to be obtained.

Thus, according to one non-limiting aspect of the invention, the amino acid sequence, Nanobody or polypeptide of the invention is glycosylated. According to another non-limiting aspect of the invention, the amino acid sequence, Nanobody or polypeptide of the invention is non-glycosylated.

According to one preferred, but non-limiting aspect of the invention, the amino acid sequence, Nanobody or polypeptide of the invention is produced in a bacterial cell, in particular a bacterial cell suitable for large scale pharmaceutical production, such as cells of the strains mentioned above.

According to another preferred, but non-limiting aspect of the invention, the amino acid sequence, Nanobody or polypeptide of the invention is produced in a yeast cell, in particular a yeast cell suitable for large scale pharmaceutical production, such as cells of the species mentioned above.

According to yet another preferred, but non-limiting aspect of the invention, the amino acid sequence, Nanobody or polypeptide of the invention is produced in a mammalian cell, in particular in a human cell or in a cell of a human cell line, and more in particular in a human cell or in a cell of a human cell line that is suitable for large scale pharmaceutical production, such as the cell lines mentioned hereinabove.

As further described on pages 138 and 139 of WO 08/020079, when expression in a host cell is used to produce the amino acid sequences, Nanobodies and the polypeptides of the invention, the amino acid sequences, Nanobodies and polypeptides of the invention can be produced either intracellullarly (e.g. in the cytosol, in the periplasma or in inclusion bodies) and then isolated from the host cells and optionally further purified; or can be produced extracellularly (e.g. in the medium in which the host cells are cultured) and then isolated from the culture medium and optionally further purified. Thus, according to one non-limiting aspect of the invention, the amino acid sequence, Nanobody or polypeptide of the invention is an amino acid sequence, Nanobody or polypeptide that has been produced intracellularly and that has been isolated from the host cell, and in particular from a bacterial cell or from an inclusion body in a bacterial cell. According to another non-limiting aspect of the invention, the amino acid sequence, Nanobody or polypeptide of the invention is an amino acid sequence, Nanobody or polypeptide that has been produced extracellularly, and that has been isolated from the medium in which the host cell is cultivated.

Some preferred, but non-limiting promoters for use with these host cells include those mentioned on pages 139 and 140 of WO 08/020079.

Some preferred, but non-limiting secretory sequences for use with these host cells include those mentioned on page 140 of WO 08/020079.

Suitable techniques for transforming a host or host cell of the invention will be clear to the skilled person and may depend on the intended host cell/host organism and the genetic construct to be used. Reference is again made to the handbooks and patent applications mentioned above.

After transformation, a step for detecting and selecting those host cells or host organisms that have been successfully transformed with the nucleotide sequence/genetic construct of the invention may be performed. This may for instance be a selection step based on a selectable marker present in the genetic construct of the invention or a step involving the detection of the amino acid sequence of the invention, e.g. using specific antibodies.

The transformed host cell (which may be in the form or a stable cell line) or host organisms (which may be in the form of a stable mutant line or strain) form further aspects of the present invention.

Preferably, these host cells or host organisms are such that they express, or are (at least) capable of expressing (e.g. under suitable conditions), an amino acid sequence, Nanobody or polypeptide of the invention (and in case of a host organism: in at least one cell, part, tissue or organ thereof). The invention also includes further generations, progeny and/or offspring of the host cell or host organism of the invention, that may for instance be obtained by cell division or by sexual or asexual reproduction.

To produce/obtain expression of the amino acid sequences of the invention, the transformed host cell or transformed host organism may generally be kept, maintained and/or cultured under conditions such that the (desired) amino acid sequence, Nanobody or polypeptide of the invention is expressed/produced. Suitable conditions will be clear to the skilled person and will usually depend upon the host cell/host organism used, as well as on the regulatory elements that control the expression of the (relevant) nucleotide sequence of the invention. Again, reference is made to the handbooks and patent applications mentioned above in the paragraphs on the genetic constructs of the invention.

Generally, suitable conditions may include the use of a suitable medium, the presence of a suitable source of food and/or suitable nutrients, the use of a suitable temperature, and optionally the presence of a suitable inducing factor or compound (e.g. when the nucleotide sequences of the invention are under the control of an inducible promoter); all of which may be selected by the skilled person. Again, under such conditions, the amino acid sequences of the invention may be expressed in a constitutive manner, in a transient manner, or only when suitably induced.

It will also be clear to the skilled person that the amino acid sequence, Nanobody or polypeptide of the invention may (first) be generated in an immature form (as mentioned above), which may then be subjected to post-translational modification, depending on the host cell/host organism used. Also, the amino acid sequence, Nanobody or polypeptide of the invention may be glycosylated, again depending on the host cell/host organism used.

The amino acid sequence, Nanobody or polypeptide of the invention may then be isolated from the host cell/host organism and/or from the medium in which said host cell or host organism was cultivated, using protein isolation and/or purification techniques known per se, such as (preparative) chromatography and/or electrophoresis techniques, differential precipitation techniques, affinity techniques (e.g. using a specific, cleavable amino acid sequence fused with the amino acid sequence, Nanobody or polypeptide of the invention) and/or preparative immunological techniques (i.e. using antibodies against the amino acid sequence to be isolated).

Generally, for pharmaceutical use, the polypeptides of the invention may be formulated as a pharmaceutical preparation or compositions comprising at least one polypeptide of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described herein.

Thus, in a further aspect, the invention relates to a pharmaceutical composition that contains at least one amino acid of the invention, at least one Nanobody of the invention or at least one polypeptide of the invention and at least one suitable carrier, diluent or excipient (i.e. suitable for pharmaceutical use), and optionally one or more further active substances.

Generally, the amino acid sequences, Nanobodies and polypeptides of the invention can be formulated and administered in any suitable manner known per se, for which reference is for example made to the general background art cited above (and in particular to WO 04/041862, WO 04/041863, WO 04/041865, WO 04/041867 and WO 08/020079) as well as to the standard handbooks, such as Remington's Pharmaceutical Sciences, 18^{th} Ed., Mack Publishing Company, USA (1990), Remington, the Science and Practice of Pharmacy, 21th Edition, Lippincott Williams and Wilkins (2005); or the Handbook of Therapeutic Antibodies (S. Dubel, Ed.), Wiley, Weinheim, 2007 (see for example pages 252-255).

For example, the amino acid sequences, Nanobodies and polypeptides of the invention may be formulated and administered in any manner known per se for conventional antibodies and antibody fragments (including ScFv's and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e. transdermal or intradermal) administration.

Preparations for parenteral administration may for example be sterile solutions, suspensions, dispersions or emulsions that are suitable for infusion or injection. Suitable carriers or diluents for such preparations for example include, without limitation, those mentioned on page 143 of WO 08/020079. Usually, aqueous solutions or suspensions will be preferred.

The amino acid sequences, Nanobodies and polypeptides of the invention can also be administered using gene therapy methods of delivery. Sec, e.g., U.S. Pat. No. 5,399,346, which is incorporated by reference in its entirety. Using a gene therapy method of delivery, primary cells transfected with the gene encoding an amino acid sequence, Nanobody or polypeptide of the invention can additionally be transfected with tissue specific promoters to target specific organs, tissue, grafts, tumors, or cells and can additionally be transfected with signal and stabilization sequences for subcellularly localized expression.

Thus, the amino acid sequences, Nanobodies and polypeptides of the invention may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the amino acid sequences, Nanobodies and polypeptides of the invention may be combined with one or more excipients and used in the form of ingestible tablets, buccal tablets, troches, capsules, elixirs, suspensions, syrups, wafers, and the like. Such compositions and preparations should contain at least 0.1% of the amino acid sequence, Nanobody or polypeptide of the invention. Their percentage in the compositions and preparations may, of course, be varied and May conveniently be between about 2 to about 60% of the weight of a given unit dosage form. The amount of the amino acid sequence, Nanobody or polypeptide of the invention in such therapeutically useful compositions is such that an effective dosage level will be obtained.

The tablets, troches, pills, capsules, and the like may also contain binders, excipients, disintegrating agents, lubricants and sweetening or flavouring agents, for example those mentioned on pages 143-144 of WO 08/020079. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the amino acid sequences, Nanobodies and polypeptides of the invention, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the amino acid sequences, Nanobodies and polypeptides of the invention may be incorporated into sustained-release preparations and devices.

Preparations and formulations for oral administration may also be provided with an enteric coating that will allow the constructs of the invention to resist the gastric environment and pass into the intestines. More generally, preparations and formulations for oral administration may be suitably formulated for delivery into any desired part of the gastrointestinal tract. In addition, suitable suppositories may be used for delivery into the gastrointestinal tract.

The amino acid sequences, Nanobodies and polypeptides of the invention may also be administered intravenously or intraperitoneally by infusion or injection, as further described on pages 144 and 145 of WO 08/020079.

For topical administration, the amino acid sequences, Nanobodies and polypeptides of the invention may be applied in pure form, i.e., when they are liquids. However, it will generally be desirable to administer them to the skin as compositions or formulations, in combination with a dermatologically acceptable carrier, which may be a solid or a liquid, as further described on page 145 of WO 08/020079.

Generally, the concentration of the amino acid sequences, Nanobodies and polypeptides of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the amino acid sequences, Nanobodies and polypeptides of the invention required for use in treatment will vary not only with the particular amino acid sequence, Nanobody or polypeptide selected but also with the route of administration, the nature of the condition being treated and the age and condition of the patient and will be ultimately at the discretion of the attendant physician or clinician. Also the dosage of the amino acid sequences, Nanobodies and polypeptides of the invention varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen could include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, PA. The dosage can also be adjusted by the individual physician in the event of any complication.

In another aspect, the invention relates to a method for the prevention and/or treatment of at least one MC4R-related diseases and disorders, said method comprising administering, to a subject in need thereof, a pharmaceutically active amount of an amino acid sequence of the invention, of a Nanobody of the invention, of a polypeptide of the invention, and/or of a pharmaceutical composition comprising the same.

In the context of the present invention, the term “prevention and/or treatment” not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any warm-blooded animal, but is in particular a mammal, and more in particular a human being. As will be clear to the skilled person, the subject to be treated will in particular be a person suffering from, or at risk of, the diseases and disorders mentioned herein.

The invention relates to a method for the prevention and/or treatment of at least one disease or disorder that is associated with MC4R, with its biological or pharmacological activity, and/or with the biological pathways or signalling in which MC4R is involved, said method comprising administering, to a subject in need thereof, a pharmaceutically active amount of an amino acid sequence of the invention, of a Nanobody of the invention, of a polypeptide of the invention, and/or of a pharmaceutical composition comprising the same. In particular, the invention relates to a method for the prevention and/or treatment of at least one disease or disorder that can be treated by modulating MC4R, its biological or pharmacological activity, and/or the biological pathways or signalling in which MC4R is involved, said method comprising administering, to a subject in need thereof, a pharmaceutically active amount of an amino acid sequence of the invention, of a Nanobody of the invention, of a polypeptide of the invention, and/or of a pharmaceutical composition comprising the same. In particular, said pharmaceutically effective amount may be an amount that is sufficient to modulate MC4R, its biological or pharmacological activity, and/or the biological pathways or signalling in which MC4R is involved; and/or an amount that provides a level of the amino acid sequence of the invention, of a Nanobody of the invention, of a polypeptide of the invention in the circulation that is sufficient to modulate MC4R, its biological or pharmacological activity, and/or the biological pathways or signalling in which MC4R is involved.

The invention furthermore relates to a method for the prevention and/or treatment of at least one disease or disorder that can be prevented and/or treated by administering an amino acid sequence of the invention, a Nanobody of the invention or a polypeptide of the invention to a patient, said method comprising administering, to a subject in need thereof, a pharmaceutically active amount of an amino acid sequence of the invention, of a Nanobody of the invention, of a polypeptide of the invention, and/or of a pharmaceutical composition comprising the same.

More in particular, the invention relates to a method for the prevention and/or treatment of at least one disease or disorder chosen from the group consisting of the diseases and disorders listed herein, said method comprising administering, to a subject in need thereof, a pharmaceutically active amount of an amino acid sequence of the invention, of a Nanobody of the invention, of a polypeptide of the invention, and/or of a pharmaceutical composition comprising the same.

In another aspect, the invention relates to a method for immunotherapy, and in particular for passive immunotherapy, which method comprises administering, to a subject suffering from or at risk of the diseases and disorders mentioned herein, a pharmaceutically active amount of an amino acid sequence of the invention, of a Nanobody of the invention, of a polypeptide of the invention, and/or of a pharmaceutical composition comprising the same.

In the above methods, the amino acid sequences, Nanobodies and/or polypeptides of the invention and/or the compositions comprising the same can be administered in any suitable manner, depending on the specific pharmaceutical formulation or composition to be used. Thus, the amino acid sequences, Nanobodies and/or polypeptides of the invention and/or the compositions comprising the same can for example be administered orally, intraperitoneally (e.g. intravenously, subcutaneously, intramuscularly, or via any other route of administration that circumvents the gastrointestinal tract), intranasally, transdermally, topically, by means of a suppository, by inhalation, again depending on the specific pharmaceutical formulation or composition to be used. The clinician will be able to select a suitable route of administration and a suitable pharmaceutical formulation or composition to be used in such administration, depending on the disease or disorder to be prevented or treated and other factors well known to the clinician.

The amino acid sequences, Nanobodies and/or polypeptides of the invention and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific amino acid sequence, Nanobody or polypeptide of the invention to be used, the specific route of administration and pharmaceutical formulation or composition to be used, the age, gender, weight, dict, general condition of the patient, and similar factors well known to the clinician.

Generally, the treatment regimen will comprise the administration of one or more amino acid sequences, Nanobodies and/or polypeptides of the invention, or of one or more compositions comprising the same, in one or more pharmaceutically effective amounts or doses. The specific amount(s) or doses to administered can be determined by the clinician, again based on the factors cited above.

Generally, for the prevention and/or treatment of the diseases and disorders mentioned herein and depending on the specific disease or disorder to be treated, the potency of the specific amino acid sequence, Nanobody and polypeptide of the invention to be used, the specific route of administration and the specific pharmaceutical formulation or composition used, the amino acid sequences, Nanobodies and polypeptides of the invention will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

Usually, in the above method, a single amino acid sequence, Nanobody or polypeptide of the invention will be used. It is however within the scope of the invention to use two or more amino acid sequences, Nanobodies and/or polypeptides of the invention in combination.

The Nanobodies, amino acid sequences and polypeptides of the invention may also be used in combination with one or more further pharmaceutically active compounds or principles, i.e. as a combined treatment regimen, which may or may not lead to a synergistic effect. Again, the clinician will be able to select such further compounds or principles, as well as a suitable combined treatment regimen, based on the factors cited above and his expert judgement. For example, when the Nanobodies, amino acid sequences and polypeptides of the invention are used for the treatment of obesity (including genetic obesity) and/or any other disease or disorder characterized by and/or associated with impaired MC4R signalling, hyperphagia and/or dysregulated energy homeostasis, such as those mentioned herein), the Nanobodies, amino acid sequences and polypeptides of the invention may be administered and/or used as part of a combination therapy with other active principles used for the treatment of such diseases, such as GLP-1R agonists.

In particular, the amino acid sequences, Nanobodies and polypeptides of the invention may be used in combination with other pharmaceutically active compounds or principles that are or can be used for the prevention and/or treatment of the diseases and disorders cited herein, as a result of which a synergistic effect may or may not be obtained. Examples of such compounds and principles, as well as routes, methods and pharmaceutical formulations or compositions for administering them will be clear to the clinician.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

Also, when two or more active substances or principles are to be used as part of a combined treatment regimen, each of the substances or principles may be administered in the same amount and according to the same regimen as used when the compound or principle is used on its own, and such combined use may or may not lead to a synergistic effect. However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmaceutical or therapeutic effect.

The effectiveness of the treatment regimen used according to the invention may be determined and/or followed in any manner known per se for the disease or disorder involved, as will be clear to the clinician. The clinician will also be able, where appropriate and on a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

In another aspect, the invention relates to the use of an amino acid sequence, Nanobody or polypeptide of the invention in the preparation of a pharmaceutical composition for prevention and/or treatment of at least one MC4R-related diseases and disorders; and/or for use in one or more of the methods of treatment mentioned herein.

The subject to be treated may be any warm-blooded animal, but is in particular a mammal, and more in particular a human being. As will be clear to the skilled person, the subject to be treated will in particular be a person suffering from, or at risk of, the diseases and disorders mentioned herein.

The invention also relates to the use of an amino acid sequence, Nanobody or polypeptide of the invention in the preparation of a pharmaceutical composition for the prevention and/or treatment of at least one disease or disorder that can be prevented and/or treated by administering an amino acid sequence, Nanobody or polypeptide of the invention to a patient.

More in particular, the invention relates to the use of an amino acid sequence, Nanobody or polypeptide of the invention in the preparation of a pharmaceutical composition for the prevention and/or treatment of MC4R-related diseases and disorders, and in particular for the prevention and treatment of one or more of the diseases and disorders listed herein.

Again, in such a pharmaceutical composition, the one or more amino acid sequences, Nanobodies or polypeptides of the invention may also be suitably combined with one or more other active principles, such as those mentioned herein.

Finally, although the use of the Nanobodies of the invention (as defined herein) and of the polypeptides of the invention is much preferred, it will be clear that on the basis of the description herein, the skilled person will also be able to design and/or generate, in an analogous manner, other amino acid sequences and in particular (single) domain antibodies against MC4R, as well as polypeptides comprising such (single) domain antibodies.

For example, it will also be clear to the skilled person that it may be possible to “graft” one or more of the CDR's mentioned above for the Nanobodies of the invention onto such (single) domain antibodies or other protein scaffolds, including but not limited to human scaffolds or non-immunoglobulin scaffolds. Suitable scaffolds and techniques for such CDR grafting will be clear to the skilled person and are well known in the art, see for example those mentioned in WO 08/020079. For example, techniques known per se for grafting mouse or rat CDR's onto human frameworks and scaffolds can be used in an analogous manner to provide chimeric proteins comprising one or more of the CDR's of the Nanobodies of the invention and one or more human framework regions or sequences.

It should also be noted that, when the Nanobodies of the inventions contain one or more other CDR sequences than the preferred CDR sequences mentioned above, these CDR sequences can be obtained in any manner known per se, for example using one or more of the techniques described in WO 08/020079.

Further uses of the amino acid sequences, Nanobodies, polypeptides, nucleic acids, genetic constructs and hosts and host cells of the invention will be clear to the skilled person based on the disclosure herein. For example, and without limitation, the amino acid sequences of the invention can be linked to a suitable carrier or solid support so as to provide a medium than can be used in a manner known per se to purify MC4R from compositions and preparations comprising the same. Derivatives of the amino acid sequences of the invention that comprise a suitable detectable label can also be used as markers to determine (qualitatively or quantitatively) the presence of MC4R in a composition or preparation or as a marker to selectively detect the presence of MC4R on the surface of a cell or tissue (for example, in combination with suitable cell sorting techniques).

In a further aspect, the invention relates to a protein or peptide that comprises or essentially consists of a linear chain of 9, 10, 11, 12 or 13 amino acid residues (and preferably 10, 11 or 12 amino acid residues; and more preferably 11 amino acid residues), in which said linear chain of amino acid residues comprises (i) at least one arginine residue (R) that is capable of interacting with the Glu 100 and/or Asp 126 residues of MC4R (and preferably both said residues); (ii) at least one arginine residue (R) that is capable of interacting with the Ser188 and/or Asp189 residues of MC4R (and preferably both said residues); and preferably also (iii) at least two amino acid residues (which preferably form part of a contiguous stretch of 3 amino acid residues), and more preferably at least three amino acid residues (which preferably form a contiguous stretch of 3 amino acid residues) that are capable of interacting with the Gln43 and/or Gln 269 residues of MC4R (and preferably both said residues).

In a further aspect, the invention relates to a protein or peptide that comprises or essentially consists of a linear chain of amino acid residues, which linear chain of amino acid residues either (i) has the amino acid sequence RTGRIVRPLDY (SEQ ID NO: 168) or (ii) is an analog of the amino acid sequence RTGRIVRPLDY that comprises (i) at least one arginine residue (R) that is capable of interacting with the Glu100 and/or Asp 126 residues of MC4R (and preferably both said residues); (ii) at least one arginine residue (R) that is capable of interacting with the Ser188 and/or Asp189 residues of MC4R (and preferably both said residues); and preferably also (iii) at least two amino acid residues (which preferably form part of a contiguous stretch of 3 amino acid residues), and more preferably at least three amino acid residues (which preferably form a contiguous stretch of 3 amino acid residues) that are capable of interacting with the Gln43 and/or Gln 269 residues of MC4R (and preferably both said residues); which analog preferably comprises less than three, preferably less than two, more preferably only one amino acid difference (as defined herein) with the amino acid sequence RTGRIVRPLDY.

In a further aspect, the invention relates to a protein or peptide that comprises or essentially consists of a linear chain of amino acid residues, which linear chain of amino acid residues either (i) has the amino acid sequence RTGRIVRPLDY (SEQ ID NO: 168) or (ii) is an analog of the amino acid sequence RTGRIVRPLDY that comprises (i) at least one arginine residue (R) that is capable of interacting with the Glu 100 and/or Asp126 residues of MC4R (and preferably both said residues); (ii) at least one arginine residue (R) that is capable of interacting with the Ser188 and/or Asp189 residues of MC4R (and preferably both said residues); and preferably also (iii) at least two amino acid residues (which preferably form part of a contiguous stretch of 3 amino acid residues), and more preferably at least three amino acid residues (which preferably form a contiguous stretch of 3 amino acid residues) that are capable of interacting with the Gln43 and/or Gln 269 residues of MC4R (and preferably both said residues); in which;
the T at position 2 may be T or may be replaced by an amino acid residue chosen from A, G, E, N, Q, S, F and Y;
the G at position 3 may be G or may be replaced by any naturally occurring amino acid residue except V, K, T, A, I, F, M, C or P;
the I at position 5 may be I or may be replaced by V or L;
the V at position 6 may be V or may be replaced by T or I;
the P at position 8 may be P or may be replaced by S, A or L;
the L at position 9 may be L or may be replaced any naturally occurring amino acid residue except R, D, M, C or P;
the D at position 10 may be D or may be replaced any naturally occurring amino acid residue except K, F, M, C or P; and
the Y at position 11 may be Y or may be replaced any naturally occurring amino acid residue any except E, D, G, M, C or P;
   in which the amino acid residues in the amino acid sequence RTGRIVRPLDY (SEQ ID NO: 168) are numbered from 1 to 11 with the first arginine (R, start of the sequence, left hand side) is numbered as 1 and the (last) tyrosine (Y, end of the sequence, right hand side) is numbered as 11.

In a further aspect, the invention relates to a protein or peptide that comprises or essentially consists of a linear chain of amino acid residues, which linear chain of amino acid residues either (i) has the amino acid sequence RTGRIVRPLDY (SEQ ID NO: 168) or (ii) is an analog of the amino acid sequence RTGRIVRPLDY that comprises (i) at least one arginine residue (R) that is capable of interacting with the Glu 100 and/or Asp126 residues of MC4R (and preferably both said residues); (ii) at least one arginine residue (R) that is capable of interacting with the Ser188 and/or Asp189 residues of MC4R (and preferably both said residues); and preferably also (iii) at least two amino acid residues (which preferably form part of a contiguous stretch of 3 amino acid residues), and more preferably at least three amino acid residues (which preferably form a contiguous stretch of 3 amino acid residues) that are capable of interacting with the Gln43 and/or Gln 269 residues of MC4R (and preferably both said residues); in which;
the T at position 2 may be T or may be replaced by an amino acid residue chosen from Q, S, F and Y;
1 the G at position 3 may be G or may be replaced by an amino acid residue chosen from H, N, Q, S, W;
the I at position 5 may be I or may be replaced by V or L;
the V at position 6 may be V or may be replaced by T or I;
the P at position 8 may be P or may be replaced by S, A or L;
the L at position 9 may be L or may be replaced any naturally occurring amino acid residue except E, G, R, D, M, C or P;
the D at position 10 may be D or may be replaced any naturally occurring amino acid residue chosen from H, E, N, Q, S, T, A, V, L, I, Y, W and G; and
the Y at position 11 may be Y or may be replaced any naturally occurring amino acid residue any except K, Q, A, D, G, E, M, C or P;
   in which the amino acid residues in the amino acid sequence RTGRIVRPLDY (SEQ ID NO: 168) are numbered from 1 to 11 with the first arginine (R, start of the sequence, left hand side) is numbered as 1 and the (last) tyrosine (Y, end of the sequence, right hand side) is numbered as 11.

It is envisaged that (poly) peptides and proteins that comprises or essentially consist of an amino acid sequence as described herein (i.e. an amino acid sequence comprising at least two arginine residues interacting with MC4R as described herein, and in particular the amino acid sequence RTGRIVRPLDY (SEQ ID NO: 168) or an analog thereof as described herein) can be used as agonists of MC4R, of MC4R-mediated signalling, of the biological pathways in which MC4R and/or MC4R-related signalling are involved, and/or more generally to modulate the biological mechanisms, responses and effects associated with such signalling or these pathways, essentially in the same way as described herein for the amino acid sequences and polypeptides of the invention. In particular, it is envisaged that such peptides and proteins that comprise such an amino acid sequence can be used for the prevention and treatment (as defined herein) of MC4R-related diseases and disorders, again essentially as described herein for the amino acid sequences and polypeptides of the invention.

It is also envisaged that that the peptides and proteins that comprise or essentially consist of an amino acid sequence as described herein (i.e. an amino acid sequence comprising at least two arginine residues interacting with MC4R as described herein, and in particular the amino acid sequence RTGRIVRPLDY (SEQ ID NO: 168) or an analog thereof as described herein) can be any form/format that is suitable for their intended use, for example in the form of a linear protein or peptide, in the form of a cyclic peptide (e.g. as known per se for other peptide agonists of MC4R, such as setmelanotide) or as part of a larger protein, polypeptide or construct (in which the amino acid sequence as described herein most preferably forms or forms part of the sequence of the protein, polypeptide or construct that interacts or is intended to interact with MC4R). For example, it is envisaged that such an amino acid can be suitably included in (i.e. made part of) a protein or polypeptide that comprises a protein “scaffold” known per se that is suitable for pharmaceutical use. Reference is for example made to the protein scaffold mentioned herein.

It is also envisaged that, for use in the prevention and treatment of MC4R-related diseases and disorders, such (poly) peptides, proteins and constructs may be formulated and administered/used in a manner known per se (for example a manner as described herein for the amino acid sequences and polypeptides of the invention). Suitable formulations and routes of administration will be clear to the skilled person (for example, based on the disclosure herein), and may depend on the specific form/format that is chosen for the (poly) peptide, protein and construct.

In one particular aspect, it is envisaged that an amino acid sequence as described herein (i.e. an amino acid sequence comprising at least two arginine residues interacting with MC4R as described herein, and in particular the amino acid sequence RTGRIVRPLDY (SEQ ID NO: 168) or an analog thereof as described herein) may be used as a CDR sequence (and in particular, as a CDR3 sequence) that is part of an immunoglobulin variable domain (such as a VH domain or VL domain) and in particular an immunoglobulin single variable domain such as a VHH/Nanobody. Such an immunoglobulin domain may then be suitably used as such (when it is an immunoglobulin single variable domain) or may suitably be part of a protein, polypeptide or construct, such as, for example and without limitation, a full sized antibody or suitable fragment thereof (such as a Fab fragment) in case of a VH or VL domain; or, in the case of an immunoglobulin single variable domain, a suitable protein, polypeptide or construct comprising one or more immunoglobulin single variable domains (such as immunoglobulin single variable domain-based proteins, polypeptides or constructs described herein). Such immunoglobulin variable domains (and in particular immunoglobulin single variable domains) and proteins, polypeptides or constructs comprising the same form further aspects of the invention.

More in particular, when an amino acid sequence as described herein (i.e. an amino acid sequence comprising at least two arginine residues interacting with MC4R as described herein, and in particular the amino acid sequence RTGRIVRPLDY (SEQ ID NO: 168) or an analog thereof as described herein) is part of an immunoglobulin single variable domain (i.e. as one of the CDRs, and in particular as CDR3), such an immunoglobulin single variable domain may suitably contain two other suitable CDRs. Even more in particular, when an amino acid sequence as described herein essentially forms the CDR3 of such an immunoglobulin single variable domain, such an immunoglobulin single variable domain may further suitably comprise a suitable CDR1 sequence and a suitable CDR2 sequence (for example, a CDR1 sequence that is chosen from the CDR1 sequences disclosed herein and a CDR2 sequence that is chosen from the CDR2 sequences disclosed herein). Again, such an immunoglobulin single variable domains as well as proteins, polypeptides or constructs comprising the same (which may essentially be in a format as described herein for the polypeptides and constructs of the invention) form further aspects of the invention.

Thus, in further aspects, the invention relates to:
a Nanobody which can bind (as further defined herein) to MC4R and which contains a CDR3 sequence that is an amino acid sequence comprising 9, 10, 11, 12 or 13 amino acid residues (and preferably 10, 11 or 12 amino acid residues; and more preferably 11 amino acid residues) and comprising at least two arginine residues interacting with MC4R (in the manner described herein);
a Nanobody which can bind (as further defined herein) to MC4R and which contains a CDR3 sequence that is an amino acid sequence comprising 9, 10, 11, 12 or 13 amino acid residues (and preferably 10, 11 or 12 amino acid residues; and more preferably 11 amino acid residues) and comprising (i) at least one arginine residue (R) that is capable of interacting with the Glu100 and/or Asp126 residues of MC4R (and preferably both said residues); (ii) at least one arginine residue (R) that is capable of interacting with the Ser188 and/or Asp189 residues of MC4R (and preferably both said residues); and preferably also (iii) at least two amino acid residues (which preferably form part of a contiguous stretch of 3 amino acid residues), and more preferably at least three amino acid residues (which preferably form a contiguous stretch of 3 amino acid residues) that are capable of interacting with the Gln43 and/or Gln 269 residues of MC4R (and preferably both said residues);
a Nanobody which can bind (as further defined herein) to MC4R and which contains a CDR3 sequence that is the amino acid sequence RTGRIVRPLDY (SEQ ID NO: 168) or an analog thereof (as described herein);
   which Nanobodies contain suitable CDR1 and CDR2 sequences, in which the CDR1 sequence is preferably chosen from:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;
and are more preferably chosen from the amino acid sequences of SEQ ID NO's: 134 to 141 and 210 to 225 (and even more preferably chosen from the amino acid sequences of SEQ ID NO's: 136 to 141); and in which the CDR2 sequence is preferably chosen from:
d) the amino acid sequences of SEQ ID NO's: 150 to 157 and 242 to 257;
e) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
and are more preferably chosen from the amino acid sequences SEQ ID NO's: 150 to 157 and 242 to 257 (and even more preferably chosen from the amino acid sequences of SEQ ID NO's: 152 to 157). In particular, in such a Nanobody, CDR1 may be chosen from the amino acid sequences of SEQ ID NO's: 136 to 141 (and may more in particular be the amino acid sequence of SEQ ID NO: 141) and CDR2 may be chosen from the amino acid sequences SEQ ID NO's: 152 to 157 (and may more in particular be the amino acid sequence of SEQ ID NO: 157). Such Nanobodies are included in the term “Nanobodies of the invention” as used herein in its broadest sense and proteins, polypeptides or constructs that comprising such a Nanobody (i.e. at least one such Nanobody) are also included in the term “polypeptides of the invention” as used herein in its broadest sense.

In a further aspect, the invention relates to a Nanobody which can bind (as further defined herein) to MC4R and which contains a CDR3 sequence that is the amino acid sequence RTGRIVRPLDY (SEQ ID NO: 168) or an analog thereof (as described herein) and which further contains suitable CDR1 and CDR2 sequences, in which the CDR1 sequence is preferably chosen from:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;
and are more preferably chosen from the amino acid sequences of SEQ ID NO's: 134 to 141 and 210 to 225 (and even more preferably chosen from the amino acid sequences of SEQ ID NO's: 136 to 141); and in which the CDR2 sequence is preferably chosen from:
d) the amino acid sequences of SEQ ID NO's: 150 to 157 and 242 to 257;
c) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
and are more preferably chosen from the amino acid sequences SEQ ID NO's: 150 to 157 and 242 to 257 (and even more preferably chosen from the amino acid sequences of SEQ ID NO's: 152 to 157). In particular, in such a Nanobody, CDR1 may be chosen from the amino acid sequences of SEQ ID NO's: 136 to 141 (and may more in particular be the amino acid sequence of SEQ ID NO: 141) and CDR2 may be chosen from the amino acid sequences SEQ ID NO's: 152 to 157 (and may more in particular be the amino acid sequence of SEQ ID NO: 157).

Such Nanobodies are included in the term “Nanobodies of the invention” as used herein in its broadest sense and proteins, polypeptides or constructs that comprising such a Nanobody (i.e. at least one such Nanobody) are also included in the term “polypeptides of the invention” as used herein in its broadest sense.

In a particular aspect, the invention relates to a Nanobody which can bind (as further defined herein) to MC4R and which contains:
a CDR3 sequence that is the amino acid sequence RTGRIVRPLDY (SEQ ID NO: 168) or an analog of the amino acid sequence RTGRIVRPLDY that comprises (i) at least one arginine residue (R) that is capable of interacting with the Glu 100 and/or Asp126 residues of MC4R (and preferably both said residues); (ii) at least one arginine residue (R) that is capable of interacting with the Ser188 and/or Asp189 residues of MC4R (and preferably both said residues); and preferably also (iii) at least two amino acid residues (which preferably form part of a contiguous stretch of 3 amino acid residues), and more preferably at least three amino acid residues (which preferably form a contiguous stretch of 3 amino acid residues) that are capable of interacting with the Gln43 and/or Gln 269 residues of MC4R (and preferably both said residues); in which;
   the T at position 2 may be T or may be replaced by an amino acid residue chosen from A, G, E, N, Q, S, F and Y;
   the G at position 3 may be G or may be replaced by any naturally occurring amino acid residue except V, K, T, A, I, F, M, C or P;
   the I at position 5 may be I or may be replaced by V or L;
   the V at position 6 may be V or may be replaced by T or I;
   the P at position 8 may be P or may be replaced by S, A or L;
   the L at position 9 may be L or may be replaced any naturally occurring amino acid residue except R, D, M, C or P;
   the D at position 10 may be D or may be replaced any naturally occurring amino acid residue except K, F, M, C or P; and
   the Y at position 11 may be Y or may be replaced any naturally occurring amino acid residue any except E, D, G, M, C or P;
   in which the amino acid residues in the amino acid sequence RTGRIVRPLDY (SEQ ID NO: 168) are numbered from 1 to 11 with the first arginine (R, start of the sequence, left hand side) is numbered as 1 and the (last) tyrosine (Y, end of the sequence, right hand side) is numbered as 11.
      and which further contains:
suitable CDR1 and CDR2 sequences, in which the CDR1 sequence is preferably chosen from:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;
and are more preferably chosen from the amino acid sequences of SEQ ID NO's: 134 to 141 and 210 to 225 (and even more preferably chosen from the amino acid sequences of SEQ ID NO's: 136 to 141); and in which the CDR2 sequence is preferably chosen from:
d) the amino acid sequences of SEQ ID NO's: 150 to 157 and 242 to 257;
c) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
and are more preferably chosen from the amino acid sequences SEQ ID NO's: 150 to 157 and 242 to 257 (and even more preferably chosen from the amino acid sequences of SEQ ID NO's: 152 to 157). In particular, in such a Nanobody, CDR1 may be chosen from the amino acid sequences of SEQ ID NO's: 136 to 141 (and may more in particular be the amino acid sequence of SEQ ID NO: 141) and CDR2 may be chosen from the amino acid sequences SEQ ID NO's: 152 to 157 (and may more in particular be the amino acid sequence of SEQ ID NO: 157).

Such Nanobodies are included in the term “Nanobodies of the invention” as used herein in its broadest sense and proteins, polypeptides or constructs that comprising such a Nanobody (i.e. at least one such Nanobody) are also included in the term “polypeptides of the invention” as used herein in its broadest sense.

In a more particular aspect, the invention relates to a Nanobody which can bind (as further defined herein) to MC4R and which contains:
a CDR3 sequence that is the amino acid sequence RTGRIVRPLDY (SEQ ID NO: 168) or an analog of the amino acid sequence RTGRIVRPLDY that comprises (i) at least one arginine residue (R) that is capable of interacting with the Glu 100 and/or Asp126 residues of MC4R (and preferably both said residues); (ii) at least one arginine residue (R) that is capable of interacting with the Ser188 and/or Asp189 residues of MC4R (and preferably both said residues); and preferably also (iii) at least two amino acid residues (which preferably form part of a contiguous stretch of 3 amino acid residues), and more preferably at least three amino acid residues (which preferably form a contiguous stretch of 3 amino acid residues) that are capable of interacting with the Gln43 and/or Gln 269 residues of MC4R (and preferably both said residues); in which;
   the T at position 2 may be T or may be replaced by an amino acid residue chosen from Q, S, F and Y;
   the G at position 3 may be G or may be replaced by an amino acid residue chosen from H, N, Q, S, W;
   1 the I at position 5 may be I or may be replaced by V or L;
   the V at position 6 may be V or may be replaced by T or I;
   the P at position 8 may be P or may be replaced by S, A or L;
   the L at position 9 may be L or may be replaced any naturally occurring amino acid residue except E, G, R, D, M, C or P;
   the D at position 10 may be D or may be replaced any naturally occurring amino acid residue chosen from H, E, N, Q, S, T, A, V, L, I, Y, W and G; and
   the Y at position 11 may be Y or may be replaced any naturally occurring amino acid residue any except K, Q, A, D, G, E, M, C or P;
in which the amino acid residues in the amino acid sequence RTGRIVRPLDY (SEQ ID NO: 168) are numbered from 1 to 11 with the first arginine (R, start of the sequence, left hand side) is numbered as 1 and the (last) tyrosine (Y, end of the sequence, right hand side) is numbered as 11.
   and which further contains:
suitable CDR1 and CDR2 sequences, in which the CDR1 sequence is preferably chosen from:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;
and are more preferably chosen from the amino acid sequences of SEQ ID NO's: 134 to 141 and 210 to 225 (and even more preferably chosen from the amino acid sequences of SEQ ID NO's: 136 to 141); and in which the CDR2 sequence is preferably chosen from:
d) the amino acid sequences of SEQ ID NO's: 150 to 157 and 242 to 257;
e) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
and are more preferably chosen from the amino acid sequences SEQ ID NO's: 150 to 157 and 242 to 257 (and even more preferably chosen from the amino acid sequences of SEQ ID NO's: 152 to 157). In particular, in such a Nanobody, CDR1 may be chosen from the amino acid sequences of SEQ ID NO's: 136 to 141 (and may more in particular be the amino acid sequence of SEQ ID NO: 141) and CDR2 may be chosen from the amino acid sequences SEQ ID NO's: 152 to 157 (and may more in particular be the amino acid sequence of SEQ ID NO: 157).

Again, such Nanobodies are included in the term “Nanobodies of the invention” as used herein in its broadest sense and proteins, polypeptides or constructs that comprising such a Nanobody (i.e. at least one such Nanobody) are also included in the term “polypeptides of the invention” as used herein in its broadest sense.

In a specific aspect, the invention relates to a Nanobody which can bind (as further defined herein) to MC4R and which contains a CDR3 sequence that is the amino acid sequence RTGRIVRPLDY (SEQ ID NO: 168) and which further contains suitable CDR1 and CDR2 sequences, in which the CDR1 sequence is preferably chosen from:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225;
c) amino acid sequences that have 3, 2, or 1 amino acid difference with at least one of the amino acid sequences of SEQ ID NO's: 134 to 141 and 210 to 225;
and are more preferably chosen from the amino acid sequences of SEQ ID NO's: 134 to 141 and 210 to 225 (and even more preferably chosen from the amino acid sequences of SEQ ID NO's: 136 to 141); and in which the CDR2 sequence is preferably chosen from:
d) the amino acid sequences of SEQ ID NO's: 150 to 157 and 242 to 257;
c) amino acid sequences that have at least 80% amino acid identity with at least one of the amino acid sequences of SEQ ID NO's: 150 to 157 and 242 to 257;
f) amino acid sequences that have 3, 2, or 1 amino acid difference with at least one of the amino acid sequences of SEQ ID NO's: 150 to 157 and 242 to 257;
and are more preferably chosen from the amino acid sequences SEQ ID NO's: 150 to 157 and 242 to 257 (and even more preferably chosen from the amino acid sequences of SEQ ID NO's: 152 to 157). In particular, in such a Nanobody, CDR1 may be chosen from the amino acid sequences of SEQ ID NO's: 136 to 141 (and may more in particular be the amino acid sequence of SEQ ID NO: 141) and CDR2 may be chosen from the amino acid sequences SEQ ID NO's: 152 to 157 (and may more in particular be the amino acid sequence of SEQ ID NO: 157).

Again, such Nanobodies are included in the term “Nanobodies of the invention” as used herein in its broadest sense and proteins, polypeptides or constructs that comprising such a Nanobody (i.e. at least one such Nanobody) are also included in the term “polypeptides of the invention” as used herein in its broadest sense.

In a particular aspect, the invention relates to a Nanobody which can bind (as further defined herein) to MC4R which contains a CDR3 sequence that is the amino acid sequence RTGRIVRPLDY (SEQ ID NO: 168) and which contains a CDR1 that is one of the amino acid sequences of SEQ ID NO's: 136 to 141 (and may more in particular be the amino acid sequence of SEQ ID NO: 141) which contains a CDR2 that is one of the amino acid sequences of SEQ ID NO's: 152 to 157 (and may more in particular be the amino acid sequence of SEQ ID NO: 157). Again, such a Nanobody is included in the term “Nanobodies of the invention” as used herein in its broadest sense and proteins, polypeptides or constructs that comprising such a Nanobody (i.e. at least one such Nanobody) are also included in the term “polypeptides of the invention” as used herein in its broadest sense.

### BRIEF DESCRIPTION OF THE FIGURES

The invention will now be further described by means of the following non-limiting examples and figures, in which the Figures show:

FIG. 1 schematically shows the Diet-induced obese (DIO) mouse studies performed in Example 2;

FIG. 2A are representative graphs showing the In vitro signaling potency of agonistic nanobody pN162 to melanocortin receptors. The graphs show dose-dependent human MC4R (graph marked “MC4R”), MC1R (graph marked “MC1R”), MC3R (graph marked “MC3R”) and MC5R (graph marked “MC5R”) induced cAMP signaling (GloSensor) of pN162 and control ligands (endogenous agonist α-MSH and clinical benchmark peptide agonist Setmelanotide). Each data point represents the mean±s.e.m. of two replicates. Experiments were performed minimally twice provides the average EC₅₀s of pN162, α-MSH and setmelanotide;

FIG. 2B shows two graphs demonstrating the selectivity of an agonist VHH of the invention (pN162) and a polypeptide of the invention (an Fc-based construct comprising two copies of pN162) for hMC4R over hMC1R, as experimentally determined using the methodology described in Example 4;

FIG. 2C shows two graphs demonstrating the induction of MC4R signaling pathways by pN162. The graphs show dose-dependent Gs ConfoSensor (left hand panel) or β-arrestin recruitment (right hand panel) for agonists pN162, α-MSH and setmelanotide in the absence (dashed lines) or presence (full lines) of 1 mM Ca²⁺. Data are depicted as the mean±s.e.m. of two replicates. Experiments were performed twice and exemplary graphs are shown.

FIGS. 3A to 3D are graphs showing the results obtained during the diet-induced obese (DIO) mouse studies (in vivo acute setting and ICV administration) as described in Example 2;

FIG. 5 is a graph showing the results of the in vitro pigmentation assays with human skin cells (2D HP-NHEM) performed in Example 5;

FIG. 6 is a graph showing the results of the in vitro pigmentation assays with human skin cells (3D-RHE-MEL) performed in Example 6;

FIGS. 7A and 7B are graphs showing the results of the Evaluation of the cardiovascular safety pharmacology profile of 2 compounds after a single treatment in conscious non-naïve telemetered rats, as performed in Example 7;

FIGS. 8A to 8D show the structure of the complex of an agonist VHH of the invention (pN162) in complex with MC4R, Confobody 35 (see EP 2723764) and a stable G-protein variant. FIG. 8B shows an extracellular (EC) view and FIG. 8B shows and intracellular (IC) view.

FIG. 8E shows the Cryo-EM structure of pN162 bound MC4R-DNGs-Cb35 complex. Left hand side: local resolution cryo-EM density map with fitted model. The local resolution of the cryo-EM density is depicted according to the heat bar ranging between 2.5 and 6.5 Å. Right hand side: final model submitted to the Protein Data Bank (accession code 8QJ2);

FIG. 8F, panels a. to f. show a structural comparison between active and inactive MC4R. panels a. and b.: Side views of the overlay between SHU9119 bound inactive state MC4R and setmelanotide or pN162 bound MC4R active state. Transmembrane domains, (TMs) and helix 8, (H8) are visible. Panel c.: Bottom view of the same overlay showing the hallmark TM6 outward movement and TM5 inward movement upon receptor activation (indicated with arrows). Panel d.: Top view of MC4R structures overlay. Panel e.: MIF motif rearrangement and impact on W258^{6.48} of CW×P toggle switch motif and L133^{3.36}. Panel f: D/NPxxY and ionic lock DRY motif rearrangements upon activation and impact on residues Y212^{5.58} and Y302^{7.53}. Side chain rearrangements between inactive and active states are indicated with arrows. Gas not shown for clarity.

FIG. 8G, panels a. to f. show a structural comparison of pN162, α-MSH and setmelanotide active state MC4R structures in the Gs alpha interaction vestibule. Panels a., b. and c.: Sidechains of interacting residues between G-protein and MC4R in complex with α-MSH (7F53), setmelanotide (7PIU) and pN163 (8QJ2). Panels d., c. and f. show the detailed interactions between helix 5 of Gs alpha and TM3/ICL2 of MC4R (water molecules shown as dots). Black dashed lines indicate H-bond interactions.

FIG. 8H, panels a. to h. show the binding modes of agonist and antagonist ligands to MC4R. Panels a. to d. show sphere representations of setmelanotide, α-MSH, pN162 CDR3 and SHU9119 in their respective MC4R binding sites. Dashed line shows the maximum depth reached by all ligands. Panels e. to h. show side views of the main interactions of each ligand with their respective receptor. The dashed line represent the same height as in panels α-d. The shaded circle in panel h. highlights the deep interaction of D-Nal4 from the antagonist SHU9119 that forces L133^{3.36} in the inactive state conformation. Panels i. to 1. show top views of the interactions showing the key interactions involved in the calcium binding or the pN162 CDR3 R101 side chain that likely is replacing the calcium in the binding pocket. Black dashed lines indicate H-bond interactions.

FIG. 9A shows the disulfide bridges in the active state pN162-MC4R-DNGs-Cb35 complex. Panel a. shows the assumed disulfide bridge between C279^{ECL3} and C40^{N-term} for which no clear electron density was identified. Density in that region is weak and disulfide bridge was modelled based on previously described MC4R structures. Panel b. shows the disulfide bridge between C271^{6.61} and C277^{ECL3} based on 3D electron density map.

FIG. 9B shows a superposition of MC4R bound to pN162 (active state) or SHU9119 inactive state indicating similar receptor activation. Side view of the structure overlay of pN162 bound active and SHU9119 enabled inactive MC4R conformations. Rearrangement of key residues involved in pN162 signal transduction, representing the active state hallmarks as described in text and depicted in detailed FIGS. 8F to 8H. Arrows indicates the accessibility of the G-protein into the cytosolic MC4R binding pocket upon signal transduction consequent to the TM6 outward movement. Antagonist peptide SHU9119 and pN162. Calcium ion is depicted as a sphere.

FIG. 9C shows a comparison of the densities in the Ca²⁺ pocket of the α-MSH bound active state MC4R structure to the corresponding pocket of the pN162 bound active state MC4R structure. Panel a. shows an Electron density map fitting the Ca²⁺ ion present in the setmelanotide bound active state MC4R structure (7PIU). Neighboring side chain or backbone atoms of setmelanotide (green) that interact with calcium ion (red). Panel b. shows an electron density map of corresponding pocket in the pN162 bound active state MC4R suggesting that the pN162 CDR3 R101 side chain takes the place of Ca²⁺.

FIGS. 9D-9I shows data obtained from a single site scanning mutagenesis assessment of pN163 CDR3 residues by cAMP production (GloSensor), indicating the important residues of pN162-MC4R interaction vestibule. Each panel represents the mutants for a given CDR3 position (R98 (FIG. 9F), T99 (FIG. 9D), G100 (FIG. 9D), R101 (FIG. 9F), I102 (FIG. 9E), V103 (FIG. 9E), R104 (FIG. 9H), P105 (FIG. 9H), L106 (FIG. 9\I), D107 (FIG. 9G) and Y108 (FIG. 9G), respectively), normalized to the fold induction obtained by parental pN162. Each mutant was tested as Phytip purified sample at 7-dilution (dots) and 700-fold dilution (squares). Each dilution is tested as duplicate and average is shown by a horizontal line. For the 32 R101 and R103 mutants tested, the minimum total protein concentration of the Phytip purified mutant at 7-fold dilution was 3.1 μM as determined spectrophotometrically. For the 32 1102 and V103 mutants tested, total protein concentration of the Phytip purified mutant at 7-fold dilution was 2.8 M minimally as determined spectrophotometrically. The protein concentration was measured via Nanodrop. To estimate the contribution of impurities in the Phytip purified nanobody samples, a random set of 15 purified nanobody mutants VHHs (CDR3 mutants) were assessed by Coomassie stained SDS-PAGE. For 14 out of 15 nanobodies tested, contaminants constitute up to 50% of the protein content estimated following visual inspection. For the other nanobody, contaminants constitute up to 75% of the protein.

### EXPERIMENTAL PART

The mouse studies described in Examples 2 and 3 were performed at a fully AAALAC accredited unit (Gubra, Denmark), and all animal experiments were conducted in accordance with the CRO's bioethical guidelines, which are fully compliant to internationally accepted principles for the care and use of laboratory animals. All experiments were licensed by the Danish Animal Experimentation Council. This animal model is based on male C57BI/6J mice that are fed for a minimum of 18 weeks a 60% high fat diet that result in rapid weight gain. The mice were obtained from commercial vendors in Europe and United States transferred to Gubra's animal unit. The animal room environment is controlled (targeted ranges: temperature 21±2° C.; relative humidity 50±10%). The animals are maintained on a reversed 12 h light/dark cycle and have access to plenty of bedding material, shelters and chewing sticks. Animals were dosed just before lights went off.

### Example 1: Generating Anti-MC4R Agonist VHH

Agonist VHH against MC4R were generated by immunizing a llama with a chimeric MC4R GPCR followed by selection via phage display, essentially as described in WO/2021/140205 (immunization and screening) and WO 2020/221768 (which describes ConfoChimers® for MC4R).

This afforded binders at a hit rate of 30%, of which 9% were found to be agonists and 21% were antagonists. Binders were characterised using a ConfoSensor® assay (see WO/2020/221769) and agonist profiles were confirmed in a cAMP assay (9% hit rate).

In particular, in vivo matured nanobody repertoires were induced by the genetic immunization of llamas with an engineered active state MC4R conformation. The MC4R active state conformation was attained by the genetic fusion of a ConfoBody to the C-terminus of a MC4R-β2AR hybrid GPCR. The β2AR G-protein mimetic Nb80 ConfoBody was used (referred from hereon as Cb80). The MC4R chimera was synthesized by grafting the C-terminus and intracellular loops of β2AR onto MC4R. The in vivo matured nanobody repertoires were enriched by phage display on custom virus like particles (VLPs) presenting active state stabilized MC4R. Similar to the immunogen, active state-stabilized MC4R-harboring VLPs were generated using an expression cassette containing the MC4R-β2AR chimera-Cb80 genetic fusion. Periplasmic extracts were prepared of E. coli-expressed monoclonal nanobodies picked from phage outputs that represented the different panning conditions. The antibody fragments were assessed for MC4R-specificity, the ability to recruit cytosolic signal transducers or to induce signaling. MC4R specificity was evaluated by homogeneous time resolved fluorescence (HTRF) and flow cytometry. HTRF on HEK293T cells transiently expressing the SNAP-tagged MC4R chimera genetically fused to Cb80 showed a 33.8% hit rate of MC4R-specific binders (227 out of 672 tested VHHs with an HTRF ratio>2 over the signal obtained by a mock nanobody). Sequence analysis of the selected MC4R binders discriminated 73 different nanobody sequence clusters based upon complementary determining region 3 (CDR3) amino acid sequence analysis. To confirm MC4R specificity and the interaction with the extracellular domains of native receptor expressed at the cell surface, periplasmic extracts of representative nanobodies belonging to these 73 sequence clusters were tested in flow cytometry for specific binding to a stable Flag-tagged MC4R chimera-Cb80 expressing CHO-K1 cell line. Following gating for intact cell populations (cells with undetectable Topro-3 staining), the vast majority of these clones (96%) showed specific binding to the stable MC4R CHO-K1 cell line while showing only background fluorescence to a stable mock GPCR CHO-K1 cell line (data not shown). To explore whether the MC4R binders were able to induce a conformational change that leads to signal transduction, a ConfoSensor assay was established that monitors nanobody-induced recruitment of G-protein mimetic Cb80 to the MC4R-β2AR chimera as described. Out of the 73 cluster variants tested as periplasmic extract, 19 (26.0%) were able to recruit Cb80. Similar recruitment to the MC4R-β2AR chimera was detected for the MC4R clinical benchmark agonist setmelanotide. To confirm signaling, sixteen nanobodies representing different sequence clusters that showed the most pronounced Cb80 recruitment were subsequently tested at a single dilution in a cAMP signaling assay (Lance Ultra HTRF) on double Twin Strep tag-modified wild type human MC4R-overexpressing HEK293 cells. A panel of sixteen Cb80-recruiting nanobodies were selected (based on Cb80 recruitment signals and initial purification yields). All tested nanobodies were confirmed to induce signaling by producing secondary messenger cAMP and they were subsequently purified for further in vitro characterization.

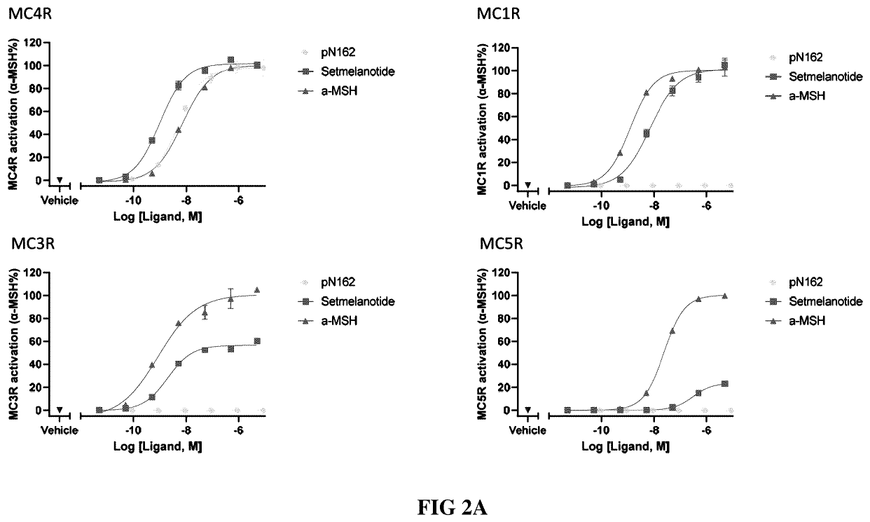

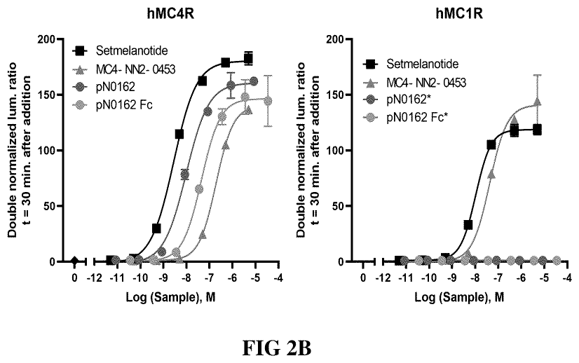

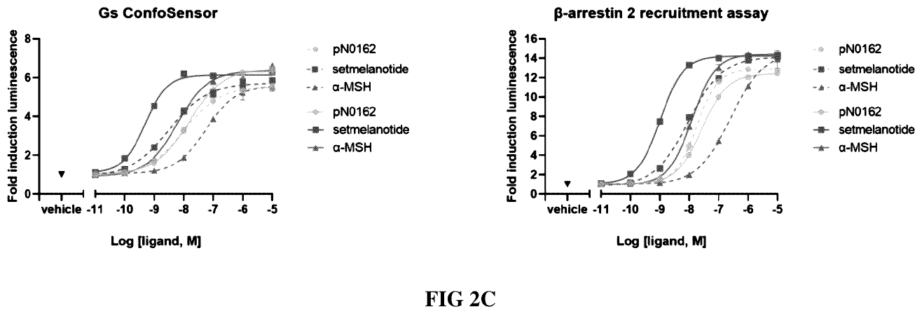

To confirm MC4R activation, dose-dependent nanobody-induced cAMP signaling was assessed (Lance Ultra HTRF). All 16 nanobodies tested showed MC4R-mediated dose-dependent cAMP accumulation with potencies ranging between 0.7 (pN162 & pN152) and 393 nM (pN161). All 16 nanobodies behaved as full agonists when compared to the maximum efficacy obtained with α-MSH. In order to rank them for potency, the agonistic nanobodies were assessed for real-time cAMP monitoring in Flag-tagged human wild type MC4R-expressing HEK293T cells (GloSensor®). pN162 was identified as the most potent full agonist nanobody with a similar EC50 as the endogenous ligand (average EC50 pN162 13.2±5.6 nM; average EC50 α-MSH 14.1±7.7 nM). Binding of pN162 to the human melanocortin receptor subtypes MC1R, MC2R, MC3R, MC4R or MC5R transiently expressed as Flag-tagged receptors in HEK293T cells was assessed by flow cytometry. While receptor presence was confirmed via anti-Flag detection, binding of 1 μM pN162 was only demonstrated for MC4R. No binding could be detected to any other melanocortin receptor paralogue, demonstrating that pN162 specifically interacts with MC4R. For a head-to-head comparison of receptor subtype activation, pN162, setmelanotide and α-MSH were tested in the GloSensor cAMP assay using HEK293 cells transiently transfected with human wild type MC1R, MC3R, MC4R or MC5R (MC2R was not included as pN162 did not interact with MC2R assessed via flow cytometry; data not shown). Contrary to setmelanotide and α-MSH, which showed activity against all melanocortin receptors tested (see FIG. 2B), pN162 was confirmed to be a specific MC4R agonist as it only induced detectable signaling on MC4R. The clinical benchmark setmelanotide is a non-specific MC4R agonist as it also activates MC1R, MC3R and to a lesser extent MC5R. The setmelanotide-induced average signaling potencies (EC50) to the distinct melanocortin subtype receptors tested in cAMP GloSensor assays were 1.96±1.14, 11.5±4.1, 2.10±0.12, 451±143 nM against human MC4R, MC1R, MC3R and MC5R, respectively. Compared to setmelanotide, pN162 showed a 6.7-fold reduced potency in cAMP signaling via human MC4R (GloSensor). Interestingly, the conserved H×RW motif of melanocortin peptide agonists and antagonists was not present in any of the hypervariable gene segments (including CDR3) of the agonist nanobody pN162.

The sequence of one of the hits obtained (pN0150=SEQ ID NO: 182 and pN0162=SEQ ID NO:184) were further optimized by humanization and potency maturation using techniques known per se (see for example the further disclosure herein). The sequences of the clones resulting from the optimization of pN162 (pN2059, pN2060, pN2061, pN2062 and pN2121) are given in Table A-2 as SEQ ID NO's: 185 to 189. The CDR and framework regions of the above agonist VHH are listed in Table A-1.

The VHH's were also formatted as Fc constructs, the sequences of which are given in Table A-2 as SEQ ID NO's: 190 to 193.

Table 1A gives the results of characterisation of pN0162 and the Fc-based construct tpN0162-huFcIgG1_N297A. Alpha-MSH (the natural agonist of MC4R) was used as reference.

Table 1B gives the human melanocortin receptor subtype in vitro cAMP potencies (EC₅₀ in nM) of pN162, α-MSH and setmelanotide (GloSensor). The number of repeats used to calculate the average EC₅₀ is indicated (n=x).). Exemplary graphs are shown in FIG. 2A.

Selectivity for hMC4R over hMC1R was tested as described in Example 4. The results are shown in FIG. 2B. The MC4R receptor agonist MC4R-NN2-0453 (Royalty et al. (2014) J. Clin. Pharmacol. 54:394-404; and Conde-Frieboes et al. (2012) J. Med. Chem. 55:1969-1977) was included as a reference.

To understand whether the primary MC4R signaling pathways are induced by pN162, recruitment of Gs and β-arrestin (Gs being the primary G-protein transducers recruited by α-MSH) were assessed using a ConfoSensor assay and β-arrestin recruitment assay in N-terminally Flag-tag modified WT human MC4R-expressing HEK293T cells. Table IC gives the in vitro potencies (EC50 in nM) of pN162, α-MSH and setmelanotide in Gs ConfoSensor or β-arrestin recruitment assays in absence and presence of Ca²⁺. Experiment was performed twice and representative graphs are shown in FIG. 2C for the ConfoSensor assay (FIG. 2C, left hand graph) and the β-arrestin recruitment assay (FIG. 2C, right hand graph). Similar to α-MSH and setmelanotide, pN162 signals via both pathways. When tested in these assays, pN162 dose-dependently recruits Gs and β-arrestin2. In the presence of cofactor Ca2+, in both assays, setmelanotide shows a higher potency compared to pN162 and α-MSH. Surprisingly, in both recruitment assays, pN162-induced recruitment seemed to be independent of Ca²⁺ ions, contrary to the recruitment induced by α-MSH and setmelanotide.

TABLE 1A

| | Assay | Natural agonist | | pN0162- |
|---|---|---|---|---|
| Assay | Principle | (alpha-MSH) | pN0162 | huFcIgG1_N297A |
| Radioligand | Radioligand | 167 ± 10 nM | 6.48 ± 1.96 nM | 0.89 ± 0.39 nM |
| | displacement | (n = 2) | (n = 2) | (n = 2) |
| | CHO stable | | | |
| | cell line | | | |
| cAMP assay | Promega | 12.3 ± 5.5 nM | 8.40 ± 3.29 nM | 34.7 ± 8.9 nM |
| (GloSensor) | GloSensor ™ | (n = 4) | (n = 7) | (n = 3) |
| | HEK293 | | | |
| | transiently | | | |
| | transfected, | | | |
| | real-time | | | |

TABLE 1B

| | MC4R | MC1R | MC3R | MC5R |
|---|---|---|---|---|
| pN162 | 13.2 ± 5.6 | No effect | No effect | No effect |
| | (n = 9) | | | |
| α-MSH | 14.1 ± 7.7 | 1.9 ± 0.7 | 0.9 ± 0.1 | 30.0 ± 6.0 |
| | (n = 18) | (n = 2) | (n = 2) | (n = 2) |
| setmelanotide | 2.0 ± 1.1 | 11.5 ± 4.1 | 2.1 ± 0.1 | 451.0 ± 143.0 |
| | (n = 34) | (n = 3) | (n = 2) | (n = 2) |

TABLE 1C

| | Gs ConfoSensor | Gs ConfoSensor | β-arrestin | β-arrestin |
|---|---|---|---|---|
| | 1 mM Ca²⁺ | No Ca²⁺ | 1 mM Ca²⁺ | No Ca²⁺ |
| pN162 | 12.0 ± 5.0 | 8.5 ± 2.1 | 23.0 ± 7.1 | 16.0 ± 2.1 |
| α-MSH | 9.6 ± 4.9 | 298.0 ± 342 | 38.0 ± 37.0 | 513.0 ± 373.0 |
| setmelanotide | 0.6 ± 0.2 | 12.0 ± 12.0 | 1.3 ± 0.5 | 18.0 ± 12.0 |

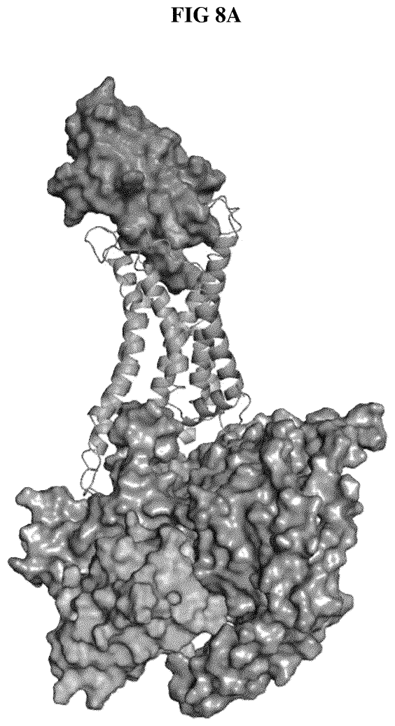

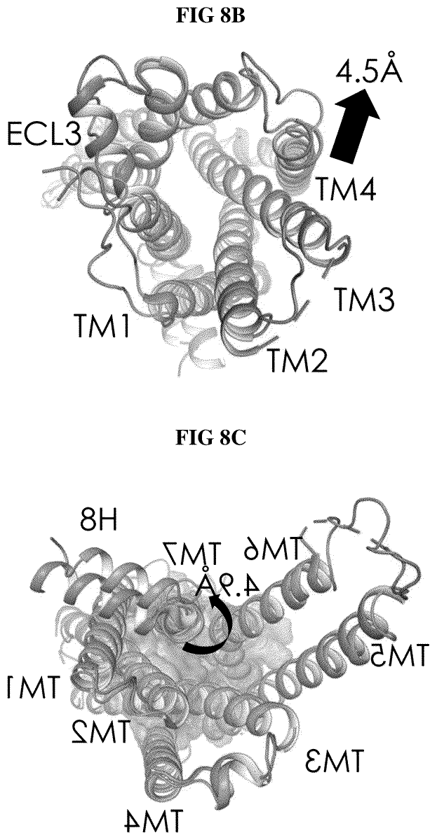

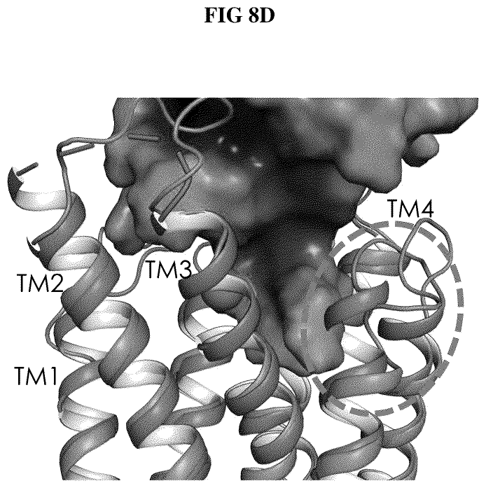

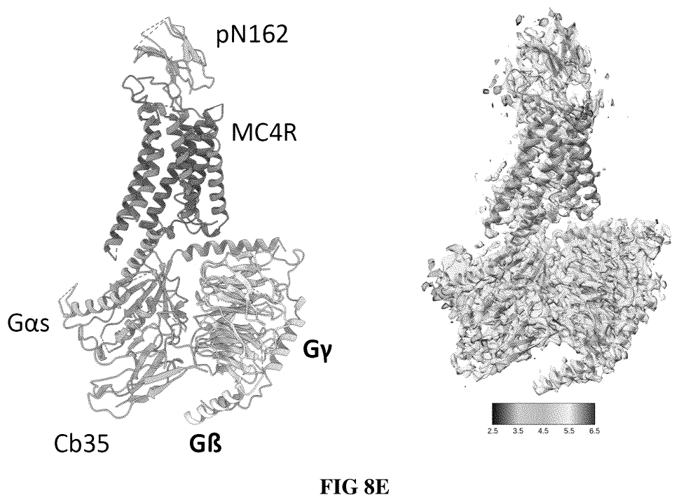

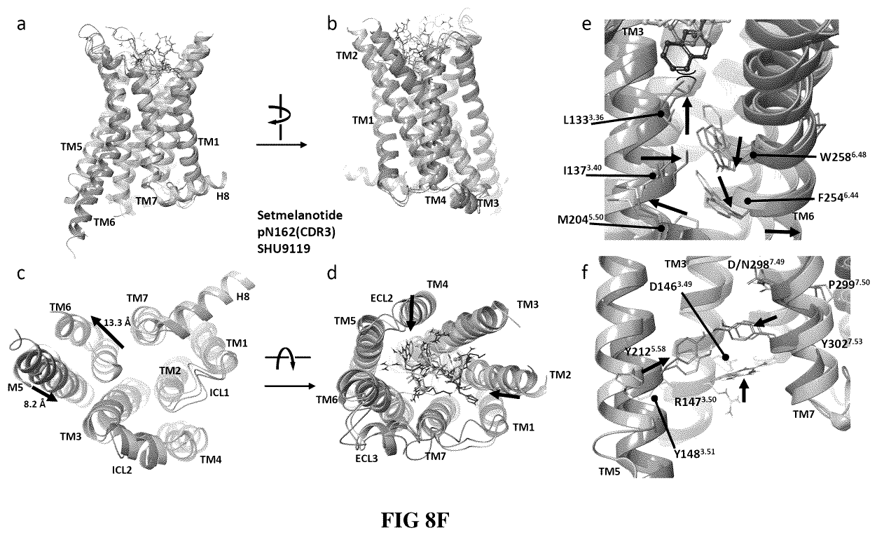

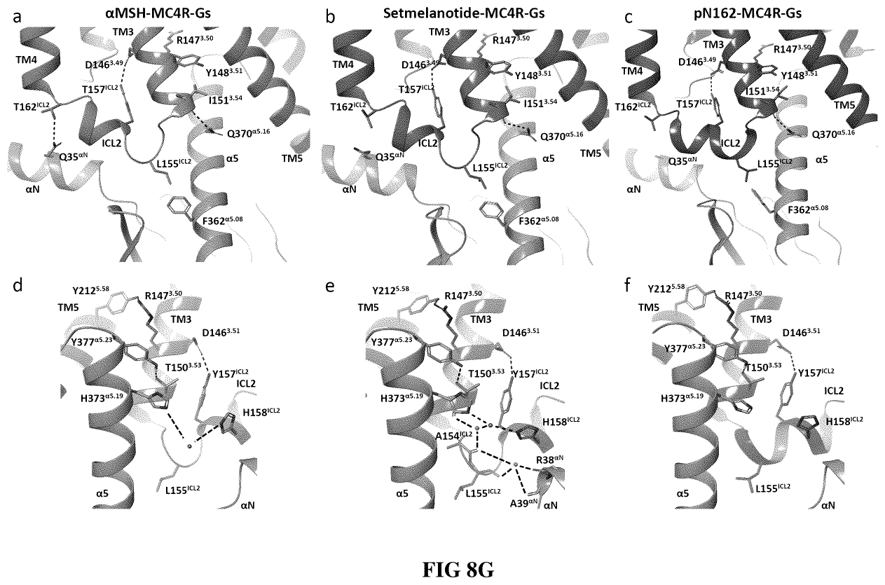

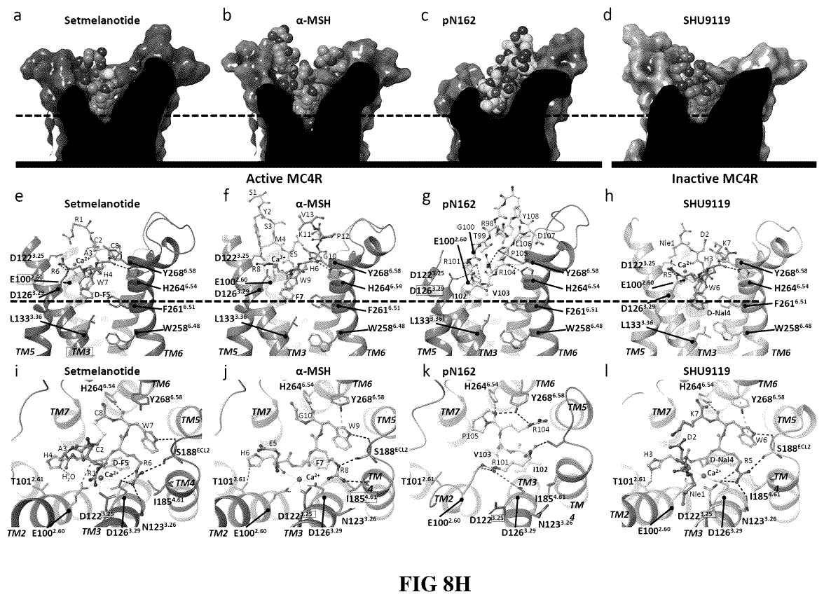

Also, the structure was determined of pN162 as part of a complex with MC4R, Cb35 (see EP 2723764) and a stable G-protein variant. The resulting structures are shown in FIGS. 8A to 8D, and show a very tight and deep interaction between receptor and CDR3 of pN162, with the key residues of CDR3 overlapping natural-ligand binding site. It can also be seen that the receptor is on fully active-state based on TM end of 5/6/7. In addition, from FIGS. 8B to 8D, it can be seen that, overall, the orthosteric binding pocket shows similar positioning of TM helices except for upper part of TM4 (i.e. an overall similar orthosteric LBP with an outward displacement of TM4), with binding of pN162 inducing an outward displacement of TM4 towards the EC aspect by up to 4.5 Å. Also, in the complex, the positions of TM1-7 towards the IC aspect appear to be consistent with the published αMSH bound structure of MC4R (Zhang et al. (2021), Cell Res 31:1163-1175). Further reference is made to Example 8 and FIGS. 8E to 8H.

### Example 2: Diet-Induced Obese (DIO) Mouse Studies—In Vivo Acute Setting and ICV Administration

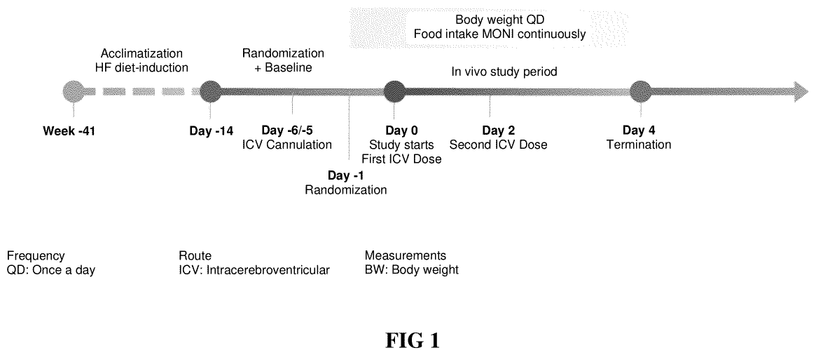

This was performed as schematically shown in FIG. 1

Male DIO mice (5 weeks of age at time of arrival and HF diet induction, 46 weeks of age at study start, n=50) had ad libitum access to 60% high fat diet (D12492, Research diets) and water. The DIO mice were acclimatized to an automated real-time food intake monitoring system (MoNI system) individually housed in the system for 14 days prior to study start. The animals went through ICV cannulation on study day −6/−5 and on study day−1, animals were randomized into 5 treatment groups (n=8 per group) based on body weight (PM, day−2). On day 0 and day 2, animals were ICV dosed with vehicle, setmelanotide low (0.5 nmol*), setmelanotide high (5 nmol, adjusted for the second dose (Day 2) to 0.05 nmol (instead of 0.5 nmol) and 0.5 nmol (instead of 5 nmol), pN162 low (0.5 nmol) or pN162 high (5 nmol) just prior to the dark phase at 3 PM. Online food intake was recorded for 94 hours post-first dose and body weight measured once daily throughout the study.

Treatment with pN162 (0.5 and 5 nmol) reduced relative body weight and cumulative food intake when compared to vehicle on study day 4. Cumulative food intake was reduced by pN162 in a dose-dependent manner after first dose, while a similar reduction in food intake between the groups was observed after the second dose. Setmelanotide treatment (0.5 and 5 nmol) reduced relative body weight after the first dose along with a significant and dose-dependent lowering in cumulative food intake. A similar effect was observed for treatment with 0.5 nmol at the second dose while the group for which the dose was lowered to 0.05 nmol showed no effect on food intake as compared to vehicle.

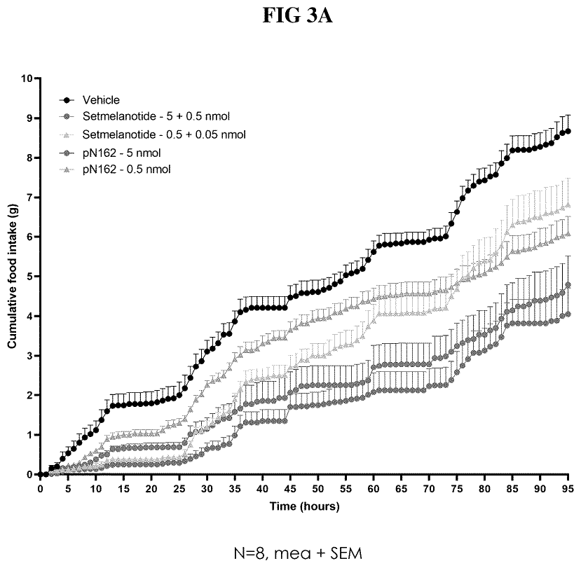

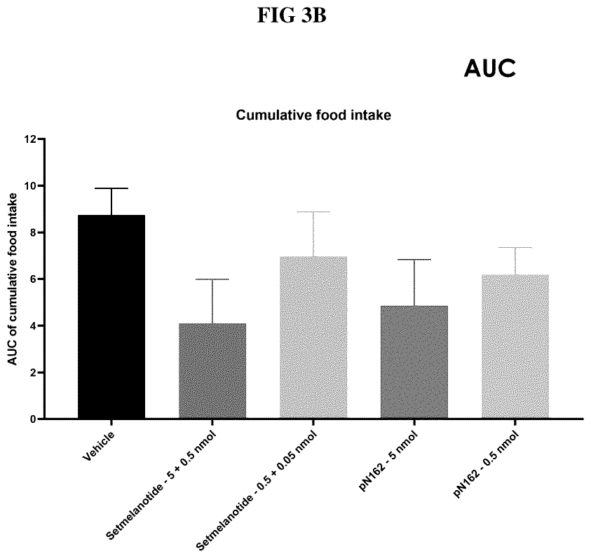

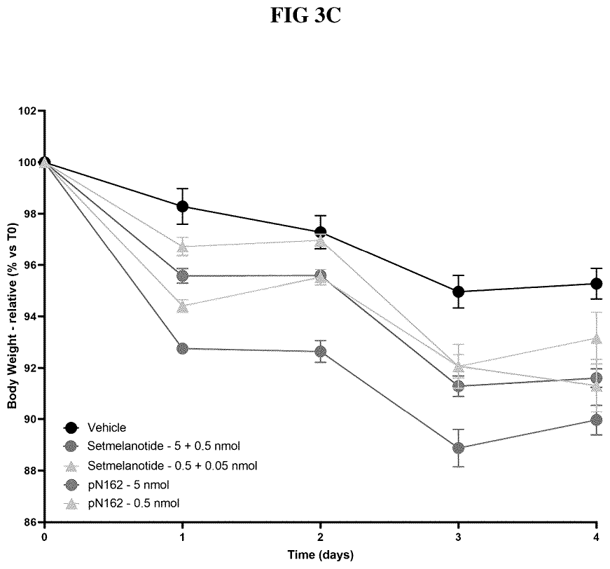

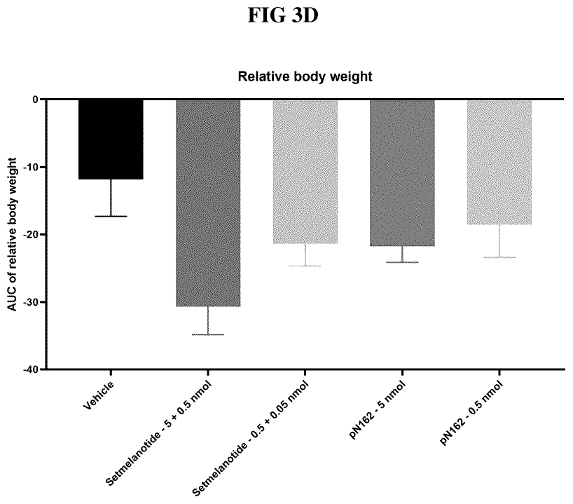

The results are also graphically shown in FIGS. 3A, 3B, 3C and 3D.

### Example 3: Diet-Induced Obese (DIO) Mouse Studies-14-Day Subcutaneous Administration

This was performed as schematically shown in FIG. 4.

Male DIO mice (5 weeks of age at time of arrival and HF diet induction, 45 weeks of age at study start, n=50) had ad libitum access to 60% high fat diet (D12492, Research diets) and water. On study day−2, the mice were randomized into five treatment groups (n=10) based on body weight. The mice were dosed with vehicle (50 mM Tris pH 8.0, 250 mM sucrose, 24 mM L-Arginine, 150 mM NaCl., SC, QD), 1 mg/kg or 5 mg/kg setmelanotide (SC, QD), 40 mg/kg or 100 mg/kg VHH-FC pN2113 (SC, QD) for 14 days (day 0-13) just prior to the dark phase at 1 PM, followed by a 7-day wash-out period (day 14-20). Body weight and food intake were measured daily starting from day−3. On the day of termination, plasma samples were collected from the groups receiving pN2113. At termination, the livers were weighed, and liver samples collected for all groups.

Treatment with pN2113 (40 and 100 mg/kg) had no significant effect on the absolute or relative body weight at the end of the treatment period (day 14) while the total area under the curve (tAUC) of the relative body weight (day 0-14) was significantly reduced for both doses. At the end of the treatment period, absolute body weight was significantly reduced when dosed for 14 days with 5 mg/kg setmelanotide and relative body weight was significantly reduced with both 1 mg/kg and 5 mg/kg setmelanotide. In addition, the tAUC of the relative body weight (day 0-14) showed a significant reduction with setmelanotide treatment (1 and 5 mg/kg). Following the wash-out period, the relative body weight remained significantly reduced for both setmelanotide dose groups (1 and 5 mg/kg) compared to vehicle. None of the compounds had an effect on liver weight.

### Example 4: HMC4R: HMC1R Selectivity Assessment Using Cyclic Adenosine Monophosphate (cAMP) GloSensor Assay (Fresh Cells, Reverse Transfection)

HEK 293T cells were routinely maintained at 37° C., 5% CO2, under humidified atmosphere in Dulbecco's modified Eagle's medium (DMEM, Gibco, cat. no. 41966-029) supplemented with 10% heat-inactivated fetal bovine serum. In preparation of the experiments, cells were plated on poly-L-lysine coated white 96-well plates (Costar: 3917) at the density of 3,00E+04 cells/well. At the moment of seeding, cells were transfected with 25 ng of human MC4R in pcDNA3.1 (+) and 50 ng of p22F GLoSensor plasmid (Promega Corporation), empty pcDNA3.1 (+) vector was added to obtain 100 ng of total DNA, using XtremeGene HP (Roche). To evaluate cross-reactivity of different VHHs for other melanocortin receptors, cells were additionally transfected with 50 ng of DNA encoding one of the three melanocortin receptors MC1R/MC3R/MC5R in pcDNA3.1 (+) and 50 ng of p22F GLoSensor plasmid (Promega Corporation), using XtremeGene HP (Roche). After 24 h post transfection, growing medium was gently removed from the cells and replaced with 90 μl/well of Assay medium (OptiMEM media containing 10% FBS HI with 2% GloSensor reagent (Promega)) in which cells were incubated for 2 h at room temperature in the dark. Next, background luminescence level was recorded using the En Vision 2104 Multilabel Plate Reader (Perkin Elmer). Samples (purified VHH produced in E. coli and/or mammalian cells with or without FC fusion) or benchmark compounds were prepared at 10× working concentration in OptiMEM containing 10% heat-inactivated Fetal Bovine Serum (FBS). After addition of 10 μl of samples luminescence was recorded in a kinetic mode using the En Vision 2104 Multilabel Plate Reader (Perkin Elmer). Data were analyzed using GraphPad Prism 8 by applying nonlinear regression (curve fit).

### Example 5: In Vitro Pigmentation Assays with Human Skin Cells (2D HP-NHEM)

In this example, the effect of compounds pN162 and Setmelanotide acetate was evaluated on melanin synthesis using both highly- and moderately-pigmented normal human epidermal melanocytes (NHEM). Highly and moderately pigmented melanocytes were seeded in 24-well plates and cultured in culture medium for 24 hours. The medium was then replaced by culture medium containing or not (control) the test compounds or the reference compounds (L-tyrosine at 1 mM, IBMX at 100 and 200 μM and NDP-MSH at 10 μM). The cells were then incubated for 10 days with treatment renewal at day 3 and day 7. All experimental conditions were performed in n=3. At the end of the incubation time, the culture supernatants were removed, and melanin was extracted by cell lysis using a 0.5 N NaOH solution. The optical density (OD) of each experimental point was measured at 405 nm and melanin quantity was determined according to melanin standard curve (standard curve ranging from 0.39 to 100 μg/ml). The results were expressed in μg/ml of melanin. The treatment of NHEM highly or moderately pigmented with 1 mM L-tyrosine, 100 μM IBMX or 10 μM NDP-MSH induced a stimulation of melanin synthesis compared to the non-stimulated control condition. NHEM-HP response to L-tyrosine and NDP-MSH was stronger than in NHEM-MP while IBMX induced a stronger response in the NHEM-MP. Compounds pN162 and Setmelanotide acetate did not modulate the melanin synthesis by NHEM-MP. When tested at 10 μM on the NHEM-HP, compound Setmelanotide acetate significantly increased the melanin synthesis (130% of the control) while compound pN162 did not increase it and even tended to reduce it (89% of the control).

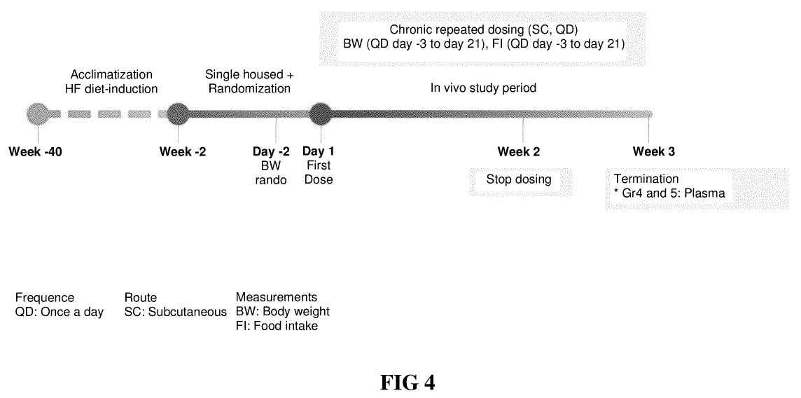

Results are shown in FIG. 5.

### Example 6: In Vitro Pigmentation Assays with Human Skin Cells-3D-RHE-MEL

In this example, the impact of compounds pN162 and Setmelanotide acetate on the skin pigmentation was investigated using reconstituted human epidermis (StratiCELL, RHE/MEL/001) containing Normal Human Epidermal Melanocytes—Darkly Pigmented (NHEM-DP; phototype IV-V).

The study was carried out on melanized human epidermis (RHE/MEL/001; batch CB0521-3) in vitro reconstituted with NHEKs (Normal human epidermal keratinocytes, Lonza 00192906) isolated from foreskin of 3 neonatal Caucasian donors and NHEM-DP (Normal Human Epidermal Melanocytes-Darkly pigmented) isolated form one donor (phototype IV-V). The tissues were cultured at the air-liquid interface in Epilife medium (Fisher Scientific, MEP1500CA) containing specific supplements (with among others Human Keratinocytes Growth Factors, Fisher Scientific S0015 or SO01K) and antibiotics (Gentamycin, Fisher Scientific, 15710049). They were maintained in a humid atmosphere at 37° C. with 5% CO₂.

RHE/MEL/001 were reconstituted and cultivated at the air-liquid interface during 14 days in Epilife medium supplemented with specific growth supplements (as described in paragraph 1) in a humid atmosphere at 37° C. with CO₂ 5%. The two test items were applied in the culture medium at one concentration (10 μM; selected as this is a concentration which gives full MC1R functionality for setmelanotide in the functional GloSensor CAMP assay), during 10 days, from day 4 to day 14 after placement at the air/liquid interface, with 5 medium refreshes (at day 7, 8, 9, 10 and 11).

IBMX (Sigma; 15879) at 100 M was used as tanning reference compound to validate the experiment. A DMSO control (corresponding to the solvent used to solubilize IBMX) at 0.02% (corresponding to the final concentration in DMSO found in the culture medium for the condition treated with IBMX) was planned as well. Four replicates were planned per condition (n=4).

At the end of the 10 days treatments (at day 14), macroscopic high-resolution pictures of tissues (n=4) were taken. This was performed using the C-Cube device (Dermoscope type tool; Pixience, France) coupled to C-Cube clinical 3 software, which uses a proprietary method of colour calibration, and provides razor-sharp images of high quality and precision. The following endpoints were calculated, each one taking into account 3 values from the mode “L* a* b*” with L* reflecting the luminance/brightness (value from 0 to 100 with high values for clear skins), a* to gives an idea about the redness of the skin (value from −60 to 60 on the red/green axis) and b* to gives an idea about the yellowness of the skin (value from −60 to 60 on the blue/yellow axis):
ITA (Individual Typology Angle) characterizing the phototype, compared to a standard scale, with ITA>55° for very clear skins and ITA<−30° for black skins.
PI (Pigmentation Index) whose value is directly proportional to the skin pigmentation, with PI from 140 for clear skins to 200 for dark skins.
Colorimetric difference (score or ΔE) allows to quantify colour variation between a test condition and a reference. This specifically allows to characterize the perceived colour difference by the human eye. Scores between 1 and 3 units represent colour differences that can be perceived by a trained eye and scores above 3 represent colour differences perceived by every eye.

ITA and PI are given directly by the software from L*, a* and b* parameters and an internal standard. The colorimetric difference (DeltaE) also named visual colorimetric difference between a sample and a reference is constructed from these parameters coming from the sample and coming from the reference as followed: DeltaE=((Lref*−L*)\hat{ }2+ (aref*−a*)\hat{ }2+ (bref*−b*)\hat{ }2)\hat{ }0.5

The two test items, hydro-soluble, were evaluated in parallel of one reference compound known to be a pigmentation inducer, namely IBMX. Their capacity to stimulate the production of melanin was evaluated by visual quantification of melanin on macroscopic high resolution pictures obtained by dermoscopy. The epidermis, cultured at the air/liquid interface, were treated during their reconstruction process from day 4 to day 14.

It was observed that Individual typology angle (ITA), pigmentation index (PI) and visual colorimetric difference (score, ΔE) calculated from high resolution images taken by dermoscopy were significantly modulated after treatment with setmelanotide acetate at 10 μM. The changes in ITA and PI observed with setmelanotide acetate both indicated a pro-pigmentation of the epidermis. No significant effects were observed on these parameters following pN162 VHH treatment.

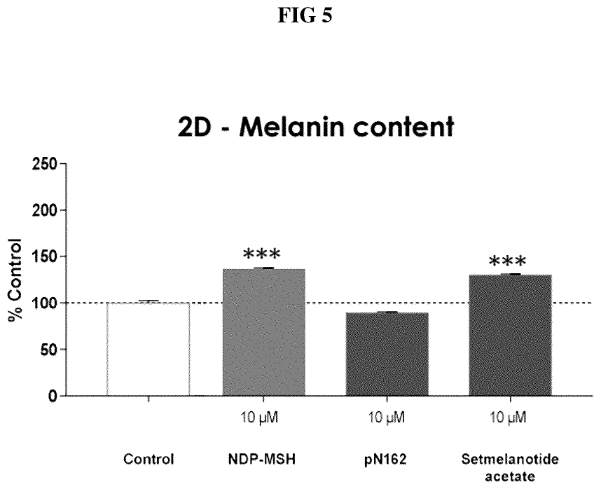

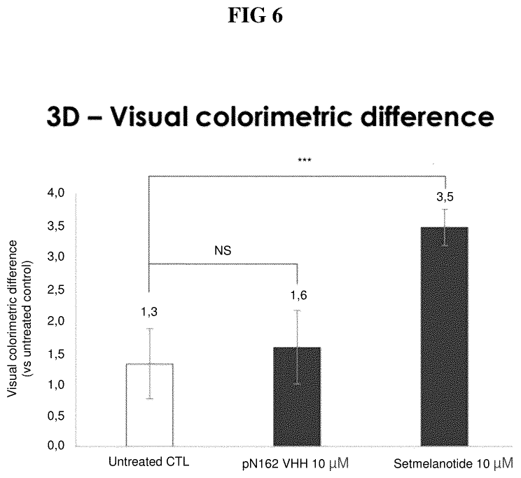

Results are shown in FIG. 6.

In conclusion, based on dermoscopy analysis, it was concluded that setmelanotide acetate applied at 10 μM has a tanning effect on the human epidermis in culture. This effect could be due to a potential action of the peptide on the activity of MC1R. With pN162 VHH, no effect on epidermis pigmentation was observed which allowed us to confirm hypothesis that this MC4R agonist is selective and is not able to modulate the activity of MC1R and is thus not influencing pigmentation in vitro.

### Example 7: Evaluation of the Cardiovascular Safety Pharmacology Profile of 2 Compounds after a Single Treatment in Conscious Non-Naïve Telemetered Rats

In this example the effects were evaluated of a single administration of the cyclic peptide Setmelanotide (1 and 10 mg/kg) and the single domain antibody (VHH) hIgG1 FC fusion pN2113 (100 mg/kg) on cardiovascular function measured using telemetry in chronically implanted conscious non-naïve rats. Cardiovascular function was analysed in the conscious unrestrained rat, after surgical implantation of telemetric devices. Using this approach, arterial pressure and heart rate was continuously monitored over the 24 hours after SC administration of the Test Item in freely moving animals. In addition the effects were evaluated of a single administration of Setmelanotide (1 or 10 mg/kg) or pN2113 (100 mg/kg) on body weight and on food consumption of these lean rats, as MC4R agonists are described in the literature to induce a decrease in body weight and food consumption in rodents (Kumar et al, 2009 and Mayer et al, 2005).

The study was conducted in AAALAC accredited facilities under EU and French animal welfare regulations for animal use in experimentation (European Directive 2010/63/EU and French decrees and orders 2013-118 of Feb. 1, 2013 and 2020-274 of Mar. 17, 2020). This experimental project is approved by the Biotrial Ethics Committee “Comité de Réflexion Ethique en Expérimentation Animale (CR2EA) (registered by the “Ministère de l'Enseignement Supérieur et de la Recherche et de l'Innovation” (French ministry of higher education and research) under No. 67.

The study was carried out using 12 non-naïve male Wistar rats (6 implanted for telemetry+6 unoperated companions) (Janvier Labs, Saint Berthevin, France), weighing 400-650 g on the day of dosing. The animals were housed in groups of 2 in polysulfone cages (floor area=1500 cm²) under standard conditions: room temperature (22±2° C.), light/dark cycle (12 h/12 h, light 4:00 am to 4:00 pm), air replacement (15-20 volumes/hour), water and food (Safe, Augy, France, ref. A04) ad libitum. The rats were allowed to habituate to environmental conditions for at least 5 days prior to surgery. The chronically instrumented rats of the telemetry study were individually housed immediately after surgery and then housed in pairs with a non-instrumented companion after recovery, and were identified by an car tattoo.

The surgery was performed under isoflurane anaesthesia (5% isoflurane/air for anaesthesia induction and 1.0-2.5% isoflurane/air for anaesthesia maintenance). A mid-ventral incision was aseptically performed and the descending aorta was exposed between the left renal artery and the iliac bifurcation. The catheter of the sensor (HDS10, HDS11 et HDS21 implant, Data Sciences International, St Paul, MN, USA) was introduced into the abdominal aorta and fixed in place with tissue adhesive. The telemetry transmitter itself was attached with nonabsorbable suture to the peritoneum. The post-surgical analgesia was ensured by SC administration of buprenorphine (10-50 μg/kg, sc) bid for 2 days (including surgery day) and meloxicam (1-2 mg/kg, sc) qd for 3 days (including surgery day).

After recording baseline haemodynamic parameters, Setmelanotide or pN2113 or vehicle was administered by SC administration to each telemetered animals, as a Latin square design, at the exception of the last session with pN2113 dosing. Administrations took place one hour before the onset of the dark phase (around 11.30 am). A minimal washout period of 7 days was respected between 2 treatments. The telemetry and data acquisition system from Data Sciences International (St Paul, MN, USA) and Notocord (France) was used. During the experimental session, the animals were housed in pairs in cages placed on the receiver panel. Arterial blood pressure and heart rate parameters were continuously monitored from at least 1 hour before dosing and over the 24 h-period post-dosing. The body weight of the rats were measured on pre-dose, the day of treatment and on T24 h and T48 h after dosing. At each session of administration, the food consumption (implanted rats and its non-treated companion) was measured from [T-24 h to T0 h], [T0 to T24 h] and [T24 h to T48 h].

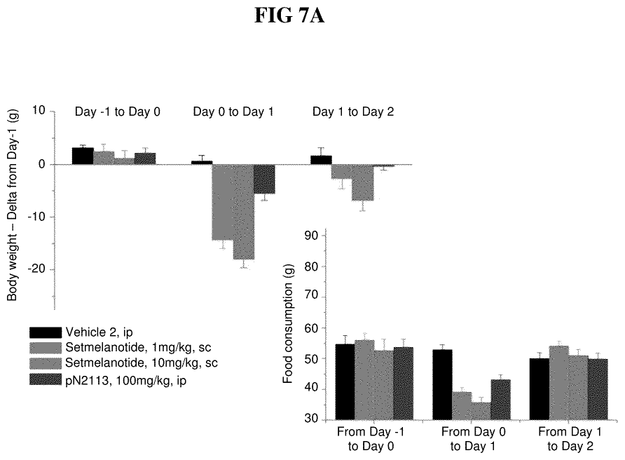

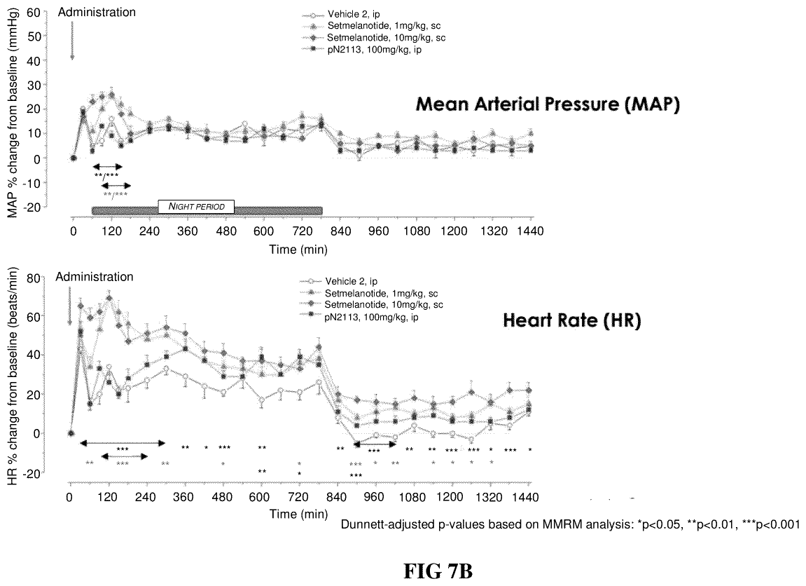

The results are shown in FIGS. 7A and 7B.

### Example 8: Structure Determination of Active State MC4R in Complex with a Potent Agonist Nanobody

To gain insights in the interaction mode between pN162 and MC4R, a cryo-EM structure was determined of pN162-bound human MC4R in complex with Gs at a global resolution of 3.4 Å. The monodisperse pN162-MC4R-Gs complex obtained via an affinity chromatography and a size exclusion polishing step remained stable after a freeze-thaw cycle as assessed by analytical size exclusion and contained all components from the complex. The complex was stabilized by Nb35, also termed Cb35. The cryo-EM map revealed densities for all complex components (see FIG. 8E), the receptor-G protein heterotrimer association mode and, critically, the orientation of the CDR3 of the agonistic nanobody pN162 within the orthosteric binding pocket. Local densities of transmembrane regions TM 3, 4, 5 and 6 ensured their sidechains to be modelled with excellent geometry. TM2 and TM7 were less well resolved and some residues sidechains could not be assigned. TM1 showed weak densities and therefore only the Ca helical backbone was included in the PDB submitted model. Therefore, only one of the MC4R-characteristic disulfide bridges could be identified in the density: C271-C277. Finally, Helix 8 had to be partially excluded from the final model due to a lack of density. Other components known for their high flexibility, such as the receptor N-(aa 1-37) and C-termini (318-332-His-avi-EPEA-mCherry), the intracellular loop 1 and 3, and the extracellular loop 1, as well as the alpha-helical domain of Gas, were not or only partially built into the final model. The core of the agonistic nanobody pN162, including CDR3, showed a local resolution of ˜3.5 Å. However, the peripheral portions of the nanobody showed significantly lower resolution (4.5-6.5 Å) which together with the lack of density or lower resolution of the N-terminus, ECLs and EC portion of the TMs of the receptor allowed only limited analysis of pN162-MC4R interactions beyond CDR3 (see below).

Superposition of the receptor with previously published MC4R active state structures and comparison with its inactive state structure (Yu, J. et al., Science, 368, 428-433 (2020)) indicate a similar activation mechanism upon pN162 binding as described for the peptide or small molecule agonists, as well as an identical conformation revealing key hallmarks of receptor activation, namely: i) outward movement of 13.3 Å of TM6 as measured at the Ca atom M241^{6.31} (FIG. 8F, panels a., c. and d.; superscript numbers according to the Ballesteros-Weinstein numbering convention); ii) the CW×P^{6.50} toggle switch motif in MC4R relies on L133^{3.36} sitting in the upward conformation and forces W2586.48 (of motif CW×P) to adopt an inward rotation conformation that, along with the rotation of F254^{6.54} leads to the characteristic TM6 outward movement (FIG. 8F, panel c.); iii) the discontinuous M204^{5.50}I137^{3.40}F254^{6.54} triad side chain rearrangement is conserved in this structure; iv) the class A GPCR active state TM7 motif NPxxY (D298^{7.49}P299^{7.50}xx Y302^{7.53} in MC4R) shows similar rearrangements as in other MC4R active state structures. Y302^{7.53} adopts an inwards conformation towards Y212^{5.58} that helps stabilize the active state orientation of the conserved DRY motif (FIG. 8F, panel f); v) finally, the hallmark upward conformation of R147^{3.50} (of the DRY motif) at the top of the G-protein binding vestibule is also observed (FIG. 8F, panel f).

As described previously, MC4R and the Gas protein interactions occur mainly between TM3, TM5 and ICL2 of the receptor and the α5 helix and C^{term}-cap loop of the Gas protein with TM7. The G-protein interaction crevice in the pN162-MC4R structure mostly overlaps with that of the reported active state MC4R structures and show the typical class A GPCR activation sidechain re-arrangement in the TM3 D^{3.49}R^{3.50}Y^{3.51} motif as compared to the inactive state structure (FIG. 8F, panel f). Comparison of the G-protein binding vestibule of MC4R with those of other agonists (αMSH-7F53 and Setmelanotide-7PIU) reveals a similar interaction network between MC4R and the Gas subunit with minor differences (FIG. 8G). While some side chains were not modelled as no clear density was seen to unambiguously assign a conformation, most of the residues previously described to participate in Gαs-coupling were visible, including the additional H-bond between Y377^{α5.23} and T150^{3.53}, peculiar to MC4R active state. While it is known that ICL2 can adopt agonist specific conformations, a similar behavior is observed as it adopt a different conformation than in the α-MSH or setmelanotide enabled MC4R structures (FIG. 8G, panels a, b and c). The interaction between the Gαs αN subunit and ICL2 (Q35^{αN}-T162^{ICIL2}), that is present for α-MSH but not setmelanotide, could not be established in the case of pN162 due to lack of clear density in that area (FIG. 8G, panel c). One notable difference is the lack of water molecules that bridge the interaction between H373^{α5.19} and H158^{ICL2}. This is both seen in α-MSH and setmelanotide structure (FIG. 8G, panels d, e and f). Giving the orientation of the abovementioned residues in the model it is still expected that water molecules are present to bridge this interaction. The absence of visible density can be expected due to the lower resolution of our map. Analysis of the G-protein vestibule shows that pN162 is able to stabilize the receptor in a conformation similar to the previously described peptidic agonists while having a different mode of binding in the orthosteric site.

Further structural insights of the pN162 MC4R interaction vestibule that can be gained from FIGS. 8A to 8H are described in the following paragraphs.

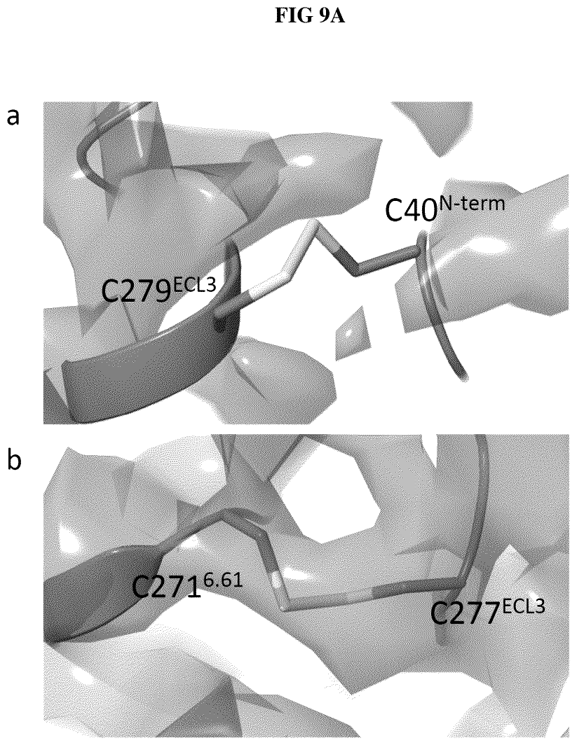

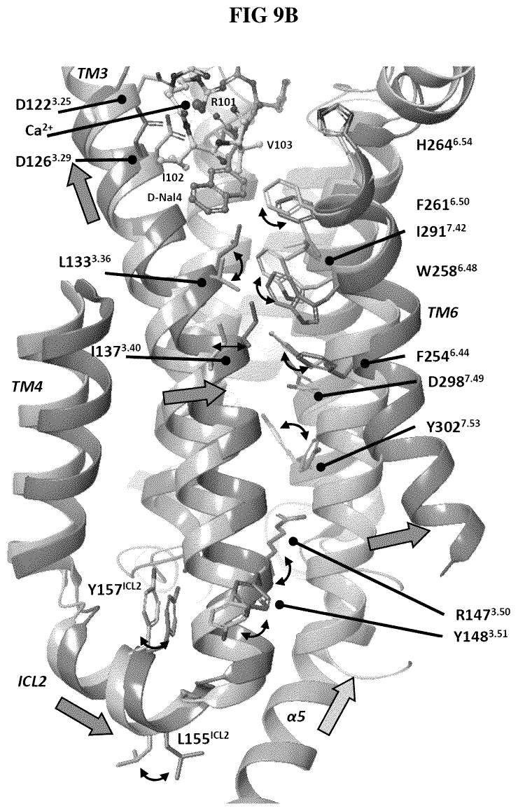

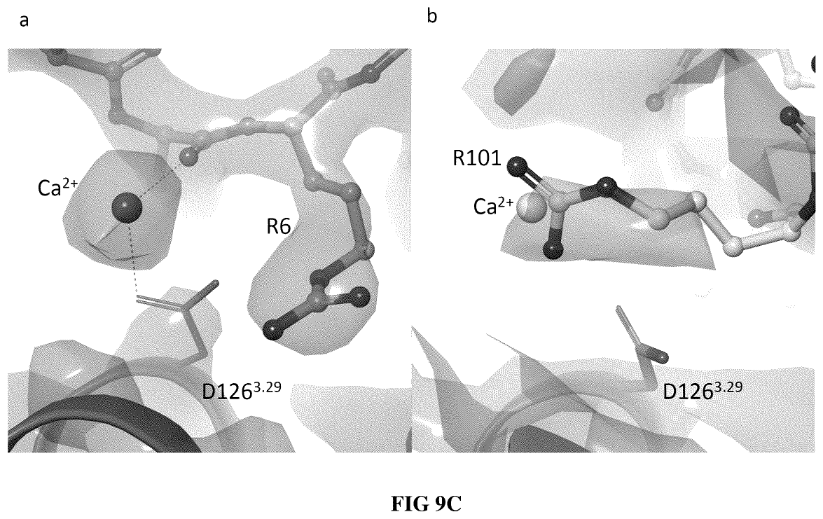

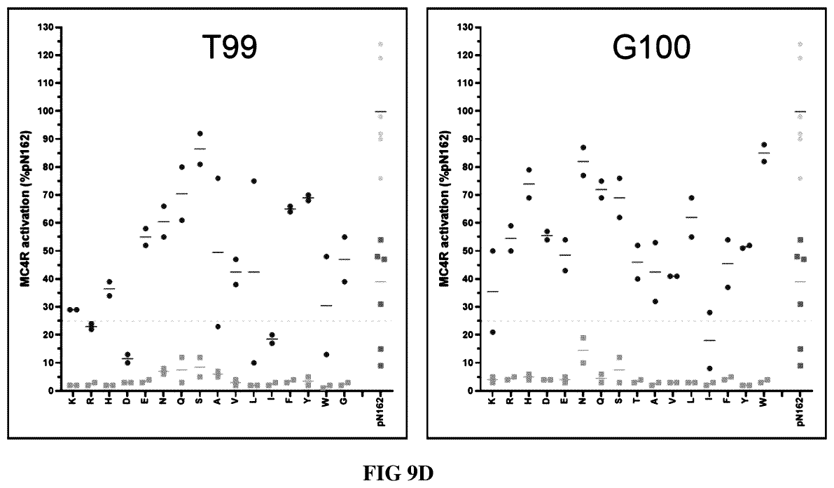

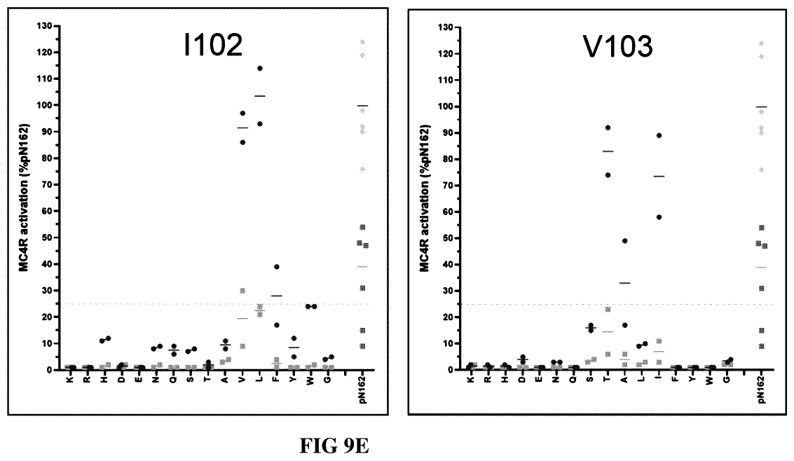

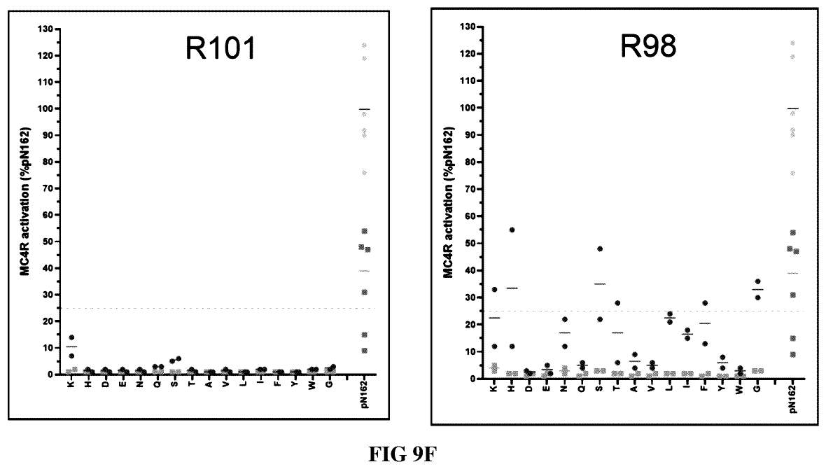

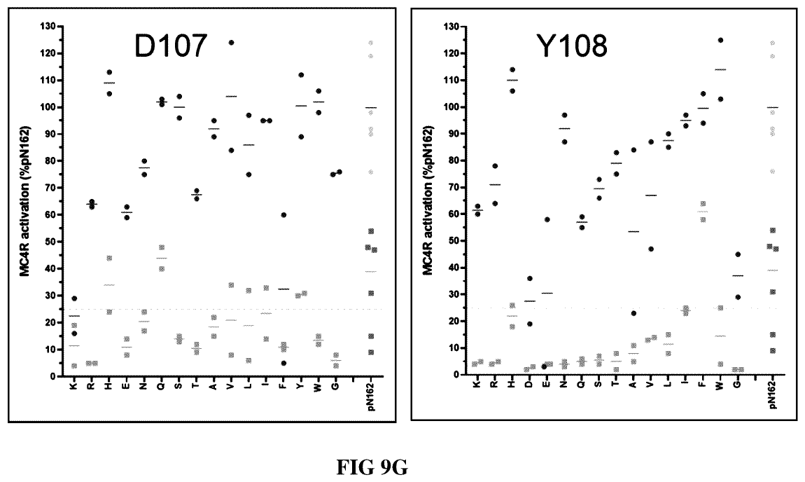

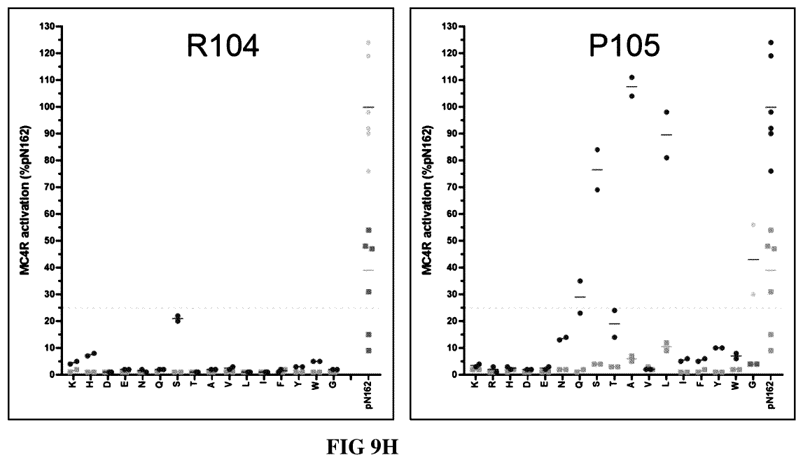

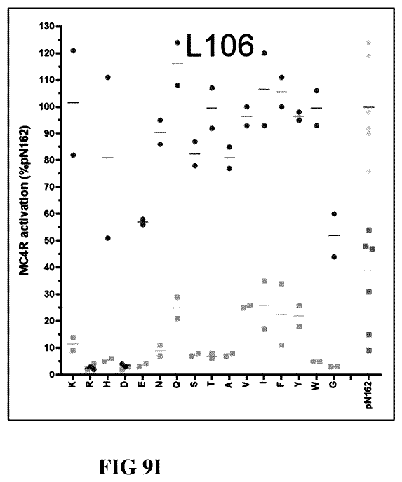

Orthosteric sites of GPCRs can be buried or obstructed by the presence of large extracellular loops (ECLs). It has been shown that the MC4R orthosteric ligand binding pocket for peptide agonists and antagonists is an open cavity as the ECL2 is only one residue long, in stark contrast with other GPCRs (but observed through the melanocortin family). Comparing the structure of MC4R with pN162 to that of setmelanotide, α-MSH or antagonist SHU9119 shows that its CDR3 occupies the same site as the endogenous ligand and its cyclic synthetic analogue (FIG. 8F, panels a, b, d and c and FIG. 8H). All ligands of MC4R described bear the H×RW motif. When looking at the pose and sequence of pN162, no such motif is present. The nanobody activates the receptor by a new set of interactions compared to those previously described for other structures with MC4R agonists (see for example the references cited herein). One of the key residues for receptor activation is the F or D-F of α-MSH or setmelanotide respectively. It allows L133^{3.36} to adopt an upward conformation that forces W258^{6.48} to pivot and trigger the TM6 outward movement. While pN162 does not have a F in that position it does fill that pocket with the presence of two hydrophobic residues (I102 and V103). The Histidine (H×RW) of setmelanotide and α-MSH is forming an interaction that is absent for pN162. TM1 inward movement does not seem to have a downstream impact on active state stabilization. Finally, a key components of the MC4R active structures described to date is the presence of a calcium ion in the orthosteric site that functions as a cofactor. It is coordinated by Glu100^{2.60}, Asp 122^{3.25} and Asp 126^{3.29} as well as some backbone residues of the ligands (reference is also made to PDB deposited structures with accession numbers 7F53, 6W25 and 7PIU). Extensive experimental evidence (mutagenesis data shown in FIGS. 9D-9I and the differential effect of Ca²⁺ on agonist induced signaling pathways (see FIG. 2C)) supports a distinct receptor activation mechanism by pN162. In the case of pN162, unlike α-MSH and setmelanotide, the presence or absence of Ca²⁺ has no effect on receptor signalling (see again FIG. 2C). The MC4R-pN162 structure reveals that R101 could potentially replace the Ca²⁺ ion and interacts with Glu100^{2.60} and Asp 126^{3.29} (FIG. 8H, panels e to g and k and FIG. 9A). Interaction of pN162 with Asp 122^{3.25} could not be confirmed as the density was too weak. Interestingly, pN162 also interacts with His264^{6.54} contrary to the other ligands (setmelanotide, α-MSH or SHU9119; see again the pertinent panels in FIG. 8H). Other distinct interactions of pN162 with MC4R are the interactions between R104 and the backbone of Ser188^{4.64} and Asp189^{ECL2} as well as the backbone atoms of pN162 residues (P105, L106, D107) with both Gln43^{N-Term} and Tyr268^{6.58} (FIG. 8H, panels g. and k.).

Single point mutations in the CDR3 region of pN162 were generated to identify critical CDR3 residues required to induce MC4R signaling. Each residue was mutated into 16 other AA residues, except for M, C and P which were not assessed (P105 was mutated into all other 17 residues, excluding M and C). Two out of the eleven residues were shown to be key for signaling: R101 and R104 (FIGS. 9F and 9H). Any mutation tested at these positions completely abolished or strongly reduced (>than 75%) cAMP production compared to the parent pN162 (see the results shown in FIGS. 9D-9I). The strong impact of R101 mutations on signaling again points to the importance of that residue for mimicking Ca²⁺ that is required for triggering the activation cascade of the receptor. It is also interesting to note that the R101K mutant is the only one that led to minimal signaling (˜10%), underlining the importance of a charged residue in that position for receptor activation. For two other residues (I102 and V103), 13 out of 16 mutations strongly reduced cAMP production compared to the parent pN162. For I102, two semi-conserved mutations to Val or Leu showed little to no impact on signaling (see again FIG. 9E), while a mutation of that position to Phe or Trp showed ˜75% reduced signaling. For V103 (FIG. 9E), mutation to Thr or Leu led to a small reduction in signaling, while replacement by a smaller side chain (Ala, Ser) had a bigger impact. Larger side chains (Phe, Leu, Tyr, Trp) completely abolished signaling which might be due to steric hindrance or interaction with L133^{3.36} in a similar fashion than what was observed for the antagonist SHU9119 (see again FIGS. 9D-9I). While the above described pN162 CDR3 AA residues mutations impact receptor signaling, it is not clear whether these mutations still allow binding to or block signaling of MC4R, as this was not tested.

1. Nanobody in which:
CDR1 is chosen from the group consisting of:
a) the amino acid sequences of SEQ ID NO's: 134 to 141 and 210 to 225;
b) amino acid sequences that have at least 80% amino acid identity with at least one of the amino acid sequences of SEQ ID NO's: 134 to 141 and 210 to 225; and
c) amino acid sequences that have 3, 2, or 1 amino acid difference with at least one of the amino acid sequences of SEQ ID NO's: 134 to 141 and 210 to 225;
and
CDR2 is chosen from the group consisting of:
d) the amino acid sequences of SEQ ID NO's: 150 to 157 and 242 to 257;
e) amino acid sequences that have at least 80% amino acid identity with at least one of the amino acid sequences of SEQ ID NO's: 150 to 157 and 242 to 257; and
f) amino acid sequences that have 3, 2, or 1 amino acid difference with at least one of the amino acid sequences of SEQ ID NO's: 150 to 157 and 242 to 257; and
CDR3 sequence is an amino acid sequence comprising 9, 10, 11, 12 or 13 amino acid residues and comprising (i) at least one arginine residue (R) that is capable of interacting with the Glu100 and/or Asp126 residues of MC4R; and (ii) at least one arginine residue (R) that is capable of interacting with the Ser188 and/or Asp189 residues of MC4R.

2. The nanobody of claim 1, in which CDR3 is the amino acid sequence RTGRIVRPLDY (SEQ ID NO: 168) or an analog thereof.

3. The nanobody of claim 1, which is chosen from the group consisting of SEQ ID NO's: 185 to 189 or from the group consisting of amino acid sequences that have more than 80%, sequence identity with at least one of the amino acid sequences of SEQ ID NO's: 185 to 189.

4. The nanobody of claim 1, wherein the nanobody is humanized.

5. The nanobody of claim 1, wherein the nanobody is an MC4R agonist.

6. The nanobody of claim 1, wherein the nanobody has the amino acid sequence of SEQ ID NO:189.

7. A compound, composition, pharmaceutical composition, construct, protein, or polypeptide, comprising at least one nanobody of claim 1.

8. The compound, composition, pharmaceutical composition, construct, protein, or polypeptide of claim 7, comprising two nanobodies of claim 1 linked to an Fc portion.

9. (canceled)

10. (canceled)

11. The nanobody of claim 1, wherein the nanobody specifically binds to MC4R.

12. The nanobody of claim 1, wherein CDR3 comprises at least two amino acid residues that interact with the Gln43 and/or Gln 269 residues of MC4R

13. The nanobody of claim 3, which is chosen from the group consisting of amino acid sequences that have more than 90%, sequence identity with at least one of the amino acid sequences of SEQ ID NO's: 185 to 189.

14. The nanobody of claim 3, which is chosen from the group consisting of amino acid sequences that have more than 95%, sequence identity with at least one of the amino acid sequences of SEQ ID NO's: 185 to 189.

15. The nanobody of claim 3, which is chosen from the group consisting of amino acid sequences that have more than 95%, sequence identity with at least one of the amino acid sequences of SEQ ID NO's: 185 to 189.

16. A method of binding MC4R is a subject, the method comprising administering to the subject the compound, composition, pharmaceutical composition, construct, protein, or polypeptide of claim 7.

17. The method of claim 16, wherein the subject suffers from an MC4R related disease or disorder.
